(19) United States
(12) Patent Application Publication
(10) Pub. No.: US 2011/0092547 A1
(43) Pub. Date: Apr. 21, 2011

(54) CHEMICAL COMPOUNDS 979

(76) Inventors: Alan Martin Birch, Cheshire (GB); Roger John Butlin, Cheshire (GB); Adrian Liam Gill, Cheshire (GB); Samuel David Groombridge, Cheshire (GB); Alleyn Thomas Plowright, Cheshire (GB); Michael James Waring, Cheshire (GB)

(21) Appl. No.: 12/673,767

(22) PCT Filed: Aug. 15, 2008
(86) PCT No.: PCT/GB08/50716

§ 371 Date: Jun. 30, 2010

Related U.S. Application Data
(60) Provisional application No. 60/956,490, filed on Aug. 17, 2007.
(60) Provisional application No. 60/990,970, filed on Nov. 29, 2007.

(51) Int. Cl. A61K 31/4439; C07D 413/12; A61P 3/10; A61P 3/04

(52) U.S. Cl. 514/340; 546/269.1

(57) ABSTRACT

Compounds of formula (I), or pharmaceutically-acceptable salts and/or pro-drugs thereof, which inhibit acetyl CoA(acetyl coenzyme A):diacylglycerol acyltransferase (DGAT1) activity are provided,

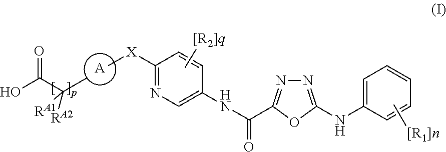

wherein n is 0 to 3; p is 0 or 1; q is 0 to 2;
R₁ and R₂ are, for example, independently fluoro, chloro, bromo, cyano or (1-4C)alkyl;
X is —O—, —S— or —NRa— wherein Ra is hydrogen or (1-4C)alkyl;
R^{A1} and R^{A2} are, for example, independently hydrogen or (1-4C)alkyl;
Ring A is a di-linked ring or ring system chosen from (4-6C)cycloalkane, (7-10C)bicycloalkane and (8-12C)tricycloalkane each optionally substituted, for example, by one substituent selected from (1-4C)alkyl, (1-4C)alkoxy and (1-4C)alkoxy(1-4C)alkyl;
or Ring A is phenylene optionally substituted, for example, by up to four substituents selected from fluoro, chloro, bromo, cyano, (1-4C)alkyl, (1-4C)alkoxy and (1-4C)alkoxy(1-4C)alkyl; together with processes for their preparation, pharmaceutical compositions containing them and their use as medicaments.

The present invention relates to compounds which inhibit acetyl CoA(acetyl coenzyme A):diacylglycerol acyltransferase (DGAT1) activity, processes for their preparation, pharmaceutical compositions containing them as the active ingredient, methods for the treatment of disease states associated with DGAT1 activity, to their use as medicaments and to their use in the manufacture of medicaments for use in the inhibition of DGAT1 in warm-blooded animals such as humans. In particular this invention relates to compounds useful for the treatment of type II diabetes, insulin resistance, impaired glucose tolerance and obesity in warm-blooded animals such as humans, more particularly to the use of these compounds in the manufacture of medicaments for use in the treatment of type II diabetes, insulin resistance, impaired glucose tolerance and obesity in warm-blooded animals such as humans.

Acyl CoA:diacylglycerol acyltransferase (DGAT) is found in the microsomal fraction of cells. It catalyzes the final reaction in the glycerol phosphate pathway, considered to be the main pathway of triglyceride synthesis in cells by facilitating the joining of a diacylglycerol with a fatty acyl CoA, resulting in the formation of triglyceride. Although it is unclear whether DGAT is rate-limiting for triglyceride synthesis, it catalyzes the only step in the pathway that is committed to producing this type of molecule [Lehner & Kuksis (1996) Biosynthesis of triacylglycerols. Prog. Lipid Res. 35: 169-201].

Two DGAT genes have been cloned and characterised. Both of the encoded proteins catalyse the same reaction although they share no sequence homology. The DGAT1 gene was identified from sequence database searches because of its similarity to acyl CoA:cholesterol acyltransferase (ACAT) genes. [Cases et al (1998) Identification of a gene encoding an acyl CoA:diacylglycerol acyltransferase, a key enzyme in triacylglycerol synthesis. Proc. Natl. Acad. Sci. USA 95: 13018-13023]. DGAT1 activity has been found in many mammalian tissues, including adipocytes.

Because of the previous lack of molecular probes, little is known about the regulation of DGAT1. DGAT1 is known to be significantly up-regulated during adipocyte differentiation.

Studies in gene knockout mice has indicated that modulators of the activity of DGAT1 would be of value in the treatment of type II diabetes and obesity. DGAT1 knockout (Dgat1^{-/-}) mice, are viable and capable of synthesizing triglycerides, as evidenced by normal fasting serum triglyceride levels and normal adipose tissue composition. Dgat1^{-/-} mice have less adipose tissue than wild-type mice at baseline and are resistant to diet-induced obesity. Metabolic rate is ˜20% higher in Dgat1^{-/-} mice than in wild-type mice on both regular and high-fat diets [Smith et al (2000) Obesity resistance and multiple mechanisms of triglyceride synthesis in mice lacking DGAT. Nature Genetics 25: 87-90]. Increased physical activity in Dgat1^{-/-} mice partially accounts for their increased energy expenditure. The Dgat1^{-/-} mice also exhibit increased insulin sensitivity and a 20% increase in glucose disposal rate. Leptin levels are 50% decreased in the Dgat1^{-/-} mice in line with the 50% decrease in fat mass.

When Dgat1^{-/-} mice are crossed with ob/ob mice, these mice exhibit the ob/ob phenotype [Chen et al (2002) Increased insulin and leptin sensitivity in mice lacking acyl CoA:diacylglycerol acyltransferase J. Clin. Invest. 109:1049-1055] indicating that the Dgat1^{-/-} phenotype requires an intact leptin pathway. When Dgat1^{-/-} mice are crossed with Agouti mice a decrease in body weight is seen with normal glucose levels and 70% reduced insulin levels compared to wild type, agouti or ob/ob/Dgat1^{-/-} mice.

Transplantation of adipose tissue from Dgat1^{-/-} mice to wild type mice confers resistance to diet-induced obesity and improved glucose metabolism in these mice [Chen et al (2003) Obesity resistance and enhanced glucose metabolism in mice transplanted with white adipose tissue lacking acyl CoA:diacylglycerol acyltransferase J. Clin. Invest. 111: 1715-1722].

International Application WO 2006/064189 describes certain oxadiazole compounds which inhibit DGAT-1. However, there remains a need for further DGAT-1 inhibitors possessing desirable properties, such as, for example, pharmaco-kinetic/dynamic and/or physico-chemical and/or toxicological profiles.

Accordingly, the present invention provides a compound of formula (I) or a pharmaceutically-acceptable salt or pro-drug thereof,

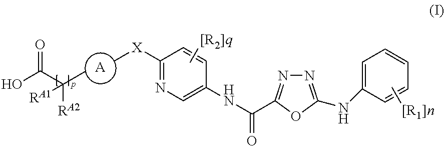

wherein n is 0, 1, 2 or 3 and R₁ is independently chosen from fluoro, chloro, bromo, cyano, (1-4C)alkyl, (3-4C)cycloalkyl, (2-4C)alkynyl, (1-4C)alkoxy, —CONRaRb, —SO₂Rc and —OSO₂Rc; wherein Ra and Rb are each independently hydrogen or (1-4C)alkyl and Rc is (1-4C)alkyl;
wherein q is 0, 1 or 2 and R₁ is independently chosen from fluoro, chloro, bromo, cyano, (1-4C)alkyl, (3-4C)cycloalkyl, (2-4C)alkynyl and (1-4C)alkoxy;
X is —O—, —S— or —NRa— wherein Ra is hydrogen or (1-4C)alkyl;
p is 0 or 1 and when p is 1 R^{A1} and R^{A2} are each independently hydrogen or (1-4C)alkyl or R^{A1} and R^{A2} are linked together to form a (3-6C)spiroalkyl ring;
Ring A is a di-linked (excluding links via the same or adjacent atoms) ring or ring system chosen from (4-6C)cycloalkane, (7-10C)bicycloalkane and (8-12C)tricycloalkane each optionally substituted on an available carbon atom, including the ring carbon atom bearing the carboxy-containing group, by one substituent selected from (1-4C)alkyl, (1-4C)alkoxy and (1-4C)alkoxy(1-4C)alkyl;
or Ring A is di-linked (excluding links via adjacent atoms) phenylene optionally substituted on an available carbon atom by up to four substituents independently selected from fluoro, chloro, bromo, cyano, (1-4C)alkyl, (1-4C)alkoxy and (1-4C)alkoxy(1-4C)alkyl;
and wherein any carbon atom in a (1-4C)alkyl or (1-4C)alkoxy group defined above may be optionally substituted by up to 3 fluoro atoms;
and wherein the defined carboxylic acid group linked to Ring A may be replaced by a mimic or bioisostere thereof.

It will be understood that Ring A is a di-linked ring or ring system which excludes links to the —X— group and the defined carboxy-containing group via the same or adjacent atoms (i.e. −1,1- and -1,2-links are excluded).

As used herein, the reference to carboxylic acid mimic or bioisostere includes groups as defined in The Practice of Medicinal Chemistry, Wermuth C. G. Ed.: Academic Press: New York, 1996, p 203. Particular examples of such groups include —SO₃H, —S(O)₂NHR¹³, S(O)₂NHC(O)R¹³, —CH₂S(O)₂R¹³, —C(O)NHS(O)₂R¹³, —C(O)NHOH, —C(O)NHCN, —CH(CF₃)OH, C(CF₃)₂OH, —P(O)(OH)₂ and groups of sub-formula (a)-(i′) below

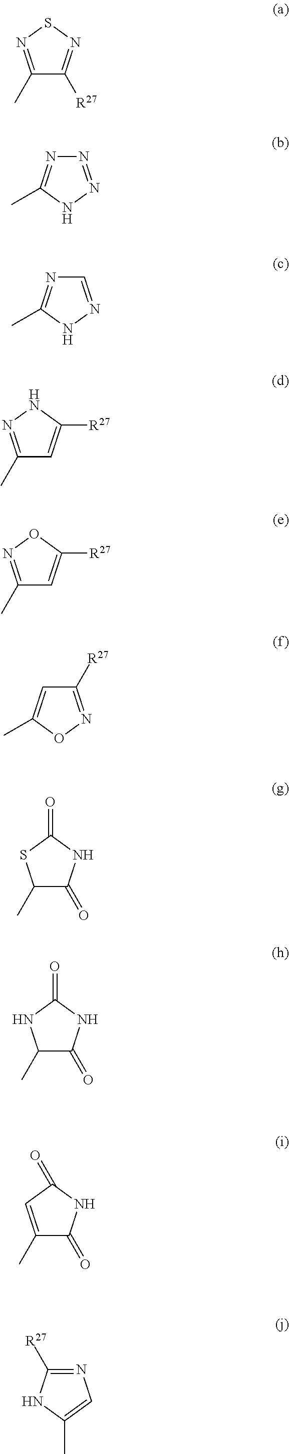

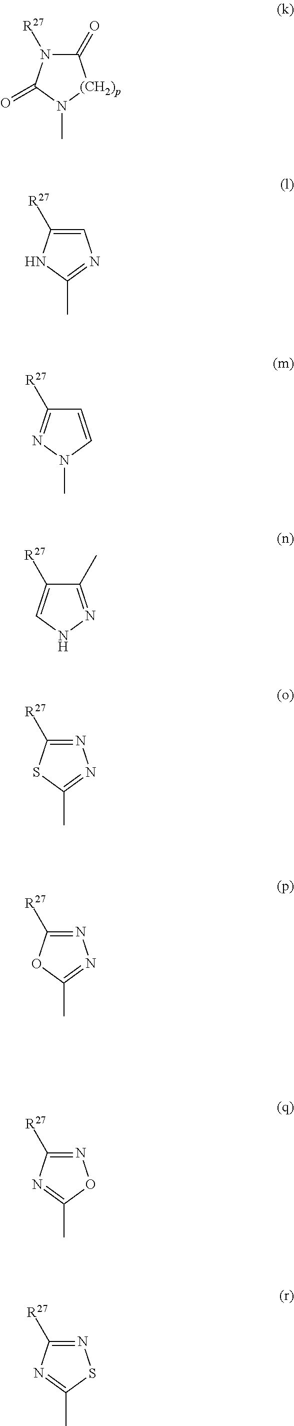

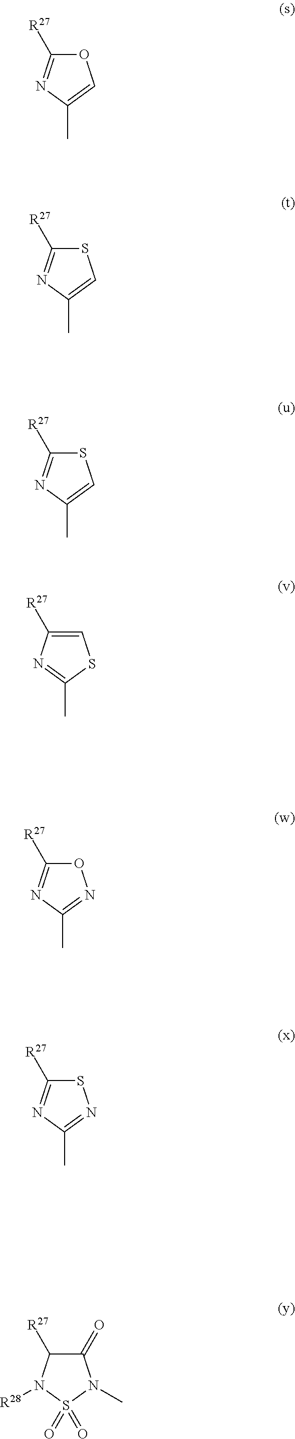

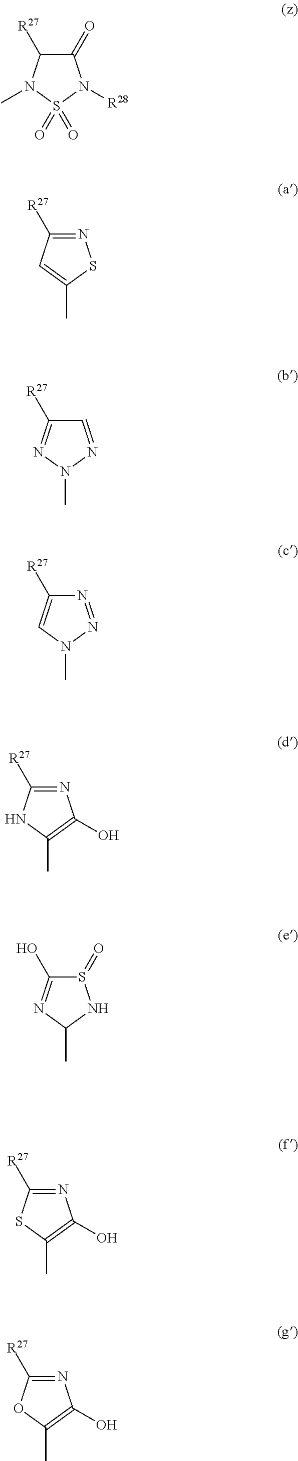

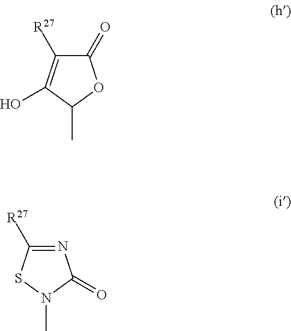

where p is 1 or 2, R²⁷ and R²⁸ are independently selected from hydrogen, hydroxy, (1-6C)alkoxy, thiol, (1-6C)alkylthio, —C(O)R²⁹, —S(O)R³⁰, —SO₂R³¹, —NR³²R³³, —NHCN, halogen and trihalomethyl, where R²⁹, R³⁰ and R³¹ are —OR³⁴, (1-6C)alkyl, —NR³²R³³ or trihalomethyl, R³² and R³³ are independently selected from hydrogen, (1-6C)alkyl, —SO₂R³⁴ and —COR³⁵, where R³⁵ is (1-6C)alkyl or trihalomethyl, and R³⁴ is hydrogen, (1-6C)alkyl or trihalomethyl and R¹³ is selected from hydrogen, (1-6C)alkyl, hydroxy, halo, amino, cyano, ((1-3C)alkyl)CONH—, carboxy, (1-6C)alkoxy, (1-6C)alkoxycarbonyl, carbamoyl, N-((1-6C)alkyl)carbamoyl, halo((1-6C)alkyl) (such as trifluoromethyl), (1-6C)alkylsulphonyl or (1-6C)alkylsulphinyl. Particular examples of R²⁷ or R²⁸ are hydroxy. A particular carboxylic acid mimic or bioisostere is tetrazole group of sub-formula (b) or the group —C(O)NHS(O)₂R¹³ wherein R¹³ is, for example, methyl.

In this specification the term “alkyl” includes both straight and branched chain alkyl groups but references to individual alkyl groups such as “propyl” are specific for the straight chain version only. An analogous convention applies to other generic terms. Unless otherwise stated the term “alkyl” advantageously refers to chains with 1-10 carbon atoms, suitably from 1-6 carbon atoms, preferably 1-4 carbon atoms.

In this specification the term “alkoxy” means an alkyl group as defined hereinbefore linked to an oxygen atom.

Particular values include for (1-4C)alkyl, methyl, ethyl, propyl and butyl; for (3-4C)cycloalkyl, cyclopropyl and cyclobutyl; for (2-4C)alkynyl, ethynyl; for (1-4C)alkoxy, methoxy and ethoxy; for —CONRaRb, —CONH₂ and —CONHMe; for —SO₂Rc, —SO₂Me and —SO₂Et; and for —OSO₂Rc, —OSO₂Me and —OSO₂Et.

Particular values include for any carbon atom in a (1-4C)alkyl or (1-4C)alkoxy group that may be optionally substituted by up to 3 fluoro atoms, a group such as, for example, trifluoromethyl, difluoromethoxy or trifluoromethoxy.

When p is 1 and R^{A1} and R^{A2} are linked together to form a (3-6C)spiroalkyl ring, such a ring may be, for example, a spiro-linked cyclopropyl or cyclobutyl.

When Ring A is a di-linked (excluding links via the same or adjacent atoms) (4-6C)cycloalkane ring this includes 1,4-cyclohexane, 1,3-cyclopentane and 1,3-cyclobutane.

When Ring A is (7-10C)bicycloalkanediyl this includes bicyclo[2.2.1]heptanediyl, 1,4-bicyclo[2.2.2]octanediyl, 1,5-bicyclo[3.2.1]octanediyl, 1,5-bicyclo[3.2.2]nonanediyl and 1,5-bicyclo[3.3.2]decanediyl.

When Ring A is (8-12C)tricycloalkanediyl this includes adamantanediyl.

For the avoidance of doubt it is to be understood that where in this specification a group is qualified by ‘hereinbefore defined’ or ‘defined hereinbefore’ the said group encompasses the first occurring and broadest definition as well as each and all of the particular definitions for that group.

If not stated elsewhere, suitable optional substituents for a particular group are those as stated for similar groups herein.

A compound of formula (I) may form stable acid or basic salts, and in such cases administration of a compound as a salt may be appropriate, and pharmaceutically acceptable salts may be made by conventional methods such as those described following.

Suitable pharmaceutically-acceptable salts include acid addition salts such as methanesulfonate, tosylate, α-glycerophosphate, fumarate, hydrochloride, citrate, maleate, tartrate and (less preferably) hydrobromide. Also suitable are salts formed with phosphoric and sulfuric acid. In another aspect suitable salts are base salts such as Group (I) (alkali metal) salt, Group (II) (alkaline earth) metal salt, an organic amine salt for example triethylamine, morpholine, N-methylpiperidine, N-ethylpiperidine, procaine, dibenzylamine, N,N-dibenzylethylamine, tris-(2-hydroxyethyl)amine, N-methyl d-glucamine and amino acids such as lysine. There may be more than one cation or anion depending on the number of charged functions and the valency of the cations or anions.

However, to facilitate isolation of the salt during preparation, salts which are less soluble in the chosen solvent may be preferred whether pharmaceutically-acceptable or not.

Within the present invention it is to be understood that a compound of the formula (I) or a salt thereof may exhibit the phenomenon of tautomerism and that the formulae drawings within this specification can represent only one of the possible tautomeric forms. It is to be understood that the invention encompasses any tautomeric form which inhibits DGAT1 activity and is not to be limited merely to any one tautomeric form utilised within the formulae drawings.

Pro-drugs of compounds of formula (I), and salts thereof, are also within the scope of the invention.

Various forms of prodrugs are known in the art. For examples of such prodrug derivatives, see:
a) Design of Prodrugs, edited by H. Bundgaard, (Elsevier, 1985) and Methods in Enzymology, Vol. 42, p. 309-396, edited by K. Widder, et al. (Academic Press, 1985);
b) A Textbook of Drug Design and Development, edited by Krogsgaard-Larsen and
H. Bundgaard, Chapter 5 “Design and Application of Prodrugs”, by H. Bundgaard p. 113-191 (1991);
c) H. Bundgaard, Advanced Drug Delivery Reviews, 8, 1-38 (1992);
d) H. Bundgaard, et al., Journal of Pharmaceutical Sciences, 77, 285 (1988); and
e) N. Kakeya, et al., Chem Pharm Bull, 32, 692 (1984).

Examples of such prodrugs are in vivo cleavable esters of a compound of the invention. An in vivo cleavable ester of a compound of the invention containing a carboxy group is, for example, a pharmaceutically-acceptable ester which is cleaved in the human or animal body to produce the parent acid. Suitable pharmaceutically-acceptable esters for carboxy include (1-6C)alkyl esters, for example methyl or ethyl; (1-6C)alkoxymethyl esters, for example methoxymethyl; (1-6C)alkanoyloxymethyl esters, for example pivaloyloxymethyl; phthalidyl esters; (3-8C)cycloalkoxycarbonyloxy(1-6C)alkyl esters, for example 1-cyclohexylcarbonyloxyethyl; 1,3-dioxolan-2-ylmethyl esters, for example 5-methyl-1,3-dioxolan-2-ylmethyl; (1-6C)alkoxycarbonyloxyethyl esters, for example 1-methoxycarbonyloxyethyl; aminocarbonylmethyl esters and mono- or di-N-((1-6C)alkyl) versions thereof, for example N,N-dimethylaminocarbonylmethyl esters and N-ethylaminocarbonylmethyl esters; and may be formed at any carboxy group in the compounds of this invention. An in vivo cleavable ester of a compound of the invention containing a hydroxy group is, for example, a pharmaceutically-acceptable ester which is cleaved in the human or animal body to produce the parent hydroxy group. Suitable pharmaceutically acceptable esters for hydroxy include (1-6C)alkanoyl esters, for example acetyl esters; and benzoyl esters wherein the phenyl group may be substituted with aminomethyl or N-substituted mono- or di-(1-6C)alkyl aminomethyl, for example 4-aminomethylbenzoyl esters and 4-N,N-dimethylaminomethylbenzoyl esters.

Particular prodrugs are (1-4C)alkyl esters of the defined carboxyclic acid in compounds of formula (I), (IA) and/or (IB).

It will be appreciated by those skilled in the art that certain compounds of formula (I) contain asymmetrically substituted carbon and/or sulfur atoms, and accordingly may exist in, and be isolated in, optically-active and racemic forms. Some compounds of formula (I) may exhibit polymorphism. It is to be understood that the present invention encompasses any racemic, optically-active, polymorphic or stereoisomeric form, or mixtures thereof, which form possesses properties useful in the inhibition of DGAT1 activity, it being well known in the art how to prepare optically-active forms (for example, by resolution of the racemic form by recrystallization techniques, by synthesis from optically-active starting materials, by chiral synthesis, by enzymatic resolution, by biotransformation, or by chromatographic separation using a chiral stationary phase) and how to determine efficacy for the inhibition of DGAT1 activity by the standard tests described hereinafter.

It is also to be understood that certain compounds of the formula (I) and salts thereof can exist in solvated as well as unsolvated forms such as, for example, hydrated forms. It is to be understood that the invention encompasses all such solvated forms which inhibit DGAT1 activity.

As stated before, we have discovered a range of compounds that have good DGAT1 inhibitory activity. They have good physical and/or pharmacokinetic properties in general. The following compounds possess particular, desirable pharmaceutical and/or physical and/or pharmacokinetic/dynamic and/or toxicological properties.

In one aspect of the invention there is a compound of formula (I), or a pharmaceutically-acceptable salt or pro-drug thereof, wherein n is 0, 1, 2 or 3 and R₁ is independently chosen from fluoro, chloro, bromo, cyano, (1-4C)alkyl, (3-4C)cycloalkyl, (2-4C)alkynyl, (1-4C)alkoxy, —CONRaRb, —SO₂Rc and —OSO₂Rc; wherein Ra and Rb are each independently hydrogen or (1-4C)alkyl and Rc is (1-4C)alkyl;

wherein q is 0, 1 or 2 and R₂ is independently chosen from fluoro, chloro, bromo, cyano, (1-4C)alkyl, (3-4C)cycloalkyl, (2-4C)alkynyl and (1-4C)alkoxy;
X is —O—, —S— or —NRa— wherein Ra is hydrogen or (1-4C)alkyl;
p is 0 or 1 and when p is 1 R^{A1} and R^{A2} are each independently hydrogen or (1-4C)alkyl or R^{A1} and R^{A2} are linked together to form a (3-6C)spiroalkyl ring;
Ring A is a di-linked (excluding links via the same or adjacent atoms) ring or ring system chosen from 1,4-cyclohexane, 1,3-cyclopentane, 1,3-cyclobutane, (7-10C)bicycloalkane and (8-12C)tricycloalkane each optionally substituted on an available carbon atom, including the ring carbon atom bearing the carboxy-containing group, by one substituent selected from (1-4C)alkyl, (1-4C)alkoxy and (1-4C)alkoxy(1-4C)alkyl;
or Ring A is 1,4-phenylene optionally substituted on an available carbon atom by up to four substituents independently selected from fluoro, chloro, bromo, cyano, (1-4C)alkyl, (1-4C)alkoxy and (1-4C)alkoxy(1-4C)alkyl;
and wherein any carbon atom in a (1-4C)alkyl or (1-4C)alkoxy group defined above may be optionally substituted by up to 3 fluoro atoms;
and wherein the defined carboxylic acid group linked to Ring A may be replaced by a mimic or bioisostere thereof.

In one aspect, when Ring A is other than phenylene it will be appreciated that formula (I) includes compounds wherein the Ring A substituent bearing the carboxy group (or suitable replacement thereof) and the —X— link are in either a cis- or a trans-arrangement across the ring, in relation to each other. Where appropriate the invention encompasses both the cis- and trans-isomers. Techniques for separation of such isomers is well known in the art.

Thus, in one aspect, when Ring A is cyclohexyl the carboxy group and —X— link are in a cis-configuration across the cyclohexyl ring, to give a compound of formula (IA), wherein the variables are as defined hereinbefore or hereinafter:

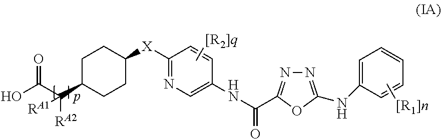

In another aspect, when Ring A is cyclohexyl the carboxy group and —X— link are in a trans-configuration across the cyclohexyl ring, to give a compound of formula (IB) wherein the variables are as defined hereinbefore or hereinafter:

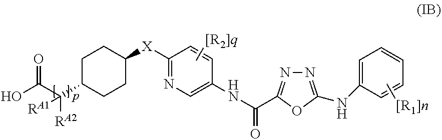

References hereinbefore or hereinafter to a compound of formula (I) should be taken to apply also to compounds of formulae (IA) and (IB). References to compounds of formulae (I), (IA) and (IB) includes compounds of formula (I), compounds of formula (IA) and compounds of formula (IB) as individual groups of compounds.

References hereinbefore or hereinafter, and in the claims, to a compound of formula (I), or a pharmaceutically-acceptable salt, or a pro-drug thereof, refer to the embodiments of (i) a compound of formula (I); (ii) a pharmaceutically-acceptable salt of a compound of formula (I) and (iii) a pro-drug of a compound of formula (I).

In one embodiment, in each of the claims hereinafter there is provided a compound of formula (I), or a pharmaceutically-acceptable salt thereof.

In one embodiment of the invention there are provided compounds of formulae (I), (IA) and (IB), in an alternative embodiment there are provided salts, particularly pharmaceutically-acceptable salts of compounds of formulae (I), (IA) and (IB). In a further embodiment, there are provided pro-drugs, particularly in-vivo cleavable esters, of compounds of formulae (I), (IA) and (IB). In a further embodiment, there are provided salts, particularly pharmaceutically-acceptable salts of pro-drugs of compounds of formulae (I), (IA) and (IB).

Particular values of substituents in compounds of formulae (I), (IA) and (IB) are as follows (such values may be used where appropriate with any of the other values, definitions, claims or embodiments defined hereinbefore or hereinafter) . . . .

1) the R₁ substituent is not ortho to the —NH— link and R₁ is particularly fluoro and n is particularly 1 or 2;

### 2) X is —O—;

3) q is 0;
4) p is 0;
4) p is 1 and R^{A1} and R^{A2} are each hydrogen;
5) Ring A is 1,4-cyclohexanediyl or 1,4-phenylene, particularly 1,4-cyclohexanediyl.
6) When Ring A is other than phenylene, the Ring A substituent bearing the carboxy group (or suitable replacement thereof) and the —X— link are in a cis-arrangement across the ring, in relation to each other.
7) When Ring A is other than phenylene, the Ring A substituent bearing the carboxy group (or suitable replacement thereof) and the —X— link are in a trans-arrangement across the ring, in relation to each other.
8) n is 1, 2 or 3 and R₁ is independently chosen from fluoro, chloro, bromo, (1-4C)alkyl and (1-4C)alkoxy and the (1-4C)alkyl or (1-4C)alkoxy groups may be optionally substituted by up to 3 fluoro atoms.
9) q is 1 and R₂ is fluoro, particularly 6-F.
10) Ring A is 1,3-cyclobutanediyl or 1,3-cyclopentanediyl.

Further preferred compounds of the invention are each of the Examples, each of which provides a further independent aspect of the invention. In further aspects, the present invention also comprises any particular compounds of the Examples.

In a further aspect, the present invention comprises the compound cis-4-[5-[[5-[(3,4-difluorophenyl)amino]1,3,4-oxadiazole-2-carbonyl]amino]pyridin-2-yl]oxycyclohexane-1-carboxylic acid or a pharmaceutically-acceptable salt thereof.

In a further aspect, the present invention comprises a compound selected from (1s,4s)-4-(5-(5-(2,4-dichlorophenylamino)-1,3,4-oxadiazole-2-carboxamido)pyridin-2-yloxy)cyclohexanecarboxylic acid; (1s,4s)-4-(5-(5-(3-chloro-4-fluorophenylamino)-1,3,4-oxadiazole-2-carboxamido)pyridin-2-yloxy)cyclohexanecarboxylic acid; 2-((1s,4s)-4-(5-(5-(3,4-difluorophenylamino)-1,3,4-oxadiazole-2-carboxamido)pyridin-2-yloxy)cyclohexyl)acetic acid; (1s,4s)-4-(5-(5-(3,4-Difluorophenylamino)-1,3,4-oxadiazole-2-carboxamido)-6-fluoropyridin-2-yloxy)cyclohexanecarboxylic acid; (1s,4s)-4-(6-Fluoro-5-(5-(2,4,5-trifluorophenylamino)-1,3,4-oxadiazole-2-carboxamido)pyridin-2-yloxy)cyclohexanecarboxylic acid;

or a pharmaceutically-acceptable salt thereof.

A compound of formula (I) and its salts may be prepared by any process known to be applicable to the preparation of chemically related compounds. Such processes, when used to prepare a compound of the formula (I), or a pharmaceutically-acceptable salt thereof, are provided as a further feature of the invention.

In a further aspect the present invention also provides that the compounds of the formula (I) and salts thereof, can be prepared by a process a) to b) as follows (wherein all variables are as hereinbefore defined for a compound of formula (I) unless otherwise stated and wherein the defined carboxylic acid group linked to Ring A may be replaced by a mimic or bioisostere thereof as appropriate);

a) reaction of an amine of formula (2) with an (activated) carboxylic acid derivative of the acid of formula (3) (such as an acid chloride or HOBt ester thereof) or reaction with a carboxylate salt (such as sodium) of the acid of formula (3) (using a suitable coupling agent), wherein R is (1-6C)alkyl (for example methyl, ethyl, isopropyl and tert-butyl) followed by hydrolysis of the R group;

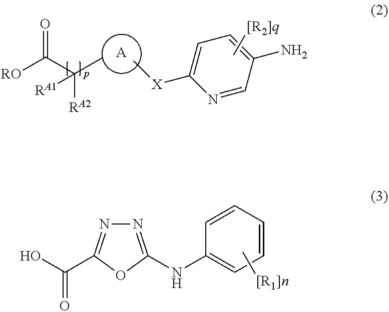

b) cyclisation of a compound of formula (4) (where X₁ is S or O) wherein R is (1-6C)alkyl, followed by hydrolysis of the R group;

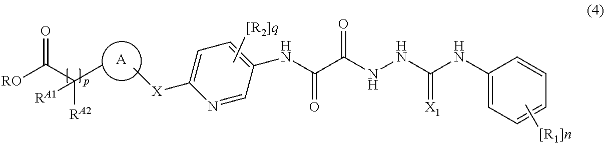

and thereafter if necessary:
1) removing any protecting groups; and/or
2) forming a salt.

### Process A)

Compounds of formula (2) may be made by application of standard synthetic methods well known in the art. In particular, compounds of formula (2) may be prepared by reduction of a compound of formula (2A) wherein Pg is a suitable protecting group.

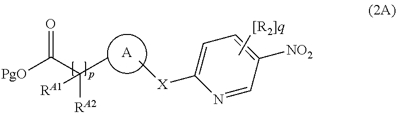

Compounds of formula (2A) may be made by S_{N}Ar chemistry as illustrated in Scheme 1, wherein R is a (1-6C)alkyl group, Pg is a suitable protecting group (such as R is a (1-6C)alkyl group) and Lg is a suitable leaving group such as halo, for example, fluoro:

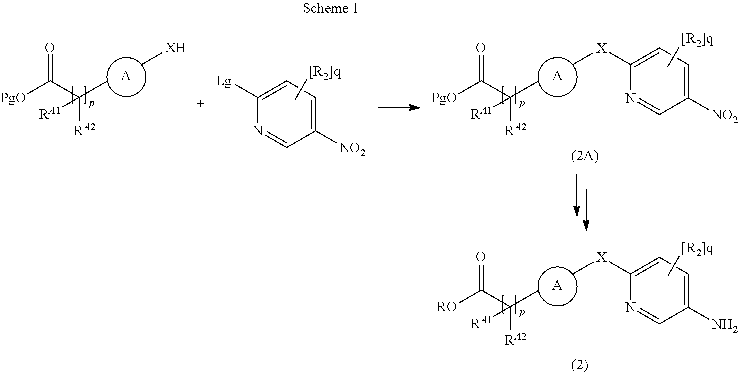

When X is —O— and Ring A is other than phenylene, compounds of formula (2A) may be made by Mitsunobu chemistry (using triphenylphosphine and Mitsunobu conditions—see, for example, J. March, p. 486, 5^{th} Ed. (2001), Wiley Interscience) as illustrated in Scheme 2, wherein R is a (1-6C)alkyl group and Pg is a suitable protecting group (such as R is a (1-6C)alkyl group):

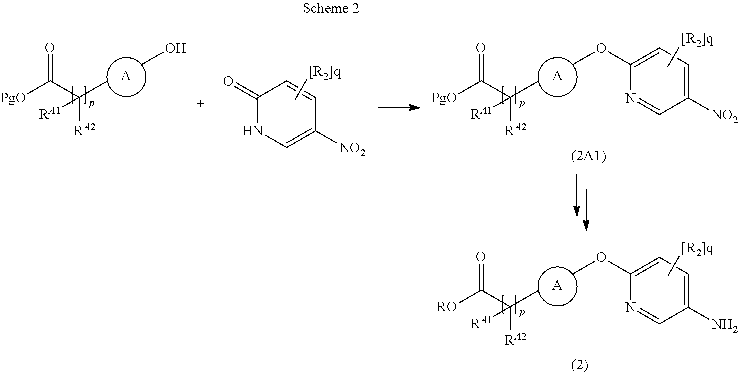

As an alternative to the nitro compounds in Schemes 1 and 2, other suitable precursors to the final amino substituent in compounds of formula (2) may be used.

Compounds of formula (3) may be made by alkaline hydrolysis of ester (5a) as prepared using a published procedure (J. Het. Chem. 1977, 14, 1385-1388). Ester (5a) may be made by cyclisation of a compound of formula (5b) (where X₁ is O or S) in a similar manner as described in process b) for compounds of formula (4).

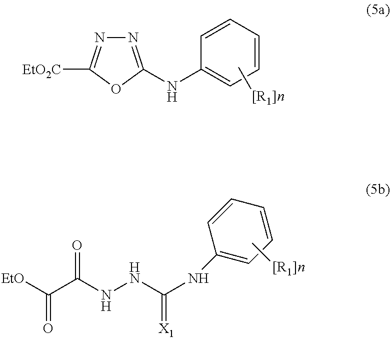

An alternative method for making compounds of formula (5a) is illustrated below:

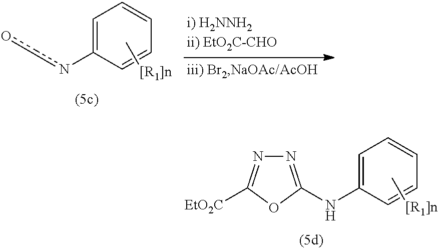

Compounds of formula (2) may be coupled with compounds of formula (3) under standard conditions for formation of amide bonds. For example using an appropriate coupling reaction, such as a carbodiimide coupling reaction performed with EDAC, optionally in the presence of DMAP, in a suitable solvent such as DCM, chloroform or DMF at room temperature. The R group may be removed by any conditions known in the art for ester hydrolysis.

### Process B)

Compounds of formula (4) and (5b) where X₁ is S may be made by reaction of an aminocarbonyl acylhydrazine or ethoxycarbonyl acylhydrazine with a thioisocyanate or thioisocyanate equivalent such as aminothiocarbonylimidazole in a suitable solvent such as DMF or MeCN at a temperature between 0 and 100° C. The preparation of aminocarbonyl acylhydrazines from anilines and of ethoxycarbonyl acylhydrazines is well known in the art. For example reaction of an aniline with methyl chlorooxoacetate in the presence of pyridine in a suitable solvent such as DCM followed by reaction with hydrazine in a suitable solvent such as ethanol at a temperature between 0 and 100° C.

The compound of formula (4) may then be cyclised using, for example agents such as carbonyldiimidazole, or tosyl chloride and a suitable base (such as triethylamine), under conditions known in the art. The R group may be removed by any conditions known in the art for ester hydrolysis.

Iso(thio)cyanates (of formula (5c) for isocyanates or, for isothiocyanates, wherein the —NCO group in (5c) is replaced by —NCS) are commercially available or may be made by reaction of the appropriate amine with, for example, (thio)phosgene or a (thio)phosgene equivalent followed by a suitable base (such as triethylamine).

It will be appreciated that certain of the various ring substituents in the compounds of the present invention, for example R₁ and R₂ may be introduced by standard aromatic substitution reactions or generated by conventional functional group modifications either prior to or immediately following the processes mentioned above, and as such are included in the process aspect of the invention. Such reactions may convert one compound of the formula (I) into another compound of the formula (I). Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogen group. Particular examples of modifications include the reduction of a nitro group an amino group by for example, catalytic hydrogenation with a nickel catalyst or treatment with iron in the presence of hydrochloric acid with heating; oxidation of alkylthio to alkanesulfinyl or alkanesulfonyl.

If not commercially available, the necessary starting materials for the procedures such as those described above may be made by procedures which are selected from standard organic chemical techniques, techniques which are analogous to the synthesis of known, structurally similar compounds, techniques which are described or illustrated in the references given above, or techniques which are analogous to the above described procedure or the procedures described in the examples. The reader is further referred to Advanced Organic Chemistry, 5^{th} Edition, by Jerry March and Michael Smith, published by John Wiley & Sons 2001, for general guidance on reaction conditions and reagents.

It will be appreciated that some intermediates to compounds of the formula (I) are also novel and these are provided as separate independent aspects of the invention. In particular, compounds of formula (4) form a further aspect of the invention. Furthermore, ester derivatives of compounds of formula (I) form a further aspect of the invention.

It will also be appreciated that in some of the reactions mentioned herein it may be necessary/desirable to protect any sensitive groups in compounds. The instances where protection is necessary or desirable are known to those skilled in the art, as are suitable methods for such protection. Conventional protecting groups may be used in accordance with standard practice (for illustration see T. W. Greene, Protective Groups in Organic Synthesis, John Wiley and Sons, 1991).

Protecting groups may be removed by any convenient method as described in the literature or known to the skilled chemist as appropriate for the removal of the protecting group in question, such methods being chosen so as to effect removal of the protecting group with minimum disturbance of groups elsewhere in the molecule.

Thus, if reactants include, for example, groups such as amino, carboxy or hydroxy it may be desirable to protect the group in some of the reactions mentioned herein.

Examples of a suitable protecting group for a hydroxy group is, for example, an acyl group, for example an alkanoyl group such as acetyl, an aroyl group, for example benzoyl, a silyl group such as trimethylsilyl or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium hydroxide. Alternatively a silyl group such as trimethylsilyl or SEM may be removed, for example, by fluoride or by aqueous acid; or an arylmethyl group such as a benzyl group may be removed, for example, by hydrogenation in the presence of a catalyst such as palladium-on-carbon.

A suitable protecting group for an amino group is, for example, an acyl group, for example an alkanoyl group such as acetyl, an alkoxycarbonyl group, for example a methoxycarbonyl, ethoxycarbonyl or tert-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine or 2-hydroxyethylamine, or with hydrazine.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

Resins may also be used as a protecting group.

The protecting groups may be removed at any convenient stage in the synthesis using conventional techniques well known in the chemical art, or they may be removed during a later reaction step or work-up.

The skilled organic chemist will be able to use and adapt the information contained and referenced within the above references, and accompanying Examples therein and also the examples herein, to obtain necessary starting materials, and products.

The removal of any protecting groups and the formation of a pharmaceutically-acceptable salt are within the skill of an ordinary organic chemist using standard techniques. Furthermore, details on the these steps has been provided hereinbefore.

When an optically active form of a compound of the invention is required, it may be obtained by carrying out one of the above procedures using an optically active starting material (formed, for example, by asymmetric induction of a suitable reaction step), or by resolution of a racemic form of the compound or intermediate using a standard procedure, or by chromatographic separation of diastereoisomers (when produced). Enzymatic techniques may also be useful for the preparation of optically active compounds and/or intermediates.

Similarly, when a pure regioisomer of a compound of the invention is required, it may be obtained by carrying out one of the above procedures using a pure regioisomer as a starting material, or by resolution of a mixture of the regioisomers or intermediates using a standard procedure.

According to a further aspect of the invention there is provided a pharmaceutical composition which comprises a compound of formula (I), (IA) or (IB) as defined hereinbefore or a pharmaceutically-acceptable salt thereof; in association with a pharmaceutically-acceptable excipient or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

Suitable pharmaceutically acceptable excipients for a tablet formulation include, for example, inert diluents such as lactose, sodium carbonate, calcium phosphate or calcium carbonate, granulating and disintegrating agents such as corn starch or algenic acid; binding agents such as starch; lubricating agents such as magnesium stearate, stearic acid or talc; preservative agents such as ethyl or propyl p-hydroxybenzoate, and anti-oxidants, such as ascorbic acid. Tablet formulations may be uncoated or coated either to modify their disintegration and the subsequent absorption of the active ingredient within the gastrointestinal tract, or to improve their stability and/or appearance, in either case, using conventional coating agents and procedures well known in the art.

Compositions for oral use may be in the form of hard gelatin capsules in which the active ingredient is mixed with an inert solid diluent, for example, calcium carbonate, calcium phosphate or kaolin, or as soft gelatin capsules in which the active ingredient is mixed with water or an oil such as peanut oil, liquid paraffin, or olive oil.

Aqueous suspensions generally contain the active ingredient in finely powdered form together with one or more suspending agents, such as sodium carboxymethylcellulose, methylcellulose, hydroxypropylmethylcellulose, sodium alginate, polyvinyl-pyrrolidone, gum tragacanth and gum acacia; dispersing or wetting agents such as lecithin or condensation products of an alkylene oxide with fatty acids (for example polyoxethylene stearate), or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives (such as ethyl or propyl p-hydroxybenzoate, anti-oxidants (such as ascorbic acid), colouring agents, flavouring agents, and/or sweetening agents (such as sucrose, saccharine or aspartame).

Oily suspensions may be formulated by suspending the active ingredient in a vegetable oil (such as arachis oil, olive oil, sesame oil or coconut oil) or in a mineral oil (such as liquid paraffin). The oily suspensions may also contain a thickening agent such as beeswax, hard paraffin or cetyl alcohol. Sweetening agents such as those set out above, and flavouring agents may be added to provide a palatable oral preparation. These compositions may be preserved by the addition of an anti-oxidant such as ascorbic acid.

Dispersible powders and granules suitable for preparation of an aqueous suspension by the addition of water generally contain the active ingredient together with a dispersing or wetting agent, suspending agent and one or more preservatives. Suitable dispersing or wetting agents and suspending agents are exemplified by those already mentioned above. Additional excipients such as sweetening, flavouring and colouring agents, may also be present.

The pharmaceutical compositions of the invention may also be in the form of oil-in-water emulsions. The oily phase may be a vegetable oil, such as olive oil or arachis oil, or a mineral oil, such as for example liquid paraffin or a mixture of any of these. Suitable emulsifying agents may be, for example, naturally-occurring gums such as gum acacia or gum tragacanth, naturally-occurring phosphatides such as soya bean, lecithin, an esters or partial esters derived from fatty acids and hexitol anhydrides (for example sorbitan monooleate) and condensation products of the said partial esters with ethylene oxide such as polyoxyethylene sorbitan monooleate. The emulsions may also contain sweetening, flavouring and preservative agents.

Syrups and elixirs may be formulated with sweetening agents such as glycerol, propylene glycol, sorbitol, aspartame or sucrose, and may also contain a demulcent, preservative, flavouring and/or colouring agent.

The pharmaceutical compositions may also be in the form of a sterile injectable aqueous or oily suspension, which may be formulated according to known procedures using one or more of the appropriate dispersing or wetting agents and suspending agents, which have been mentioned above. A sterile injectable preparation may also be a sterile injectable solution or suspension in a non-toxic parenterally-acceptable diluent or solvent, for example a solution in 1,3-butanediol.

Compositions for administration by inhalation may be in the form of a conventional pressurised aerosol arranged to dispense the active ingredient either as an aerosol containing finely divided solid or liquid droplets. Conventional aerosol propellants such as volatile fluorinated hydrocarbons or hydrocarbons may be used and the aerosol device is conveniently arranged to dispense a metered quantity of active ingredient.

For further information on formulation the reader is referred to Chapter 25.2 in Volume 5 of Comprehensive Medicinal Chemistry (Corwin Hansch; Chairman of Editorial Board), Pergamon Press 1990.

The amount of active ingredient that is combined with one or more excipients to produce a single dosage form will necessarily vary depending upon the host treated and the particular route of administration. For example, a formulation intended for oral administration to humans will generally contain, for example, from 0.5 mg to 2 g of active agent compounded with an appropriate and convenient amount of excipients which may vary from about 5 to about 98 percent by weight of the total composition. Dosage unit forms will generally contain about 1 mg to about 500 mg of an active ingredient. For further information on Routes of Administration and Dosage Regimes the reader is referred to Chapter 25.3 in Volume 5 of Comprehensive Medicinal Chemistry (Corwin Hansch; Chairman of Editorial Board), Pergamon Press 1990.

According to a further aspect of the present invention there is provided a compound of formula (I), (IA) and/or (IB) or a pharmaceutically acceptable salt thereof as defined hereinbefore for use in a method of treatment of the human or animal body by therapy.

We have found that compounds of the present invention inhibit DGAT1 activity and are therefore of interest for their blood glucose-lowering effects.

A further feature of the present invention is a compound of formula (I), (IA) and/or (IB) or a pharmaceutically-acceptable salt thereof for use as a medicament.

Conveniently this is a compound of formula (I), (IA) and/or (IB) or a pharmaceutically-acceptable salt thereof, for (use as a medicament for) producing an inhibition of DGAT1 activity in a warm-blooded animal such as a human being.

Particularly this is a compound of formula (I), (IA) and/or (IB) or a pharmaceutically-acceptable salt thereof, for (use as a medicament for) treating diabetes mellitus and/or obesity in a warm-blooded animal such as a human being.

Thus according to a further aspect of the invention there is provided the use of a compound of formula (I), (IA) and/or (IB) or a pharmaceutically-acceptable salt thereof in the manufacture of a medicament for use in the production of an inhibition of DGAT1 activity in a warm-blooded animal such as a human being.

Thus according to a further aspect of the invention there is provided the use of a compound of formula (I), (IA) and/or (IB) or a pharmaceutically-acceptable salt thereof in the manufacture of a medicament for use in the treatment of diabetes mellitus and/or obesity in a warm-blooded animal such as a human being.

According to a further aspect of the invention there is provided a pharmaceutical composition which comprises a compound of formula (I), (IA) and/or (IB) as defined hereinbefore or a pharmaceutically-acceptable salt thereof, in association with a pharmaceutically-acceptable excipient or carrier for use in producing an inhibition of DGAT1 activity in an warm-blooded animal, such as a human being.

According to a further aspect of the invention there is provided a pharmaceutical composition which comprises a compound of formula (I), (IA) and/or (IB) as defined hereinbefore or a pharmaceutically-acceptable salt thereof, in association with a pharmaceutically-acceptable excipient or carrier for use in the treatment of diabetes mellitus and/or obesity in an warm-blooded animal, such as a human being.

According to a further feature of the invention there is provided a method for producing an inhibition of DGAT1 activity in a warm-blooded animal, such as a human being, in need of such treatment which comprises administering to said animal an effective amount of a compound of formula (I), (IA) and/or (IB) or a pharmaceutically-acceptable salt thereof as defined hereinbefore.

According to a further feature of the invention there is provided a method of treating diabetes mellitus and/or obesity in a warm-blooded animal, such as a human being, in need of such treatment which comprises administering to said animal an effective amount of a compound of formula (I), (IA) and/or (IB) or a pharmaceutically-acceptable salt thereof as defined hereinbefore.

As stated above the size of the dose required for the therapeutic or prophylactic treatment of a particular disease state will necessarily be varied depending on the host treated, the route of administration and the severity of the illness being treated. Preferably a daily dose in the range of 1-50 mg/kg is employed. However the daily dose will necessarily be varied depending upon the host treated, the particular route of administration, and the severity of the illness being treated. Accordingly the optimum dosage may be determined by the practitioner who is treating any particular patient.

As stated above compounds defined in the present invention are of interest for their ability to inhibit the activity of DGAT1. A compound of the invention may therefore be useful for the prevention, delay or treatment of a range of disease states including diabetes mellitus, more specifically type 2 diabetes mellitus (T2DM) and complications arising there from (for example retinopathy, neuropathy and nephropathy), impaired glucose tolerance (IGT), conditions of impaired fasting glucose, metabolic acidosis, ketosis, dysmetabolic syndrome, arthritis, osteoporosis, obesity and obesity related disorders, (which include peripheral vascular disease, (including intermittent claudication), cardiac failure and certain cardiac myopathies, myocardial ischaemia, cerebral ischaemia and reperfusion, hyperlipidaemias, atherosclerosis, infertility and polycystic ovary syndrome); the compounds of the invention may also be useful for muscle weakness, diseases of the skin such as acne, various immunomodulatory diseases (such as psoriasis), HIV infection, inflammatory bowel syndrome and inflammatory bowel disease such as Crohn's disease and ulcerative colitis.

In particular, the compounds of the present invention are of interest for the prevention, delay or treatment of diabetes mellitus and/or obesity and/or obesity related disorders. In one aspect, the compounds of the invention are used for prevention, delay or treatment of diabetes mellitus. In another aspect, the compounds of the invention are used for prevention, delay or treatment of obesity. In a further aspect, the compounds of the invention are used for prevention, delay or treatment of obesity related disorders.

The inhibition of DGAT1 activity described herein may be applied as a sole therapy or in combination with one or more other substances and/or treatments for the indication being treated. Such conjoint treatment may be achieved by way of the simultaneous, sequential or separate administration of the individual components of the treatment. Simultaneous treatment may be in a single tablet or in separate tablets. For example such conjoint treatment may be beneficial in the treatment of metabolic syndrome [defined as abdominal obesity (as measured by waist circumference against ethnic and gender specific cut-points) plus any two of the following: hypertriglyceridemia (>150 mg/dl; 1.7 mmol/l); low HDLc (<40 mg/dl or <1.03 mmol/l for men and <50 mg/dl or 1.29 mmol/l for women) or on treatment for low HDL (high density lipoprotein); hypertension (SBP≧130 mmHg DBP≧85 mmHg) or on treatment for hypertension; and hyperglycemia (fasting plasma glucose ≧100 mg/dl or 5.6 mmol/l or impaired glucose tolerance or pre-existing diabetes mellitus)—International Diabetes Federation & input from IAS/NCEP].

Such conjoint treatments may include the following main categories:

1) Anti-obesity therapies such as those that cause weight loss by effects on food intake, nutrient absorption or energy expenditure, such as orlistat, sibutramine and the like.
2) Insulin secretagogues including sulphonylureas (for example glibenclamide, glipizide), prandial glucose regulators (for example repaglinide, nateglinide);
3) Agents that improve incretin action (for example dipeptidyl peptidase IV inhibitors, and GLP-1 agonists);
4) Insulin sensitising agents including PPARgamma agonists (for example pioglitazone and rosiglitazone), and agents with combined PPARalpha and gamma activity;
5) Agents that modulate hepatic glucose balance (for example metformin, fructose 1, 6 bisphosphatase inhibitors, glycogen phosphorylase inhibitors, glycogen synthase kinase inhibitors, glucokinase activators);
6) Agents designed to reduce the absorption of glucose from the intestine (for example acarbose);
7) Agents that prevent the reabsorption of glucose by the kidney (SGLT inhibitors);
8) Agents designed to treat the complications of prolonged hyperglycaemia (for example aldose reductase inhibitors);
9) Anti-dyslipidaemia agents such as, HMG-CoA reductase inhibitors (eg statins); PPAR α-agonists (fibrates, eg gemfibrozil); bile acid sequestrants (cholestyramine); cholesterol absorption inhibitors (plant stanols, synthetic inhibitors); bile acid absorption inhibitors (IBATi) and nicotinic acid and analogues (niacin and slow release formulations);
10) Antihypertensive agents such as β-blockers (eg atenolol, inderal); ACE inhibitors (eg lisinopril); Calcium antagonists (eg. nifedipine); Angiotensin receptor antagonists (eg candesartan), α-antagonists and diuretic agents (eg. furosemide, benzthiazide);
11) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin;
12) Agents which antagonise the actions of glucagon; and
13) Anti-inflammatory agents, such as non-steroidal anti-inflammatory drugs (eg. aspirin) and steroidal anti-inflammatory agents (eg. cortisone).

In addition to their use in therapeutic medicine, compounds of formula (I) and their pharmaceutically-acceptable salts are also useful as pharmacological tools in the development and standardisation of in vitro and in vivo test systems for the evaluation of the effects of inhibitors of DGAT1 activity in laboratory animals such as cats, dogs, rabbits, monkeys, rats and mice, as part of the search for new therapeutic agents.

In the above other pharmaceutical composition, process, method, use and medicament manufacture features, the alternative and preferred embodiments of the compounds of the invention described herein also apply.

As indicated above, all of the compounds, and their corresponding pharmaceutically-acceptable salts, are useful in inhibiting DGAT1. The ability of the compounds of formula (I), and their corresponding pharmaceutically-acceptable (acid addition) salts, to inhibit DGAT1 may be demonstrated employing the following enzyme assay:

### Human Enzyme Assay

The in vitro assay to identify DGAT1 inhibitors uses human DGAT1 expressed in insect cell membranes as the enzyme source (Proc. Natl. Acad. Sci. 1998, 95, 13018-13023). Briefly, sf9 cells were infected with recombinant baculovirus containing human DGAT1 coding sequences and harvested after 48 h. Cells were lysed by sonication and membranes isolated by centrifuging at 28000 rpm for 1 h at 4° C. on a 41% sucrose gradient. The membrane fraction at the interphase was collected, washed, and stored in liquid nitrogen.

DGAT1 activity is assayed by a modification of the method described by Coleman (Methods in Enzymology 1992, 209, 98-102). Compound at 1-10 μM is incubated with 0.4 μg membrane protein, 5 mM MgCl₂, and 100 μM 1,2 dioleoyl-sn-glycerol in a total assay volume of 200 μl in plastic tubes. The reaction is started by adding ¹⁴C oleoyl coenzyme A (30 μM final concentration) and incubated at room temperature for 30 minutes. The reaction is stopped by adding 1.5 mL 2-propanol:heptane:water (80:20:2). Radioactive triolein product is separated into the organic phase by adding 1 mL heptane and 0.5 mL 0.1 M carbonate buffer pH 9.5. DGAT1 activity was quantified by counting aliquots of the upper heptane layer by liquid scintillography.

In a further developed assay, DGAT1 activity was assayed by a modification of the method described by Coleman (Methods in Enzymology 1992, 209, 98-102). Compound at 0.00003-10 μM (final cone) was incubated with 25 μg/ml (final cone) membrane protein, 5 mM MgCl₂, and 100 μM 1,2 dioleoyl-sn-glycerol in a total assay volume of 200 μl in a 96 well plate. The reaction was started by adding ¹⁴C oleoyl coenzyme A (30 μM final concentration) and incubated at room temperature for 30 minutes. The reaction was stopped by adding 300 μl 2-propanol:heptane 7:1. Radioactive triolein product was separated into the organic phase by adding 200 μl heptane and 200 μl 0.1 M carbonate buffer pH 9.5. DGAT1 activity was quantified by counting aliquots of the upper heptane layer by liquid scintillography.

Using this assay the compounds generally show activity with IC₅₀<10 μM, preferably <1 μM, more preferably <0.1 μM, particularly, <0.05 μM, and more particularly <0.01 μM. In the further developed assay Example 1 showed an IC₅₀=0.011 μM. Examples 2 to 37 showed, respectively, IC₅₀=0.494 μM; 0.035 μM; 0.015 μM; 0.031 μM; 0.023 μM; 0.01 μM; 0.01 μM; 0.055 μM; 0.019 μM; 0.0072 μM; 0.0074 μM; 0.011 μM; 0.011 μM; 0.008 μM; 0.0038 μM; 0.027 μM; 0.021 μM; 0.012 μM; 0.0098 μM; 0.011 μM; 0.036 μM; 0.021 μM; 0.02 μM; 0.052 μM; 0.013 μM; 0.019 μM; 0.016 μM; 0.025 μM; 0.017 μM; 0.048 μM; 0.026 μM; 0.022 μM; 0.021 μM; 0.087 μM; 0.0096 μM; 0.018 μM. Examples 38 to 71 showed, respectively, IC₅₀=0.0054 μM; 0.0075 μM; 0.0035 μM; 0.0034 μM; 0.0011 μM; 0.05 μM; 0.054 μM; 0.0064 μM; 0.022 μM; 0.022 μM; 0.014 μM; 0.018 μM; 0.0034 μM; 0.029 μM; 0.023 μM; 0.0031 μM; 0.0083 μM; 0.0021 μM; 0.15 μM; 0.41 μM; 0.0061 μM; 0.0086 μM; 0.014 μM; 0.019 μM; 0.058 μM; 0.03 μM; 0.0019 μM; 0.026 μM; 0.0021 μM; 0.0072 μM; 0.0083 μM; 0.0043 μM; 0.0041 μM; 0.034 μM.

The ability of the compounds of formula (I), and their corresponding pharmaceutically-acceptable (acid) salts, to inhibit DGAT1 may further be demonstrated employing the following whole cell assays 1), 2) and/or 3):

### 1) Measurement of Triglyceride Synthesis in 3T3 Cells

Mouse adipocyte 3T3 cells were cultured to confluency in 6 well plates in new born calf serum containing media. Differentiation of the cells was induced by incubating in medium containing 10% foetal calf serum, 1 μg/mL insulin, 0.25 μM dexamethasone and 0.5 mM isobutylmethyl xanthine. After 48 h the cells were maintained in medium containing 10% foetal calf serum and 1 μg/mL insulin for a further 4-6 days. For the experiment, the medium was changed to serum-free medium and the cells pre-incubated with compound solubilised in DMSO (final concentration 0.1%) for 30 minutes. De novo lipogenesis was measured by the addition of 0.25 mM sodium acetate plus 1 μCi/mL ¹⁴C-sodium acetate to each well for a further 2 h (J. Biol. Chem., 1976, 251, 6462-6464). The cells were washed in phosphate buffered saline and solubilised in 1% sodium dodecyl sulfate. An aliquot was removed for protein determination using a protein estimation kit (Perbio) based on the method of Lowry (J. Biol. Chem., 1951, 193, 265-275). The lipids were extracted into the organic phase using a heptane:propan-2-ol:water (80:20:2) mixture followed by aliquots of water and heptane according to the method of Coleman (Methods in Enzymology, 1992, 209, 98-104). The organic phase was collected and the solvent evaporated under a stream of nitrogen. The extracts solubilised in iso-hexane:acetic acid (99:1) and lipids separated via normal phase high performance liquid chromatography (HPLC) using a Lichrospher diol-5, 4×250 mm column and a gradient solvent system of iso-hexane:acetic acid (99:1) and iso-hexane:propan-2-ol:acetic acid (85:15:1), flow rate of 1 mL/minute according to the method of Silversand and Haux (1997). Incorporation of radiolabel into the triglyceride fraction was analysed using a Radiomatic Flo-one Detector (Packard) connected to the HPLC machine.

### 2) Measurement of Triglyceride Synthesis in MCF7 Cells

Human mammary epithelial (MCF7) cells were cultured to confluency in 6 well plates in foetal calf serum containing media. For the experiment, the medium was changed to serum-free medium and the cells pre-incubated with compound solubilised in DMSO (final concentration 0.1%) for 30 minutes. De novo lipogenesis was measured by the addition of 50 μM sodium acetate plus 3 μCi/mL ¹⁴ C-sodium acetate to each well for a further 3 h (J. Biol. Chem., 1976, 251, 6462-6464). The cells were washed in phosphate buffered saline and solubilised in 1% sodium dodecyl sulfate. An aliquot was removed for protein determination using a protein estimation kit (Perbio) based on the method of Lowry (J. Biol. Chem., 1951, 193, 265-275). The lipids were extracted into the organic phase using a heptane:propan-2-ol:water (80:20:2) mixture followed by aliquots of water and heptane according to the method of Coleman (Methods in Enzymology, 1992, 209, 98-104). The organic phase was collected and the solvent evaporated under a stream of nitrogen. The extracts solubilised in iso-hexane:acetic acid (99:1) and lipids separated via normal phase high performance liquid chromatography (HPLC) using a Lichrospher diol-5, 4×250 mm column and a gradient solvent system of iso-hexane:acetic acid (99:1) and iso-hexane:propan-2-ol:acetic acid (85:15:1), flow rate of 1 mL/minute according to the method of Silversand and Haux (J. Chromat. B, 1997, 703, 7-14). Incorporation of radiolabel into the triglyceride fraction was analysed using a Radiomatic Flo-one Detector (Packard) connected to the HPLC machine.

### 3) Measurement of Triglyceride Synthesis in HuTu 80 Cells

HuTu80 cells were cultured to confluency in 6 well plates in minimum essential media containing foetal calf serum. For the experiment, the medium was changed to serum-free medium and the cells pre-incubated with compound solubilised in DMSO (final concentration 0.1%) for 30 minutes. De novo lipogenesis was measured either by the addition of 0.12 mM sodium oleate plus 1 μCi/mL ₁₄C-sodium oleate complexed to 0.03 mM BSA to each well for a further 2 h or by the addition of 0.05 mM sodium acetate plus 1 μCi/mL ₁₄C-sodium acetate to each well for a further 3 h. The cells were washed in phosphate buffered saline and solubilised in 1% sodium dodecyl sulfate. An aliquot was removed for protein determination using a protein estimation kit (Perbio) based on the method of Lowry (J. Biol. Chem., 1951, 193, 265-275). The lipids were extracted into the organic phase using a heptane:propan-2-ol:water (80:20:2) mixture followed by aliquots of water and heptane according to the method of Coleman (Methods in Enzymology, 1992, 209, 98-104). The organic phase was collected and the solvent evaporated under a stream of nitrogen. The extracts solubilised in iso-hexane:acetic acid (99:1) and lipids separated via normal phase high performance liquid chromatography (HPLC) using a Lichrospher diol-5, 4×250 mm column and a gradient solvent system of iso-hexane:acetic acid (99:1) and isohexane:propan-2-ol:acetic acid (85:15:1), flow rate of 1 mL/minute according to the method of Silversand and Haux (1997). Incorporation of radiolabel into the triglyceride fraction was analysed using a Radiomatic Flo-one Detector (Packard) connected to the HPLC machine.

### EXAMPLES

The following examples are for illustration purposes and are not intended to limit the scope of this application. A number of chemical nomenclature software packages, such as ACDName and Struc=Name/CambridgeSoft ELN, have been used in the naming of compounds.

Each exemplified compound represents a particular and independent aspect of the invention. Further aspects are the product/s obtainable by any of the Examples and/or processes described herein.

In the following non-limiting Examples, unless otherwise stated:
(i) evaporations were carried out by rotary evaporation under reduced pressure and work-up procedures were carried out after removal of residual solids such as drying agents by filtration;
(ii) operations were carried out at room temperature, that is in the range 18-25° C. and under an atmosphere of an inert gas such as argon or nitrogen;
(iii) yields are given for illustration only, and are not necessarily the maximum attainable;
(iv) the structures of the end-products of the Formula (I) were confirmed by nuclear (generally proton) magnetic resonance (NMR) and mass spectral techniques; proton magnetic resonance chemical shift values were measured on the delta scale and peak multiplicities are shown as follows: s, singlet; d, doublet; t, triplet; m, multiplet; br, broad; q, quartet, quin, quintet;
(v) intermediates were not generally fully characterised and purity was assessed by thin layer chromatography (TLC), high-performance liquid chromatography (HPLC), infra-red (IR) or NMR analysis;
(vi) flash chromatography was carried out on silica unless otherwise stated.

### ABBREVIATIONS

Aq. aqueous;
Conc. concentrated;
DCM dichloromethane;
DMA dimethylacetamide
DMSO dimethyl sulphoxide;
DMF dimethylformamide;
HPLC high pressure liquid chromatography;
LCMS liquid chromatography/mass spectroscopy;
NMR nuclear magnetic resonance spectroscopy;
pH −log₁₀[H⁺];
RT room temperature;
TFA trifluoroacetic acid;
THF tetrahydrofuran

### Example 1

### cis-4-[5-[[5-[(3,4-Difluorophenyl)amino]-1,3,4-oxadiazole-2-carbonyl]amino]pyridin-2-yl]oxycyclohexane-1-carboxylic acid

### Alternative Name: (1s,4s)-4-(5-(5-(3,4-difluorophenylamino)-1,3,4-oxadiazole-2-carboxamido)pyridin-2-yloxy)cyclohexanecarboxylic acid

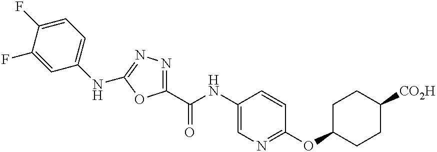

Sodium hydroxide (2M aq., 13.50 mL, 27 mmol) was added to a stirred solution of methyl cis-4-[5-[[5-[(3,4-difluorophenyl)amino]1,3,4-oxadiazole-2-carbonyl]amino]pyridin-2-yl]oxycyclohexane-1-carboxylate (Intermediate 1) (3.20 g, 6.76 mmol) in methanol (50 mL) at ambient temperature. The resulting solution was stirred at ambient temperature for 3 hours.

The reaction mixture was cooled in an ice bath and acidified with 2M hydrochloric acid. The precipitate was collected by filtration, washed with water (10 mL) and dried under vacuum to afford the crude product which was purified by crystallisation from acetic acid to give the title compound as a crystalline solid (2.20 g, 71%).

¹H NMR (400 MHz, DMSO) δ 1.62-1.90 (m, 8H), 2.32-2.43 (m, 1H), 5.09-5.15 (m, 1H), 6.82 (d, 1H), 7.32-7.39 (m, 1H), 7.48 (q, 1H), 7.65-7.74 (m, 1H), 8.02-8.08 (m, 1H), 8.51 (d, 1H), 11.08 (s, 1H), 11.23 (s, 1H), 12.04 (s, 1H). m/z 460.35 (M+H)⁺

### Example 2

### (1r,4r)-4-(5-(5-(3,4-difluorophenylamino)-1,3,4-oxadiazole-2-carboxamido)pyridin-2-yloxy)-1-methylcyclohexanecarboxylic acid

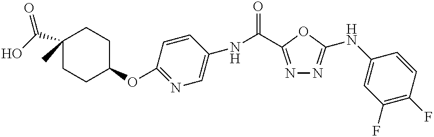

Ethyl 4-(5-(5-(3,4-difluorophenylamino)-1,3,4-oxadiazole-2-carboxamido)pyridin-2-yloxy)-1-methylcyclohexanecarboxylate (Intermediate 8) (0.129 g, 0.26 mmol) and potassium trimethylsilanolate (0.165 g, 1.29 mmol) were suspended in THF (2.57 mL) and sealed into a microwave tube. The reaction was heated to 90° C. for 20 minutes in a microwave reactor and cooled to RT. The reaction mixture was diluted with water and extracted with ethyl acetate. Product precipitated out of water layer overnight. The precipitate was collected by filtration, washed with isohexane (1 mL) and dried under vacuum to afford crude product which was purified by crystallisation in hot ethanol to yield the title compound (0.012 g, 9.9%). m/z 474 (M+H)⁺

### Example 3

### 1-(Methoxymethyl)-4-(5-(5-(2,4,5-trifluorophenylamino)-1,3,4-oxadiazole-2-carboxamido)pyridin-2-yloxy)cyclohexanecarboxylic acid

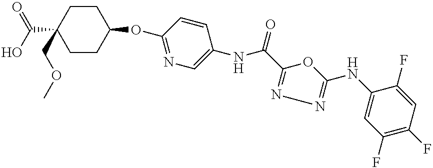

Trifluoroacetic acid (2.9 mL, 38 mmol) was added to tert-butyl 1-(methoxymethyl)-4-(5-(5-(2,4,5-trifluorophenylamino)-1,3,4-oxadiazole-2-carboxamido)pyridin-2-yloxy)cyclohexanecarboxylate (Intermediate 17) (0.22 g, 0.38 mmol). The resulting suspension was stirred at ambient temperature for 1 hour. The reaction mixture was evaporated. The crude product was purified by preparative HPLC eluting with a gradient of 90 to 10% water (containing 0.1% formic acid) in acetonitrile. The resulting solid was further purified by recrystallisation from ethanol to afford the title compound (18 mg, 3.5%).

¹H NMR (300 MHz, DMSO) δ 1.61-1.82 (8H, m), 3.23 (3H, s), 3.36-3.63 (2H, m), 5.07 (1H, s), 6.79 (1H, m), 7.64-7.74 (1H, m), 8.01-8.05 (1H, m), 8.10-8.19 (1H, m), 8.48 (1H, d), 11.05 (1H, br s), 11.09 (1H, s), 12.21 (1H, br s). m/z 522 (M+H)⁺

### Example 4

### (1r,4r)-4-(5-(5-(3,4-Difluorophenylamino)-1,3,4-oxadiazole-2-carboxamido)pyridin-2-yloxy)-1-(methoxymethyl)cyclohexanecarboxylic acid

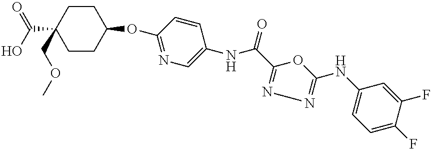

Trifluoroacetic acid (9.6 mL, 124 mmol) was added to (1r,4r)-tert-butyl 4-(5-(5-(3,4-difluorophenylamino)-1,3,4-oxadiazole-2-carboxamido)pyridin-2-yloxy)-1-(methoxymethyl)cyclohexanecarboxylate (Intermediate 16) (1.39 g, 2.48 mmol). The resulting solution was stirred at ambient temperature for 2 hours. The reaction mixture was evaporated and the residue was purified by preparative HPLC, eluting with a gradient of 90 to 10% water (containing 0.1% formic acid) in acetonitrile to afford the title compound (0.50 g, 40%). This was recrystallized from hot ethanol.

¹H NMR (400 MHz, DMSO) δ 1.34-1.52 (1H, m), 1.62-1.68 (2H, m), 1.73-1.84 (5H, m), 1.94-1.98 (1H, m), 2.08 (1H, d), 3.24 (3H, d), 3.35 (2H, s), 5.08 (1H, t), 6.77-6.83 (1H, m), 7.33-7.36 (1H, m), 7.43-7.51 (1H, m), 7.66-7.72 (1H, m), 8.01-8.06 (1H, m), 8.49 (1H, d), 11.07 (1H, s), 11.22 (1H, s). m/z 504 (M+H)⁺

### Example 5

### (1s,4s)-4-(5-(5-(2,4-difluoro-5-(trifluoromethyl)phenylamino)-1,3,4-oxadiazole-2-carboxamido)pyridin-2-yloxy)cyclohexanecarboxylic acid

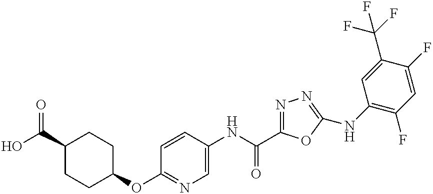

Sodium hydroxide (2M, 4 mL, 8.00 mmol) was added to (1s,4s)-methyl 4-(5-(5-(2,4-difluoro-5-(trifluoromethyl)phenylamino)-1,3,4-oxadiazole-2-carboxamido)pyridin-2-yloxy)cyclohexanecarboxylate (Intermediate 26) (310 mg, 0.57 mmol), in methanol (10 mL). The resulting solution was stirred at 22° C. for 3 days. The methanol was removed by evaporation. The residue was neutralised with 2M hydrochloric acid. The precipitate formed was collected by filtration, washed with water (5 mL), ethanol (5 mL) and dried under vacuum. The crude product was purified by preparative HPLC (Phenomenex Gemini C18 110A (axia) column, 5μ silica, 30 mm diameter, 100 mm length), eluting with a gradient 10 to 90% acetonitrile in water (containing 0.1% TFA). Fractions containing the desired compound were evaporated to dryness to afford the title compound (77 mg, 26%).

¹H NMR (400 MHz, DMSO) δ 1.56-1.81 (8H, m), 2.28-2.36 (1H, m), 5.06 (1H, s), 6.78 (1H, d), 7.76 (1H, t), 7.99 (1H, dd), 8.44 (1H, d), 8.49 (1H, t), 11.09 (1H, s), 11.20 (1H, s), 12.10 (1H, s). m/z 528 (M+H)⁺

### Example 6

### (1s,4s)-4-(5-(5-(3-(trifluoromethoxy)phenylamino)-1,3,4-oxadiazole-2-carboxamido)pyridin-2-yloxy)cyclohexanecarboxylic acid

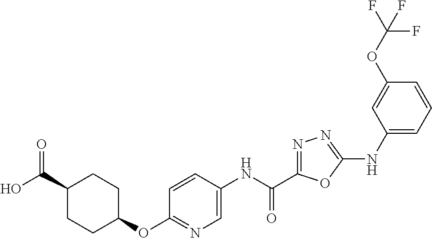

Sodium hydroxide (2M, 4 mL, 8.00 mmol) was added to (1s,4s)-methyl 4-(5-(5-(3-(trifluoromethoxy)phenylamino)-1,3,4-oxadiazole-2-carboxamido)pyridin-2-yloxy)cyclohexanecarboxylate (Intermediate 33) (630 mg, 1.21 mmol), in methanol (10 mL). The resulting solution was stirred at 22° C. for 3 days. The methanol was removed by evaporation. The residue was neutralised with 2M hydrochloric acid. The precipitate formed was collected by filtration. This was washed with water (5 mL), ethanol (5 mL) and dried under vacuum to afford a solid. This was purified by crystallisation from acetic acid to afford the product (190 mg, 31%).

¹H NMR (400 MHz, DMSO) δ 1.63-1.89 (8H, m), 2.32-2.43 (1H, m), 5.12 (1H, s), 6.84 (1H, d), 7.06 (1H, d), 7.50-7.59 (2H, m), 7.71 (1H, s), 8.06 (1H, dd), 8.51 (1H, d), 11.17 (1H, s), 11.40 (1H, s), 12.16 (1H, s). m/z 508 (M+H)⁺

### Example 7

### (1s,4s)-4-(5-(5-(3-chlorophenylamino)-1,3,4-oxadiazole-2-carboxamido)pyridin-2-yloxy)cyclohexanecarboxylic acid

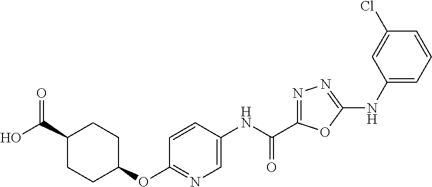

Sodium hydroxide (2M, 4.5 mL, 9.00 mmol) was added to (1s,4s)-methyl 4-(5-(5-(3-chlorophenylamino)-1,3,4-oxadiazole-2-carboxamido)pyridin-2-yloxy)cyclohexanecarboxylate (Intermediate 34) (810 mg, 1.72 mmol), in methanol (10 mL) at 22° C. The resulting solution was stirred at 22° C. for 16 hours. The reaction mixture was concentrated by evaporation. The reaction mixture was neutralised with 2M hydrochloric acid (4.5 mL). A precipitate was collected by filtration. This was washed with in water (25 mL) then methanol (25 mL) and dried under vacuum to afford a white solid. The crude product was purified by crystallisation from acetic acid to afford the product (441 mg, 56%).

¹H NMR (400 MHz, DMSO) δ 1.61-1.89 (8H, m), 2.34-2.44 (1H, m), 5.12 (1H, s), 6.84 (1H, d), 7.13 (1H, d), 7.43 (1H, t), 7.49-7.55 (1H, m), 7.75 (1H, s), 8.06 (1H, d), 8.51 (1H, s), 11.16 (1H, s), 11.29 (1H, s), 12.16 (1H, s). m/z 458 (M+H)⁺

### Example 8

### (1s,4s)-4-(5-(5-(4-fluoro-3-(trifluoromethyl)phenylamino)-1,3,4-oxadiazole-2-carboxamido)pyridin-2-yloxy)cyclohexanecarboxylic acid

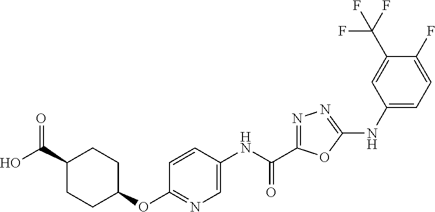

2M Sodium hydroxide (4.5 mL, 9.0 mmol) was added to (1s,4s)-methyl 4-(5-(5-(4-fluoro-3-(trifluoromethyl)phenylamino)-1,3,4-oxadiazole-2-carboxamido)pyridin-2-yloxy)cyclohexanecarboxylate (Intermediate 35) (1.65 g, 1.64 mmol), in methanol (10 mL) at 22° C. The resulting solution was stirred at 22° C. for 16 hours. The reaction mixture was concentrated by evaporation. The residue was neutralised with 2M hydrochloric acid (4.5 mL). A precipitate was collected by filtration. This was washed with water (25 mL) then methanol (50 mL) and dried under vacuum to afford a white solid. The crude product was purified by crystallisation from acetic acid to afford the product (0.379 g, 45.4%).

¹H NMR (300 MHz, DMSO) δ 1.60-1.87 (8H, m), 2.31-2.43 (1H, m), 5.11 (1H, s), 6.82 (1H, d), 7.57 (1H, t), 7.84-7.92 (1H, m), 7.97-8.07 (2H, m), 8.49 (1H, d), 11.09 (1H, s), 11.37 (1H, s), 12.00 (1H, s). m/z 510 (M+H)⁺

### Example 9

### (1s,4s)-4-(5-(5-(4-tert-butylphenylamino)-1,3,4-oxadiazole-2-carboxamido)pyridin-2-yloxy)cyclohexanecarboxylic acid

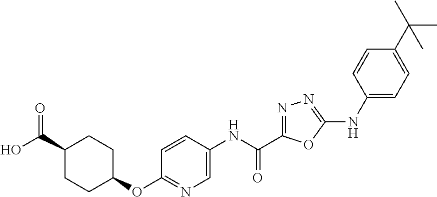

Sodium hydroxide (2M, 4.5 mL, 9.00 mmol) was added to (1s,4s)-methyl 4-(5-(5-(4-tert-butylphenylamino)-1,3,4-oxadiazole-2-carboxamido)pyridin-2-yloxy)cyclohexanecarboxylate (Intermediate 36) (1.38 g, 1.76 mmol), in methanol (10 mL) at 22° C. The resulting solution was stirred at 22° C. for 16 hours. The reaction mixture was concentrated by evaporation. The residue was neutralised with 2M hydrochloric acid solution (4.5 mL). A precipitate was collected by filtration. This was washed with water (25 mL) then methanol (50 mL) and dried under vacuum to afford a white solid. The crude product was purified by crystallisation from acetic acid to afford the product (325 mg, 38.5%).

¹H NMR (300 MHz, DMSO) δ 1.27 (9H, s), 1.60-1.89 (8H, m), 2.32-2.43 (1H, m), 5.11 (1H, s), 6.82 (1H, d), 7.39 (2H, d), 7.50 (2H, d), 8.04 (1H, dd), 8.49 (1H, d), 10.84 (1H, s), 11.05 (1H, s) 12.08 (1H, br s). m/z 480 (M+H)⁺

### Example 10

### (1s,4s)-4-(5-(5-(4-isopropylphenylamino)-1,3,4-oxadiazole-2-carboxamido)pyridin-2-yloxy)cyclohexanecarboxylic acid

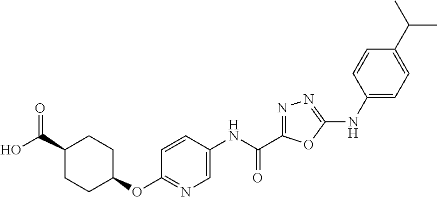

Sodium hydroxide (2M, 4.5 mL, 9.00 mmol) was added to (1s,4s)-methyl 4-(5-(5-(4-isopropylphenylamino)-1,3,4-oxadiazole-2-carboxamido)pyridin-2-yloxy)cyclohexanecarboxylate (Intermediate 37) (1.01 g, 1.78 mmol) in methanol (10 mL) at 22° C. The resulting solution was stirred at 22° C. for 16 hours. The reaction mixture was concentrated by evaporation. The residue was neutralised with 2M hydrochloric acid solution (4.5 mL). A precipitate was collected by filtration. This was washed with water (25 mL), then methanol (50 mL) and dried under vacuum. The crude product was purified by crystallisation from acetic acid to afford the product (427 mg, 51.5%).

¹H NMR (300 MHz, DMSO) δ 1.19 (6H, d), 1.61-1.88 (8H, m), 2.31-2.44 (1H, m), 2.80-2.92 (1H, m), 5.11 (1H, s), 6.81 (1H, d), 7.25 (2H, d), 7.49 (2H, d), 8.04 (1H, dd), 8.49 (1H, d), 10.84 (1H, s), 11.04 (1H, s), 12.07 (1H, s). m/z 466 (M+H)⁺

### Example 11

### (1s,4s)-4-(5-(5-(4-(trifluoromethoxy)phenylamino)-1,3,4-oxadiazole-2-carboxamido)pyridin-2-yloxy)cyclohexanecarboxylic acid

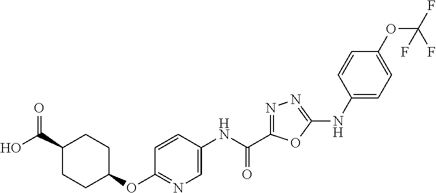

Sodium hydroxide (2M, 4.5 mL, 9.0 mmol) was added to (1s,4s)-methyl 4-(5-(5-(4-(trifluoromethoxy)phenylamino)-1,3,4-oxadiazole-2-carboxamido)pyridin-2-yloxy)cyclohexanecarboxylate (Intermediate 38) (1.17 g, 1.78 mmol) in methanol (10 mL) at 22° C. The resulting solution was stirred at 22° C. for 16 hours. The reaction mixture was concentrated by evaporation. The residue was neutralised with 2M hydrochloric acid solution (4.5 mL). A precipitate was collected by filtration. This was washed with water (25 mL) then methanol (50 mL) and dried under vacuum to afford crude product. The crude product was purified by crystallisation from acetic acid to afford the product as the acetic acid solvate (1:1) (0.454 g, 50.3%).

¹H NMR (300 MHz, DMSO) δ 1.60-1.88 (8H, m), 2.31-2.44 (1H, m), 5.11 (1H, s), 6.82 (1H, d), 7.41 (2H, d), 7.69 (2H, d), 8.05 (1H, dd), 8.49 (1H, d), 11.08 (1H, s). m/z 508 (M+H)⁺

### Example 12

### (1s,4s)-4-(5-(5-(2,4-dichlorophenylamino)-1,3,4-oxadiazole-2-carboxamido)pyridin-2-yloxy)cyclohexanecarboxylic acid

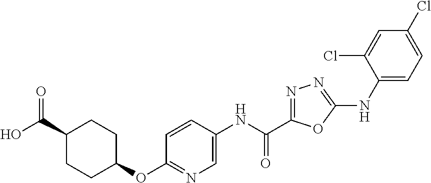

Sodium hydroxide (2M, 4.5 mL, 9.0 mmol) was added to (1s,4s)-methyl 4-(5-(5-(2,4-dichlorophenylamino)-1,3,4-oxadiazole-2-carboxamido)pyridin-2-yloxy)cyclohexanecarboxylate (Intermediate 39) (1.19 g, 1.78 mmol) in methanol (10 mL) at 22° C. The resulting solution was stirred at 22° C. for 16 hours. The reaction mixture was concentrated by evaporation. The residue was neutralised with 2M hydrochloric acid (4.5 mL). A precipitate was collected by filtration. This was then washed with water (25 mL), methanol (50 mL), and dried under vacuum to afford crude product. The crude product was purified by crystallisation from acetic acid to afford the product as the acetic acid solvate (1:1) (0.490 g, 56%).

¹H NMR (300 MHz, DMSO) δ 1.60-1.88 (8H, m), 2.32-2.43 (1H, m), 5.11 (1H, s), 6.82 (1H, d), 7.51 (1H, dd), 7.70 (1H, d), 7.98-8.06 (2H, m), 8.49 (1H, d), 10.55 (1H, s), 10.55 (1H, s), 11.99 (1H, s). m/z 492 (M+H)⁺

### Example 13

### (1s,4s)-4-(5-(5-(4-bromo-2-chlorophenylamino)-1,3,4-oxadiazole-2-carboxamido)pyridin-2-yloxy)cyclohexanecarboxylic acid

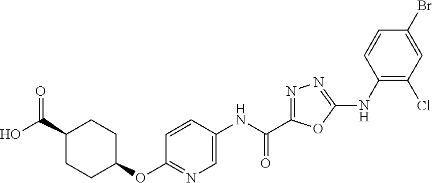

Sodium hydroxide (2M, 4.5 mL, 9.00 mmol) was added to (1s,4s)-methyl 4-(5-(5-(4-bromo-2-chlorophenylamino)-1,3,4-oxadiazole-2-carboxamido)pyridin-2-yloxy)cyclohexanecarboxylate (Intermediate 40) (0.903 g, 1.64 mmol), in methanol (10 mL) at 22° C. The resulting solution was stirred at 22° C. for 16 hours. The reaction mixture was concentrated by evaporation. The residue was neutralised with 2M hydrochloric acid (4.5 mL). A precipitate was collected by filtration, washed with water (25 mL) then methanol (50 mL) and dried under vacuum to afford a white solid. The crude product was purified by crystallisation from acetic acid to afford the product (0.624 g, 71%).

¹H NMR (400 MHz, DMSO) δ 1.63-1.87 (8H, m), 2.31-2.43 (1H, m), 5.11 (1H, s), 6.84 (1H, d), 7.65 (1H, d), 7.83 (1H, s), 7.99 (1H, d), 8.05 (1H, d), 8.50 (1H, d), 10.62 (1H, s), 11.14 (1H, s), 12.07 (1H, s). m/z 538 (M+H)⁺

### Example 14

### (1s,4s)-4-(5-(5-(2,3-dichlorophenylamino)-1,3,4-oxadiazole-2-carboxamido)pyridin-2-yloxy)cyclohexanecarboxylic acid

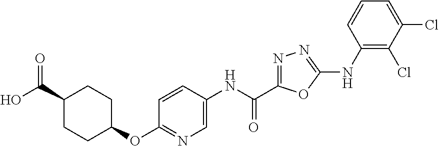

Sodium hydroxide (2M, 4.5 mL, 9.00 mmol) was added to (1s,4s)-methyl 4-(5-(5-(2,3-dichlorophenylamino)-1,3,4-oxadiazole-2-carboxamido)pyridin-2-yloxy)cyclohexanecarboxylate (Intermediate 41) (550 mg, 1.09 mmol), in methanol (10 mL) at 22° C. The resulting solution was stirred at 22° C. for 16 hours. The reaction mixture was concentrated by evaporation. The residue was neutralised with 2M hydrochloric acid (4.5 mL). A precipitate was collected by filtration. This was washed with water (25 mL), then methanol (25 mL), and dried under vacuum. The crude product was purified by crystallisation from acetic acid to afford the product (509 mg, 95%).

¹H NMR (400 MHz, DMSO) δ 1.62-1.88 (8H, m), 2.35-2.43 (1H, m), 5.12 (1H, s), 6.84 (1H, d), 7.43-7.50 (2H, m), 7.97-8.08 (2H, m), 8.51 (1H, d), 10.69 (1H, s), 11.15 (1H, s), 12.15 (1H, s). m/z 492 (M+H)⁺

### Example 15

### (1s,4s)-4-(5-(5-(3-chloro-4-methylphenylamino)-1,3,4-oxadiazole-2-carboxamido)pyridin-2-yloxy)cyclohexanecarboxylic acid

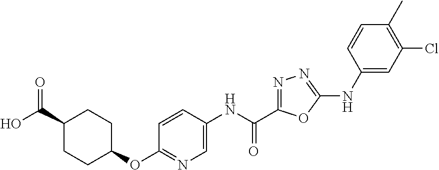

Sodium hydroxide (2M, 4.5 mL, 9.00 mmol) was added to (1s,4s)-methyl 4-(5-(5-(3-chloro-4-methylphenylamino)-1,3,4-oxadiazole-2-carboxamido)pyridin-2-yloxy)cyclohexanecarboxylate (Intermediate 42) (0.946 g, 1.64 mmol) in methanol (10 mL) at 22° C. The resulting solution was stirred at 22° C. for 16 hours. The reaction mixture was concentrated by evaporation. The residue was neutralised with 2M hydrochloric acid (4.5 mL). A precipitate was collected by filtration. This was washed with water (25 mL) then methanol (50 mL) and dried under vacuum. The crude product was purified by crystallisation from acetic acid to afford the product (453 mg, 59%).

¹H NMR (400 MHz, DMSO) δ 1.62-1.88 (8H, m), 2.30 (3H, s), 2.35-2.43 (1H, m), 5.12 (1H, s), 6.84 (1H, d), 7.34-7.45 (2H, m), 7.73 (1H, s), 8.06 (1H, d), 8.51 (1H, s), 11.15 (2H, s), 12.16 (1H, s). m/z 472 (M+H)⁺

### Example 16

### (1s,4s)-4-(5-(5-(3-chloro-4-fluorophenylamino)-1,1,4-oxadiazole-2-carboxamido)pyridin-2-yloxy)cyclohexanecarboxylic acid

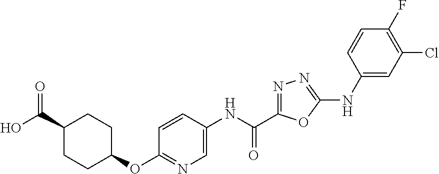

Sodium hydroxide (2M, 4.5 mL, 9.00 mmol) was added to (1s,4s)-methyl 4-(5-(5-(3-chloro-4-fluorophenylamino)-1,3,4-oxadiazole-2-carboxamido)pyridin-2-yloxy)cyclohexanecarboxylate (Intermediate 43) (853 mg, 1.74 mmol) in methanol (10 mL) at 22° C. The resulting solution was stirred at 22° C. for 16 hours. The reaction mixture was concentrated by evaporation. The residue was neutralised with 2M hydrochloric acid (4.5 mL). A precipitate was collected by filtration. This was washed with water (25 mL), then methanol (50 mL) and dried under vacuum to afford crude product as a white solid. The crude product was purified by crystallisation from acetic acid to afford the product as its acetic acid solvate (1:1) (390 mg, 47%).

¹H NMR (300 MHz, DMSO) δ 1.60-1.88 (8H, m), 2.32-2.44 (1H, m), 5.11 (1H, s), 6.82 (1H, d), 7.41-7.56 (2H, m), 7.82 (1H, dd), 8.04 (1H, dd), 8.49 (1H, d), 11.09 (1H, s) 1 equiv acetic acid. m/z 476 (M+H)⁺

### Example 17

### (1s,4s)-4-(5-(5-(3,4-difluorophenylamino)-1,3,4-oxadiazole-2-carboxamido)-3-methylpyridin-2-yloxy)cyclohexanecarboxylic acid

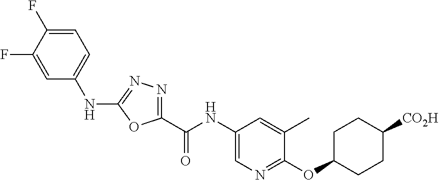

Sodium hydroxide (2M aq., 2.00 mL, 4 mmol) was added to a stirred solution of (1s,4s)-methyl 4-(5-(5-(3,4-difluorophenylamino)-1,3,4-oxadiazole-2-carboxamido)-3-methylpyridin-2-yloxy)cyclohexanecarboxylate (Intermediate 44) (0.487 g, 1.0 mmol) in methanol (10 mL) at ambient temperature. The resulting solution was stirred at ambient temperature for 3 hours.

The reaction mixture was cooled in an ice bath and acidified with 2M hydrochloric acid. The precipitate was collected by filtration, washed with water (10 mL) and dried under vacuum to afford the crude product which was purified by crystallisation from acetic acid to give the title compound as a crystalline solid (0.172 g, 36%).

¹H NMR (400 MHz, DMSO) δ 1.63-1.91 (m, 8H), 2.18 (s, 3H), 2.32-2.42 (m, 1H), 5.17-5.24 (m, 1H), 7.33-7.39 (m, 1H), 7.49 (q, 1H), 7.66-7.74 (m, 1H), 7.93 (d, 1H), 8.32 (d, 1H), 11.01 (s, 1H), 11.23 (s, 1H), 12.02 (s, 1H). m/z 474.44 (M+H)⁺

### Example 18

### (1s,4s)-4-(5-(5-(3-chlorophenylamino)-1,3,4-oxadiazole-2-carboxamido)-3-methylpyridin-2-yloxy)cyclohexanecarboxylic acid

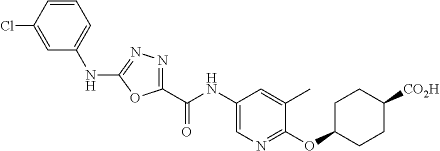

Sodium hydroxide (2M aq., 2.24 mL, 4.48 mmol) was added to a stirred solution of (1s,4s)-methyl 4-(5-(5-(3-chlorophenylamino)-1,3,4-oxadiazole-2-carboxamido)-3-methylpyridin-2-yloxy)cyclohexanecarboxylate (Intermediate 51) (0.544 g, 1.12 mmol) in methanol (10 mL) at ambient temperature. The resulting solution was stirred at ambient temperature for 3 hours.

The reaction mixture was cooled in an ice bath and acidified with 2M hydrochloric acid. The precipitate was collected by filtration, washed with water (10 mL) and dried under vacuum to afford the crude product which was purified by crystallisation from acetic acid to give the title compound as a crystalline solid (0.174 g, 33%).

¹H NMR (400 MHz, DMSO) δ 1.62-1.93 (m, 8H), 2.18 (s, 3H), 2.31-2.43 (m, 1H), 5.18-5.24 (m, 1H), 7.10-7.16 (m, 1H), 7.43 (t, 1H), 7.49-7.55 (m, 1H), 7.74 (t, 1H), 7.93 (d, 1H), 8.32 (d, 1H), 11.01 (s, 1H), 11.22 (s, 1H), 12.03 (s, 1H). m/z 472.40 (M+H)⁺

### Example 19

### 4-({5-[({5-[(2,4,5-trifluorophenyl)amino]-1,3,4-oxadiazol-2-yl}carbonyl)amino]pyridin-2-yl}oxy)cyclohexanecarboxylic acid

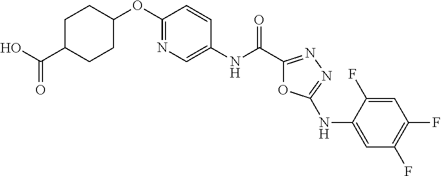

Trifluoroacetic acid (1.5 mL, 20.17 mmol) was added to a stirred solution of tert-butyl 4-({5-[({5-[(2,4,5-trifluorophenyl)amino]-1,3,4-oxadiazol-2-yl}carbonyl)amino]pyridin-2-yl}oxy)cyclohexanecarboxylate (Intermediate 52) (474 mg, 0.89 mmol) in THF (4 mL). The reaction mixture was stirred for 2 h at ambient temperature. More trifluoroacetic acid (5 mL, 67.23 mmol) was added and the mixture stirred for a further 16 h. The reaction mixture was taken to pH7 with saturated K₂CO₃ solution and then acidified with 1M citric acid until a white solid precipitated. The solid was filtered and washed with water before being dried and recrystallised from ethanol/water (4 mL/1 mL) to yield the title compound (288 mg, 68%).

¹H NMR (400 MHz, DMSO) δ1.41-1.51 (3H, m), 1.73 (2H, d), 1.96 (1H, d), 2.09 (1H, d), 2.20-2.45 (1H, m), 4.89 and 5.1 (1H, m), 6.79-6.85 (1H, m), 7.68 (1H, d), 8.04-8.07 (1H, m), 8.18 (1H, q), 8.51 (1H, d), 11.13 (1H, s). m/z 477.9 (M+H)⁺

### Example 20

### 4-4-({5-[({5-[(4-fluorophenyl)amino]-1,3,4-oxadiazol-2-yl}-carbonyl)amino]pyridin-2-yl}oxy)cyclohexanecarboxylic acid

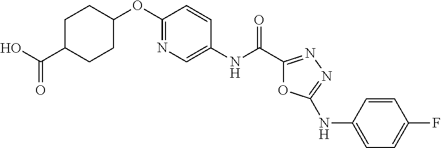

Trifluoroacetic acid (1.185 mL, 15.92 mmol) was added to a stirred solution of tert-butyl 4-({5-[({5-[(4-fluorophenyl)amino]-1,3,4-oxadiazol-2-yl}carbonyl)amino]pyridin-2-yl}oxy)cyclohexanecarboxylate (Intermediate 53) (396 mg, 0.80 mmol) in THF (4 mL). The reaction mixture was stirred for 2 h at ambient temperature. More trifluoroacetic acid (5 mL, 67.23 mmol) was added and the mixture stirred for a further 16 h. The reaction mixture was taken to pH7 with saturated K₂CO₃ solution and then acidified with 1M citric acid until a white solid precipitated. The solid was filtered and washed with water before being dried and recrystallised from acetic acid (6 mL) to yield the title compound (52 mg, 15%).

1H NMR (400 MHz, DMSO) δ 1.37 (3H, m), 1.55 (1H, m), 1.78 (1H, m), 1.88 (2H, m), 2.04 (2H, m), 4.84 and 5.05 (1H, m), 6.74 (1H, m), 7.05 (2H, m), 7.48-7.52 (2H, m), 8.04-8.07 (1H, m), 8.50 (1H, m), 10.75 (1H, br s). m/z 441.9 (M+H)⁺

### Example 21

### 4-[(5-{[(5-{[4-(difluoromethoxy)phenyl]amino}-1,3,4-oxadiazol-2-yl)carbonyl]amino}pyridin-2-yl)oxy]cyclohexanecarboxylic acid

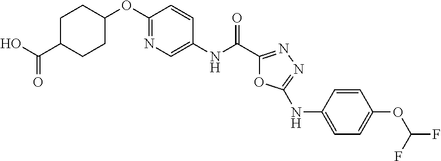

Potassium trimethylsilanolate (462 mg, 3.59 mmol) was added to a stirred solution of ethyl 4-[(5-{[(5-{[4-(difluoromethoxy)phenyl]amino}-1,3,4-oxadiazol-2-yl)carbonyl]-amino}pyridin-2-yl)oxy]cyclohexanecarboxylate (Intermediate 58) (372 mg, 0.72 mmol) in dry THF (10 mL). The reaction mixture was heated at 90° C. in the microwave for 15 min and then concentrated in vacuo. 1M citric acid (8 mL) was added to the mixture stirred before the solid was filtered and washed with water. The solid was recrystallised from acetic acid (25 mL) to yield the title compound as a white solid (68 mg, 19%).

¹H NMR (400 MHz, DMSO) δ 1.45 (3H, m), 1.73 (3H, m), 1.96 (1H, m), 2.09 (1H, m), 2.29 (1H, m), 2.60-2.68 (1H, m), 4.93 (1H, m), 6.79-6.85 (1H, m), 7.25 (2H, d), 7.62-7.66 (2H, m), 8.04-8.08 (1H, m), 8.52 (1H, m), 11.12 (1H, d), 12.15 (1H, br s).

m/z 489.9 (M+H)⁺

### Example 22

### (1r,4r)-4-(5-(5-(2,4,5-trifluorophenylamino)-1,3,4-oxadiazole-2-carboxamido)pyridin-2-yloxy)cyclohexanecarboxylic acid

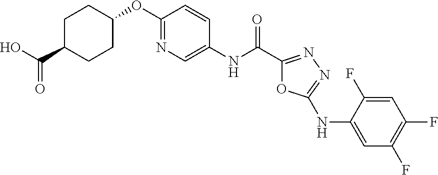

2M aq. Sodium hydroxide (2.93 mL, 5.86 mmol) was added to a stirred suspension of (1r,4r)-methyl 4-(5-(5-(2,4,5-trifluorophenylamino)-1,3,4-oxadiazole-2-carboxamido)pyridin-2-yloxy)cyclohexanecarboxylate (Intermediate 63) (576 mg, 1.17 mmol) in methanol (10 mL) at ambient temperature. The resulting solution was stirred at ambient temperature for 20 hours. Half the methanol was evaporated and the reaction mixture cooled to 0° C. The reaction mixture was acidified with 2M hydrochloric acid. The precipitate was collected by filtration, washed with water (50 mL) and dried under vacuum to afford crude product which was purified by crystallisation from acetic acid to afford r,4r)-4-(5-(5-(2,4,5-trifluorophenylamino)-1,3,4-oxadiazole-2-carboxamido)pyridin-2-yloxy)cyclohexanecarboxylic acid (252 mg, 45.0%).

¹H NMR (400 MHz, DMSO) δ 1.41-1.51 (4H, m), 1.95-2.00 (2H, m), 2.05-2.13 (2H, m), 2.22-2.32 (1H, m), 4.85-4.95 (1H, m), 6.80 (1H, d), 7.74 (1H, q), 8.02-8.06 (1H, m), 8.14-8.23 (1H, m), 8.51 (1H, d), 11.13 (1H, s), 11.17 (1H, s), 12.07 (1H, s). m/z 478 (M+H)⁺

Examples 23-33 were synthesised in an analogous fashion from the appropriate intermediate (64-74)

| Example | Structure | m/z | NMR |
|---|---|---|---|
| 23 (1r,4r)-4- (5-(5-(2,4- dichloro- phenylamino)- 1,3,4- oxadiazole-2- carboxamido) pyridin- 2-yloxy)cyclo- hexane- carboxylic acid | 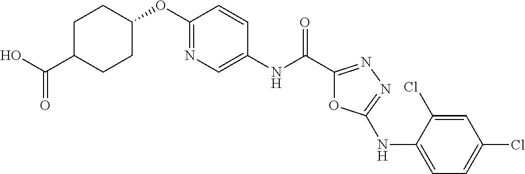 | 492 (M + H)⁺ | 1H NMR (400 MHz, DMSO) δ 1.20-1.36 (4H, m), 1.77-1.80 (2H, m), 1.86-1.95 (2H, m), 2.07-2.12 (1H, m), 4.66-4.74 (1H, m), 6.63 (1H, d), 7.35-7.38 (1H, m), 7.56 (1H, d), 7.86-7.89 (2H, m), 8.33 (1H, d), 10.44 (1H, s), 10.97 (1H, s), 11.89 (1H, s) |
| 24 (1r,4r)-4- (5-(5-(3- chloro-4- fluoro- phenylamino)- 1,3,4- oxadiazole-2- carboxamido) pyridin- 2-yloxy)cyclo- hexane- carboxylic acid | 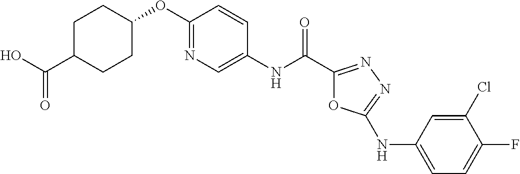 | 476 (M + H)⁺ | 1H NMR (400 MHz, DMSO) δ 1.38-1.55 (4H, m), 1.94-1.98 (2H, m), 2.07-2.12 (2H, m), 2.22-2.30 (1H, m), 4.89-4.95 (1H, m), 6.81 (1H, d), 7.48 (1H, t), 7.53-7.57 (1H, m), 7.82-7.84 (1H, m), 8.04-8.09 (1H, m), 8.51 (1H, d), 11.18 (1H, s), 11.29 (1H, s), 12.07 (1H, s) |
| 25 (1r,4r)-4- (5-(5-(4- (trifluoro- methoxy) phenylamino)- 1,3,4- oxadiazole-2- carboxamido) pyridin- 2-yloxy)cyclo- hexane- carboxylic acid | 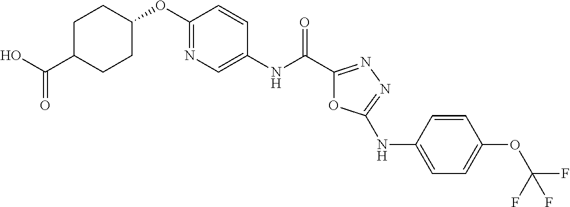 | 508 (M + H)⁺ | 1H NMR (400 MHz, DMSO) δ 1.39-1.53 (4H, m), 1.90-1.98 (2H, m), 2.08-2.15 (2H, m), 2.22-2.32 (1H, m), 4.84-5.00 (1H, m), 6.80 (1H, d), 7.44 (2H, d), 7.71 (2H, d), 8.05-8.08 (1H, m), 8.52 (1H, d), 11.16 (1H, s), 11.26 (1H, s), 12.14 (1H, s) |
| 26 (1r,4r)-4- (5-(5-(3- chloro-4- methyl- phenylamino)- 1,3,4- oxadiazole-2- carboxamido) pyridin- 2-yloxy)cyclo- hexane- carboxylic acid | 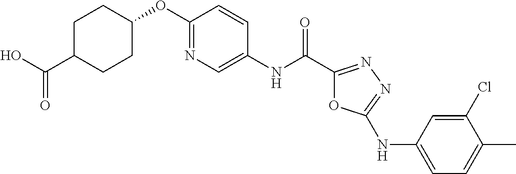 | 472 (M + H)⁺ | 1H NMR (400 MHz, DMSO) δ 1.37-1.55 (4H, m), 1.94-1.99 (2H, m), 2.07-2.13 (2H, m), 2.23-2.27 (1H, m), 2.29 (3H, s), 4.87-4.94 (1H, m), 6.80 (1H, d), 7.37 (1H, d), 7.41-7.44 (1H, m), 7.73 (1H, d), 8.04-8.07 (1H, m), 8.51 (1H, d), 11.15 (1H, s), 11.16 (1H, s), 12.12 (1H, s) |
| 27 (1r,4r)-4- (5-(5-(3- chloro- phenylamino)- 1,3,4- oxadiazole-2- carboxamido) pyridin- 2-yloxy)cyclo- hexane- carboxylic acid | 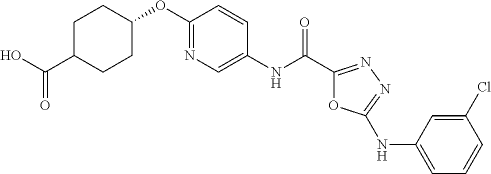 | 458 (M + H)⁺ | 1H NMR (400 MHz, DMSO) δ 1.36-1.56 (4H, m), 1.92-2.01 (2H, m), 2.06-2.13 (2H, m), 2.23-2.31 (1H, m), 4.84-4.93 (1H, m), 6.80 (1H, d), 7.11-7.14 (1H, m), 7.43 (1H, t), 7.51-7.54 (1H, m), 7.73-7.76 (1H, m), 8.04-8.08 (1H, m), 8.52 (1H, d), 11.18 (1H, s), 11.30 (1H, s), 12.10 (1H, s) |
| 28 (1r,4r)-4- (5-(5-(2,3- dichloro- phenylamino)- 1,3,4- oxadiazole-2- carboxamido) pyridin- 2-yloxy)cyclo- hexane- carboxylic acid | 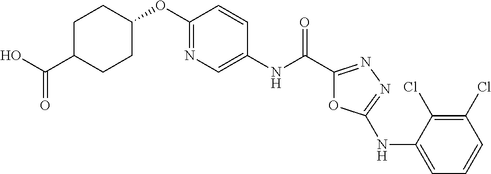 | 492 (M + H)⁺ | 1H NMR (400 MHz, DMSO) δ 1.37-1.53 (4H, m), 1.92-2.00 (2H, m), 2.07-2.12 (2H, m), 2.23-2.30 (1H, m), 4.86-4.94 (1H, m), 6.81 (1H, d), 7.44-7.48 (2H, m), 7.98-8.06 (2H, m), 8.51 (1H, d), 10.70 (1H, s), 11.16 (1H, s), 12.16 (1H, s) |
| 29 (1r,4r)-4- (5-(5-(4- fluoro-3- (trifluoro- methyl) phenylamino)- 1,3,4- oxadiazole-2- carboxamido) pyridin- 2-yloxy)cyclo- hexane- carboxylic acid | 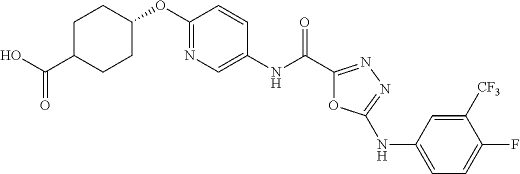 | 510 (M + H)⁺ | 1H NMR (400 MHz, DMSO) δ 1.39-1.54 (4H, m), 1.94-2.01 (2H, m), 2.07-2.13 (2H, m), 2.23-2.30 (1H, m), 4.85-4.91 (1H, m), 6.80 (1H, d), 7.59 (1H, t), 7.88- 7.92 (1H, m), 8.00- 8.07 (2H, m), 8.51 (1H, d), 11.16 (1H, s), 11.41 (1H, s), 12.12 (1H, s) |
| 30 (1r,4r)-4- (5-(5-(4- isopropyl- phenylamino)- 1,3,4- oxadiazole-2- carboxamido) pyridin- 2-yloxy)cyclo- hexane- carboxylic acid | 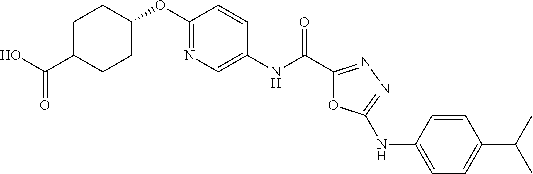 | 466 (M + H)⁺ | 1H NMR (400 MHz, DMSO) δ 1.20 (6H, d), 1.37-1.55 (4H, m), 1.93-1.99 (2H, m), 2.06-2.14 (2H, m), 2.23-2.31 (1H, m), 2.87 (1H, septet), 4.87- 4.94 (1H, m), 6.80 (1H, d), 7.27 (2H, d), 7.52 (2H, d), 8.04-8.08 (1H, m), 8.51 (1H, d), 10.90 (1H, s), 11.11 (1H, s), 12.14 (1H, s) |
| 31 (1r,4r)-4-(5- (5-(4-tert- butylphenyl- amino)- 1,3,4- oxadiazole-2- carboxamido) pyridin- 2-yloxy)cyclo- hexane- carboxylic acid | 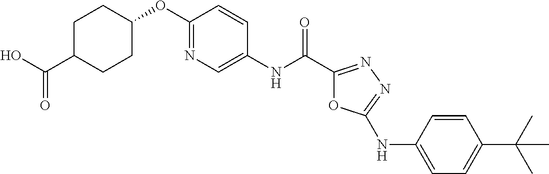 | 480 (M + H)⁺ | 1H NMR (400 MHz, DMSO) δ 1.29 (9H, s), 1.36-1.54 (4H, m), 1.91-2.00 (2H, m), 2.07-2.13 (2H, m), 2.23-2.29 (1H, m), 4.87-4.92 (1H, m), 6.80 (1H, d), 7.42 (2H, d), 7.52 (2H, d), 8.04- 8.08 (1H, m), 8.51 (1H, d), 10.91 (1H, s), 11.11 (1H, s), 12.15 (1H, s) |
| 32 (1r,4r)-4- (5-(5-(3,4- difluoro- phenylamino)- 1,3,4- oxadiazole-2- carboxamido) pyridin- 2-yloxy)cyclo- hexane- carboxylic acid | 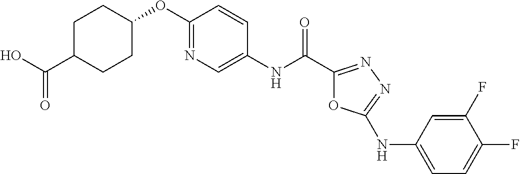 | 460 (M + H)⁺ | 1H NMR (400 MHz, DMSO) δ 1.38-1.55 (4H, m), 1.94-1.99 (2H, m), 2.07-2.15 (2H, m), 2.23-2.31 (1H, m), 4.86-4.94 (1H, m), 6.80 (1H, d), 7.33-7.39 (1H, m), 7.48 (1H, q), 7.66-7.74 (1H, m), 8.03-8.07 (1H, m), 8.51 (1H, d), 11.09 (1H, s), 11.23 (1H,s), 12.00 (1H, s) |
| 33 (1r,4r)-4- (5-(5-(4- bromo-2- chloro- phenylamino)- 1,3,4- oxadiazole-2- carboxamido) pyridin- 2-yloxy)cyclo- hexane- carboxylic acid | 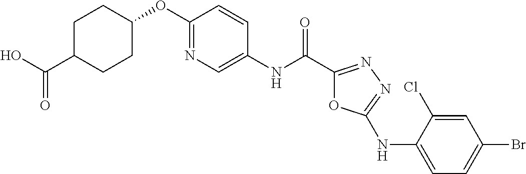 | 538 (M + H)⁺ | 1H NMR (400 MHz, DMSO) δ 1.38-1.55 (4H, m), 1.95-1.98 (2H, m), 2.07-2.13 (2H, m), 2.24-2.31 (1H, m), 4.85-4.95 (1H, m), 6.80 (1H, d), 7.61-7.68 (1H, m), 7.80-7.84 (1H, m), 7.97 (1H, d), 8.03-8.07 (1H, m), 8.51 (1H, d), 10.55 (1H, s), 11.09 (1H, s), 12.00 (1H, s) |

### Example 34

### (1r,4r)-4-(5-(5-(3-(trifluoromethoxy)phenylamino)-1,3,4-oxadiazole-2-carboxamido)pyridin-2-yloxy)cyclohexanecarboxylic acid

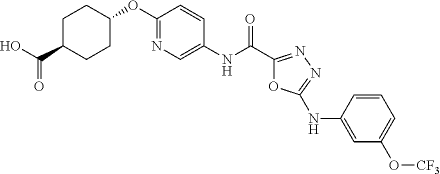

2M aq. Sodium hydroxide (2.88 mL, 5.75 mmol) was added to (1r,4r)-methyl 4-(5-(5-(3-(trifluoromethoxy)phenylamino)-1,3,4-oxadiazole-2-carboxamido)pyridin-2-yloxy)cyclohexanecarboxylate (Intermediate 80) (0.6 g, 1.15 mmol) in methanol (10 mL) at ambient temperature. The resulting solution was stirred at ambient temperature for 20 hours. Half the methanol was evaporated and the reaction mixture cooled to 0° C. The reaction mixture was acidified with 2M hydrochloric acid. The precipitate was collected by filtration, washed with water (50 mL) and methanol (50 mL) and dried under vacuum to afford crude product. The crude product was purified by crystallisation from acetic acid to afford (1r,4r)-4-(5-(5-(3-(trifluoromethoxy)phenylamino)-1,3,4-oxadiazole-2-carboxamido)pyridin-2-yloxy)cyclohexanecarboxylic acid (0.075 g, 12.85%).

¹H NMR (400 MHz, DMSO) δ 1.37-1.57 (4H, m), 1.91-2.00 (2H, m), 2.06-2.15 (2H, m), 2.24-2.32 (1H, m), 4.85-4.94 (1H, m), 6.81 (1H, d), 7.06 (1H, d), 7.51-7.58 (2H, m), 7.71 (1H, s), 8.05-8.08 (1H, m), 8.52 (1H, d), 11.16 (1H, s), 11.38 (1H, s), 12.14 (1H, s). m/z 508 (M+H)⁺

Example 35 was synthesised in an analogous fashion from Intermediate 81

| Example | Structure | m/z | NMR |
|---|---|---|---|
| 35 (1r,4r)-4- (5-(5-(2,4- difluoro-5- (trifluoromethyl) phenylamino)- 1,3,4- oxadiazole-2- carboxamido) pyridin-2- yloxy) cyclohexane- carboxylic acid | 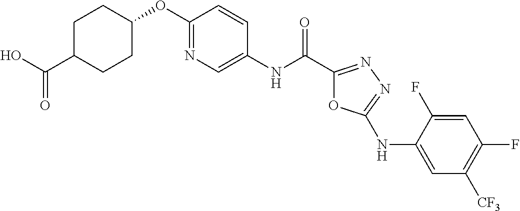 | 528 (M + H)⁺ | 1H NMR (400 MHz, DMSO) δ 1.38-1.54 (4H, m), 1.93-2.00 (2H, m), 2.09-2.12 (2H, m), 2.21-2.30 (1H, m), 4.84-4.93 (1H, m), 6.81 (1H, d), 7.82 (1H, t), 8.02-8.08 (1H, m), 8.51 (1H, d), 8.56 (1H, t), 11.17 (1H, s), 11.26 (1H, s), 12.14 (1H, s) |

### Example 36

### 2-((1s,4s)-4-(5-(5-(3,4-difluorophenylamino)-1,3,4-oxadiazole-2-carboxamido)pyridin-2-yloxy)cyclohexyl)acetic acid

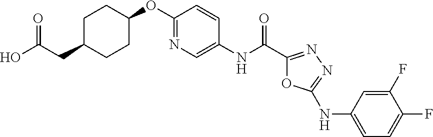

To a suspension of methyl 2-((1s,4s)-4-(5-(5-(3,4-difluorophenylamino)-1,3,4-oxadiazole-2-carboxamido)pyridin-2-yloxy)cyclohexyl)acetate (Intermediate 83) (378 mg, 0.78 mmol) in methanol (10 mL) was added 2N sodium hydroxide (1.939 mL, 3.88 mmol). The resulting solution was stirred at ambient temperature over the weekend. The reaction mixture was evaporated and the aqueous residue was adjusted to pH 2 with 2M hydrochloric acid. The suspension was filtered and dried to afford the product (331 mg, 90%).

¹H NMR (300 MHz, DMSO) δ 1.27-1.41 (2H, m), 1.53-1.66 (4H, m), 1.75-1.91 (3H, m), 2.16 (2H, d), 5.13-5.16 (1H, m), 6.81 (1H, d), 7.32-7.36 (1H, m), 7.42-7.53 (1H, m), 7.65-7.73 (1H, m), 8.04 (1H, d), 8.49 (1H, s), 11.08 (1H, s), 11.25 (1H, s); CO₂H not seen; m/z 474 (M+H)⁺.

### Example 37

### 2-((1r,4r)-4-(5-(5-(3,4-difluorophenylamino)-1,3,4-oxadiazole-2-carboxamido)pyridin-2-yloxy)cyclohexyl)acetic acid

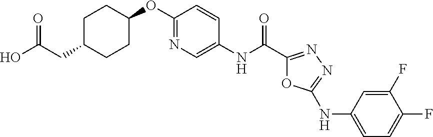

To a solution of methyl 2-((1r,4r)-4-(5-(5-(3,4-difluorophenylamino)-1,3,4-oxadiazole-2-carboxamido)pyridin-2-yloxy)cyclohexyl)acetate (Intermediate 90) (233 mg, 0.48 mmol) in methanol (10 mL) was added 2N sodium hydroxide (1.195 mL, 2.39 mmol). The resulting solution was stirred at ambient temperature overnight. The reaction mixture was evaporated and the aqueous residue was adjusted to pH 2 with 2M hydrochloric acid. The suspension was filtered and dried to afford crude product. The crude product was purified by preparative HPLC using decreasingly polar mixtures of water (containing 0.1% formic acid) and MeCN as eluents. Fractions containing the desired compound were evaporated to dryness to afford the product (42.0 mg, 18.6%).

¹H NMR (300 MHz, DMSO) δ 1.05-1.19 (2H, m), 1.31-1.44 (2H, m), 1.61-1.81 (3H, m), 2.03-2.10 (2H, m), 2.13 (2H, d), 4.81-4.91 (1H, m), 6.77 (1H, d), 7.32-7.37 (1H, m), 7.43-7.52 (1H, m), 7.64-7.72 (1H, m), 8.03 (1H, d), 8.49 (1H, s), 11.08 (1H, s), 11.24 (1H, s), 11.99 (1H, s); m/z 474 (M+H)⁺.

### Example 38

### 4-(5-(5-(3,4-Difluorophenylamino)-1,3,4-oxadiazole-2-carboxamido)-Pyridin-2-yloxy)benzoic acid

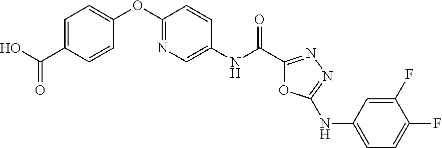

2M Sodium hydroxide (4.84 mL, 9.69 mmol) was added to methyl 4-(5-(5-(3,4-difluorophenylamino)-1,3,4-oxadiazole-2-carboxamido)pyridin-2-yloxy)benzoate (intermediate 95) (566 mg, 1.21 mmol) in methanol (40 mL). The resulting solution was stirred at ambient temperature for 24 hours. The mixture was acidified to ˜pH 2 with 2M HCl and the resulting solid was filtered off and washed with water. The solid was dried under high vac then recrystallised from acetic acid to yield the title compound (520 mg, 95%) as a white solid.

¹H NMR (400 MHz, DMSO-d6) δ 7.17-7.23 (3H, m), 7.34-7.38 (1H, m), 7.45-7.52 (1H, m), 7.67-7.73 (1H, m), 7.97-8.01 (2H, m), 8.29-8.32 (1H, m), 8.61 (1H, d), 11.26 (1H, s), 11.31 (1H, s), 12.50 (1H, s). m/z (ESI+) (M+H)+=454.

### Example 39

### 4-(5-(5-(3-Chloro-4-fluorophenylamino)-1,3,4-oxadiazole-2-carboxamido)pyridin-2-yloxy)benzoic acid

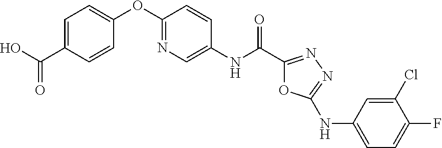

2M Sodium hydroxide (4.84 mL, 9.69 mmol) was added to methyl 4-(5-(5-(3-chloro-4-fluorophenylamino)-1,3,4-oxadiazole-2-carboxamido)pyridin-2-yloxy)benzoate (intermediate 96) (586 mg, 1.21 mmol) in methanol (40 mL). The resulting solution was stirred at ambient temperature for 24 hours. The mixture was acidified to ˜pH 2 with 2M HCl and the resulting solid was filtered off and washed with water. The solid was dried under high vac then recrystallised from acetic acid to yield the title compound (150 mg, 26.4%) as a white solid.

¹H NMR (400 MHz, DMSO-d6) δ 7.17-7.23 (3H, m), 7.47 (1H, t), 7.52-7.56 (1H, m), 7.82-7.84 (1H, m), 7.97-8.01 (2H, m), 8.29-8.32 (1H, m), 8.61 (1H, d), 11.24 (1H, s), 11.30 (1H, s), 12.48 (1H, s). m/z (ESI+) (M+H)+=470.

### Example 40

### (1s,4s)-4-(5-(5-(3,4-Difluorophenylamino)-1,3,4-oxadiazole-2-carboxamido)-4-methylpyridin-2-yloxy)cyclohexanecarboxylic acid

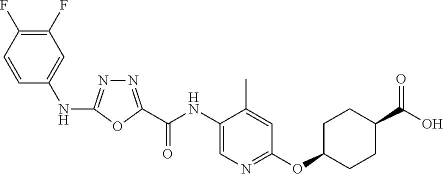

2M Sodium hydroxide (2.86 mL, 5.71 mmol) was added to (1s,4s)-methyl 4-(5-(5-(3,4-difluorophenylamino)-1,3,4-oxadiazole-2-carboxamido)-4-methylpyridin-2-yloxy)cyclohexanecarboxylate (intermediate 101) (696 mg, 1.43 mmol) in methanol (20 mL) under air. The resulting solution was stirred at ambient temperature for 16 hours. The reaction was acidified with 2M HCl and the resulting precipitate was filtered, washed with water (15 mL) then dried under high vac. This material was recrystallised from acetic acid to yield the title compound (0.65 acetic acid solvate) as a white solid (435 mg, 64.4%).

¹H NMR (400 MHz, DMSO-d6) δ 1.66-1.86 (8H, m), 2.18 (3H, s), 2.32-2.40 (1H, m), 5.09-5.19 (1H, m), 6.75 (1H, s), 7.33-7.36 (1H, m), 7.43-7.51 (1H, m), 7.66-7.72 (1H, m), 8.00 (1H, s), 10.58 (1H, s), 11.21 (1H, s), 12.00 (2H, s). m/z (ESI+) (M+H)+=474.

Examples 41-42 were prepared in an analogous way to Example 40, from intermediates 106-107 respectively.

| Example | Structure | Mass spec | ¹H NMR |
|---|---|---|---|
| 41: (1s,4s)-4-(5-(5-(3- Chloro-4- fluorophenylamino)-1,3,4- oxadiazole-2- carboxamido)- 4-methylpyridin-2- yloxy) cyclohexanecarboxylic acid (1.1 acetic acid solvate) | 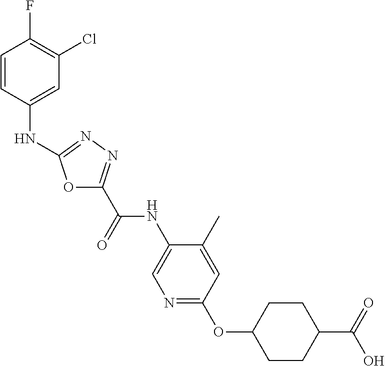 | m/z (ESI+) (M + H)+ = 490 | (400 MHz, DMSO-d6) δ 1.66-1.86 (8H, m), 2.18 (3H, s), 2.35-2.39 (1H, m), 5.10-5.18 (1H, m), 6.75 (1H, s), 7.45 (1H, t), 7.50-7.54 (1H, m), 7.81- 7.84 (1H, m), 8.00 (1H, s), 10.57 (1H, s), 11.20 (1H, s), 11.98 (2H, s) |
| 42: (1s,4s)-4-(5-(5-(4 - Chloro-3- fluorophenylamino)-1,3,4- oxadiazole-2- carboxamido)- 4-methylpyridin-2- yloxy) cyclohexanecarboxylic acid | 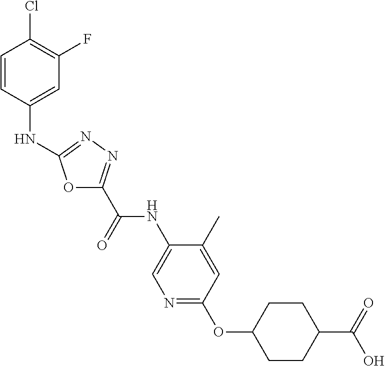 | m/z (ESI+) (M + H)+ = 490 | (400 MHz, DMSO-d6) δ 1.66-1.86 (8H, m), 2.18 (3H, s), 2.35-2.40 (1H, m), 5.08-5.14 (1H, m), 6.75 (1H, s), 7.36-7.39 (1H, m), 7.59 (1H, t), 7.67- 7.70 (1H, m), 8.00 (1H, s), 10.60 (1H, s), 11.36 (1H, s), 12.07 (1H, s) |

### Example 43

### (1s,4s)-4-(5-(5-(3,4-Difluorophenylamino)-1,3,4-oxadiazole-2-carboxamido)-4-methoxypyridin-2-yloxy)cyclohexanecarboxylic acid

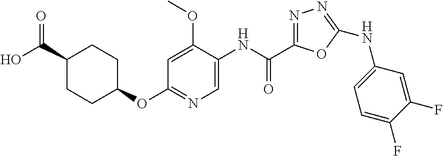

Lithium hydroxide mono hydrate (79 mg, 1.89 mmol) was added to a stirred suspension of (1s,4s)-methyl 4-(5-(5-(3,4-difluorophenylamino)-1,3,4-oxadiazole-2-carboxamido)-4-methoxypyridin-2-yloxy)cyclohexanecarboxylate (intermediate 108) (317 mg, 0.63 mmol) in MeOH (5 mL)/water (3 mL) at ambient temperature. The resulting solution was stirred at ambient temperature for 18 hours. The bulk of the organic solvent was removed in vacuo and the resulting aqueous solution was acidified to ˜pH 3 with 2M HCl. The resulting suspension was filtered and the solid was dried under high vac to yield the title compound (238 mg, 77%) as a white solid.

¹H NMR (400 MHz, DMSO-d6) δ 1.65-1.77 (4H, m), 1.77-1.89 (4H, m), 2.37-2.42 (1H, m), 3.88 (3H, s), 5.12-5.20 (1H, m), 6.51 (1H, s), 7.33-7.36 (1H, m), 7.49 (1H, q), 7.67-7.73 (1H, m), 8.13 (1H, s), 10.09 (1H, s), 11.26 (1H, s), 12.13 (1H, s). m/z (ESI+) (M+H)+=490.

### Example 44

### (1s,4s)-4-(5-(5-(4-Bromo-2-chlorophenylamino)-1,3,4-oxadiazole-2-carboxamido)-4-methoxypyridin-2-yl)oxy)cyclohexanecarboxylic acid

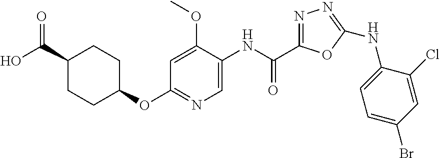

Lithium hydroxide mono hydrate (103 mg, 2.46 mmol) was added to a stirred suspension of (1s,4s)-methyl 4-(5-(5-(4-bromo-2-chlorophenylamino)-1,3,4-oxadiazole-2-carboxamido)-4-methoxypyridin-2-yloxy)cyclohexanecarboxylate (intermediate 113) (476 mg, 0.82 mmol) in MeOH (5 mL)/water (3 mL) at ambient temperature. The resulting solution was stirred at ambient temperature for 18 hours. The bulk of the organic solvent was removed in vacuo and the resulting aqueous solution was acidified to ˜pH 3 with 2M HCl. The resulting suspension was filtered and the solid was dried under high vac. The solid was recrystallised from acetic acid to yield the title compound (1.0 acetic acid solvate) as a white solid (116 mg, 24.97%).

¹H NMR (300 MHz, DMSO-d6) δ 1.66-1.90 (8H, m), 1.91 (3H, s), 2.37-2.41 (1H, m), 3.87 (3H, s), 5.10-5.20 (1H, m), 6.50 (1H, s), 7.62-7.66 (1H, m), 7.82 (1H, d), 7.97 (1H, d), 8.12 (1H, s), 10.04 (1H, s), 10.50 (1H, s), 12.01 (2H, s)

m/z (ESI+) (M+H)+=568

### Example 45

### 5-(3,4-Difluorophenylamino)-N-(6-((1s,4s)-4-(methylsulfonylcarbamoyl)cyclohexyloxy)pyridin-3-yl)-1,3,4-oxadiazole-2-carboxamide

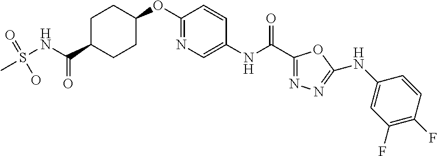

1-(3-Dimethylaminopropyl)-3-ethylcarbodiimide hydrochloride (47.6 mg, 0.25 mmol) was added to (1s,4s)-4-(5-(5-(3,4-difluorophenylamino)-1,3,4-oxadiazole-2-carboxamido)pyridin-2-yloxy)cyclohexanecarboxylic acid (Example 1) (57 mg, 0.12 mmol), methanesulfonamide (23.60 mg, 0.25 mmol) and 4-Dimethylaminopyridine (60.6 mg, 0.50 mmol) in DMF (5 mL). The resulting solution was stirred at ambient temperature for 100 hours. The solvent was evaporated in vacuo to yield crude material. The crude product was purified by preparative HPLC (Phenomenex Gemini C18 110A (axia) column, 5μ silica, 30 mm diameter, 100 mm length), using decreasingly polar mixtures of water (containing 0.1% formic acid) and MeCN as eluent. Fractions containing the desired compound were evaporated to dryness to afford the title compound (32.0 mg, 48.1%) as a white solid.

¹H NMR (400 MHz, DMSO-d6) δ 1.55-1.70 (4H, m), 1.70-1.85 (2H, m), 1.90-2.00 (2H, m), 2.40-2.45 (1H, m), 3.23 (3H, s), 5.15-5.20 (1H, m), 6.85 (1H, d), 7.35-7.38 (1H, m), 7.43-7.53 (1H, m), 7.67-7.73 (1H, m), 8.05-8.07 (1H, m), 8.50-8.53 (1H, m), 11.10 (1H, s), 11.24 (1H, s), 11.61 (1H, s). m/z (ESI+) (M+H)+=537.

### Example 46

### (1s,4s)-4-(3-Chloro-5-(5-(2,4-dichlorophenylamino)-1,3,4-oxadiazole-2-carboxamido)pyridin-2-yloxy)cyclohexanecarboxylic acid

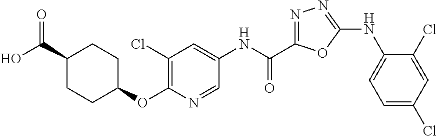

Sodium hydroxide (2 mL, 4.00 mmol) was added to (1s,4s)-methyl 4-(3-chloro-5-(5-(2,4-dichlorophenylamino)-1,3,4-oxadiazole-2-carboxamido)pyridin-2-yloxy)cyclohexanecarboxylate, intermediate 114, (410 mg, 0.76 mmol) in MeOH (5 mL). The resulting solution was stirred for 16 hours. The MeOH was removed by evaporation. The residue was neutralised with 2M HCl (2 mL), washed with water (5 mL), MeOH (10 mL) and then filtered off to give a solid. The crude product was purified by crystallisation from AcOH to afford the title compound (276 mg). ¹H NMR (400 MHz, DMSO) δ 1.64-1.91 (8H, m), 2.31-2.41 (1H, m), 5.21 (1H, s), 7.51 (1H, dd), 7.70 (1H, d), 8.01 (1H, d), 8.27 (1H, d), 8.47 (1H, d), 10.58 (1H, s), 11.23 (1H, s), 12.08 (1H, s). m/z (ESI+) (M+H)⁺=528.

### Example 47

### (1s,4s)-4-(3-Chloro-5-(5-(3,4-difluorophenylamino)-1,3,4-oxadiazole-2-carboxamido)pyridin-2-yloxy)cyclohexanecarboxylic acid

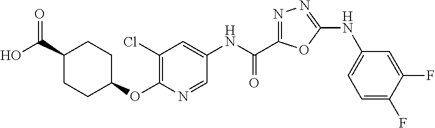

Sodium hydroxide (2 mL, 4.00 mmol) was added to (1s,4s)-methyl 4-(3-chloro-5-(5-(3,4-difluorophenylamino)-1,3,4-oxadiazole-2-carboxamido)pyridin-2-yloxy)cyclohexanecarboxylate (intermediate 119) (400 mg, 0.79 mmol) in MeOH (5 mL). The resulting solution was stirred for 16 hours. The MeOH was removed by evaporation. The residue was neutralised with 2M HCl (2 mL), washed with water (5 mL), MeOH (10 mL) and then filtered off to give a solid. The crude product was purified by crystallisation from acetic acid to afford (1s,4s)-4-(3-chloro-5-(5-(3,4-difluorophenylamino)-1,3,4-oxadiazole-2-carboxamido)pyridin-2-yloxy)cyclohexanecarboxylic acid (231 mg).

¹H NMR (400 MHz, DMSO) δ 1.63-1.91 (8H, m), 2.31-2.41 (1H, m), 5.22 (1H, s), 7.31-7.37 (1H, m), 7.47 (1H, q), 7.64-7.72 (1H, m), 8.28 (1′-1, d), 8.48 (1H, d), 11.25 (2H, s), 12.07 (1H, s). m/z (ESI+) (M+H)⁺=494.

### Example 48

### (1s,4s)-4-(3-Chloro-5-(5-(3-chloro-4-fluorophenylamino)-1,3,4-oxadiazole-2-carboxamido)pyridin-2-yloxy)cyclohexanecarboxylic acid

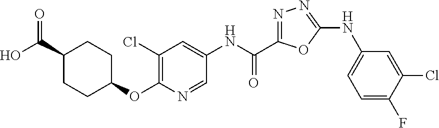

Sodium hydroxide (2 mL, 4.00 mmol) was added to (1s,4s)-methyl 4-(3-chloro-5-(5-(3-chloro-4-fluorophenylamino)-1,3,4-oxadiazole-2-carboxamido)pyridin-2-yloxy)cyclohexanecarboxylate (intermediate 120) (410 mg, 0.78 mmol) in MeOH (5 mL). The resulting solution was stirred for 16 hours. The MeOH was removed by evaporation. The residue was neutralised with 2M HCl (2 mL), washed with water (5 mL), MeOH (10 mL) and then filtered off to give a solid. The crude product was purified by crystallisation from AcOH to afford (1s,4s)-4-(3-chloro-5-(5-(3-chloro-4-fluorophenylamino)-1,3,4-oxadiazole-2-carboxamido)pyridin-2-yloxy)cyclohexanecarboxylic acid (143 mg).

¹H NMR (400 MHz, DMSO) δ 1.63-1.92 (8H, m), 2.31-2.42 (1H, m), 5.22 (1H, s), 7.46 (1H, t), 7.50-7.55 (1H, m), 7.81 (1H, d), 8.28 (1H, d), 8.48 (1H, d), 11.25 (2H, s), 12.07 (1H, s), m/z (ESI+) (M+H)⁺=510.

### Example 49

### (1R,3R)-3-(5-(5-(3,4-Difluorophenylamino)-1,3,4-oxadiazole-2-carboxamido)pyridin-2-yloxy)cyclopentanecarboxylic acid

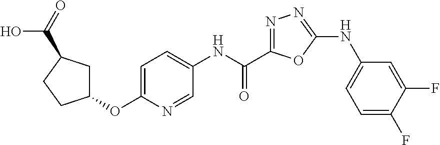

2M Sodium hydroxide (2 mL, 4.00 mmol) was added to (1R,3R)-methyl 3-(5-(5-(3,4-difluorophenylamino)-1,3,4-oxadiazole-2-carboxamido)pyridin-2-yloxy)cyclopentanecarboxylate (intermediate 121) (423 mg, 0.92 mmol) in MeOH (5 mL). The resulting solution was stirred for 16 hours. The reaction mixture was concentrated under vacuum. 2M HCl (2 mL), then water (5 mL) added. A solid was filtered off. This was washed with MeOH (5 mL), and dried under vacuum to give crude product. The crude product was purified by crystallisation from acetic acid to afford (1R,3R)-3-(5-(5-(3,4-difluorophenylamino)-1,3,4-oxadiazole-2-carboxamido)pyridin-2-yloxy)cyclopentanecarboxylic acid (207 mg).

¹H NMR (400 MHz, DMSO) δ 1.68-1.83 (2H, m), 1.93-2.16 (4H, m), 2.85-2.96 (1H, m), 5.34-5.41 (1H, m), 6.80 (1H, d), 7.32-7.37 (1H, m), 7.47 (1H, q), 7.65-7.73 (1H, m), 8.04 (1H, dd), 8.52 (1H, d), 11.10 (1H, s), 11.25 (1H, s), m/z (ESI+) (M+H)⁺=446.

### Example 50

### (1s,4s)-4-(5-(5-(3,4-Difluorophenylamino)-1,3,4-oxadiazole-2-carboxamido)-6-fluoropyridin-2-yloxy)cyclohexanecarboxylic acid

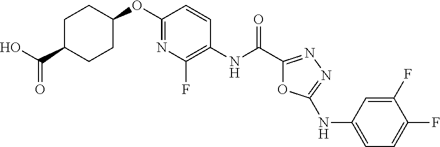

To a suspension of (1s,4s)-methyl 4-(5-(5-(3,4-difluorophenylamino)-1,3,4-oxadiazole-2-carboxamido)-6-fluoropyridin-2-yloxy)cyclohexanecarboxylate (intermediate 126) (0.973 g, 1.98 mmol) in MeOH (20 mL) was added 2M sodium hydroxide (4.95 mL, 9.90 mmol). The resulting solution was stirred at ambient temperature overnight. The reaction was incomplete so the temperature was increased to 50° C. and further 2M sodium hydroxide (0.5 mL, 1.0 mmol) was added and the solution was stirred at 50° C. for a further 7 hours and allowed to stir at ambient temperature overnight. The reaction mixture was evaporated and the aqueous residue was adjusted to pH 2 with 2M HCl. The suspension was filtered and dried to afford the desired product. The solid was slurried in acetonitrile (7 mL) at 50° C. overnight and the resulting solid filtered and dried to yield (1s,4s)-4-(5-(5-(3,4-difluorophenylamino)-1,3,4-oxadiazole-2-carboxamido)-6-fluoropyridin-2-yloxy)cyclohexanecarboxylic acid (0.541 g, 57.2%) as a white solid.

¹H NMR (400 MHz, DMSO) δ 1.67-1.84 (8H, m), 2.37-2.40 (1H, m), 5.00 (1H, m), 6.80 (1H, d), 7.33-7.36 (1H, m), 7.43-7.51 (1H, m), 7.66-7.71 (1H, m), 7.87-7.92 (1H, m), 10.77 (1H, s), 11.23 (1H, s), 12.09 (1H, s). m/z 478 (M+H)⁺

### Example 51

### (1s,4s)-4-(5-(5-(3,4-Difluorophenylamino)-1,3,4-oxadiazole-2-carboxamido)-6-methoxypyridin-2-yloxy)cyclohexanecarboxylic acid

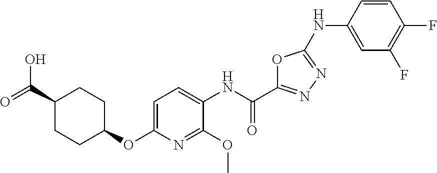

A solution of 2M Sodium hydroxide (aq) (1.291 mL, 2.58 mmol) was added to a stirred suspension of (1s,4s)-methyl 4-(5-(5-(3,4-difluorophenylamino)-1,3,4-oxadiazole-2-carboxamido)-6-methoxypyridin-2-yloxy)cyclohexanecarboxylate (intermediate 133) (0.26 g, 0.52 mmol) in methanol (10 mL) at 20° C. The resulting solution was stirred at 20° C. for 20 hours. Half the methanol was evaporated and the reaction mixture cooled to 0° C. The reaction mixture was acidified with 2M HCl. The precipitate was collected by filtration, washed with water (50 mL) and methanol (50 mL) and dried under vacuum to afford crude product. The crude product was purified by crystallisation from AcOH to afford (1s,4s)-4-(5-(5-(3,4-difluorophenylamino)-1,3,4-oxadiazole-2-carboxamido)-6-methoxypyridin-2-yloxy)cyclohexanecarboxylic acid (0.110 g, 43.5%).

¹H NMR (400 MHz, DMSO) δ 1.66-1.90 (8H, m), 2.35-2.43 (1H, m), 3.91 (3H, s), 5.07-5.13 (1H, m), 6.42 (1H, d), 7.33-7.38 (1H, m), 7.47 (1H, q), 7.65-7.74 (1H, m), 7.90 (1H, d), 9.89 (1H, s), 11.21 (1H, s), 12.07 (1H, s). m/z 490 (M+H)⁺

### Example 52

### (1s,4s)-4-(5-(5-(3-Chloro-4-fluorophenylamino)-1,3,4-oxadiazole-2-carboxamido)-6-methoxypyridin-2-yloxy)cyclohexanecarboxylic acid

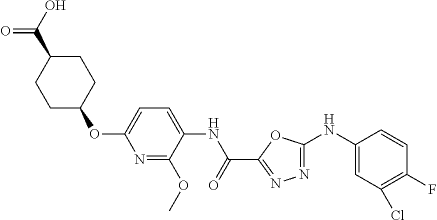

Synthesised by the method of Example 51 from the appropriate ester (intermediate 138).

¹H NMR (400 MHz, DMSO) δ 1.67-1.89 (8H, m), 2.36-2.43 (1H, m), 3.91 (3H, s), 5.07-5.13 (1H, m), 6.42 (1H, d), 7.44-7.55 (2H, m), 7.82-7.85 (1H, m), 7.90 (1H, d), 9.92 (1H, s), 11.21 (1H, s), 12.06 (1H, s). m/z 506 (M+H)⁺

### Example 53

### 2-((1s,4s)-4-(5-(5-(3-Chloro-4-fluorophenylamino)-1,3,4-oxadiazole-2-carboxamido)pyridin-2-yloxy)cyclohexyl)acetic acid

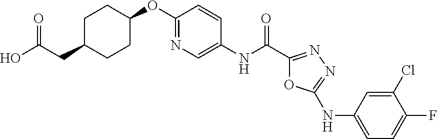

To a suspension of methyl 2-((1s,4s)-4-(5-(5-(3-chloro-4-fluorophenylamino)-1,3,4-oxadiazole-2-carboxamido)pyridin-2-yloxy)cyclohexyl)acetate (intermediate 139) (1.007 g, 2.00 mmol) in MeOH (15 mL) was added 2M sodium hydroxide (5.00 mL, 9.99 mmol). The resulting solution was stirred at ambient temperature for 4 hours. The reaction mixture was evaporated and the aqueous residue was adjusted to pH 2 with 2M HCl. The suspension was filtered and dried to afford crude product as a white solid, 210 mgs. This was recrystallised from glacial acetic acid (6 mL).

The crude product was dissolved in DMSO/MeCN/water (7:2:1, ˜15 mL), but it didn't completely dissolve so the suspension was filtered and dried to leave 273 mg of pure product and the filtrate was purified by preparative HPLC, using decreasingly polar mixtures of water (containing 0.1% formic acid) and MeCN as eluents. The fractions containing the desired compound were evaporated to dryness to afford 2-((1s,4s)-4-(5-(5-(3-chloro-4-fluorophenylamino)-1,3,4-oxadiazole-2-carboxamido)pyridin-2-yloxy)cyclohexyl)acetic acid (134 mg, 13.69%) as a cream solid.

¹H NMR (400 MHz, DMSO) δ 1.31-1.42 (2H, m), 1.52-1.67 (4H, m), 1.77-1.93 (3H, m), 2.17 (2H, d), 5.14-5.18 (1H, m), 6.82 (1H, d), 7.43-7.56 (2H, m), 7.83 (1H, d), 8.06 (1H, d), 8.50 (1H, s), 11.09 (1H, s), 11.23 (1H, s), 11.98 (1H, s); m/z 490 (M+H)⁺.

### Example 54

### 2-((1s,4s)-4-(5-(5-(2,4-Dichlorophenylamino)-1,3,4-oxadiazole-2-carboxamido)pyridin-2-yloxy)cyclohexyl)acetic acid

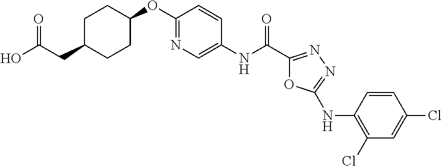

To a suspension of methyl 2-((1s,4s)-4-(5-(5-(2,4-dichlorophenylamino)-1,3,4-oxadiazole-2-carboxamido)pyridin-2-yloxy)cyclohexyl)acetate (intermediate 140) (1.040 g, 2.00 mmol) in MeOH (15 mL) was added 2M sodium hydroxide (5.00 mL, 9.99 mmol). The resulting solution was stirred at ambient temperature for 4 hours. The reaction mixture was evaporated and the aqueous residue was adjusted to pH 2 with 2M HCl. The suspension was filtered and dried to afford crude product as a white solid. The crude product was dissolved in DMSO/MeCN/water (7:2:1, ˜20 mL) and purified by preparative HPLC using decreasingly polar mixtures of water (containing 0.1% formic acid) and MeCN as eluents. Fractions containing the desired compound were evaporated to dryness to afford 2-((1s,4s)-4-(5-(5-(2,4-dichlorophenylamino)-1,3,4-oxadiazole-2-carboxamido)pyridin-2-yloxy)cyclohexyl)acetic acid (91 mg, 8.94%) as a beige solid.

¹H NMR (400 MHz, DMSO) δ 1.31-1.41 (2H, m), 1.52-1.66 (4H, m), 1.77-1.92 (3H, m), 2.17 (2H, d), 5.14-5.18 (1H, m), 6.82 (1H, d), 7.52 (1H, d), 7.71 (1H, s), 8.02-8.06 (2H, m), 8.49 (1H, s), 10.56 (1H, s), 11.08 (1H, s), 12.00 (1H, s); m/z 506 (M+H)⁺.

### Example 55

### (1s,4s)-4-(5-(5-(3-Chloro-4-fluorophenylamino)-1,3,4-oxadiazole-2-carboxamido)-6-fluoropyridin-2-yloxy)cyclohexanecarboxylic acid

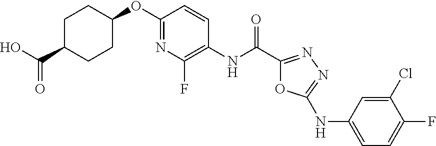

To a suspension of (1s,4s)-methyl 4-(5-(5-(3-chloro-4-fluorophenylamino)-1,3,4-oxadiazole-2-carboxamido)-6-fluoropyridin-2-yloxy)cyclohexanecarboxylate (intermediate 141) (0.452 g, 0.89 mmol) in MeOH (15 mL) was added 2M sodium hydroxide (2.225 mL, 4.45 mmol). The resulting solution was stirred at ambient temperature overnight. The reaction was incomplete and further 2M sodium hydroxide (1 mL, 2 mmol) was added and the solution was stirred at ambient temperature for a further 5 hours. The reaction mixture was evaporated and the aqueous residue was adjusted to pH 2 with 2M HCl. The suspension was filtered and dried to afford the desired product. This recrystallised from boiling EtOH (80 mL), the solution was allowed to slowly cool and the suspension was filtered and dried to afford (1s,4s)-4-(5-(5-(3-chloro-4-fluorophenylamino)-1,3,4-oxadiazole-2-carboxamido)-6-fluoropyridin-2-yloxy)cyclohexanecarboxylic acid (0.150 g, 34.1%) as a white solid.

¹H NMR (400.132 MHz, DMSO) δ 1.60-1.81 (8H, m), 2.29-2.36 (1H, m), 4.92-4.97 (1H, m), 6.74 (1H, d), 7.40 (1H, t), 7.44-7.48 (1H, m), 7.76-7.78 (1H, m), 7.84 (1H, t), 10.71 (1H, s), 11.16 (1H, s), 12.04 (1H, s); m/z 494 (M+H)⁺.

### Example 56

### (1s,4s)-4-(5-(5-(3-Chloro-4-fluorophenylamino)-1,3,4-oxadiazole-2-carboxamido)-3-methoxypyridin-2-yloxy)cyclohexanecarboxylic acid

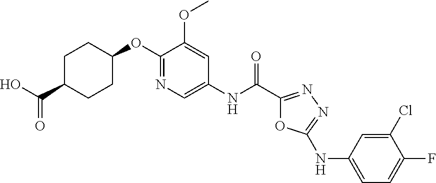

To a suspension of (1s,4s)-methyl 4-(5-(5-(3-chloro-4-fluorophenylamino)-1,3,4-oxadiazole-2-carboxamido)-3-methoxypyridin-2-yloxy)cyclohexanecarboxylate (intermediate 142) (0.395 g, 0.76 mmol) in MeOH (15 mL) was added 2M sodium hydroxide (1.900 mL, 3.80 mmol). The resulting solution was stirred at ambient temperature over the weekend. The reaction mixture was evaporated and the aqueous residue was adjusted to pH 2 with 2M HCl. The suspension was filtered and dried to afford the desired product. This recrystallised from boiling glacial AcOH (30 mL), the solution was allowed to slowly cool and the suspension was filtered and dried to afford (1s,4s)-4-(5-(5-(3-chloro-4-fluorophenylamino)-1,3,4-oxadiazole-2-carboxamido)-3-methoxypyridin-2-yloxy)cyclohexanecarboxylic acid (0.158 g, 41.1%) as a white solid. ¹H NMR (400 MHz, DMSO) δ 1.64-1.85 (8H, m), 2.35-2.42 (1H, m), 3.79 (3H, s), 5.13-5.18 (1H, m), 7.48 (1H, t), 7.53-7.56 (1H, m), 7.73 (1H, s), 7.84 (1H, d), 8.13 (1H, s), 11.09 (1H, s), 11.29 (1H, s); CO₂H not seen; m/z 506 (M+H)⁺.

### Example 57

### (1s,4s)-4-(5-(5-(3,4-Difluorophenylamino)-1,3,4-oxadiazole-2-carboxamido)-3-methoxypyridin-2-yloxy)cyclohexanecarboxylic acid

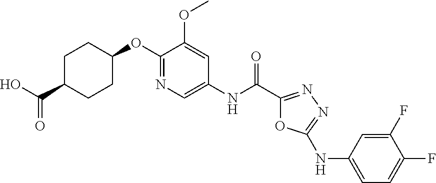

To a suspension of (1s,4s)-methyl 4-(5-(5-(3,4-difluorophenylamino)-1,3,4-oxadiazole-2-carboxamido)-3-methoxypyridin-2-yloxy)cyclohexanecarboxylate (intermediate 143) (0.388 g, 0.77 mmol) in MeOH (10 mL) was added 2M sodium hydroxide (1.925 mL, 3.85 mmol). The resulting solution was stirred at ambient temperature over the weekend. The reaction mixture was evaporated and the aqueous residue was adjusted to pH 2 with 2M HCl. The suspension was filtered and dried to afford the desired product. This recrystallised from boiling glacial AcOH (70 mL), the solution was allowed to slowly cool and the suspension was filtered and dried to afford (1s,4s)-4-(5-(5-(3,4-difluorophenylamino)-1,3,4-oxadiazole-2-carboxamido)-3-methoxypyridin-2-yloxy)cyclohexanecarboxylic acid (0.095 g, 25.2%) as a white solid.

¹H NMR (400 MHz, DMSO) δ 1.63-1.85 (8H, m), 2.35-2.43 (1H, m), 3.80 (3H, s), 5.13-5.18 (1H, m), 7.35-7.39 (1H, m), 7.49 (1H, q), 7.68-7.72 (1H, m), 7.74 (1H, s), 8.12 (1H, s), 11.09 (1H, s), 11.31 (1H, s); CO₂H not seen; m/z 490 (M+H)⁺.

### Example 58

### (1s,4s)-4-(4-Fluoro-5-(5-(2,4,5-trifluorophenylamino)-1,3,4-oxadiazole-2-carboxamido)pyridin-2-yloxy)cyclohexanecarboxylic acid

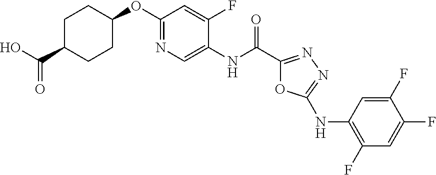

2M sodium hydroxide (2.037 mL, 4.07 mmol) was added to a stirred suspension of (1s,4s)-methyl 4-(4-fluoro-5-(5-(2,4,5-trifluorophenylamino)-1,3,4-oxadiazole-2-carboxamido)pyridin-2-yloxy)cyclohexanecarboxylate (intermediate 149) (415 mg, 0.81 mmol). The resulting red solution was stirred at ambient temperature for 4 hours and then at 50° C. for 3 hours and then at ambient temperature overnight. The reaction mixture was evaporated and the aqueous residue was adjusted to pH 2 with 2M HCl. The suspension was filtered and dried to afford the desired product. This recrystallised from boiling EtOH (7 mL), the solution was allowed to slowly cool and the suspension was filtered and dried to afford (1s,4s)-4-(4-fluoro-5-(5-(2,4,5-trifluorophenylamino)-1,3,4-oxadiazole-2-carboxamido)pyridin-2-yloxy)cyclohexanecarboxylic acid (190 mg, 47.1%) as a white solid.

¹H NMR (400 MHz, DMSO) δ 1.66-1.90 (8H, m), 2.36-2.40 (1H, m), 5.13-5.16 (1H, m), 6.87 (1H, d), 7.66-7.73 (1H, m), 8.11-8.17 (1H, m), 8.20 (1H, d), 10.84 (1H, s), 11.07 (1H, s), 12.09 (1H, s); m/z 496 (M+H)⁺.

### Example 59

### (1s,4s)-4-(5-(5-(3,4-Difluorophenylamino)-1,3,4-oxadiazole-2-carboxamido)-4-fluoropyridin-2-yloxy)cyclohexanecarboxylic acid

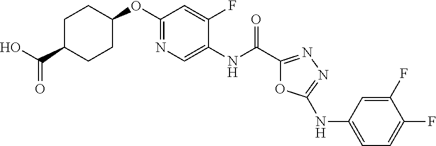

A solution of lithium hydroxide monohydrate (168 mg, 4.00 mmol) in water (7.5 mL) was added to a stirred suspension of (1s,4s)-methyl 4-(5-(5-(3,4-difluorophenylamino)-1,3,4-oxadiazole-2-carboxamido)-4-fluoropyridin-2-yloxy)cyclohexanecarboxylate (intermediate 150) (393 mg, 0.80 mmol) in MeOH (15 mL). The resulting solution was stirred at ambient temperature overnight. The reaction mixture was evaporated and the aqueous residue was adjusted to pH 2 with 1N citric acid. The suspension was filtered and dried to afford the desired product. This recrystallised from boiling EtOH (7 mL), the solution was allowed to slowly cool and the suspension was filtered and dried to afford (1s,4s)-4-(5-(5-(3,4-difluorophenylamino)-1,3,4-oxadiazole-2-carboxamido)-4-fluoropyridin-2-yloxy)cyclohexanecarboxylic acid (205 mg, 53.7%) as a white solid.

¹H NMR (400 MHz, DMSO) δ 1.66-1.93 (8H, m), 2.36-2.40 (1H, m), 5.14-5.18 (1H, m), 6.85-6.88 (1H, m), 7.33-7.35 (1H, m), 7.44-7.51 (1H, m), 7.66-7.71 (1H, m), 8.21 (1H, d), 10.83 (1H, s), 11.24 (1H, s), 12.09 (1H, s); m/z 478 (M+H)⁺.

### Example 60

### (1s,4s)-4-(5-(5-(3,4-Difluorophenylamino)-1,3,4-oxadiazole-2-carboxamido)-3-fluoropyridin-2-yloxy)cyclohexanecarboxylic acid

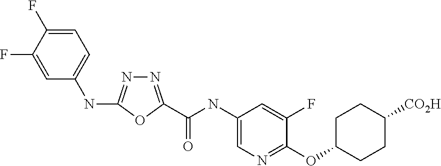

2M Sodium hydroxide (2.125 mL, 4.25 mmol) was added to (1s,4s)-methyl 4-(5-(5-(3,4-difluorophenylamino)-1,3,4-oxadiazole-2-carboxamido)-3-fluoropyridin-2-yloxy)cyclohexanecarboxylate (intermediate 157) (0.418 g, 0.85 mmol) in MeOH (5 mL) at 20° C. The resulting solution was stirred at room temperature for 5 hours. The reaction mixture was cooled to 0° C. and acidified with 2M HCl.

The precipitate was collected by filtration, washed with water (10 mL) and dried under vacuum to afford the crude product as a cream coloured solid.

The crude product was purified by crystallisation from AcOH to afford (1s,4s)-4-(5-(5-(3,4-difluorophenylamino)-1,3,4-oxadiazole-2-carboxamido)-3-fluoropyridin-2-yloxy)cyclohexanecarboxylic acid (0.250 g, 61.6%) as a white crystalline solid.

¹H NMR (400 MHz, DMSO) δ1.65-1.90 (m, 8H), 2.35-2.45 (m, 1H), 5.19-5.25 (m, 1H), 7.32-7.38 (m, 1H), 7.50 (q, 1H), 7.66-7.74 (m, 1H), 8.06-8.12 (m, 1H), 8.39 (d, 1H), 11.32 (s, 1H), 11.34 (s, 1H), 12.16 (s, 1H). m/z (ESI+) (M+H)⁺=478.22.

### Example 61

### (1s,4s)-4-(5-(5-(3-Chloro-4-fluorophenylamino)-1,3,4-oxadiazole-2-carboxamido)-3-fluoropyridin-2-yloxy)cyclohexanecarboxylic acid

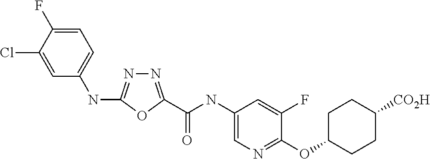

2M Sodium hydroxide (2.125 mL, 4.25 mmol) was added to (1s,4s)-methyl 4-(5-(5-(3-chloro-4-fluorophenylamino)-1,3,4-oxadiazole-2-carboxamido)-3-fluoropyridin-2-yloxy)cyclohexanecarboxylate (intermediate 164) (0.432 g, 0.85 mmol) in methanol (5 mL) at 20° C. The resulting solution was stirred at room temperature for 4 hours.

The reaction mixture was cooled to 0° C. and acidified with 2M HCl.

The precipitate was collected by filtration, washed with water (10 mL) and dried under vacuum to afford the crude product as a cream coloured solid.

The crude product was purified by crystallisation from AcOH to afford (1s,4s)-4-(5-(5-(3-chloro-4-fluorophenylamino)-1,3,4-oxadiazole-2-carboxamido)-3-fluoropyridin-2-yloxy)cyclohexanecarboxylic acid (0.190 g, 45.3%) as a white crystalline solid.

¹H NMR (400 MHz, DMSO) δ1.66-1.92 (m, 8H), 2.35-2.45 (m, 1H), 5.18-5.25 (m, 1H), 7.45-7.57 (m, 2H), 7.81-7.85 (m, 1H), 8.06-8.12 (m, 1H), 8.39 (d, 1H), 11.30 (s, 1H), 11.34 (s, 1H), 12.16 (s, 1H). m/z (ESI+) (M+H)+=494.17.

### Example 62

### (1s,4s)-4-(5-(5-(3,4-Difluorophenylamino)-1,3,4-oxadiazole-2-carboxamido)-6-methylpyridin-2-yloxy)cyclohexanecarboxylic acid

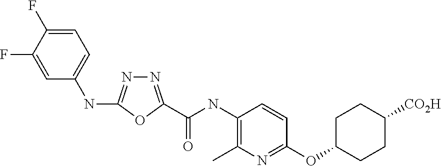

2M Sodium hydroxide (1.195 mL, 2.39 mmol) was added to (1s,4s)-methyl 4-(5-(5-(3,4-difluorophenylamino)-1,3,4-oxadiazole-2-carboxamido)-6-methylpyridin-2-yloxy)cyclohexanecarboxylate (intermediate 165) (233 mg, 0.48 mmol) in methanol (5 mL) at room temperature. The resulting solution was stirred at room temperature for 5 hours. The reaction mixture was cooled in an ice bath and acidified with 2M HCl. The precipitate was collected by filtration, washed with water (10 mL) and dried under vacuum to afford crude product. This was purified by crystallisation from acetic acid to give pure (1s,4s)-4-(5-(5-(3,4-difluorophenylamino)-1,3,4-oxadiazole-2-carboxamido)-6-methylpyridin-2-yloxy)cyclohexanecarboxylic acid (27.0 mg, 11.93%).

¹H NMR (400.132 MHz, DMSO) δ1.64-1.91 (m, 8H), 2.32 (s, 3H), 2.34-2.43 (m, 1H), 5.11-5.19 (m, 1H), 6.67 (d, 1H), 7.31-7.53 (m, 2H), 7.59 (d, 1H), 7.66-7.76 (m, 1H), 10.61 (s, 1H), 11.23 (s, 1H), CO₂H not observed. m/z (ESI+) (M+H)⁺=474.42.

### Examples 63 and 64

### cis- and trans-3-(5-(5-(4-Isopropylphenylamino)-1,3,4-oxadiazole-2-carboxamido)pyridin-2-yloxy)cyclobutanecarboxylic acid

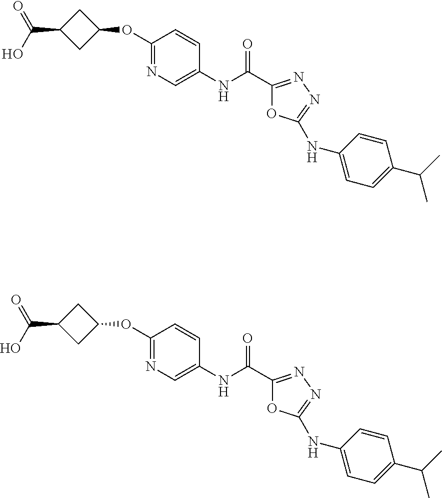

An aqueous solution of sodium hydroxide (5.88 mL, 11.76 mmol) was added to a stirred suspension of phenethyl 3-(5-(5-(4-isopropylphenylamino)-1,3,4-oxadiazole-2-carboxamido)pyridin-2-yloxy)cyclobutanecarboxylate (cis/trans mixture; intermediate 172) (1.274 g, 2.35 mmol) in ethanol (20 mL) at room temperature. The resulting solution was stirred at room temperature for 7 hours. The reaction mixture was evaporated to dryness and the residue neutralised with 1M HCl (11.8 mL). The formed precipitate was collected by filtration, washed with water and dried under vacuum to afford crude product. The crude product was purified by crystallisation from AcOH to afford 3-(5-(5-(4-isopropylphenylamino)-1,3,4-oxadiazole-2-carboxamido)pyridin-2-yloxy)cyclobutanecarboxylic acid (491 mg, 78%) as a white solid (mixture of isomers). The crude product was purified by preparative chiral-HPLC on a Chiralpak AD column, eluting isocratically with EtOH/HOAC 99.9/0.1 as eluent. The fractions containing the desired compounds were evaporated to dryness to afford cis-3-(5-(5-(4-isopropylphenylamino)-1,3,4-oxadiazole-2-carboxamido)pyridin-2-o yloxy)cyclobutanecarboxylic acid (235 mg) as a white solid and trans-3-(5-(5-(4-isopropylphenylamino)-1,3,4-oxadiazole-2-carboxamido)pyridin-2-yloxy)cyclobutanecarboxylic acid (135 mg) as a white solid.

Cis- (Example 63): δ_{H}(400 MHz, DMSO-d₆) 1.20 (6H, d), 2.14-2.21 (2H, m), 2.63-2.69 (2H, m), 2.73-2.82 (1H, m), 2.83-2.90 (1H, m), 5.02-5.10 (1H, m), 6.84 (1H d, J=8.9 Hz), 7.27 (2H d, J=8.9 Hz), 7.51 (2H d, J=8.9 Hz), 8.06-8.08 (1H, m), 8.52 (1H d, J=2.7 Hz), 10.90 (1H, s), 11.12 (1H, s), 12.22 (1H, s). m/z (ESI+) (M+H)⁺=438.

Trans- (Example 64): δ_{H}(400 MHz, DMSO-d₆) 1.20 (6H, d), 2.27-2.34 (2H, m), 2.57-2.63 (2H, m), 2.83-2.90 (1H, m), 2.98-3.04 (1H, m), 5.22-5.29 (1H, m), 6.84 (1H d, J=8.9 Hz), 7.26 (2H d, J=8.9 Hz), 7.50-7.53 (2H, m), 8.06-8.09 (1H, m), 8.51 (1H d, J=2.7 Hz), 11.11 (2H, s), 12.50 (1H, s). m/z (ESI+) (M+H)⁺=438.

### Examples 65 and 66: cis- and trans-3-(5-(5-(3,4-Difluorophenylamino)-1,3,4-oxadiazole-2-carboxamido)pyridin-2-yloxy)cyclobutanecarboxylic acid

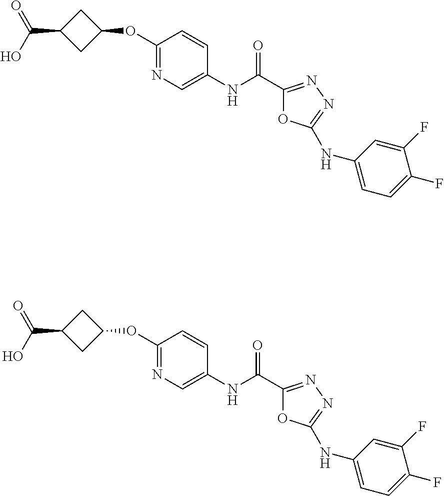

The mixture of isomers was synthesised from phenethyl 3-(5-(5-(3,4-difluorophenylamino)-1,3,4-oxadiazole-2-carboxamido)pyridin-2-yloxy)cyclobutanecarboxylate (cis/trans mixture; intermediate 179) in an analogous manner to that described for Examples 63 and 64 above.

The crude product was purified by preparative chiral-HPLC on a Chiralpak AD column, eluting isocratically with EtOH/MeOH/HOAC 50/50/0.1. as eluent. The fractions containing the desired compounds were evaporated to dryness to afford cis-3-(5-(5-(3,4-difluorophenylamino)-1,3,4-oxadiazole-2-carboxamido)pyridin-2-yloxy)cyclobutanecarboxylic acid (250 mg) and trans-3-(5-(5-(3,4-difluorophenylamino)-1,3,4-oxadiazole-2-carboxamido)pyridin-2-yloxy)cyclobutanecarboxylic acid (145 mg) as a white solids.

Cis-(Example 65) δ_{H}(400 MHz, DMSO-d₆) 2.14-2.21 (2H, m), 2.64-2.69 (2H, m), 2.73-2.80 (1H, m), 5.02-5.10 (1H, m), 6.84 (1H d, 8.9 Hz), 734-7.37 (1H, m), 7.46-7.51 (1H, m), 7.67-7.73 (1H, m), 8.06-8.09 (1H, m), 8.52 (1H d, J=2.7 Hz), 11.17 (1H, s), 11.20-12.50 (2H, m). m/z (ESI+) (M+H)⁺=432.

Trans-(Example 66) δ_{H}(400 MHz, DMSO-d₆) 2.28-2.35 (2H, m), 2.57-2.63 (2H, m), 3.00-3.07 (1H, m), 5.22-5.29 (1H, m), 6.84 (1H d, J=8.9 Hz), 7.33-7.36 (1H, m), 7.48 (1H d, J=10.7 Hz), 7.67-7.73 (1H, m), 8.06-8.09 (1H, m), 8.51 (1H d, J=2.7 Hz), 11.16 (1H, s), 11.85 (2H, br s). m/z (ESI+) (M+H)⁺=432.

### Example 67

### 4-(5-((5-((2,4-dichlorophenyl)amino)-1,3,4-oxadiazole-2-carbonyl)amino)pyridin-2-yl)oxy-1-methylcyclohexane-1-carboxylic acid

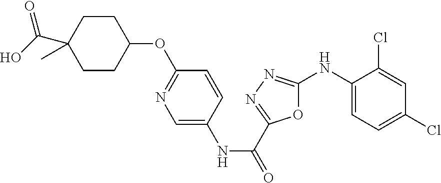

Ethyl 4-(5-((5-((2,4-dichlorophenyl)amino)1,3,4-oxadiazole-2-carbonyl)amino)pyridin-2-yl)oxy-1-methylcyclohexane-1-carboxylate (intermediate 180) (0.245 g, 0.46 mmol) and potassium trimethylsilanolate (0.412 g, 3.21 mmol) were suspended in THF (4.58 mL) and sealed into a microwave tube. The reaction was heated to 90° C. for 2 hours in the microwave reactor and cooled to RT. The product precipitated out of solution upon cooling. The precipitate was collected by filtration, washed with THF (5 mL) and dried under vacuum to afford 4-(5-((5-((2,4-dichlorophenyl)amino)1,3,4-oxadiazole-2-carbonyl)amino)pyridin-2-yl)oxy-1-methylcyclohexane-1-carboxylic acid as a yellow solid. The crude product was purified by preparative HPLC, using decreasingly polar mixtures of water (containing 0.1% formic acid) and MeCN as eluents. Fractions containing the desired compound were evaporated to dryness to afford 4-(5-((5-((2,4-dichlorophenyl)amino)1,3,4-oxadiazole-2-carbonyl)amino)pyridin-2-yl)oxy-1-methylcyclohexane-1-carboxylic acid as a white solid which was recrystallized in hot ethanol to yield (0.067 g, 28.9%) with a cis:trans ratio of 5:1.

Cis isomer: ¹H NMR (400 MHz, DMSO-d₆) δ 1.17 (3H, s), 1.53-1.60 (2H, m), 1.75 (3H, q), 1.81-1.87 (2H, m), 5.04-5.09 (1H, m), 6.82 (1′-1, d), 7.46-7.52 (1H, m), 7.69-7.69 (1H, m), 8.02-8.08 (2H, m), 8.50 (1H, d), 10.63 (1H, s), 11.05 (1H, s), 12.12 (1H, s). m/z 506 (M+H)+

Trans isomer: ¹H NMR (400 MHz, DMSO-d₆) δ 1.15-1.17 (3H, m), 1.28-1.36 (2H, m), 1.41-1.47 (2H, m), 1.93-1.98 (2H, m), 2.07-2.12 (2H, m), 4.90-4.97 (1H, m), 6.78 (1H, d), 7.46-7.52 (1H, m), 7.69-7.69 (1H, m), 8.02-8.08 (2H, m), 8.50 (1H, d), 10.63 (1H, s), 11.05 (1H, s), 12.12 (1H, s). m/z 506 (M+H)+

### Example 68

### (1s,4s)-4-(5-(5-(3,4-Difluorophenylamino)-1,3,4-oxadiazole-2-carboxamido)pyridin-2-yloxy)-1-methylcyclohexanecarboxylic acid

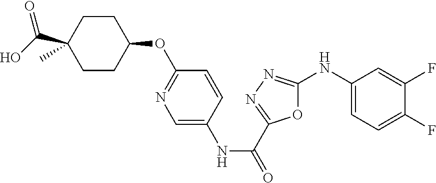

(1s,4s)-Ethyl 4-(5-(5-(3,4-difluorophenylamino)-1,3,4-oxadiazole-2-carboxamido)pyridin-2-yloxy)-1-methylcyclohexanecarboxylate (intermediate 181) (0.25 g, 0.50 mmol) and potassium trimethylsilanolate (0.640 g, 4.99 mmol) were suspended in THF (4.99 mL) and sealed into a microwave tube. The reaction was heated to 90° C. for 20 minutes in the microwave reactor and cooled to RT. The reaction mixture was diluted with water and the product precipitated out of water layer overnight. The precipitate was collected by filtration, washed with isohexane (1 mL) and dried under vacuum to afford crude product which was purified by crystallisation in ethanol to yield (1s,4s)-4-(5-(5-(3,4-difluorophenylamino)-1,3,4-oxadiazole-2-carboxamido)pyridin-2-yloxy)-1-methylcyclohexanecarboxylic acid (0.086 g, 36.4%) as a yellow solid.

¹H NMR (400 MHz, DMSO-d₆) δ 1.17 (3H, s), 1.52-1.59 (2H, m), 1.72-1.79 (4H, m), 1.81-1.87 (2H, m), 5.04-5.08 (1H, m), 6.78-6.84 (1H, m), 7.34-7.37 (1H, m), 7.46-7.51 (1H, m), 7.67-7.73 (1H, m), 8.03-8.07 (1H, m), 8.51 (1H, d), 11.14 (1H, s), 11.28 (1H, s), 12.23 (1H, s). m/z 474 (M+H)+

### Example 69

### 4-(5-(5-(3-Chloro-4-fluorophenylamino)-1,3,4-oxadiazole-2-carboxamido)pyridin-2-yloxy)-1-methylcyclohexanecarboxylic acid (cis/trans mixture)

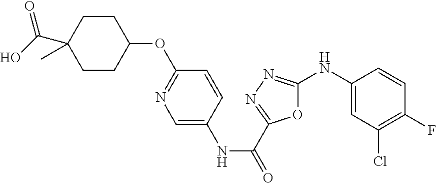

Sodium hydroxide (0.097 mL, 0.97 mmol) was added to a stirring solution of ethyl 4-(5-(5-(3-chloro-4-fluorophenylamino)-1,3,4-oxadiazole-2-carboxamido)pyridin-2-yloxy)-1-methylcyclohexanecarboxylate (cis/trans mixture; intermediate 182) (0.100 g, 0.19 mmol) in methanol (0.5 mL). The reaction was stirred at room temperature for 2 hours. The reaction was neutralized with 1M HCl. 4-(5-(5-(3-Chloro-4-fluorophenylamino)-1,3,4-oxadiazole-2-carboxamido)pyridin-2-yloxy)-1-methylcyclohexanecarboxylic acid precipitated out of solution as a yellow solid, which was purified by crystallisation in ethanol to yield (0.070 g, 74.0%) of 4-(5-(5-(3-chloro-4-fluorophenylamino)-1,3,4-oxadiazole-2-carboxamido)pyridin-2-yloxy)-1-methylcyclohexanecarboxylic acid (cis:trans=97:3) as a white solid.

¹H NMR (400 MHz, DMSO-d₆) δ 1.01 (3H, s), 1.36-1.41 (2H, m), 1.61 (2H, d), 1.77-1.83 (4H, m), 4.97 (1H, m), 6.74 (1H, d), 7.04 (1H, m), 7.16-7.20 (1H, m), 7.68-7.71 (1H, m), 7.98-8.02 (1H, m), 8.46 (1H, d). m/z 490 (M+H)+

### Example 70

### (1s,4s)-4-(6-Fluoro-5-(5-(2,4,5-trifluorophenylamino)-1,3,4-oxadiazole-2-carboxamido)pyridin-2-yloxy)cyclohexanecarboxylic acid

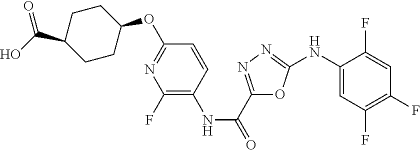

Sodium hydroxide (2.307 mL, 4.61 mmol) was added to (1s,4s)-methyl 4-(6-fluoro-5-(5-(2,4,5-fluorophenylamino)-1,3,4-oxadiazole-2-carboxamido)pyridin-2-yloxy)cyclohexanecarboxylate (intermediate 183) (0.47 g, 0.92 mmol) in MeOH (25.6 mL). The resulting solution was stirred at ambient temperature for 24 hours. The reaction mixture was neutralised with 2M HCl. The MeOH was removed in vaccu and the resulting solid was purified by crystallisation from EtOH to afford (1s,4s)-4-(6-fluoro-5-(5-(2,4,5-trifluorophenylamino)-1,3,4-oxadiazole-2-carboxamido)pyridin-2-yloxy)cyclohexanecarboxylic acid (0.242 g, 52.9%) as a white crystalline solid.

¹H NMR (400 MHz, DMSO-d₆) δ 1.63-1.87 (8H, m), 2.31-2.44 (1H, m), 5.00 (1H, s), 6.80 (1H, d), 7.65-7.73 (1H, m), 7.87-7.91 (1H, m), 8.11-8.18 (1H, m), 10.78 (1H, s), 11.06 (1H, s), 12.10 (1H, s). m/z 496 (M+H)+

### Example 71

### (1s,4s)-4-(5-(5-(4-Chloro-2-methoxyphenylamino)-1,3,4-oxadiazole-2-carboxamido)pyridin-2-yloxy)cyclohexanecarboxylic acid

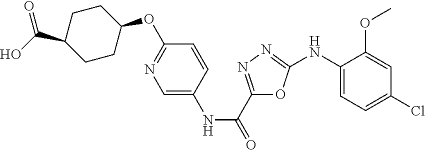

Sodium hydroxide (3.10 mL, 6.2 mmol) was added portionwise to (1s,4s)-methyl 4-(5-(5-(4-chloro-2-methoxyphenylamino)-1,3,4-oxadiazole-2-carboxamido)pyridin-2-yloxy)cyclohexanecarboxylate (intermediate 184) (0.304 g, 0.61 mmol) in methanol (4 mL). The resulting solution was stirred at ambient temperature for 12 hours. The reaction mixture was neutralized with 1M HCl (6.2 mL, 6.2 mmol) and evaporated to dryness to afford crude product which was purified by preparative HPLC, using decreasingly polar mixtures of water (containing 0.1% formic acid) and MeCN as eluents. Fractions containing the desired compound were evaporated to dryness to afford (1s,4s)-4-(5-(5-(4-chloro-3-fluorophenylamino)-1,3,4-oxadiazole-2-carboxamido)pyridin-2-yloxy)cyclohexanecarboxylic acid (0.025 g, 8.08%) as a white solid.

¹H NMR (400 MHz, DMSO-d₆) δ1.17-1.23 (1H, m), 1.65-1.86 (9H, m), 2.38 (1H, m), 5.12 (1H, s), 6.85 (1H, d), 7.08-7.11 (1H, m), 7.18 (1H, d), 7.99 (1H, d), 8.05-8.07 (1H, m), 8.51 (1H, d), 10.30 (1H, s), 11.12 (1H, s). m/z 488 (M+H)⁺.

### INTERMEDIATES

### Intermediate 1

### Methyl cis-4-[5-[[5-[(3,4-difluorophenyl)amino]1,3,4-oxadiazole-2-carbonyl]amino]pyridin-2-yl]oxycyclohexane-1-carboxylate

### Alternative Name—(1s,4s)-methyl 4-(5-(5-(3,4-difluorophenylamino)-1,3,4-oxadiazole-2-carboxamido)pyridin-2-yloxy)cyclohexanecarboxylate

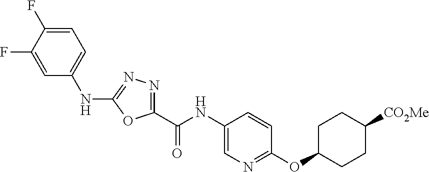

3,4-Difluorophenylisocyanate (CAS no. 42601-04-7) (1.63 g, 9.53 mmol) was added to a stirred solution of methyl cis-4-[5-[(hydrazinecarbonylformyl)amino]pyridin-2-yl]oxycyclohexane-1-carboxylate (Intermediate 2) (2.67 g, 7.94 mmol) in DMF (50 mL) at 65° C. under nitrogen. The resulting solution was stirred at 65° C. for 30 minutes. 1-(3-Dimethylaminopropyl)-3-ethylcarbodiimide hydrochloride (1.827 g, 9.53 mmol) was added to the reaction mixture and the resulting solution was stirred at 85° C. for 30 minutes. The reaction mixture was allowed to cool to ambient temperature before adding water (50 mL).

The precipitate was collected by filtration, washed with water (25 mL) and dried under vacuum to afford the title compound (3.20 g), which was used without further purification.

1H NMR (400 MHz, DMSO) δ1.63-1.91 (m, 8H), 2.44-2.57 (m, 1H), 3.63 (s, 3H), 5.10-5.18 (m, 1H), 6.84 (d, 1H), 7.33-7.39 (m, 1H), 7.44-7.54 (m, 1H), 7.66-7.75 (m, 1H), 8.03-8.09 (m, 1H), 8.51 (d, 1H), 11.09 (s, 1H), 11.24 (s, 1H). m/z 474.30 (M+H)⁺

### Intermediate 2

### Methyl cis-4-[5-[(hydrazinecarbonylformyl)amino]pyridin-2-yl]oxycyclohexane-1-carboxylate

### Alternative Name—(1s,4s)-methyl 4-(5-(2-hydrazinyl-2-oxoacetamido)pyridin-2-yloxy)cyclohexanecarboxylate

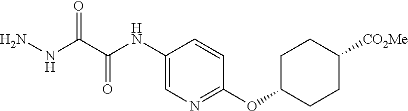

Hydrazine hydrate (0.735 mL, 15.2 mmol) was added to a stirred solution of methyl cis-4-[5-[(methoxycarbonylformyl)amino]pyridin-2-yl]oxycyclohexane-1-carboxylate (Intermediate 3) (4.63 g, 13.8 mmol) in ethanol (50 mL) warmed to 70° C. The resulting solution was stirred at 70° C. for 30 minutes.

The precipitate was collected by filtration, washed with diethyl ether (100 mL) and dried under vacuum to afford the title compound (2.67 g), which was used without further purification.

1H NMR (400 MHz, DMSO) δ 1.64-1.88 (m, 8H), 2.44-2.54 (m, 1H), 3.62 (s, 3H), 4.56-4.64 (m, 2H), 5.08-5.15 (m, 1H), 6.80 (d, 1H), 8.02-8.08 (m, 1H), 8.54 (d, 1H), 10.23 (s, 1H), 10.65 (s, 1H). m/z 337.33 (M+H)⁺

### Intermediate 3

### Methyl cis-4-[5-[(methoxycarbonylformyl)amino]pyridin-2-yl]oxycyclohexane-1-carboxylate

### Alternative Name—(1s,4s)-methyl 4-(5-(2-methoxy-2-oxoacetamido)pyridin-2-yloxy)cyclohexanecarboxylate

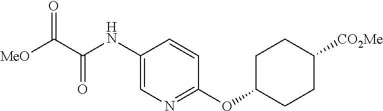

Methyl oxalyl chloride (1.66 mL, 18.0 mmol) was added to a stirred solution of methyl cis-4-(5-aminopyridin-2-yl)oxycyclohexane-1-carboxylate (Intermediate 4) (3.75 g, 15 mmol), and pyridine (2.42 mL, 30.0 mmol) in DCM (50 mL) cooled to 0° C. under nitrogen. The resulting solution was stirred at ambient temperature for 1 hour.

The reaction mixture was quenched with water (20 mL), extracted with DCM (2×20 mL), the organic layer was dried (MgSO₄), filtered and evaporated to afford the crude product (4.63 g), which was used without further purification.

1H NMR (400 MHz, DMSO) δ 1.62-1.90 (m, 8H), 2.43-2.58 (m, 1H), 3.62 (s, 3H), 3.86 (s, 3H), 5.08-5.16 (m, 1H), 6.80-6.85 (m, 1H), 7.98-8.03 (m, 1H), 8.46 (d, 1H), 10.84 (s, 1H). m/z 337.38 (M+H)⁺

### Intermediate 4

### Methyl cis-4-(5-aminopyridin-2-yl)oxycyclohexane-1-carboxylate

### Alternative Name—(1s,4s)-methyl 4-(5-aminopyridin-2-yloxy)cyclohexanecarboxylate

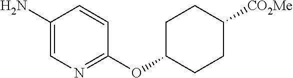

2M hydrochloric acid (5 mL) was added to a stirred solution of methyl cis-4-[5-(benzhydrylideneamino)pyridin-2-yl]oxycyclohexane-1-carboxylate (Intermediate 5) (0.800 g, 1.93 mmol) in THF (10 mL) at 20° C. This was stirred at ambient temperature for 2 hours.

The reaction mixture was adjusted to pH 7 with 2M NaOH. The reaction mixture was evaporated, and the resulting aqueous solution was extracted with ethyl acetate (50 mL). The organic layer was dried (MgSO₄), filtered and evaporated to afford crude product.

The crude product was purified by flash silica chromatography, eluting with 0 to 80% ethyl acetate in isohexane to afford the pure product (0.350 g, 72.5%).

1H NMR (400 MHz, DMSO) δ 1.55-1.86 (m, 8H), 2.42-2.49 (m, 1H), 3.62 (s, 3H), 4.68 (s, 2H), 4.90-4.97 (m, 1H), 6.51 (d, 1H), 6.96-7.01 (m, 1H), 7.49 (d, 1H). m/z 251.40 (M+H)⁺

### Intermediate 5

### Methyl cis-4-[5-(benzhydrylideneamino)pyridin-2-yl]oxycyclohexane-1-carboxylate

### Alternative Name—(1s,4s)-methyl 4-(5-(diphenylmethyleneamino)pyridin-2-yloxy)cyclohexanecarboxylate

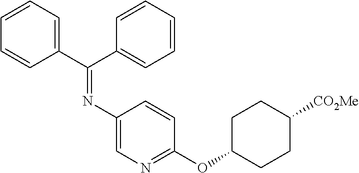

Benzophenone imine (0.400 mL, 2.39 mmol) was added to cis-methyl 4-(5-bromopyridin-2-yl)oxycyclohexane-1-carboxylate (Intermediate 6) (0.500 g, 1.59 mmol), palladium(II) acetate (0.021 g, 0.10 mmol), cesium carbonate (0.178 mL, 2.23 mmol) and (S)-(−)-2,2′-Bis(diphenylphosphino)-1,1′-binaphthyl (0.059 g, 0.10 mmol) in THF (10 mL) at 20° C. under nitrogen. The resulting solution was stirred at reflux for 8 hours.

The reaction mixture was evaporated to dryness and redissolved in ethyl acetate (10 mL), and washed sequentially with water (10 mL), and saturated brine (10 mL). The organic layer was dried (MgSO₄), filtered and evaporated to afford crude product which was used without further purification. m/z 415.37 (M+H)⁺

### Intermediate 6

### Methyl cis-4-(5-bromopyridin-2-yl)oxycyclohexane-1-carboxylate

### Alternative Name—(1s,4s)-methyl 4-(5-bromopyridin-2-yloxy)cyclohexanecarboxylate

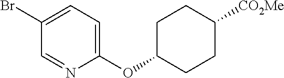

A few drops of conc. hydrochloric acid were added to a stirred solution of cis-4-(5-bromopyridin-2-yl)oxycyclohexane-1-carboxylic acid (Intermediate 7) (6.76 g, 22.5 mmol) in methanol at 20° C. The resulting solution was stirred at 60° C. for 3 hours.

The solvent was evaporated to afford the crude product (6.35 g) that was used with out further purification.

1H NMR (400 MHz, DMSO) δ 1.63-1.86 (m, 8H), 2.45-2.55 (m, 1H), 3.61 (s, 3H), 5.07-5.13 (m, 1H), 6.81 (d, 1H), 7.85-7.90 (m, 1H), 8.25 (d, 1H). m/z 316.2 (M+H)⁺

### Intermediate 7

### cis-4-(5-Bromopyridin-2-yl)oxycyclohexane-1-carboxylic acid

### Alternative Name—(1s,4s)-4-(5-bromopyridin-2-yloxy)cyclohexanecarboxylic acid

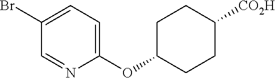

A solution of cis-4-hydroxycyclohexanecarboxylic acid (CAS no. 3685-22-1) (4.98 g, 34.53 mmol) in DMA (20 mL) was added to a stirred mixture of sodium hydride (2.76 g, 69.00 mmol) in DMA (80 mL) cooled to 0° C. The resulting suspension was stirred at ambient temperature for 30 minutes. 5-Bromo-2-fluoropyridine (CAS no. 766-11-0) (6.08 g, 34.53 mmol) was added and the resulting suspension was stirred at 100° C. reflux for 1 hour.

The reaction mixture was allowed to cool to room temperature, diluted with water (50 mL) and acidified with 2M hydrochloric acid. The precipitate was collected by filtration, washed with water (50 mL) and dried under vacuum to afford the title compound (6.76 g), which was used without further purification.

1H NMR (400 MHz, DMSO) δ 1.61-1.87 (m, 8H), 2.32-2.42 (m, 1H), 5.05-5.13 (m, 1H), 6.81 (d, 1H), 7.85-7.90 (m, 1H), 8.25 (d, 1H), 12.15 (s, 1H). m/z 302.2, (M+H)⁺

### Intermediate 8

### Ethyl 4-(5-(5-(3,4-difluorophenylamino)-1,3,4-oxadiazole-2-carboxamido)pyridin-2-yloxy)-1-methylcyclohexanecarboxylate

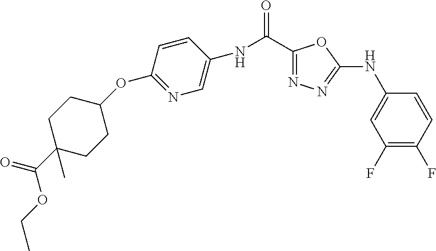

3,4-Difluorophenyl isothiocyanate (0.045 g, 0.26 mmol) was added to ethyl 4-(5-(2-hydrazinyl-2-oxoacetamido)pyridin-2-yloxy)-1-methylcyclohexanecarboxylate (Intermediate 9) (0.091 g, 0.25 mmol) in DMF (2.5 mL). The resulting solution was stirred at 40° C. until starting material was consumed, approx. 25 minutes. N-(3-dimethylaminopropyl)-N′-ethylcarbodiimide hydrochloride (0.053 g, 0.27 mmol) was added to the above solution. The resulting solution was stirred at 80° C. until the intermediate was consumed, approx. 90 minutes. The reaction was then cooled to room temperature and water (6 mL) was added to the reaction mixture which caused a precipitate to form. The precipitate was collected by filtration, washed with water (2 mL) and dried under vacuum to afford ethyl 4-(5-(5-(3,4-difluorophenylamino)-1,3,4-oxadiazole-2-carboxamido)pyridin-2-yloxy)-1-methylcyclohexanecarboxylate (0.129 g) as a yellow solid, which was used without further purification. m/z 502 (M+H)⁺

### Intermediate 9

### Ethyl 4-(5-(2-hydrazinyl-2-oxoacetamido)pyridin-2-yloxy)-1-methylcyclohexanecarboxylate

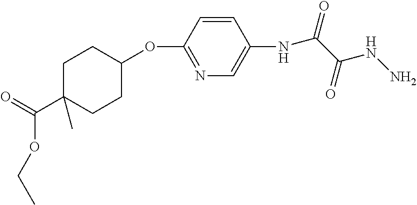

Hydrazine hydrate (0.421 mL, 8.64 mmol) was added dropwise to ethyl 4-(5-(2-methoxy-2-oxoacetamido)pyridin-2-yloxy)-1-methylcyclohexanecarboxylate (Intermediate 10) (3.0 g, 8.2 mmol) in ethanol (50 mL) under air. The resulting solution was stirred at ambient temperature for 14 hours, upon which time the product precipitated out of solution. The precipitate was collected by filtration, washed with ethanol (10 mL) and isohexanes (10 mL) to yield the product (2.51 g, 84%) which was carried through without further purification.

1H NMR (400 MHz, DMSO) δ 1.14-1.22 (6H, m), 1.38 (1H, m), 1.55-1.61 (2H, m), 1.67-1.79 (3H, m), 1.83-1.89 (2H, m), 1.96 (1H, d, major), 2.10 (1H, m, minor), 4.08-4.15 (2H, m), 4.61 (2H, s), 4.93 (1H, t, minor), 5.02-5.07 (1H, m, major), 6.74-6.80 (1H, m), 8.02-8.07 (1H, m), 8.53 (1H, d), 10.23 (1H, s), 10.65 (1H, s). m/z 365 (M+H)⁺

### Intermediate 10

### Ethyl 4-(5-(2-methoxy-2-oxoacetamido)pyridin-2-yloxy)-1-methylcyclohexanecarboxylate

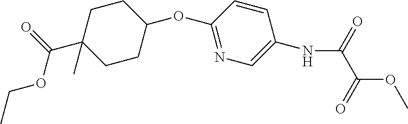

Methyl oxalyl chloride (0.78 mL, 8.5 mmol) was added dropwise to ethyl 4-(5-aminopyridin-2-yloxy)-1-methylcyclohexanecarboxylate (Intermediate 11) (2.3 g, 8.1 mmol) and triethylamine (1.2 mL, 8.5 mmol) in DCM (80 mL) under nitrogen. The resulting solution was stirred at ambient temperature for 14 hours.

The reaction mixture was washed sequentially with water (20 mL) and saturated brine (20 mL). The organic layer was dried (MgSO₄), filtered and evaporated to afford the product (3.0 g) which was used without further purification.

1H NMR (400 MHz, CDCl₃) δ 1.25 (6H, m), 1.51-1.68 (3H, m), 1.74-1.88 (3H, m), 1.93-1.95 (1H, m, major), 1.97-2.01 (1H, m), 2.26 (1H, d, minor), 3.97 (3H, s), 4.14-4.20 (2H, m), 4.97 (1H, m, minor), 5.10-5.14 (1H, m, major), 6.69-6.75 (1H, m), 7.92-7.99 (1H, m), 8.28-8.31 (1H, m), 8.73 (1H, s). m/z 365 (M+H)⁺

### Intermediate 11

### Ethyl 4-(5-aminopyridin-2-yloxy)-1-methylcyclohexanecarboxylate

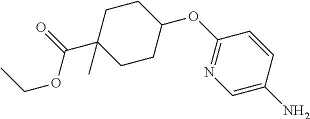

Ethyl 1-methyl-4-(5-nitropyridin-2-yloxy)cyclohexanecarboxylate (Intermediate 12) (2.55 g, 8.27 mmol), and palladium (10% on activated carbon) (0.88 g) in ethanol (200 mL) were stirred under an atmosphere of hydrogen at 1 atm and at ambient temperature for 14 hours. The reaction mixture was filtered and the filtrate evaporated to afford the title compound (2.25 g, 98%) which was used without further purification. m/z 279 (M+H)⁺

### Intermediate 12

### Ethyl 1-methyl-4-(5-nitropyridin-2-yloxy)cyclohexanecarboxylate

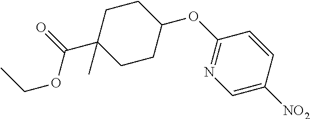

Diisopropyl azodicarboxylate (1.0 mL, 5.1 mmol) was added to triphenylphosphine (1.62 g, 6.17 mmol), and 5-nitropyridin-2-ol (0.41 g, 3.0 mmol) in THF (15 mL) at 20° C. under nitrogen. The resulting suspension was stirred at 20° C. for 25 minutes. Ethyl 4-hydroxy-1-methylcyclohexanecarboxylate (Intermediate 13) (0.5 g, 2.7 mmol) was then added to the solution. The reaction was heated to 150° C. for 30 minutes in the microwave reactor and cooled to RT. The reaction mixture was concentrated, diluted with ethyl acetate (50 mL), and washed with water (50 mL), and saturated brine (50 mL). The organics were dried (MgSO₄), filtered and evaporated to afford crude product. The crude product was purified by flash silica chromatography, eluting with 10 to 50% ethyl acetate in isohexane to afford the title compound (0.44 g, 53%).

1H NMR (400 MHz, CDCl₃) δ 1.21-1.30 (6H, m), 1.59-1.67 (2H, m), 1.77-1.92 (3H, m), 1.97-2.06 (2H, m), 2.27-2.30 (1H, m), 4.12-4.21 (2H, q), 5.10-5.17 (1H, m, minor), 5.29-5.33 (1H, m, major), 6.73-6.81 (1H, m), 8.30-8.35 (1H, m), 9.04-9.06 (1H, m). m/z 309 (M+H)⁺

### Intermediate 13

### Ethyl 4-hydroxy-1-methylcyclohexanecarboxylate

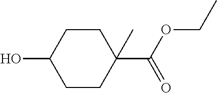

Tetrabutylammonium fluoride (1M in THF) (54 mL, 54 mmol) was added portion wise to ethyl 4-(tert-butyldimethylsilyloxy)-1-methylcyclohexanecarboxylate (Intermediate 14) (8.2 g, 27 mmol) in tetrahydrofuran (54 mL). The resulting solution was stirred at ambient temperature for 35 hours. The reaction mixture was washed sequentially with saturated ammonium chloride solution (50 mL), and saturated brine (50 mL). The organic layer was dried (MgSO₄), filtered and evaporated to afford crude product. The crude material was purified by flash silica chromatography, eluting with 10 to 70% ethyl acetate in isohexane to afford the title compound (3.20 g, 63%).

1H NMR (400 MHz, CDCl₃) δ 1.23-1.28 (6H, m), 1.44-1.56 (2H, m), 1.61-1.68 (3H, m), 1.99-2.03 (2H, m), 2.20-2.27 (1H, m, major), 2.35-2.42 (1H, m, minor), 3.58-3.65 (1H, m, major), 3.90-3.92 (1H, m, minor), 4.10-4.18 (2H, m)

### Intermediate 14

### Ethyl 4-(tert-butyldimethylsilyloxy)-1-methylcyclohexane-carboxylate

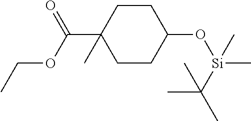

n-Butyllithium (1.6 M in hexane) (39.8 mL, 63.7 mmol) was added dropwise to diisopropylamine (7.9 mL, 58 mmol) in THF (193 mL) at −78° C. under nitrogen. The resulting solution was stirred at −78° C. for 30 minutes, warmed to 0° C. and stirred at 0° C. for 30 minutes. The reaction mixture was cooled to −78° and ethyl 4-(tert-butyldimethylsilyloxy)cyclohexanecarboxylate (Intermediate 15) (16.6 g, 57.9 mmol) was added dropwise to the above solution. The resulting solution was stirred at −78° C. for 1 hour then warmed to 0° C. and stirred for 20 minutes, recooled to −78° C. and methyl iodide (4.0 mL, 64 mmol) was added dropwise to the reaction mixture. The resulting solution was allowed to warm naturally to room temperature. The reaction mixture was washed sequentially with saturated ammonium chloride solution (75 mL), saturated brine (75 mL). The organic layer was dried (MgSO₄), filtered and evaporated to afford crude product.

The crude product was purified by flash silica chromatography, eluting with 0 to 10% ethyl acetate in isohexane to afford the title compound (16.2 g, 93%).

1H NMR (400 MHz, CDCl₃) δ 0.78-0.88 (15H, m), 1.09 (3H, s, major), 1.13 (3H, s, minor), 1.17-1.26 (3H, m), 1.55-1.62 (2H, m), 1.65-1.71 (2H, m), 1.83-1.95 (21-1, m), 2.13-2.19 (2H, m), 3.50-3.56 (1H, m), 4.05-4.18 (2H, m)

### Intermediate 15

### Ethyl 4-(tert-butyldimethylsilyloxy)cyclohexanecarboxylate

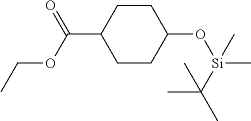

tert-Butyldimethylchlorosilane (9.71 g, 64.45 mmol) was added portion wise to ethyl 4-hydroxycyclohexanecarboxylate (CAS no. 17159-80-7) (10 g, 58 mmol) and imidazole (7.9 g, 116 mmol) in DMF (58 mL) under nitrogen. The resulting solution was stirred at room temperature for 18 hours. The reaction mixture was diluted with diethyl ether (250 mL), and washed with saturated brine (500 mL). The organic layer was dried (MgSO₄), filtered and evaporated to afford the product (16.3 g). This was used without any further purification.

1H NMR (300 MHz, CDCl₃) δ 0.02-0.07 (5H, m), 0.87-0.90 (9H, m), 0.92 (1H, s), 1.22-1.29 (3H, m), 1.30-1.54 (3H, m), 1.59-1.70 (2H, m), 1.87-2.04 (3H, m), 2.21-2.32 (1H, m), 3.70-3.89 (1H, m), 4.07-4.16 (2H, m).

### Intermediate 16

### (1r,4r)-tert-butyl 4-(5-(5-(3,4-difluorophenylamino)-1,3,4-oxadiazole-2-carboxamido)pyridin-2-yloxy)-1-(methoxymethyl)cyclohexanecarboxylate

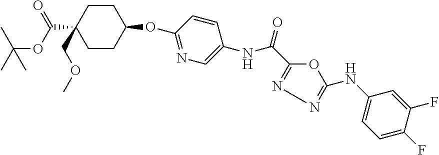

3,4-Difluorophenyl isothiocyanate (0.396 g, 2.32 mmol) was added to (1r,4r)-tert-butyl 4-(5-(2-hydrazinyl-2-oxoacetamido)pyridin-2-yloxy)-1-(methoxymethyl)cyclohexanecarboxylate (Intermediate 18) (0.932 g, 2.21 mmol) in DMF (22 mL). The resulting solution was stirred at 40° C. for 25 minutes. N-(3-Dimethylaminopropyl)-N′-ethylcarbodiimide hydrochloride (0.465 g, 2.43 mmol) was added to the above solution. The resulting solution was stirred at 80° C. for 50 minutes. The reaction was then cooled to room temperature and water (10 mL) was slowly added to the reaction mixture which caused a precipitate to form. The precipitate was collected, washed with water and isohexane, dried under vacuum and used without further purification.

m/z 423 (M+H)⁺

### Intermediate 17

### (1r,4r)-tert-butyl 1-(methoxymethyl)-4-(5-(5-(2,4,5-trifluorophenylamino)-1,3,4-oxadiazole-2-carboxamido)pyridin-2-yloxy)cyclohexanecarboxylate

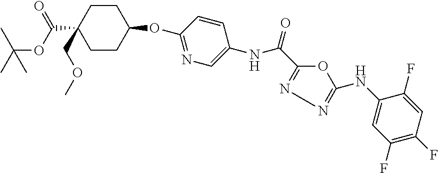

2,4,5-Trifluorophenyl isothiocyanate (0.081 g, 0.43 mmol) was added to (1r,4r)-tert-butyl 4-(5-(2-hydrazinyl-2-oxoacetamido)pyridin-2-yloxy)-1-(methoxymethyl)cyclohexanecarboxylate (Intermediate 18) (0.182 g, 0.43 mmol) in DMF (4.3 mL) under nitrogen. The resulting solution was stirred at 40° C. for 90 minutes. N-(3-Dimethylaminopropyl)-N′-ethylcarbodiimide hydrochloride (0.087 g, 0.45 mmol) was added to the above solution under nitrogen. The resulting solution was stirred at 80° C. for 90 minutes. The reaction was cooled to room temperature and water (10 mL) was slowly added to the reaction mixture. A precipitate formed which was collected by filtration and washed with water and hexanes to afford the product (0.15 g) which was used without further purification. m/z 578 (M+H)⁺

### Intermediate 18

### (1r,4r)-tert-butyl 4-(5-(2-hydrazinyl-2-oxoacetamido)pyridin-2-yloxy)-1-(methoxymethyl)cyclohexanecarboxylate

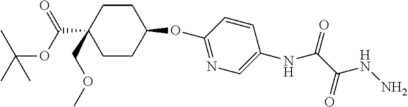

Hydrazine hydrate (0.18 mL, 3.8 mmol) was added dropwise to (1r,4r)-tert-butyl 4-(5-(2-methoxy-2-oxoacetamido)pyridin-2-yloxy)-1-(methoxymethyl)cyclohexanecarboxylate (Intermediate 19) (1.52 g, 3.60 mmol) in ethanol (36 mL) under air. The resulting solution was stirred at ambient temperature for 2 hours, upon which time the product precipitated out of solution. The precipitate was collected by filtration, washed with ethanol (20 mL) to yield (1r,4r)-tert-butyl 4-(5-(2-hydrazinyl-2-oxoacetamido)pyridin-2-yloxy)-1-(methoxymethyl)cyclohexanecarboxylate (1.24 g, 82%), which was used without further purification.

1H NMR (300 MHz, CDCl₃) δ 1.43-1.48 (9H, s), 1.65-1.94 (8H, m), 3.32 (3H, d), 3.43 in (2H, s), 4.02 (1H, s), 5.16 (1H, m), 6.73 (1H, d), 7.92-7.96 (1H, m), 8.29 (1H, d), 8.55 (1H, s), 9.01 (1H, s). m/z 423 (M+H)⁺

### Intermediate 19

### (1r,4r)-tert-butyl 4-(5-(2-methoxy-2-oxoacetamido)pyridin-2-yloxy)-1-(methoxymethyl)cyclohexanecarboxylate

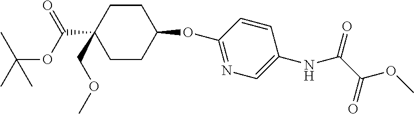

Methyl oxalyl chloride (1.7 mL, 19 mmol) was added dropwise to a solution of (1r,4r)-tert-butyl 4-(5-aminopyridin-2-yloxy)-1-(methoxymethyl)cyclohexanecarboxylate (Intermediate 20) (1.56 g, 4.64 mmol) and triethyl amine (0.43 mL, 5.6 mmol) in DCM (46 mL) under nitrogen. The resulting solution was stirred at ambient temperature for 90 minutes. The reaction mixture was then washed sequentially with water (20 mL) and saturated brine (20 mL). The organic layer was dried (MgSO₄), filtered and evaporated. The residue was purified by flash chromatography, eluting with 0 to 50% ethyl acetate in isohexane to afford the title compound (1.52 g, 78%).

1H NMR (300 MHz, CDCl₃) δ 1.46 (9H, s), 1.67-1.76 (2H, m), 1.80-1.93 (6H, m), 3.32 (3H, s), 3.43 (2H, s), 3.98 (3H, s), 5.16 (1H, m), 6.73 (1H, d), 7.96-8.00 (1H, m), 8.28 (1H, d), 8.73 (1H, s). m/z 423 (M+H)⁺

### Intermediate 20

### (1r,4r)-tert-butyl 4-(5-aminopyridin-2-yloxy)-1-(methoxymethyl)-cyclohexanecarboxylate

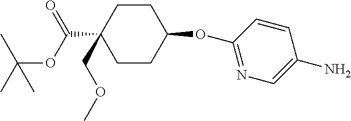

(1r,4r)-tert-Butyl 1-(methoxymethyl)-4-(5-nitropyridin-2-yloxy)cyclohexanecarboxylate (Intermediate 21) (2.46 g, 6.71 mmol) and palladium black (246 mg, 2.31 mmol) were suspended in ethanol (100 mL) and stirred under an atmosphere of hydrogen at room temperature for 14 hours. The reaction mixture was filtered, the filtrate was rinsed with ethanol (200 mL) and the solid discarded. The organics were combined and evaporated to afford the title compound, which was used without further purification.

1H NMR (300 MHz, CDCl₃) δ 1.46 (9H, s), 1.68-1.82 (6H, m), 1.86-1.91 (2H, m), 3.31 (3H, d), 3.43 (2H, s), 3.72 (1H, s), 4.97-5.02 (1H, m), 6.55 (1H, d), 6.98-7.02 (1H, m), 7.63 (1H, d). m/z 337 (M+H)⁺

### Intermediate 21

### (1r,4r)-tert-butyl 1-(methoxymethyl)-4-(5-nitropyridin-2-yloxy)cyclohexanecarboxylate

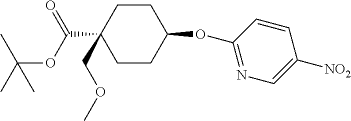

Diisopropyl azodicarboxylate (0.77 mL, 3.9 mmol) was added to triphenylphosphine (1.24 g, 4.71 mmol), 5-nitropyridin-2-ol (0.287 g, 2.05 mmol) in THF (15 mL) at 20° C. under nitrogen. The resulting suspension was stirred at 20° C. for 25 minutes. (1s,4s)-tert-butyl 4-hydroxy-1-(methoxymethyl)cyclohexanecarboxylate (Intermediate 22) (0.501 g, 2.05 mmol) was added to the solution and the reaction was heated to 150° C. for 30 minutes in a microwave reactor and cooled to RT. The reaction mixture concentrated, diluted with ethyl acetate (50 mL) and washed with saturated brine (50 mL). The aqueous layer was re-extracted with ethyl acetate (50 mL). The organic layers were combined, dried (MgSO₄), filtered and evaporated. The residue was purified by flash chromatography, eluting with 10 to 50% ethyl acetate in isohexane to afford the title compound (0.525 g, 69%).

1H NMR (300 MHz, CDCl₃) δ 1.47 (9H, s), 1.44-1.51 (1H, m), 1.64-1.76 (2H, m), 1.83-1.98 (6H, m), 3.33 (3H, s), 3.42 (2H, s), 5.35-5.37 (1H, m), 6.77 (1H, d), 8.30-8.34 (1H, m), 9.04 (1H, d). m/z 367 (M+H)⁺

### Intermediate 22

### (1s,4s)-tert-butyl 4-hydroxy-1-(methoxymethyl)cyclohexanecarboxylate

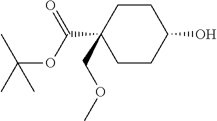

(1s,4s)-tert-Butyl 4-(tert-butyldimethylsilyloxy)-1-(methoxymethyl)cyclohexanecarboxylate (Intermediate 23) (5.65 g, 15.8 mmol) was added to (1s,4s)-tert-butyl 4-(tert-butyldimethylsilyloxy)-1-(methoxymethyl)cyclohexanecarboxylate (5.65 g, 15.8 mmol) in THF (31.5 mL). The resulting solution was stirred under air, at ambient temperature for 12 hours. The reaction mixture was washed with saturated ammonium chloride solution (25 mL), and saturated brine (25 mL). The organic layer was dried (MgSO₄), filtered and evaporated. The residue was purified by flash silica chromatography, eluting with 20 to 80% ethyl acetate in isohexane to afford the title compound (2.82 g, 73.3%).

1H NMR (300 MHz, CDCl₃) δ 1.37-1.43 (2H, m), 1.45-1.46 (9H, m), 1.71-1.77 (2H, m), 1.80-1.90 (2H, m), 2.16-2.24 (2H, m), 3.28-3.32 (5H, m), 3.53-3.57 (1H, m), 3.87 (1H, s).

### Intermediate 23

### (1s,4s)-tert-butyl 4-(tert-butyldimethylsilyloxy)-1-(methoxymethyl)cyclohexanecarboxylate

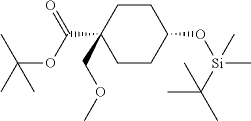

n-Butyllithium (1.6 M in hexane) (21.9 mL, 35.0 mmol) was added dropwise to diisopropylamine (4.36 mL, 31.8 mmol) in THF (106 mL) at −78° C. under nitrogen upon completion of addition the reaction was warmed slowly to 0° C. over 1 hour. The reaction was then cooled to −78° C. and tert-butyl 4-(tert-butyldimethylsilyloxy)cyclohexanecarboxylate (Intermediate 24) (10.0 g, 31.8 mmol) was added slowly to the above solution. The resulting solution was stirred at −78° C. for 1 hour, warmed to 0° C., retooled to −78° C. and bromomethyl methyl ether (2.60 mL, 31.8 mmol) was added dropwise to the reaction mixture. The resulting solution was allowed to warm to room temperature. The reaction was quenched with saturated ammonium chloride solution (70 mL) and washed with water (50 mL) and saturated brine (75 mL). The organic layer was dried (MgSO₄), filtered and evaporated. The residue was purified by flash chromatography, eluting with 10 to 60% ethyl acetate in isohexane to afford the title compound (10.20 g, 89%).

1H NMR (300 MHz, CDCl₃) δ 0.03-0.05 (6H, m), 0.85-0.91 (9H, m), 1.17-1.30 (3H, m), 1.35-1.39 (1H, m), 1.46 (9H, s), 1.69-1.75 (2H, m), 2.13 (2H, d), 3.28-3.01 (5H, m), 3.47-3.54 (1H, m).

### Intermediate 24

### tert-butyl 4-(dimethyl-tert-butyl-silyl)oxycyclohexane-1-carboxylate

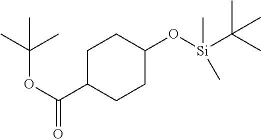

Di-tert-butyl dicarbonate (3.70 g, 17.0 mmol), followed by 4-(dimethylamino)pyridine (311 mgs, 2.54 mmol) was added to a solution of 4-(dimethyl-tert-butyl-silyl)oxycyclohexane-1-carboxylic acid (Intermediate 25) (2.19 g, 8.47 mmol) in tert-butanol (20 mL), the reaction mixture was allowed to stir at ambient temperature under nitrogen for 1 hour. The solvent was evaporated and the residue was purified by flash chromatography, eluting with 0 to 10% ethyl acetate in isohexane to afford the title compound (as a 50:50 mix of cis and trans isomers).

1H NMR (300 MHz, CDCl₃) δ 0.03 (6H, s), 0.05 (6H, s), 0.88 (9H, s), 1.20-1.36 (2H, m), 1.43 (9H, s), 1.44 (9H, s), 1.54-1.66 (2H, m), 1.82-1.97 (3H, m), 2.03-2.24 (2H, m), 3.50-3.60 (1H, m), 3.84-3.90 (1H, m)

### Intermediate 25

### 4-(dimethyl-tert-butyl-silyl)oxycyclohexane-1-carboxylic acid

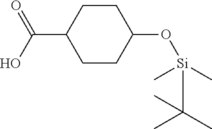

A solution of ethyl 4-(dimethyl-tert-butyl-silyl)oxycyclohexane-1-carboxylate (Intermediate 15) (43.3 g, 151 mmol) in THF (300 mL) and 2N NaOH (150 mL, 300 mmol) was allowed to stir at ambient temperature overnight, further 2N NaOH (150 mL) was added and the resulting mixture was heated to 50° C. for 2 hours and then at 70° C. for 1.5 hours. Further of 2N NaOH (75 mL) was added and heating was continued for a further 3 hours and then stirred at ambient temperature for 3 days. The THF was evaporated under reduced pressure and the residue was washed with isohexane (300 mL), ether (300 mL) and then the aqueous phase was adjusted to pH ˜5 by the addition of 1N citric acid solution. The aqueous phase was re extracted into ether (4×500 mL). The combined organic extracts were dried (MgSO₄) and concentrated to afford the product (34.8 g, 89%) which was used without purification.

1H NMR (300 MHz, CDCl₃) δ 0.04 (3H, s), 0.06 (3H, s), 0.89 (9H, s), 1.25-1.39 (1H, m), 1.43-1.57 (2H, m), 1.63-1.72 (2H, m), 1.87-2.04 (3H, m), 2.23-2.40 (1H, m), 3.53-3.62 (0.5H, m), 3.88-3.92 (0.5H, m); CO2H not seen.

### Intermediate 26

### (1s,4s)-Methyl 4-(5-(5-(2,4-difluoro-5-(trifluoromethyl)-phenylamino)-1,3,4-oxadiazole-2-carboxamido)pyridin-2-yloxy)cyclohexane-carboxylate

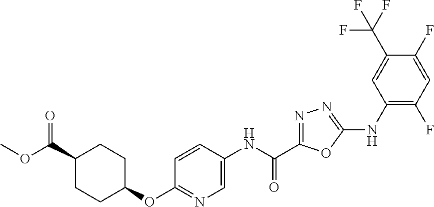

O-Perfluorophenyl 2,4-difluoro-5-(trifluoromethyl)phenylcarbamothioate (Intermediate 27) (430 mg, 1.02 mmol) was added to (1s,4s)-methyl 4-(5-(2-hydrazinyl-2-oxoacetamido)pyridin-2-yloxy)cyclohexanecarboxylate (Intermediate 28) (285 mg, 0.85 mmol), in DMF (8 mL). The resulting solution was stirred at 55° C. for 1 hour. 1-(3-Dimethylaminopropyl)-3-ethylcarbodiimide hydrochloride (195 mg, 1.02 mmol) was added and the mixture was stirred at 75° C. for a further 17 hours. The DMF was removed by evaporation. The crude oil was triturated under water to give a solid which was collected by filtration, washed with ether (10 mL), and dried under vacuum to afford the product (310 mg, 68%). m/z 542 (M+H)⁺

### Intermediate 27

### O-perfluorophenyl 2,4-difluoro-5-(trifluoromethyl)phenylcarbamothioate

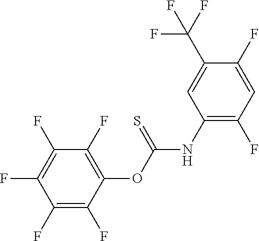

Pentafluorophenyl chlorothionoformate (CAS no. 135192-53-9) (1.6 mL, 10 mmol) in dichloromethane (20 mL) was added dropwise to 2,4-difluoro-5-(trifluoromethyl)aniline (CAS no. 261944-56-3) (1.80 g, 9.13 mmol) and pyridine (1.1 mL, 14 mmol) in dichloromethane (100 mL) at 0° C. The resulting solution was allowed to warm to 20° C. and was stirred for 20 hours. The reaction mixture was washed sequentially with 1M citric acid (100 mL), saturated NaHCO₃ (100 mL), and saturated brine (100 mL). The organic layer was dried (MgSO₄) and evaporated to afford crude product. The crude product was purified by flash chromatography on silica eluting with a gradient of 0 to 5% ethyl acetate in isohexane to afford the product (0.897 g, 23%).

¹H NMR (300 MHz, DMSO) δ 4.25 (1H, s), 7.91 (1H, t), 8.06 (1H, t)

### Intermediate 28

### (1s,4s)-methyl 4-(5-(2-hydrazinyl-2-oxoacetamido)pyridin-2-yloxy)cyclohexanecarboxylate

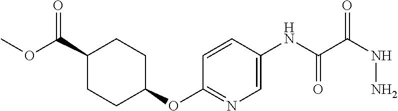

Hydrazine monohydrate (0.30 mL, 6.1 mmol) was added dropwise to (1s,4s)-methyl 4-(5-(2-methoxy-2-oxoacetamido)pyridin-2-yloxy)cyclohexanecarboxylate (Intermediate 29) (2.1 g, 6.1 mmol), in ethanol (100 mL) at 22° C. The resulting suspension was stirred at 22° C. for 16 hours. A precipitate was collected by filtration, washed with ethanol (100 mL), and dried under vacuum to afford the product (1.11 g, 54%), which was used without further purification.

¹H NMR (300 MHz, DMSO) δ 1.59-1.87 (8H, m), 3.62 (3H, s), 4.59 (2H, s), 5.10 (1H, s), 6.78 (1H, d), 8.03 (1H, d), 8.52 (1H, s), 10.24 (1H, s), 10.65 (1H, s). m/z 337 (M+H)⁺

### Intermediate 29

### (1s,4s)-methyl 4-(5-(2-methoxy-2-oxoacetamido)pyridin-2-yloxy)cyclohexanecarboxylate

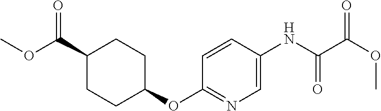

Methyl oxalyl chloride (4.5 mL, 49 mmol) was added dropwise to a stirred solution of (1s,4s)-methyl 4-(5-aminopyridin-2-yloxy)cyclohexanecarboxylate (Intermediate 30) (11.18 g, 44.7 mmol), and pyridine (4.34 mL, 53.60 mmol) in dichloromethane (200 mL) at 22° C. The resulting solution was stirred at 22° C. for 16 hours. The reaction mixture was diluted with DCM (200 mL). Washed with water (100 mL) and brine (100 mL), dried (MgSO₄) and evaporated to afford crude product. This was purified by flash chromatography on silica eluting with a gradient of 0 to 60% ethyl acetate in isohexane to afford the product (13 g, 87%).

¹H NMR (300 MHz, DMSO) δ 1.62-1.88 (8H, m), 3.61 (3H, s), 3.84 (3H, s), 5.10 (1H, s), 6.80 (1H, d), 7.98 (1H, dd), 8.44 (1H, d), 10.84 (1H, s). m/z 337 (M+H)⁺

### Intermediate 30

### (1s,4s)-methyl 4-(5-aminopyridin-2-yloxy)cyclohexanecarboxylate

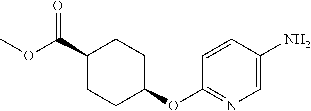

(1s,4s)-methyl 4-(5-nitropyridin-2-yloxy)cyclohexanecarboxylate (Intermediate 31) (12.55 g, 44.78 mmol), and 10% palladium on carbon (1.2 g, 1.13 mmol) in ethyl acetate were stirred under an atmosphere of hydrogen supplied by a balloon at 22° C. for 16 hours. The reaction mixture was filtered through celite. The solution was evaporated to afford the product (11.20 g, 100%).

¹H NMR (300 MHz, DMSO) δ 1.54-1.83 (8H, m), 2.39-2.55 (1H+DMSO, m), 3.60 (3H, s), 4.68 (2H, s), 4.91 (1H, m), 6.50 (1H, d), 6.97 (1H, dd), 7.47 (1H, d).

m/z 251 (M+H)⁺

### Intermediate 31

### (1s,4s)-methyl 4-(5-nitropyridin-2-yloxy)cyclohexanecarboxylate

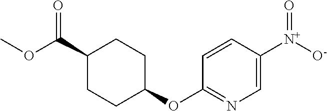

Diisopropyl azodicarboxylate (15.25 mL, 77.44 mmol) was added to a stirred solution of 2-hydroxy-5-nitropyridine (CAS no. 5418-51-9) (8.68 g, 62.0 mmol), and triphenylphosphine (24.37 g, 92.93 mmol) in tetrahydrofuran (385 mL) under nitrogen. The resulting solution was stirred at 20° C. for 30 minutes and then (1r,4r)-methyl 4-hydroxycyclohexanecarboxylate (Intermediate 32) (12.25 g, 77.44 mmol) in tetrahydrofuran (15 mL) was added. The resulting solution was stirred for 20 hours under nitrogen. The solvent was evaporated and 33% ethyl acetate in isohexane (200 mL) added. Stirred for 1 hour then the solution was filtered and evaporated. The residue was redissolved in ethyl acetate (200 mL), and washed sequentially with water (100 mL) and saturated brine (100 mL). The organic layer was dried (MgSO₄) and evaporated to afford crude product. This was purified by flash silica chromatography, elution gradient 20 to 50% ethyl acetate in isohexane to afford the product (12.6 g, 72.6%).

¹H NMR (300 MHz, DMSO) δ 1.67-1.92 (9H, m), 3.61 (3H, s), 5.30 (1H, m), 7.01 (1H, d), 8.44 (1H, dd), 9.05 (1H, d). m/z 281 (M+H)⁺

### Intermediate 32

### (1r,4r)-methyl 4-hydroxycyclohexanecarboxylate

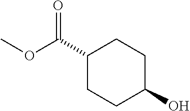

2M (Trimethylsilyl)diazomethane solution (83 mL, 166.47 mmol) in diethyl ether was added dropwise to a stirred solution of (1r,4r)-4-hydroxycyclohexanecarboxylic acid (CAS no. 3685-26-5) (20 g, 139 mmol), in methanol (100 mL) and toluene (100 mL) at ambient temperature, over a period of 30 minutes under air. The resulting solution was stirred at ambient temperature for 2 hours. The reaction mixture was concentrated then diluted with ethyl acetate (400 mL), and washed sequentially with 2M NaOH (200 mL), and saturated brine (200 mL). The organic layer was dried (MgSO₄) and evaporated to afford the product (12.25 g, 56%).

¹H NMR (300 MHz, DMSO) δ 1.04-1.21 (2H, m), 1.25-1.45 (2H, m), 1.72-1.92 (4H, m), 2.13-2.27 (1H, m), 3.26-3.40 (1H+water, m), 3.57 (3H, s), 4.52 (1H, d).

m/z 159 (M+H)⁺

### Intermediate 33

### (1s,4s)-methyl 4-(5-(5-(3-(trifluoromethoxy)phenylamino)-1,3,4-oxadiazole-2-carboxamido)pyridin-2-yloxy)cyclohexanecarboxylate

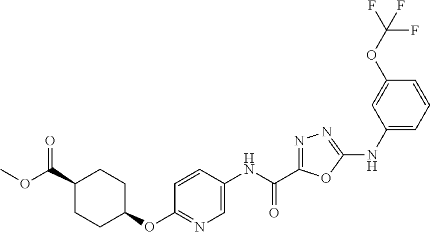

O-Perfluorophenyl 3-(trifluoromethoxy)phenylcarbamothioate (Intermediate 82) (599 mg, 1.49 mmol) was added to (1s,4s)-methyl 4-(5-(2-hydrazinyl-2-oxoacetamido)pyridin-2-yloxy)cyclohexanecarboxylate (Intermediate 28) (500 mg, 1.49 mmol), in DMF (8 mL). The resulting solution was stirred at 55° C. for 1 hour. 1-(3-Dimethylaminopropyl)-3-ethylcarbodiimide hydrochloride (285 mg, 1.49 mmol) was added and the solution was stirred at 75° C. for a further 17 hours. The DMF was removed by evaporation. The residue was triturated with water to give a solid which was collected by filtration. This washed with ether (10 mL) and dried under vacuum to afford the product (630 mg, 81%).

m/z 522 (M+H)⁺

### Intermediate 34

### (1s,4s)-methyl 4-(5-(5-(3-chlorophenylamino)-1,3,4-oxadiazole-2-carboxamido)pyridin-2-yloxy)cyclohexanecarboxylate

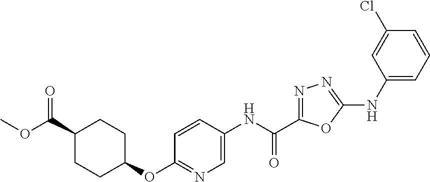

1-Chloro-3-isothiocyanatobenzene (CAS no. 2392-68-9) (333 mg, 1.96 mmol) was added to (1s,4s)-methyl 4-(5-(2-hydrazinyl-2-oxoacetamido)pyridin-2-yloxy)cyclohexanecarboxylate (Intermediate 28) (550 mg, 1.64 mmol), in DMF (8 mL) at 22° C. The resulting mixture was stirred at 55° C. for 1 hour. 1-(3-Dimethylaminopropyl)-3-ethylcarbodiimide hydrochloride (376 mg, 1.96 mmol) was added to the mixture. The resulting solution was stirred at 75° C. for 16 hours. The reaction mixture was concentrated and diluted with water (10 mL). A precipitate was collected by filtration. This was washed with water (10 mL) and dried under vacuum to afford the product (813 mg), which was used without further purification. m/z 472 (M+H)⁺

### Intermediate 35

### (1s,4s)-methyl 4-(5-(5-(4-fluoro-3-(trifluoromethyl)phenylamino)-1,3,4-oxadiazole-2-carboxamido)pyridin-2-yloxy)cyclohexanecarboxylate

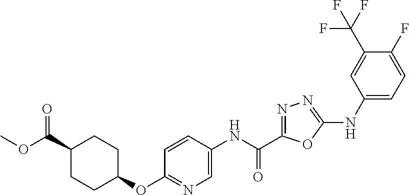

1-Fluoro-4-isothiocyanato-2-(trifluoromethyl)benzene (CAS no. 302912-43-2) (473 mg, 2.14 mmol) was added to (1s,4s)-methyl 4-(5-(2-hydrazinyl-2-oxoacetamido)pyridin-2-yloxy)cyclohexanecarboxylate (Intermediate 28) (550 mg, 1.64 mmol), in DMF (8 mL) at 22° C. The resulting suspension was stirred at 55° C. for 1 hour. 1-(3-Dimethylaminopropyl)-3-ethylcarbodiimide hydrochloride (410 mg, 2.14 mmol) was added to the mixture. The resulting solution was stirred at 75° C. for 16 hours. The reaction mixture was concentrated and diluted with water (10 mL). A precipitate was collected by filtration. This was washed with water (10 mL) and dried under vacuum to afford the product (1.65 g), which was used without further purification. m/z 524 (M+H)⁺

### Intermediate 36

### (1s,4s)-methyl 4-(5-(5-(4-tert-butylphenylamino)-1,3,4-oxadiazole-2-carboxamido)pyridin-2-yloxy)cyclohexanecarboxylate

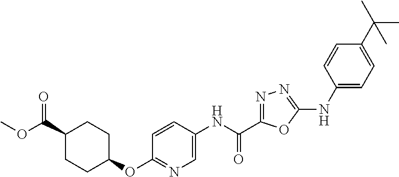

1-tert-Butyl-4-isothiocyanatobenzene (CAS no. 19241-24-8) (409 mg, 2.14 mmol) was added to (1s,4s)-methyl 4-(5-(2-hydrazinyl-2-oxoacetamido)pyridin-2-yloxy)cyclohexanecarboxylate (Intermediate 28) (550 mg, 1.64 mmol), in DMF (8 mL) at 22° C. The resulting suspension was stirred at 55° C. for 1 hour. 1-(3-Dimethylaminopropyl)-3-ethylcarbodiimide hydrochloride (410 mg, 2.14 mmol) was added to the mixture. The resulting solution was stirred at 75° C. for 16 hours. The reaction mixture was concentrated, then diluted with water (10 mL). A precipitate was collected by filtration. This was washed with water (10 mL) and dried under vacuum to afford the product (1.38 g), which was used without further purification. m/z 494 (M+H)⁺

### Intermediate 37

### (1s,4s)-methyl 4-(5-(5-(4-isopropylphenylamino)-1,3,4-oxadiazole-2-carboxamido)pyridin-2-yloxy)cyclohexanecarboxylate

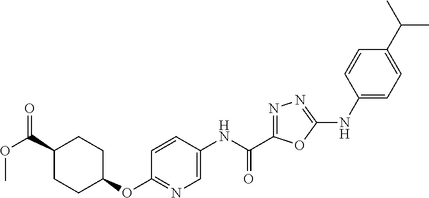

1-Isopropyl-4-isothiocyanatobenzene (CAS no. 89007-45-4) (379 mg, 2.14 mmol) was added to (1s,4s)-methyl 4-(5-(2-hydrazinyl-2-oxoacetamido)pyridin-2-yloxy)cyclohexanecarboxylate (Intermediate 28) (550 mg, 1.64 mmol), in DMF (8 mL) at 22° C. The resulting suspension was stirred at 55° C. for 1 hour. 143-Dimethylaminopropyl)-3-ethylcarbodiimide hydrochloride (410 mg, 2.14 mmol) was added to the mixture. The resulting solution was stirred at 75° C. for 16 hours. The reaction mixture was concentrated and diluted with water (10 mL). A precipitate was collected by filtration, washed with water (10 mL) and dried under vacuum to afford the product (1.01 g), which was used without further purification. m/z 480 (M+H)+

### Intermediate 38

### (1s,4s)-methyl 4-(5-(5-(4-(trifluoromethoxy)phenylamino)-1,3,4-oxadiazole-2-carboxamido)pyridin-2-yloxy)cyclohexanecarboxylate

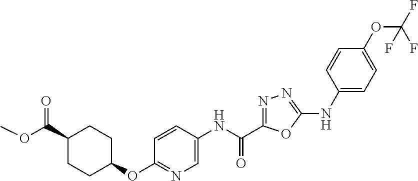

1-Isothiocyanato-4-(trifluoromethoxy)benzene (CAS no. 64285-95-6) (469 mg, 2.14 mmol) was added to (1s,4s)-methyl 4-(5-(2-hydrazinyl-2-oxoacetamido)pyridin-2-yloxy)cyclohexanecarboxylate (550 mg, 1.64 mmol), in DMF (8 mL) at 22° C. The resulting suspension was stirred at 55° C. for 1 hour. 1-(3-Dimethylaminopropyl)-3-ethylcarbodiimide hydrochloride (410 mg, 2.14 mmol) was added to the mixture. The resulting solution was stirred at 75° C. for 16 hours. The reaction mixture was concentrated, then diluted with water (10 mL). A precipitate was collected by filtration. This was washed with water (10 mL) and dried under vacuum to afford the product (1.17 g), which was used without further purification. m/z 522 (M+H)⁺

### Intermediate 39

### (1s,4s)-methyl 4-(5-(5-(2,4-dichlorophenylamino)-1,3,4-oxadiazole-2-carboxamido)pyridin-2-yloxy)cyclohexanecarboxylate

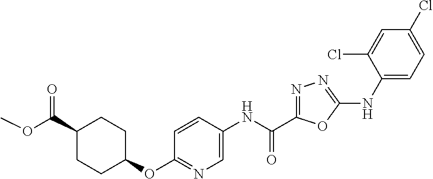

2,4-dichloro-1-isothiocyanatobenzene (CAS no. 6590-96-1) (437 mg, 2.14 mmol) was added to (1s,4s)-methyl 4-(5-(2-hydrazinyl-2-oxoacetamido)pyridin-2-yloxy)cyclohexanecarboxylate (Intermediate 28) (550 mg, 1.64 mmol), in DMF (8 mL) at 22° C. The resulting suspension was stirred at 55° C. for 1 hour. 1-(3-Dimethylaminopropyl)-3-ethylcarbodiimide hydrochloride (410 mg, 2.14 mmol) was added to the mixture. The resulting solution was stirred at 75° C. for 16 hours. The reaction mixture was concentrated, then diluted with water (10 mL). A precipitate was collected by filtration. This was then washed with water (10 mL), and dried under vacuum to afford the product (1.20 g), which was used without further purification. m/z 506 (M+H)⁺

### Intermediate 40

### (1s,4s)-methyl 4-(5-(5-(4-bromo-2-chlorophenylamino)-1,3,4-oxadiazole-2-carboxamido)pyridin-2-yloxy)cyclohexanecarboxylate

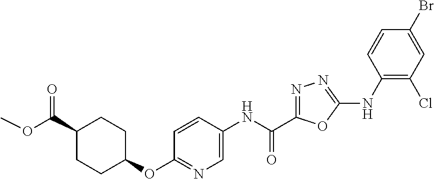

4-bromo-2-chloro-1-isothiocyanatobenzene (CAS no. 98041-69-1) (532 mg, 2.14 mmol) was added to (1s,4s)-methyl 4-(5-(2-hydrazinyl-2-oxoacetamido)pyridin-2-yloxy)cyclohexanecarboxylate (Intermediate 28) (550 mg, 1.64 mmol), in DMF (8 mL) at 22° C. The resulting suspension was stirred at 55° C. for 1 hour. 1-(3-Dimethylaminopropyl)-3-ethylcarbodiimide hydrochloride (410 mg, 2.14 mmol) was added to the mixture. The resulting solution was stirred at 75° C. for 16 hours. The reaction mixture was concentrated then diluted with water (10 mL). A precipitate was collected by filtration. This was washed with water (10 mL) and dried under vacuum to afford the product (936 mg, 95%), which was used without further purification. m/z 552 (M+H)⁺

### Intermediate 41

### (1s,4s)-methyl 4-(5-(5-(2,3-dichlorophenylamino)-1,3,4-oxadiazole-2-carboxamido)pyridin-2-yloxy)cyclohexanecarboxylate

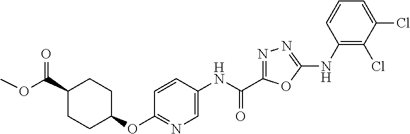

1,2-dichloro-3-isothiocyanatobenzene (CAS no. 6590-97-2) (400 mg, 1.96 mmol) was added to (1s,4s)-methyl 4-(5-(2-hydrazinyl-2-oxoacetamido)pyridin-2-yloxy)cyclohexanecarboxylate (Intermediate 28) (550 mg, 1.64 mmol), in DMF (8 mL) at 22° C. The resulting suspension was stirred at 55° C. for 1 hour. 1-(3-Dimethylaminopropyl)-3-ethylcarbodiimide hydrochloride (376 mg, 1.96 mmol) was added to the mixture. The resulting solution was stirred at 75° C. for 16 hours. The reaction mixture was concentrated by evaporation, then diluted with water (10 mL). A precipitate was collected by filtration. This was washed with water (10 mL) and dried under vacuum to afford the product (553 mg, 67%), which was used without further purification.

m/z 506 (M+H)⁺

### Intermediate 42

### (1s,4s)-methyl 4-(5-(5-(3-chloro-4-methylphenylamino)-1,3,4-oxadiazole-2-carboxamido)pyridin-2-yloxy)cyclohexanecarboxylate

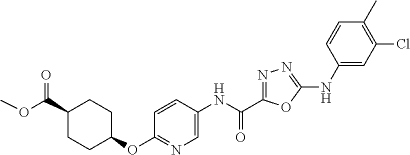

2-Chloro-4-isothiocyanato-1-methylbenzene (CAS no. 19241-37-3) (393 mg, 2.14 mmol) was added to (1s,4s)-methyl 4-(5-(2-hydrazinyl-2-oxoacetamido)pyridin-2-yloxy)cyclohexanecarboxylate (Intermediate 28) (550 mg, 1.64 mmol), in DMF (8 mL) at 22° C. under air. The resulting suspension was stirred at 55° C. for 1 hour. 1-(3-Dimethylaminopropyl)-3-ethylcarbodiimide hydrochloride (410 mg, 2.14 mmol) was added to the mixture. The resulting solution was stirred at 75° C. for 16 hours. The reaction mixture was concentrated by evaporation and was then diluted with water (10 mL). A precipitate was collected by filtration. This was washed with water (10 mL), ether (10 mL) and dried under vacuum to afford the product (946 mg), which was used without further purification. m/z 486 (M+H)⁺

### Intermediate 43

### (1s,4s)-methyl 4-(5-(5-(3-chloro-4-fluorophenylamino)-1,3,4-oxadiazole-2-carboxamido)pyridin-2-yloxy)cyclohexanecarboxylate

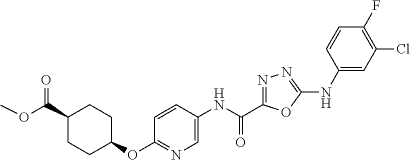

2-Chloro-1-fluoro-4-isothiocyanatobenzene (CAS no. 137724-66-4) (402 mg, 2.14 mmol) was added to (1s,4s)-methyl 4-(5-(2-hydrazinyl-2-oxoacetamido)pyridin-2-yloxy)cyclohexanecarboxylate (Intermediate 28) (550 mg, 1.64 mmol), in DMF (8 mL) at 22° C. The resulting suspension was stirred at 55° C. for 1 hour. 1-(3-Dimethylaminopropyl)-3-ethylcarbodiimide hydrochloride (410 mg, 2.14 mmol) was added to the mixture. The resulting solution was stirred at 75° C. for 16 hours. The reaction mixture was concentrated by evaporation, then diluted with water (10 mL). A precipitate was collected by filtration. This was washed with water (10 mL) and dried under vacuum to afford the product (853 mg, 98%), which was used without further purification.

m/z 490 (M+H)⁺

### Intermediate 44

### (1s,4s)-methyl 4-(5-(5-(3,4-difluorophenylamino)-1,3,4-oxadiazole-2-carboxamido)-3-methylpyridin-2-yloxy)cyclohexanecarboxylate

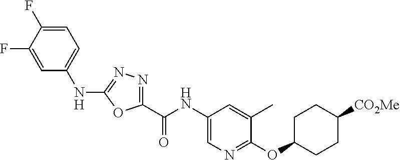

3,4-Difluorophenylisocyanate (CAS no. 42601-04-7) (0.205 g, 1.20 mmol) was added to a stirred solution of (1s,4s)-methyl 4-(5-(2-hydrazinyl-2-oxoacetamido)-3-methylpyridin-2-yloxy)cyclohexanecarboxylate (Intermediate 45) (0.350 g, 1.0 mmol) in DMF (10 mL) at 65° C. under nitrogen. The resulting solution was stirred at 65° C. for 30 minutes. 1-(3-Dimethylaminopropyl)-3-ethylcarbodiimide hydrochloride (0.230 g, 1.20 mmol) was added to the reaction mixture and the resulting solution was stirred at 85° C. for 30 minutes. The reaction mixture was allowed to cool to ambient temperature before adding water (10 mL).

The precipitate was collected by filtration, washed with water (10 mL) and dried under vacuum to afford the title compound (0.487 g), which was used without further purification.

1H NMR (400 MHz, DMSO) δ 1.63-1.93 (m, 8H), 2.17 (s, 3H), 2.44-2.54 (m, 1H), 3.63 (s, 3H), 5.18-5.24 (m, 1H), 7.33-7.39 (m, 1H), 7.49 (q, 1H), 7.66-7.74 (m, 1H), 7.91-7.99 (m, 1H), 8.32 (d, 1H), 11.01 (s, 1H), 11.23 (s, 1H). m/z 488.28 (M+H)⁺

### Intermediate 45

### (1s,4s)-methyl 4-(5-(2-hydrazinyl-2-oxoacetamido)-3-methylpyridin-2-yloxy)cyclohexanecarboxylate

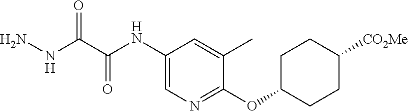

Hydrazine hydrate (0.085 mL, 1.76 mmol) was added to a stirred solution of methyl (1s,4s)-methyl 4-(5-(2-methoxy-2-oxoacetamido)-3-methylpyridin-2-yloxy)cyclohexanecarboxylate (Intermediate 46) (0.561 g, 1.6 mmol) in ethanol (20 mL) warmed to 70° C. The resulting solution was stirred at 70° C. for 10 minutes.

The precipitate was collected by filtration, washed with diethyl ether (20 mL) and dried under vacuum to afford the title compound (0.561 g), which was used without further purification.

1H NMR (400 MHz, DMSO) δ 1.62-1.90 (m, 8H), 2.15 (s, 3H), 2.44-2.49 (m, 1H), 3.62 (s, 3H), 4.59 (s, 2H), 5.16-5.22 (m, 1H), 7.91-7.93 (m, 1H), 8.35 (d, 1H), 10.54 (s, 1H). m/z 351.26 (M+H)⁺

### Intermediate 46

### (1s,4s)-methyl 4-(5-(2-methoxy-2-oxoacetamido)-3-methylpyridin-2-yloxy)cyclohexanecarboxylate

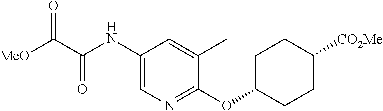

Methyl oxalyl chloride (0.322 mL, 3.50 mmol) was added to a stirred solution of (1s,4s)-methyl 4-(5-amino-3-methylpyridin-2-yloxy)cyclohexanecarboxylate (Intermediate 47) (0.772 g, 2.92 mmol), and pyridine (0.472 mL, 5.84 mmol) in DCM (20 mL) cooled to 0° C. under nitrogen. The resulting solution was stirred at ambient temperature for 1 hour. The reaction mixture was quenched with water (10 mL), extracted with DCM (2×20 mL), the organic layer was dried (MgSO₄), filtered and evaporated to afford the'crude product (1.023 g), which was used without further purification.

1H NMR (400 MHz, DMSO) δ 1.60-1.91 (m, 8H), 2.15 (s, 3H), 2.44-2.50 (m, 1H), 3.62 (s, 3H), 3.86 (s, 3H), 5.17-5.23 (m, 1H), 7.86 (d, 1H), 8.28 (d, 1H), 10.75 (s, 1H)

m/z 351.40 (M+H)⁺

### Intermediate 47

### (1s,4s)-methyl 4-(5-amino-3-methylpyridin-2-yloxy)cyclohexanecarboxylate

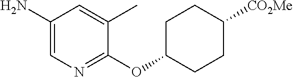

2M hydrochloric acid (110 mL) was added to a stirred solution of (1s,4s)-methyl 4-(5-(diphenylmethyleneamino)-3-methylpyridin-2-yloxy)cyclohexanecarboxylate (Intermediate 48) (1.556 g, 3.63 mmol) in methanol (20 mL) at 20° C. This was stirred at ambient temperature for 2 hours.

The reaction mixture was adjusted to pH 7 with 2M NaOH. The reaction mixture was evaporated, and the resulting aqueous solution was extracted with ethyl acetate (50 mL). The organic layer was dried (MgSO₄), filtered and evaporated to afford crude product. The crude product was purified by flash silica chromatography, eluting with 0 to 8% methanol in DCM to afford the pure product (0.772 g, 80%).

1H NMR (400 MHz, DMSO) δ 1.55-1.86 (m, 8H), 2.05 (s, 3H), 2.39-2.50 (m, 1H), 3.62 (s, 3H), 4.58 (s, 2H), 4.99-5.05 (m, 1H), 6.84 (d, 1H), 7.32 (d, 1H). m/z 265.39 (M+H)⁺

### Intermediate 48

### (1s,4s)-methyl 4-(5-(diphenylmethyleneamino)-3-methylpyridin-2-yloxy)cyclohexanecarboxylate

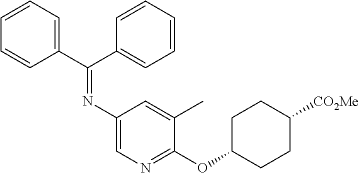

Benzophenone imine (0.914 mL, 5.45 mmol) was added to (1s,4s)-methyl-4-(5-bromo-3-methylpyridin-2-yloxy)cyclohexane carboxylate (Intermediate 49) (1.191 g, 3.63 mmol), palladium(II) acetate (0.049 g, 0.22 mmol), cesium carbonate (1.656 g, 5.08 mmol) and (S)-(−)-2′-bis(diphenylphosphino)-1,1′-binaphthyl (0.136 g, 0.22 mmol) in THF (40 mL) at 20° C. under nitrogen. The resulting solution was stirred at reflux for 8 hours.

The reaction mixture was evaporated to dryness and redissolved in ethyl acetate (50 mL), and washed sequentially with water (25 mL), and saturated brine (25 mL). The organic layer was dried (MgSO₄), filtered and evaporated to afford crude product which was used without further purification. m/z 429.27 (M+H)⁺

### Intermediate 49

### (1s,4s)-methyl-4-(5-bromo-3-methylpyridin-2-yloxy)cyclohexane carboxylate

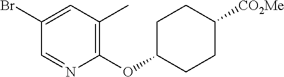

A few drops of conc. hydrochloric acid were added to a stirred solution of (1s,4s)-4-(5-s bromo-3-methylpyridin-2-yloxy)cyclohexanecarboxylic acid (Intermediate 50) (2.40 g, 7.64 mmol) in methanol at 20° C. The resulting solution was stirred at 60° C. for 3 hours. The reaction mixture was basified with saturated aqueous NaHCO₃ and the methanol was removed by evaporation. The aqueous solution was extracted with ethyl acetate (3×50 mL), the organic layer was dried over MgSO₄, filtered and evaporated to afford yellow oil. The crude product was purified by flash silica chromatography, elution gradient 0 to 30% ethyl acetate in isohexane. Pure fractions were evaporated to dryness to afford the title compound (2.06 g).

1H NMR (400 MHz, DMSO) δ 1.63-1.90 (m, 8H), 2.15 (s, 3H), 2.45-2.49 (m, 1H), 3.62 (s, 3H), 5.15-5.21 (m, 1H), 7.75-7.78 (m, 1H), 8.05-8.08 (m, 1H). m/z 330.3 (M+H)⁺

### Intermediate 50

### (1s,4s)-4-(5-bromo-3-methylpyridin-2-yloxy)cyclohexanecarboxylic acid

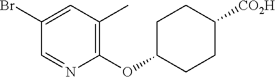

A solution of cis-4-hydroxycyclohexanecarboxylic acid (CAS no. 3685-22-1) (3.60 g, 25.00 mmol) in DMA (20 mL) was added to a stirred mixture of sodium hydride (2.00 g, 50.00 mmol) in DMA (80 mL) cooled to 0° C. The resulting suspension was stirred at ambient temperature for 30 minutes. 2,5-Dibromo-3-methylpyridine (CAS no. 3430-18-0) (6.27 g, 25.00 mmol) was added and the resulting suspension was stirred at 120° C. for 24 hours.

The reaction mixture was allowed to cool to room temperature, diluted with water (50 mL) and neutralised with 2M hydrochloric acid.

The reaction mixture was extracted with ethyl acetate (3×50 mL), the organic layer was dried over MgSO4, filtered and evaporated to afford brown oil.

The crude product was purified by flash silica chromatography, elution gradient 0 to 10% methanol in DCM. Pure fractions were evaporated to dryness to afford the title compound (2.4 g).

1H NMR (400 MHz, DMSO) δ 1.61-1.90 (m, 8H), 2.15 (s, 3H), 2.32-2.41 (m, 1H), 5.13-5.20 (m, 1H), 7.75-7.78 (m, 1H), 8.05-8.08 (m, 1H), 12.05 (s, 1H). m/z 316.1 (M+H)⁺

### Intermediate 51

### (1s,4s)-methyl 4-(5-(5-(3-chlorophenylamino)-1,3,4-oxadiazole-2-carboxamido)-3-methylpyridin-2-yloxy)cyclohexanecarboxylate

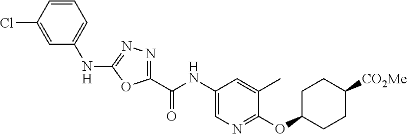

3-Chlorophenylisocyanate (CAS no. 2392-68-9) (0.190 g, 1.12 mmol) was added to a stirred solution of (1s,4s)-methyl 4-(5-(2-hydrazinyl-2-oxoacetamido)-3-methylpyridin-2-yloxy)cyclohexanecarboxylate (Intermediate 45) (0.392 g, 1.12 mmol) in DMF (10 mL) at 65° C. under nitrogen. The resulting solution was stirred at 65° C. for 30 minutes. 1-(3-Dimethylaminopropyl)-3-ethylcarbodiimide hydrochloride (0.258 g, 1.34 mmol) was added to the reaction mixture and the resulting solution was stirred at 85° C. for 30 minutes. The reaction mixture was allowed to cool to ambient temperature before adding water (10 mL).

The precipitate was collected by filtration, washed with water (10 mL) and dried under vacuum to afford the title compound (0.544 g), which was used without further purification.

1H NMR (400 MHz, DMSO) δ 1.64-1.92 (m, 8H), 2.17 (s, 3H), 2.44-2.51 (m, 1H), 3.63 (s, 3H), 5.18-5.25 (m, 1H), 7.10-7.15 (m, 1H), 7.43 (t, 1H), 7.50-7.55 (m, 1H), 7.74 (t, 1H), 7.92-7.95 (m, 1H), 8.32 (d, 1H), 11.01 (s, 1H), 11.22 (s, 1H). m/z 486.25 (M+H)⁺

### Intermediate 52

### tert-butyl 4-({5-[({5-[(2,4,5-trifluorophenyl)amino]-1,3,4-oxadiazol-2-yl}carbonyl)amino]pyridin-2-yl}oxy)cyclohexanecarboxylate

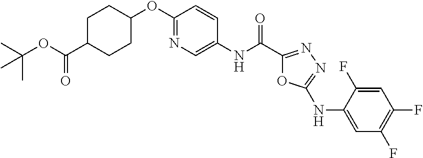

2,4,5-trifluorophenylisothiocyanate (130 μl, 0.98 mmol) was added to a stirred solution of tert-butyl 4-[(5-{[hydrazino(oxo)acetyl]amino}pyridin-2-yl)oxy]cyclohexanecarboxylate (Intermediate 54) (337 mg, 0.89 mmol) in 7 mL DMF. The reaction mixture was stirred for 3 h at ambient temperature before adding N-(3-Dimethylaminopropyl)-N′-ethylcarbodiimide hydrochloride (205 mg, 1.07 mmol) as a solid. The reaction mixture was stirred for a further 16 h before being concentrated in vacuo and triturated with water. The yellow solid was filtered and further washed with water to yield the title compound (474 mg, 100%). m/z 433.96 (M+H)⁺

### Intermediate 53

### tert-butyl 4-({5-[({5-[(4-fluorophenyl)amino]-1,3,4-oxadiazol-2-yl}carbonyl)amino]pyridin-2-yl}oxy)cyclohexanecarboxylate

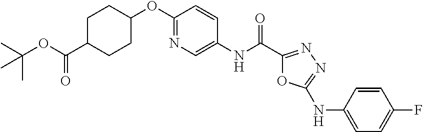

4-fluorophenylisothiocyanate (119 μl, 0.98 mmol) was added to a stirred solution of tert-butyl 4-[(5-{[hydrazino(oxo)acetyl]amino}pyridin-2-yl)oxy]cyclohexanecarboxylate (Intermediate 54) (337 mg, 0.89 mmol) in 7 mL DMF. The reaction mixture was stirred for 3 h at ambient temperature before adding N-(3-Dimethylaminopropyl)-N′-ethylcarbodiimide hydrochloride (205 mg, 1.07 mmol) as a solid. The reaction mixture was stirred for a further 16 h before being concentrated in vacuo and triturated with water. The yellow solid was filtered and further washed with water to yield the title compound (396 mg, 89%). m/z 497.99 (M+H)⁺

### Intermediate 54

### tert-butyl 4-[(5-{[hydrazino(oxo)acetyl]amino}pyridin-2-yl)oxy]cyclohexanecarboxylate

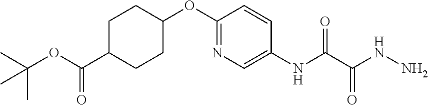

Hydrazine hydrate (483 μl, 9.95 mmol) was added to a stirred solution of tert-butyl 4-[(5-{[methoxy(oxo)acetyl]amino}pyridin-2-yl)oxy]cyclohexanecarboxylate (Intermediate 55) (754 mg, 1.99 mmol) in ethanol/DCM (15 mL/2 mL). The reaction mixture was filtered after 30 min and washed with ethanol. The solid was dried and yielded the title compound as a pink solid (674 mg, 89%). m/z 378.94 (M+H)⁺

### Intermediate 55

### tert-butyl 4-[(5-{[methoxy(oxo)acetyl]-amino}pyridin-2-yl)oxy]cyclohexanecarboxylate

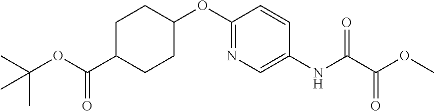

Methyl oxalyl chloride (250 μl, 2.72 mmol) was added to a stirred mixture of tert-butyl 4-[(5-aminopyridin-2-yl)oxy]cyclohexanecarboxylate (721 mg, 2.47 mmol) (Intermediate 56) and Diisopropylaminomethyl-Polystyrene (1.852 g, 7.41 mmol) in DCM (15 mL) under nitrogen. The reaction mixture was stirred at ambient temperature for 30 minutes before filtering and washing with DCM. The filtrate was concentrated in vacuo. Attempted separation of cis and trans isomers by chromatography failed. The title compound was isolated as a pink solid (754 mg, 81%). m/z 378.94 (M+H)⁺

### Intermediate 56

### tert-butyl 4-[(5-aminopyridin-2-yl)oxy]cyclohexanecarboxylate (721 mg, 2.47 mmol)

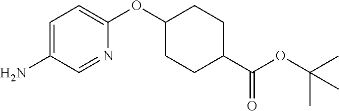

10% Palladium on carbon (150 mg) was added to a stirred solution of tert-butyl 4-[(5-nitropyridin-2-yl)oxy]cyclohexanecarboxylate (Intermediate 57) (1.35 g, 4.19 mmol) in ethyl acetate (75 mL) under nitrogen. The reaction mixture was put under a hydrogen atmosphere and stirred for 16 h before filtering through celite and washing with ethyl acetate. The filtrate was concentrated in vacuo and the crude product was purified by flash silica chromatography, eluting with 0 to 20% ethyl acetate in isohexane to afford the title compound as an oil (mixture of cis and trans isomers) (721 mg, 59%).

¹H NMR (400 MHz, CDCl₃) δ1.39-1.48 (1H, m), 1.44 (9H, s), 1.58 (3H, m), 1.99-2.03 (2H, m), 2.15-2.21 (3H, m), 3.35 (2H, s), 4.82 (1H, s), 6.53-6.55 (1H, m), 7.00-7.03 (1H, m), 7.64 (1H, d). m/z 293.01 (M+H)⁺

### Intermediate 57

### tert-butyl 4-[(5-nitropyridin-2-yl)oxy]cyclohexanecarboxylate

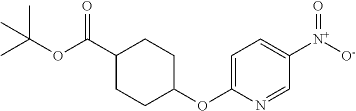

Potassium tert-butoxide (2.71 g, 24.17 mmol) was added to a stirred solution of tert-butyl 4-hydroxycyclohexanecarboxylate (2.42 g, 12.08 mmol) and 2-fluoro-5-nitropyridine (1.72 g, 12.08 mmol) in THF (18 mL) under nitrogen. The reaction mixture was heated in the microwave at 90° C. for 4 min before being cooled and quenched with saturated ammonium chloride solution (50 mL). The mixture was extracted with ethyl acetate (2×50 mL) and the organics washed with water (50 mL). The organic phase was dried (MgSO₄) and solvent removed in vacuo. The crude product was purified by flash silica chromatography, eluting with 0 to 20% ethyl acetate in isohexane to afford the title compound as yellow solid (1.35 g, 38%).

¹H NMR (400 MHz, CDCl₃) δ1.45-1.49 (9H, m), 1.51-2.35 (9H, m), 5.18-5.38 (1H, m), 6.78 (1H, m), 8.31-8.35 (1H, m), 9.05 (1H, d). m/z 266.92 (M-tBu)

### Intermediate 58

### ethyl 4-[(5-{[(5-{[4-(difluoromethoxy)phenyl]amino}-1,3,4-oxadiazol-2-yl)carbonyl]amino}pyridin-2-yl)oxy]cyclohexanecarboxylate

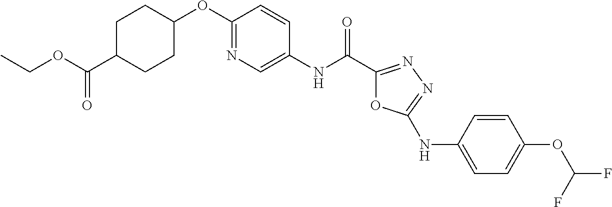

4-difluoromethoxyphenylisothiocyanate (186 mg, 0.92 mmol) was added to a stirred solution of ethyl 4-[(5-{[hydrazino(oxo)acetyl]amino}pyridin-2-yl)oxy]cyclohexanecarboxylate (Intermediate 59) (294 mg, 0.84 mmol) in DMF (4 mL). The reaction mixture was stirred for 3 h at ambient temperature before adding N-(3-Dimethylaminopropyl)-N′-ethylcarbodiimide hydrochloride (194 mg, 1.01 mmol) as a solid. The reaction mixture was stirred for a further 16 h before being concentrated in vacuo and triturated with water. The yellow solid was filtered and further washed with water to yield the title compound (385 mg, 86%).

¹H NMR (400 MHz, DMSO) δ1.19 (3H, t), 1.46 (3H, d), 1.69-1.83 (4H, m), 1.95-1.98 (1H, m), 2.10 (1H, d), 2.35 (1H, d), 4.04-4.11 (2H, m), 4.90-5.13 (1H, m), 6.79-6.85 (1H, m), 7.25 (2H, d), 7.64 (2H, d), 8.04-8.07 (1H, m), 8.52 (1H, t), 11.12 (1H, d).

m/z 517.95 (M+H)⁺

### Intermediate 59

### ethyl 4-[(5-{[hydrazino(oxo)acetyl]amino}pyridin-2-yl)oxy]cyclohexanecarboxylate

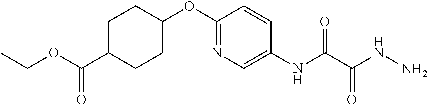

Hydrazine hydrate (366 μl, 7.55 mmol) was added to a stirred solution of ethyl 4-[(5-{[methoxy(oxo)acetyl]amino}pyridin-2-yl)oxy]cyclohexanecarboxylate (Intermediate 60) (571 mg, 1.51 mmol) in ethanol (15 mL). The reaction mixture was filtered after 2 h and washed with ethanol. The solid was dried and yielded the title compound as a white solid (294 mg, 56%). The compound was used directly without further purification.

### Intermediate 60

### ethyl 4-[(5-{[methoxy(oxo)acetyl]amino}pyridin-2-yl)oxy]cyclohexanecarboxylate

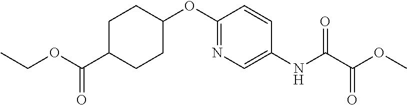

Methyl oxalyl chloride (153 μl, 1.66 mmol) was added to a stirred mixture of ethyl 4-[(5-aminopyridin-2-yl)oxy]cyclohexanecarboxylate (400 mg, 1.51 mmol) (Intermediate 61) and Diisopropylaminomethyl-Polystyrene (1.132 g, 4.53 mmol) in DCM (15 mL) under nitrogen. The reaction mixture was stirred at ambient temperature for 30 minutes before filtering and washing with DCM. The filtrate was concentrated in vacuo and the crude product used directly. m/z 351.06 (M+H)⁺

### Intermediate 61

### ethyl 4-[(5-aminopyridin-2-yl)oxy]cyclohexanecarboxylate

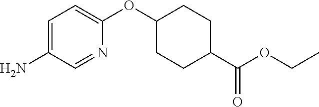

10% Palladium on carbon (150 mg) was added to a stirred solution of ethyl 4-[(5-nitropyridin-2-yl)oxy]cyclohexanecarboxylate (Intermediate 62) (1.109 g, 3.77 mmol) in ethyl acetate (75 mL) under nitrogen. The reaction mixture was put under a hydrogen atmosphere and stirred for 16 h before filtering through celite and washing with ethyl acetate. The filtrate was concentrated in vacuo and purification of the crude product was attempted by flash silica chromatography, but yielded a mixture of cis and trans isomers (400 mg, 40%). m/z 264.99 (M+H)⁺

### Intermediate 62

### ethyl 4[(5-nitropyridin-2-yl)oxy]cyclohexanecarboxylate

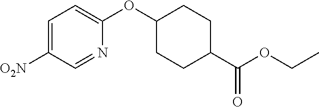

Potassium tert-butoxide (3.95 g, 35.2 mmol) was added to a stirred solution of ethyl 4-hydroxycyclohexanecarboxylate (3.03 g, 17.6 mmol) and 2-fluoro-5-nitropyridine (2.5 g, 17.6 mmol) in THF (40 mL) under nitrogen at 5° C. The reaction mixture was allowed to warm to ambient temperature and stirred for 1 h. The mixture was then quenched with water (100 mL) and extracted with ethyl acetate (2×100 mL) and the organics washed with water (100 mL). The organic phase was dried (MgSO₄) and solvent removed in vacuo. The crude product was purified by flash silica chromatography, eluting with 0 to 30% ethyl acetate in isohexane to afford the title compound as a yellow oil (907 mg, 18%).

¹H NMR (400 MHz, CDCl₃) δ1.28 (3H, m), 1.51-2.5 (10H, m), 4.15 (2H, m), 5.10-5.4 (1H, m), 6.78 (1H, m), 8.32 (1H, m), 9.05 (1H, d). m/z 294.9 (M+H)⁺

### Intermediate 63

### (1r,4r)-methyl 4-(5-(5-(2,4,5-trifluorophenylamino)-1,3,4-oxadiazole-2-carboxamido)pyridin-2-yloxy)cyclohexanecarboxylate

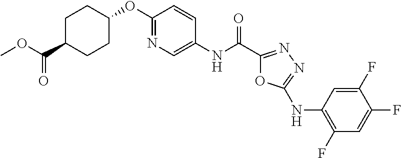

2,4,5-Trifluorophenyl isothiocyanate (270 mg, 1.43 mmol) was added to (1r,4r)-methyl 4-(5-(2-hydrazinyl-2-oxoacetamido)pyridin-2-yloxy)cyclohexanecarboxylate (Intermediate 75) (400 mg, 1.19 mmol) in DMF (8 mL) at ambient temperature. The resulting solution was stirred at 50° C. for 1 hour. 1-(3-Dimethylaminopropyl)-3-ethylcarbodiimide hydrochloride (274 mg, 1.43 mmol) was added and the solution stirred at 85° C. for 3 hours. The reaction mixture was allowed to cool to ambient temperature and half the DMF was evaporated before adding water (8 mL).

The precipitate was collected by filtration, washed with water (25 mL) and dried under vacuum to afford (1r,4r)-methyl 4-(5-(5-(2,4,5-trifluorophenylamino)-1,3,4-oxadiazole-2-carboxamido)pyridin-2-yloxy)cyclohexanecarboxylate (576 mg, 99%) as a yellow solid which was used without further purification.

1H NMR (400 MHz, DMSO) δ 1.38-1.58 (4H, m), 1.92-2.01 (2H, m), 2.07-2.14 (2H, m), 2.36-2.42 (1H, m), 3.61 (3H, s), 4.86-4.96 (1H, m), 6.81 (1H, d), 7.70-7.77 (1H, m), 8.04-8.06 (1H, m), 8.14-8.22 (1H, m), 8.51 (1H, d), 11.08 (1H, s), 11.17 (1H, s).

m/z 492 (M+H)⁺

Intermediates 64-74 were prepared in a similar manner:

| Intermediatate | Structure | m/z | NMR |
|---|---|---|---|
| 64 (1r,4r)-methyl 4-(5-(5- (2,4- dichloro- phenylamino)- 1,3,4- oxadiazole-2- carboxamido) pyridin-2- yloxy) cyclohexane- carboxylate | 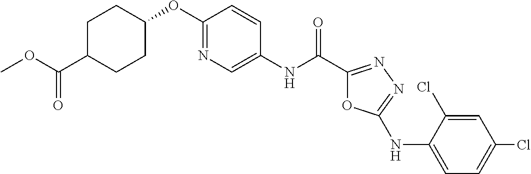 | 506 (M + H)⁺ | 1H NMR (400 MHz, DMSO) δ 1.40-1.55 (4H, m), 1.94-2.00 (2H, m), 2.08-2.14 (2H, m), 2.35-2.43 (1H, m), 3.62 (3H, s), 4.85-4.94 (1H, m), 6.81 (1H, d), 7.52-7.56 (1H, m), 7.73 (1H, d), 8.02-8.07 (2H, m), 8.51 (1H, d), 10.59 (1H s), 11.15 (1H, s) |
| 65 (1r,4r)-methyl 4-(5-(5-(3- chloro-4- fluoro- phenylamino)- 1,3,4- oxadiazole-2- carboxamido) pyridin-2- yloxy) cyclohexane- carboxylate | 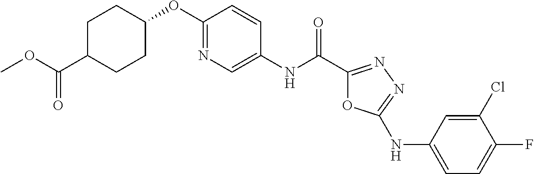 | 490 (M + H)⁺ | 1H NMR (400 MHz, DMSO) δ 1.37-1.56 (4H, m), 1.91-2.01 (2H, m), 2.06-2.13 (2H, m), 2.31-2.44 (1H, m), 3.62 (3H, s), 4.85-4.94 (1H, m), 6.81 (1H, d), 7.48 (1H, t), 7.51-7.56 (1H, m), 7.82-7.85 (1H, m), 8.05-8.08 (1H, m), 8.51 (1H, d), 11.17 (1H s), 11.28 (1H, s) |
| 66 (1r,4r)-methyl 4-(5-(5-(4- (trifluoro- methoxy) phenylamino)- 1,3,4- oxadiazole-2- carboxamido) pyridin-2- yloxy) cyclohexane- carboxylate | 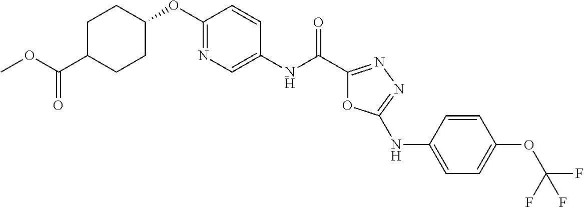 | 522 (M + H)⁺ | 1H NMR (400 MHz, DMSO) δ 1.40-1.58 (4H, m), 1.93-2.00 (2H, m), 2.07-2.12 (2H, m), 2.32-2.44 (1H, m), 3.63 (3H, s), 4.88-4.93 (1H, m), 6.80 (1H, d), 7.44 (2H, d), 7.71 (2H, d), 8.05-8.08 (1H, m), 8.52 (1H, d), 11.16 (1H, s), 11.26 (1H, s) |
| 67 (1r,40-methyl 4-(5-(5-(3- chloro-4- methyl- phenylamino)- 1,3,4- oxadiazole-2- carboxamido) pyridin-2- yloxy) cyclohexane- carboxylate | 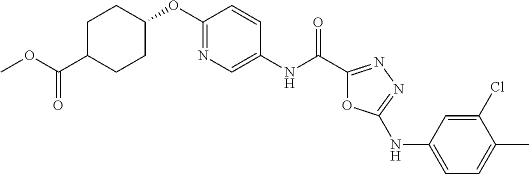 | 486 (M + H)⁺ | 1H NMR (400 MHz, DMSO) δ 1.39-1.58 (4H, m), 1.93-2.01 (2H, m), 2.06-2.12 (2H, m), 2.29 (3H, s), 2.37-2.43 (1H, m), 3.61 (3H, s), 4.86- 4.94 (1H, m), 6.80 (1H, d), 7.35-7.39 (1H, m), 7.41-7.44 (1H, m), 7.73 (1H, d), 8.04-8.07 (1H, m), 8.51 (1H, d), 11.15 (2H, s) |
| 68 (1r,4r)-methyl 4-(5-(5-(3- chlorophenyl- amino)-1,3,4 oxadiazole-2- carboxamido) pyridin-2- yloxy) cyclohexane- carboxylate | 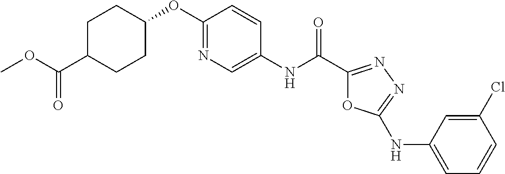 | 472 (M + H)⁺ | 1H NMR (400 MHz, DMSO) δ 1.38-1.59 (4H, m), 1.92-2.00 (2H, m), 2.06-2.14 (2H, m), 2.34-2.42 (1H, m), 3.61 (3H, s), 4.86-4.93 (1H, m), 6.81 (1H, d), 7.13 (1H, d), 7.43 (1H, t), 7.50- 7.54 (1H, m), 7.74 (1H, t), 8.04-8.08 (1H, m), 8.52 (1H, d), 11.17(1H s), 11.29 (1H, s) |
| 69 (1r,4r)-methyl 4-(5-(5- (2,3- dichloro- phenylamino)- 1,3,4- oxadiazole-2- carboxamido) pyridin-2- yloxy) cyclohexane- carboxylate | 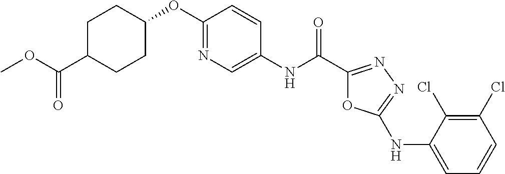 | 506 (M + H)⁺ | 1H NMR (400 MHz, DMSO) δ 1.37-1.59 (4H, m), 1.94-2.02 (2H, m), 2.07-2.13 (2H, m), 2.32-2.44 (1H, m), 3.60 (3H, s), 4.86-4.94 (1H, m), 6.80 (1H, d), 7.44- 7.48 (2H, m), 7.98- 8.02 (1H, m), 8.04- 8.08 (1H, m), 8.51 (1H, d), 10.67 (1H, s), 11.15 (1H, s) |
| 70 (1r,4r)-methyl 4-(5-(5-(4- fluoro-3- (trifluoromethyl) phenylamino)- 1,3,4- oxadiazole-2- carboxamido) pyridin-2- yloxy) cyclohexane- carboxylate | 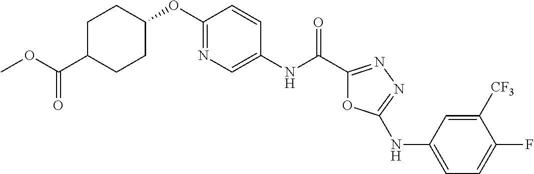 | 524 (M + H)⁺ | 1H NMR (400 MHz, DMSO) δ 1.39-1.57 (4H, m), 1.94-2.02 (2H, m), 2.07-2.14 (2H, m), 2.33-2.44 (1H, m), 3.60 (3H, s), 4.83-4.93 (1H, m), 6.80 (1H, d), 7.59 (1H, t), 7.86-7.92 (1H, m), 7.99-8.09 (2H, m), 8.51 (1H, d), 11.17 (1H, s), 11.42 (1H, s) |
| 71 (1r,4r)-methyl 4-(5-(5-(4- isopropyl- phenylamino)- 1,3,4- oxadiazole-2- carboxamido) pyridin-2- yloxy) cyclohexane- carboxylate | 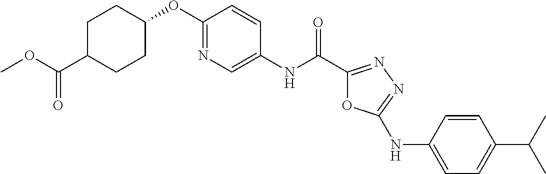 | 480 (M + H)⁺ | 1H NMR (400 MHz, DMSO) δ 1.21 (6H, d), 1.40-1.58 (4H, m), 1.93-2.01 (2H, m), 2.06-2.14 (2H, m), 2.36-2.42 (1H, m), 2.82-2.95 (1H, m), 3.62 (3H, s), 4.88-4.97 (1H, m), 6.79 (1H, d), 7.26 (2H, d), 7.51 (2H, d), 8.04-8.07 (1H, m), 8.51-8.52 (1H, m), 10.84 (1H, s), 11.04 (1H, s) |
| 72 (1r,4r)-methyl 4-(5-(5-(4- tert-butyl- phenylamino)- 1,3,4- oxadiazole-2- carboxamido) pyridin-2- yloxy) cyclohexane- carboxylate | 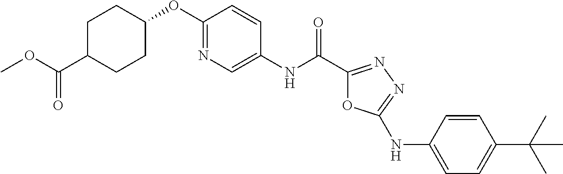 | 494 (M + H)⁺ | 1H NMR (400 MHz; DMSO) δ 1.29 (9H, s), 1.41-1.57 (4H, m), 1.96-2.02 (2H, m), 2.07-2.15 (2H, m), 2.34-2.42 (1H, m), 3.61 (3H, s), 4.84-4.97 (1H, m), 6.80 (1H, d), 7.41 (2H, d), 7.52 (2H, d), 8.04-8.07 (1H, m), 8.51-8.52 (1H, m), 10.84 (1H, s), 11.04 (1H, s) |
| 73 (1r,4r)-methyl 4-(5-(5- (3,4- difluoro- phenylamino)- 1,3,4- oxadiazole-2- carboxamido) pyridin-2- yloxy) cyclohexane- carboxylate | 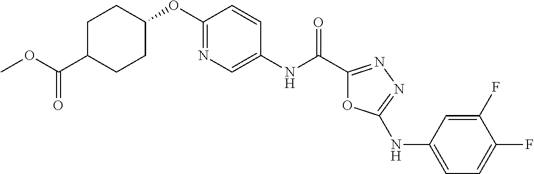 | 474 (M + H)⁺ | 1H NMR (400 MHz, DMSO) δ 1.39-1.59 (4H, m), 1.93-2.00 (2H, m), 2.08-2.14 (2H, m), 2.35-2.42 (1H, m), 3.61 (3H, s), 4.87-5.00 (1H, m), 6.79 (1H, d), 7.32-7.38 (1H, m), 7.48 (1H, q), 7.66-7.74 (1H, m), 8.03-8.08 (1H, m), 8.50-8.54 (1H, m), 11.09 (1H, s), 11.23 (1H, s) |
| 74 (1r,4r)-methyl 4-(5-(5-(4- bromo-2- chloro- phenylamino)- 1,3,4- oxadiazole-2- carboxamido) pyridin-2- yloxy) cyclohexane- carboxylate | 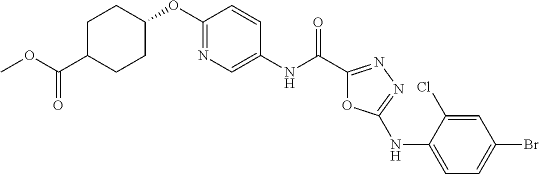 | 552 (M + H)⁺ | 1H NMR (400 MHz, DMSO) δ 1.40-1.58 (4H, m), 1.93-2.02 (2H, m), 2.06-2.14 (2H, m), 2.34-2.43 (1H, m), 3.61 (3H, s), 4.87-4.97 (1H, m), 6.80 (1H, d), 7.62- 7.66 (1H, m), 7.81 (1H, d), 7.98 (1H, d), 8.03-8.06 (1H, m), 8.51 (1H, d), 10.54 (1H, s), 11.08 (1H, s) |

### Intermediate 75

### (1r,4r)-methyl 4-(5-(2-hydrazinyl-2-oxoacetamido)pyridin-2-yloxy)cyclohexanecarboxylate

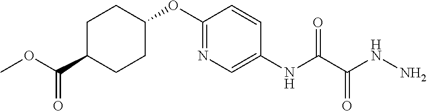

Hydrazine monohydrate (1.973 mL, 40.67 mmol) was added to (1r,4r)-methyl 4-(5-(2-methoxy-2-oxoacetamido)pyridin-2-yloxy)cyclohexanecarboxylate (Intermediate 76) (11.4 g, 33.89 mmol) in ethanol (50 mL) at ambient temperature. The resulting suspension was stirred at ambient temperature for 1 hour and at 60° C. for 2 hours. The precipitate was collected by filtration, washed with ethanol (300 mL) and ether (500 mL) and dried under vacuum to afford (1r,4r)-methyl 4-(5-(2-hydrazinyl-2-oxoacetamido)pyridin-2-yloxy)cyclohexanecarboxylate (8.39 g, 73.6%) as a white solid, which was used without further purification.

1H NMR (300 MHz, DMSO) δ 1.35-1.57 (4H, m), 1.90-1.99 (2H, m), 2.03-2.11 (2H, m), 2.32-2.41 (1H, m), 3.60 (3H, s), 4.60 (2H, d), 4.83-4.93 (1H, m), 6.75 (1H, d), 8.02-8.06 (1H, m), 8.52 (1H, d), 10.24 (1H, s), 10.65 (1H, s). m/z 337 (M+H)⁺

### Intermediate 76

### (1r,4r)-methyl 4-(5-(2-methoxy-2-oxoacetamido)pyridin-2-yloxy)cyclohexanecarboxylate

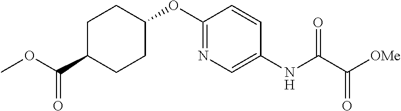

Methyl oxalyl chloride (2.56 mL, 27.81 mmol) was added to (1r,4r)-methyl 4-(5-aminopyridin-2-yloxy)cyclohexanecarboxylate (Intermediate 77) (5.8 g, 23.17 mmol) and Pyridine (3.75 mL, 46.35 mmol) in DCM (75 mL) cooled to 0° C. under nitrogen. The resulting solution was stirred at ambient temperature for 1 hour.

The reaction mixture was quenched with water (50 mL), extracted with DCM (2×75 mL), the organic layer was washed with citric acid (50 mL), brine (50 mL), dried over MgSO4, filtered and evaporated to afford crude product.

The crude product was purified by flash silica chromatography, elution gradient 20 to 70% ethyl acetate in isohexane. Pure fractions were evaporated to dryness to afford (1r,4r)-methyl 4-(5-(2-methoxy-2-oxoacetamido)pyridin-2-yloxy)cyclohexanecarboxylate (6.57 g, 84%).

1H NMR (400 MHz, CDCl3) δ 1.41-1.54 (2H, m), 1.59-1.70 (2H, m), 2.03-2.10 (2H, m), 2.17-2.24 (2H, m), 2.35 (1H, tt), 3.68 (3H, s), 3.97 (3H, s), 4.97 (1H, tt), 6.70 (1H, d), 7.96 (1H, dd), 8.29 (1H, d), 8.73 (1H, s). m/z 337 (M+H)⁺

### Intermediate 77

### (1r,4r)-methyl 4-(5-aminopyridin-2-yloxy)cyclohexanecarboxylate

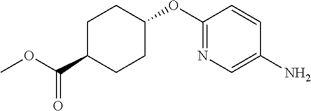

(1r,4r)-methyl 4-(5-nitropyridin-2-yloxy)cyclohexanecarboxylate (Intermediate 78) (7.3 g, 26.05 mmol) and 10% Palladium on carbon (730 mg, 0.69 mmol) in ethanol (200 mL) and THF (200 mL) were stirred under an atmosphere of hydrogen for 20 hours. The reaction mixture was filtered through celite and the solvent evaporated to give crude product. The crude product was purified by flash silica chromatography, elution gradient 20 to 70% ethyl acetate in isohexane. Pure fractions were evaporated to dryness to afford (1r,4r)-methyl 4-(5-aminopyridin-2-yloxy)cyclohexanecarboxylate (4.79 g, 73.5%) as a beige solid.

1H NMR (300.072 MHz, CDCl3) δ 1.36-1.48 (2H, m), 1.54-1.71 (2H, m), 1.99-2.08 (2H, m), 2.14-2.23 (2H, m), 2.28-2.39 (1H, m), 3.34 (2H, s), 3.68 (3H, s), 4.78-4.88 (1H, m), 6.53 (1H, d), 6.98-7.02 (1H, m), 7.63 (1H, d). m/z 251 (M+H)⁺

### Intermediate 78

### (1r,4r)-methyl 4-(5-nitropyridin-2-yloxy)cyclohexanecarboxylate

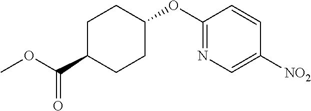

Diisopropyl azodicarboxylate (19.52 mL, 99.13 mmol) was added to a stirred solution of 2-Hydroxy-5-nitropyridine (11.11 g, 79.30 mmol), and Triphenylphosphine (31.2 g, 118.95 mmol) in THF (385 mL) under nitrogen. The resulting solution was stirred at ambient temperature for 30 minutes and then cis-methyl 4-hydroxycyclohexanecarboxylate (Intermediate 79) (15.68 g, 99.13 mmol) in THF (15 mL) was added. The resulting solution was stirred for 20 hrs under nitrogen. The solvent was evaporated and 33% ethyl acetate in isohexane (200 mL) added. Stirred for 1 hour then triphenylphosphine oxide was filtered off. The filtrate was evaporated to dryness and redissolved in ethyl acetate (100 mL), and washed sequentially with water (100 mL) and saturated brine (100 mL). The organic layer was dried over MgSO4, filtered and evaporated to afford crude product.

The crude product was purified by flash silica chromatography, elution gradient 20 to 50% ethyl acetate in isohexane. Pure fractions were evaporated to dryness to afford (1r,4r)-methyl 4-(5-nitropyridin-2-yloxy)cyclohexanecarboxylate (7.90 g, 36%) as a white solid.

1H NMR (300.072 MHz, CDCl₃) δ 1.45-1.76 (4H, m), 2.00-2.13 (2H, m), 2.19-2.26 (2H, m), 2.34-2.42 (1H, m), 3.69 (3H, s), 5.11-5.21 (1H, m), 6.75 (1H, d), 8.31-8.35 (1H, m), 9.04 (1H, d).

### Intermediate 79

### (1s,4s)-methyl 4-hydroxycyclohexanecarboxylate

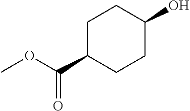

Sulfuric acid (0.074 mL, 1.39 mmol) was added to (1s,4s)-4-hydroxycyclohexanecarboxylic acid (20 g, 138.73 mmol) in methanol (500 mL) at ambient temperature. The resulting solution was stirred at 60° C. for 2 hours. The reaction mixture was evaporated to dryness and redissolved in ethyl acetate (150 mL), and washed sequentially with 2M NaOH (75 mL), water (75 mL), and saturated brine (75 mL). The organic layer was dried over MgSO4, filtered and evaporated to afford (1s,4s)-methyl 4-hydroxycyclohexanecarboxylate (19.80 g, 90%).

1H NMR (300.073 MHz, DMSO) δ 1.46-1.55 (6H, m), 1.74-1.87 (2H, m), 2.31-2.38 (1H, m), 3.58 (3H, s), 3.62-3.70 (1H, m), 4.34 (1H, d).

### Intermediate 80

### (1r,4r)-methyl 4-(5-(5-(3-(trifluoromethoxy)phenylamino)-1,3,4-oxadiazole-2-carboxamido)pyridin-2-yloxy)cyclohexanecarboxylate

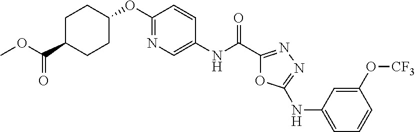

O-perfluorophenyl 3-(trifluoromethoxy)phenylcarbamothioate (Intermediate 82) (575 mg, 1.43 mmol) was added to (1r,4r)-methyl 4-(5-(2-hydrazinyl-2-oxoacetamido)pyridin-2-yloxy)cyclohexanecarboxylate (400 mg, 1.19 mmol) in DMF (8 mL) at ambient temperature. The resulting solution was stirred at 50° C. for 1 hour. 1-(3-Dimethylaminopropyl)-3-ethylcarbodiimide hydrochloride (274 mg, 1.43 mmol) was added and the solution stirred at 85° C. for 3 hours. The reaction mixture was allowed to cool to ambient temperature and half the DMF was evaporated off before adding water (8 mL). The precipitate was collected by filtration, washed with water (25 mL) and dried under vacuum to afford (1r,4r)-methyl 4-(5-(5-(3-(trifluoromethoxy)phenylamino)-1,3,4-oxadiazole-2-carboxamido)pyridin-2-yloxy)cyclohexanecarboxylate (600 mg, 97%) as a yellow solid which was used without further purification.

1H NMR (400 MHz, DMSO) δ 1.40-1.56 (4H, m), 1.92-1.99 (2H, m), 2.09-2.11 (2H, m), 2.33-2.45 (1H, m), 3.61 (3H, s), 4.88-4.93 (1H, m), 6.81 (1H, d), 7.07 (1H, d), 7.50-7.61 (2H, m), 7.71 (1H, s), 8.07 (1H, d), 8.52 (1H, d), 11.17 (1H, s), 11.39 (1H, s).

m/z 522 (M+H)⁺

Intermediate 81 was prepared in a similar manner:

| Intermediate | Structure | m/z |
|---|---|---|
| 81 (1r,4r)-methyl 4-(5-(5-(2,4- difluoro-5- (trifluoromethyl) phenylamino)-1,3,4- oxadiazole-2- carboxamido)pyridin-2- yloxy) cyclohexanecarboxylate | 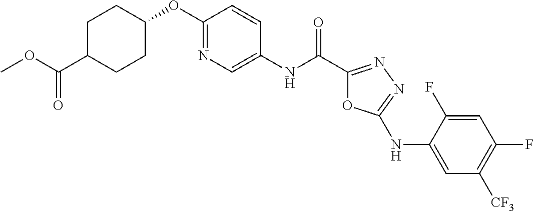 | 542 (M + H)⁺ |

### Intermediate 82

### O-perfluorophenyl 3-(trifluoromethoxy)phenylcarbamothioate

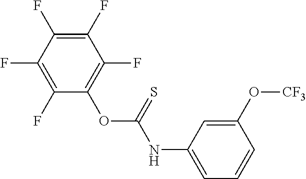

Pentafluorophenyl chlorothionoformate (2.89 mL, 18.01 mmol) in DCM (10 mL) was added dropwise to 3-(Trifluoromethoxy)aniline (2.189 mL, 16.37 mmol) and Pyridine (1.986 mL, 24.56 mmol) in dichloromethane (180 mL) at 0° C. The resulting solution was stirred at ambient temperature for 20 hours. The reaction mixture was washed sequentially with 1M citric acid (100 mL), saturated NaHCO3 (100 mL), and saturated brine (100 mL). The organic layer was dried over MgSO4, filtered and evaporated to afford crude product. The crude product was purified by flash silica chromatography, elution gradient 5 to 15% ethyl acetate in isohexane. Pure fractions were evaporated to dryness to afford O-perfluorophenyl 3-(trifluoromethoxy)phenylcarbamothioate (3.70 g, 56.0%) as a yellow oil. ¹H NMR (400 MHz, CDCl3) δ 7.08 (1H, s), 7.13-7.19 (2H, m), 7.38 (1H, t).

### Intermediate 83

### Methyl 2-((1s,4s)-4-(5-(5-(3,4-difluorophenylamino)-1,3,4-oxadiazole-2-carboxamido)pyridin-2-yloxy)cyclohexyl)acetate

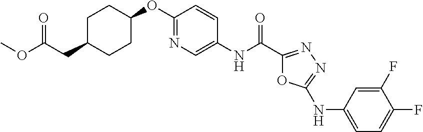

To a solution of methyl 2-((1s,4s)-4-(5-(2-hydrazinyl-2-oxoacetamido)pyridin-2-yloxy)cyclohexyl)acetate (Intermediate 84) (272 mg, 0.78 mmol) in DMF (5 mL) was added 3,4-difluorophenyl isothiocyanate (159 mg, 0.93 mmol). The resulting mixture was stirred at 45° C. for 45 minutes, 1-(3-Dimethylaminopropyl)-3-ethylcarbodiimide hydrochloride (179 mg, 0.93 mmol) was added and the mixture was stirred at 85° C. for 45 minutes. The reaction mixture was allowed to cool to ambient temperature, water (15 mL) was added and the suspension stirred for 2 hours and then filtered, washed with water (10 mL) and dried to leave methyl 2-((1s,4s)-4-(5-(5-(3,4-difluorophenylamino)-1,3,4-oxadiazole-2-carboxamido)pyridin-2-yloxy)cyclohexyl)acetate (378 mg, 100%) as a yellow solid.

1H NMR (300.073 MHz, DMSO) δ 1.30-1.42 (2H, m), 1.49-1.66 (4H, m), 1.77-1.92 (3H, m), 2.26 (2H, d), 3.58 (3H, s), 5.12-5.15 (1H, m), 6.80 (1H, d), 7.31-7.36 (1H, m), 7.41-7.52 (1H, m), 7.63-7.73 (1H, m), 8.03 (1H, dd), 8.50 (1H, s), 11.07 (1H, s), 11.23 (1H, s); m/z 488 (M+H)⁺.

### Intermediate 84

### Methyl 2-(1s,4s)-4-(5-(2-hydrazinyl-2-oxoacetamido)pyridin-2-yloxy)cyclohexyl)acetate

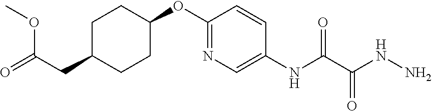

To a solution of methyl 2-(6-((1s,4s)-4-(2-methoxy-2-oxoethyl)cyclohexyloxy)pyridin-3-ylamino)-2-oxoacetate (Intermediate 85) (2.43 g, 6.94 mmol) in ethanol (100 mL) was added hydrazine monohydrate (0.370 mL, 7.63 mmol). A thick suspension quickly formed which was vigorously stirred at ambient temperature for 60 minutes. The reaction mixture was evaporated to afford methyl 2-((1s,4s)-4-(5-(2-hydrazinyl-2-oxoacetamido)pyridin-2-yloxy)cyclohexyl)acetate (2.400 g, 99%) as a white solid.

1H NMR (300.073 MHz, DMSO) δ 1.26-1.41 (2H, m), 1.47-1.65 (4H, m), 1.79-1.90 (3H, m), 2.25 (2H, d), 3.58 (3H, s), 4.59 (2H, s), 5.10-5.14 (1H, m), 6.77 (1H, d), 8.03 (1H, d), 8.51 (1H, s), 10.13 (1H, s), 10.64 (1H, s); m/z 351 (M+H)⁺.

### Intermediate 85

### Methyl 2-(6-((1s,4s)-4-(2-methoxy-2-oxoethyl)cyclohexyloxy)pyridin-3-ylamino)-2-oxoacetate

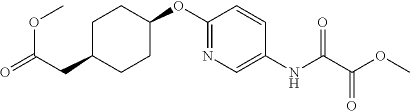

To a solution of methyl 2-((1s,4s)-4-(5-aminopyridin-2-yloxy)cyclohexyl)acetate (Intermediate 86) (2.31 g, 8.74 mmol) in DCM (30 mL) was added pyridine (0.847 mL, 10.49 mmol) and methyl oxalyl chloride (1.045 mL, 11.36 mmol). The resulting solution was stirred at ambient temperature for 1 hour. The reaction mixture was diluted with DCM (100 mL), and washed with saturated brine (100 mL), the aqueous layer was washed with DCM (3×50 mL). The organic extracts were combined, dried over MgSO4, filtered and evaporated to afford crude product. The crude product was purified by flash silica chromatography, elution gradient 0 to 10 to 30% ethyl acetate in isohexane. Pure fractions were evaporated to dryness to afford methyl 2-(6-((1s,4s)-4-(2-methoxy-2-oxoethyl)cyclohexyloxy)pyridin-3-ylamino)-2-oxoacetate (2.430 g, 79%) as a pale yellow oil which solidified on standing.

1H NMR (400 MHz, CDCl₃) δ 1.40-1.50 (2H, m), 1.57-1.66 (4H, m), 1.88-1.96 (1H, m), 1.96-2.04 (2H, m), 2.28 (2H, d), 3.67 (3H, s), 3.97 (3H, s), 5.20-5.23 (1H, m), 6.74 (1H, d), 7.97 (1H, dd), 8.29 (1H, d), 8.73 (1H, s); m/z 351 (M+H)⁺.

### Intermediate 86

### Methyl 2-((s,4s)-4-(5-aminopyridin-2-yloxy)cyclohexyl)acetate

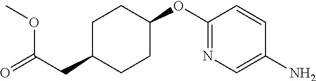

A suspension of methyl 2-((1s,4s)-4-(5-nitropyridin-2-yloxy)cyclohexyl)acetate (Intermediate 87) (2.9 g, 9.85 mmol) and 10% Palladium on carbon (0.304 g, 2.86 mmol) in ethyl acetate (200 mL) was evacuated with hydrogen (3 cycles) and stirred under an atmosphere of hydrogen at Pressure and ambient temperature overnight. The reaction mixture was evaporated to afford methyl 2-((1s,4s)-4-(5-aminopyridin-2-yloxy)cyclohexyl)acetate (2.60 g, 100%) as a light brown gum.

1H NMR (400 MHz, CDCl₃) δ 1.40-1.50 (2H, m), 1.53-1.63 (4H, m), 1.84-2.00 (3H, m), 2.27 (2H, d), 3.33 (2H, s), 3.66 (3H, s), 5.06-5.08 (1H, m), 6.57 (1H, d), 7.01 (1H, d), 7.64 (1H, s); m/z 265 (M+H)⁺.

### Intermediate 87

### Methyl 2-((1r,4r)-4-(5-nitropyridin-2-yloxy)cyclohexyl)acetate

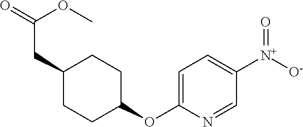

Diisopropyl azodicarboxylate (1.335 mL, 6.78 mmol) was added to a stirred solution of 5-nitropyridin-2-ol (500 mg, 3.57 mmol) and Triphenylphosphine (2153 mg, 8.21 mmol) in THF (15 mL), after ˜5 minutes methyl 2-((1r,4r)-4-hydroxycyclohexyl)acetate (Intermediate 89) (615 mg, 3.57 mmol) in THF (1 mL) was added. The reaction was heated to 180° C. for 45 minutes in the microwave reactor and cooled to RT. The reaction mixture was evaporated and the crude product was purified by flash silica chromatography (40 g crawford cartridge, loading in DCM), elution gradient 20 to 50% ethyl acetate in isohexane. Pure fractions were evaporated to dryness to afford methyl 2-(1r,4r)-4-(5-nitropyridin-2-yloxy)cyclohexyl)acetate as a yellow solid.

Reaction carried out 6 times (21.42 mmol in total, isolated 3.38 g (11.48 mmol, 53.5%).

1H NMR (300.072 MHz, CDCl₃) δ 1.40-1.50 (2H, m), 1.60-1.73 (4H, m), 1.88-2.07 (3H, m), 2.29 (2H, d), 3.68 (3H, s), 5.39-5.43 (1H, m), 6.78 (1H, d), 8.33 (1H, d), 9.05 (1H, s); m/z 295 (M+H)⁺.

### Intermediate 88

### Methyl 2-((1s,4s)-4-hydroxycyclohexyl)acetate and Intermediate 89-Methyl 2-(1r,4r)-4-hydroxycyclohexyl)acetate

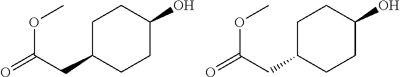

Methyl 2-(4-hydroxyphenyl)acetate (81.3 g, 489.25 mmol) and Rhodium (5% on Alumina) (8.13 g, 3.95 mmol) in methanol (800 mL) were stirred under an atmosphere of hydrogen at 3 bar and 25° C. for 3 hours. The reaction mixture was filtered through celite and concentrated under reduced pressure afford desired product (84 g, 100%) as a mixture of cis and trans isomers. 15 g of the material was purified by flash silica chromatography (330 g Crawford cartridge, loading in isohexane with a few drops of ethyl acetate), elution gradient 30 to 50% ethyl acetate in isohexane. Pure fractions were evaporated to dryness to afford methyl 2-((1s,4s)-4-hydroxycyclohexyl)acetate (8.31 g) as a colourless oil and methyl 2-(1r,4r)-4-hydroxycyclohexyl)acetate (5.57 g) as a colourless oil.

### methyl 2-((1s,4s)-4-hydroxycyclohexyl)acetate

1H NMR (300.072 MHz, CDCl₃) δ 1.37-1.76 (9H, m), 1.80-1.94 (1H, m), 2.26 (2H, d), 3.67 (3H, s), 3.96-4.00 (1H, m)

### methyl 2-((1r,4r)-4-hydroxycyclohexyl)acetate

1H NMR (300.072 MHz, CDCl₃) δ 0.98-1.13 (2H, m), 1.22-1.38 (2H, m), 1.67-1.83 (3H, m), 1.92-2.01 (2H, m), 2.20 (2H, d), 3.50-3.60 (1H, m), 3.67 (3H, s).

### Intermediate 90

### Methyl 2-((1r,4r)-4-(5-(5-(3,4-difluorophenylamino)-1,3,4-oxadiazole-2-carboxamido)pyridin-2-yloxy)cyclohexyl)acetate

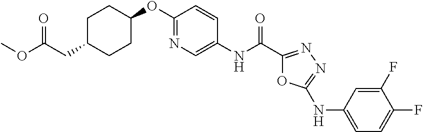

To a solution of methyl 2-((1r,4r)-4-(5-(2-hydrazinyl-2-oxoacetamido)pyridin-2-yloxy)cyclohexyl)acetate (Intermediate 91) (310 mg, 0.88 mmol) in DMF (5 mL) was added 3,4-Difluorophenyl isothiocyanate (182 mg, 1.06 mmol). The resulting mixture was stirred at 45° C. for 45 minutes, 1-(3-Dimethylaminopropyl)-3-ethylcarbodiimide hydrochloride (204 mg, 1.06 mmol) was added and the mixture was stirred at 85° C. for 70 minutes. The reaction was incomplete and further EDAC (100 mg) was added and the mixture was stirred at 85° C. for a further 40 minutes. The reaction mixture was allowed to cool to ambient temperature, water (5 mL) was added and the suspension stirred for 5 minutes and then filtered and dried to leave methyl 2-((1r,4r)-4-(5-(5-(3,4-difluorophenylamino)-1,3,4-oxadiazole-2-carboxamido)pyridin-2-yloxy)cyclohexyl)acetate (233 mg, 54.0%) as a brown solid.

1H NMR (300.073 MHz, DMSO) δ 1.07-1.23 (2H, m), 1.31-1.44 (2H, m), 1.67-1.79 (3H, m), 2.02-2.09 (2H, m), 2.23 (2H, d), 3.59 (3H, s), 4.81-4.91 (1H, m), 6.77 (1H, d), 7.31-7.37 (1H, m), 7.43-7.52 (1H, m), 7.64-7.72 (1H, m), 8.03 (1H, dd), 8.49 (1H, s), 11.09 (1H, s), 11.25 (1H, s); m/z 488 (M+H)⁺.

### Intermediate 91

### Methyl 2-((1r,4r)-4-(5-(2-hydrazinyl-2-oxoacetamido)pyridin-2-yloxy)cyclohexyl)acetate

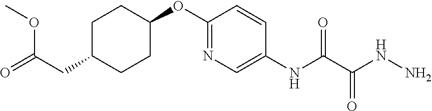

To a solution of methyl 2-(6-((1r,4r)-4-(2-methoxy-2-oxoethyl)cyclohexyloxy)pyridin-3-ylamino)-2-oxoacetate (Intermediate 92) (2.78 g, 7.93 mmol) in ethanol (150 mL) was added hydrazine monohydrate (0.423 mL, 8.73 mmol). The suspension was stirred at ambient temperature for a further 90 minutes. The reaction mixture was evaporated to afford methyl 2-((1r,4r)-4-(5-(2-hydrazinyl-2-oxoacetamido)pyridin-2-yloxy)cyclohexyl)acetate (2.68 g, 96%) as a white solid.

1H NMR (400 MHz, DMSO) δ 1.08-1.20 (2H, m), 1.32-1.43 (2H, m), 1.67-1.79 (3H, m), 2.03-2.07 (2H, m), 2.24 (2H, d), 3.59 (3H, s), 4.61 (2H, s), 4.81-4.89 (1H, m), 6.75 (1H, d), 8.03 (1H, d), 8.54 (1H, s), 10.24 (1H, s), 10.65 (1H, s); m/z 351 (M+H)⁺.

### Intermediate 92

### Methyl 2-(6-((1r,4r)-4-(2-methoxy-2-oxoethyl) cyclohexyloxy)pyridin-3-ylamino)-2-oxoacetate

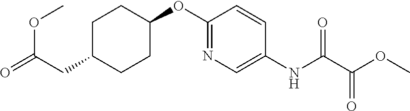

To a solution of methyl 2-((1r,4r)-4-(5-aminopyridin-2-yloxy)cyclohexyl)acetate (Intermediate 93) (2.10 g, 7.94 mmol) in DCM (30 mL) was added pyridine (0.770 mL, 9.53 mmol) and methyl oxalyl chloride (0.950 mL, 10.33 mmol). The resulting solution was stirred at ambient temperature for 1 hour. The reaction mixture was diluted with DCM (100 mL), and washed with saturated brine (100 mL), the aqueous layer was washed with DCM (3×50 mL). The organic extracts were combined, dried over MgSO4, filtered and evaporated to afford to afford methyl 2-(6-((1r,4r)-4-(2-methoxy-2-oxoethyl)cyclohexyloxy)pyridin-3-ylamino)-2-oxoacetate (2.78 g, 100%) as a pale brown solid.

1H NMR (400 MHz, CDCl₃) δ 1.14-1.26 (2H, m), 1.40-1.52 (2H, m), 1.80-1.88 (3H, m), 2.11-2.18 (2H, m), 2.24 (2H, d), 3.67 (3H, s), 3.97 (3H, s), 4.88-4.97 (1H, m), 6.71 (1H, d), 7.94 (1H, dd), 8.30 (1H, s), 8.73 (1H, s); m/z 351 (M+H)⁺.

### Intermediate 93

### Methyl 2-((1r,4r)-4-(5-aminopyridin-2-yloxy)cyclohexyl)acetate

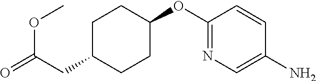

A suspension of methyl 2-((1r,4r)-4-(5-nitropyridin-2-yloxy)cyclohexyl)acetate (Intermediate 94) (2.99 g, 10.16 mmol) and 10% Palladium on carbon (0.314 g, 2.95 mmol) in ethyl acetate (200 mL) was evacuated with hydrogen (3 cycles) and stirred under an atmosphere of hydrogen at ambient temperature overnight. The reaction mixture was evaporated to afford crude product as a light brown gum. The crude product was purified by flash silica chromatography, elution gradient 30 to 70% ethyl acetate in isohexane. Pure fractions were evaporated to dryness to afford methyl 2-((1r,4r)-4-(5-aminopyridin-2-yloxy)cyclohexyl)acetate (2.400 g, 89%) as a pale brown oil which solidified on standing.

1H NMR (400 MHz, CDCl₃) δ 1.12-1.24 (2H, m), 1.37-1.48 (2H, m), 1.78-1.87 (3H, m), 2.10-2.16 (2H, m), 2.23 (2H, d), 3.32 (2H, s), 3.67 (3H, s), 4.75-4.83 (1H, m), 6.53 (1H, d), 7.00 (1H, dd), 7.63 (1H, d); m/z 265 (M+H)⁺.

### Intermediate 94

### Methyl 2-((1r,4r)-4-(5-nitropyridin-2-yloxy)cyclohexyl)acetate

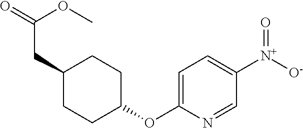

Diisopropyl azodicarboxylate (1.335 mL, 6.78 mmol) was added to a stirred solution of 5-nitropyridin-2-ol (500 mg, 3.57 mmol) and triphenylphosphine (2153 mg, 8.21 mmol) in THF (15 mL), exotherm occurred and the suspension was allowed to stir at ambient temperature for ˜5 minutes. Methyl 2-((1s,4s)-4-hydroxycyclohexyl)acetate (Intermediate 88) (615 mg, 3.57 mmol) was added and the reaction was heated to 180° C. for 45 minutes in the microwave reactor and cooled to RT. The reaction mixture was evaporated and the crude product was purified by flash silica chromatography (330 g crawford cartridge, loading in DCM), elution gradient 20 to 50% ethyl acetate in isohexane. Pure fractions were evaporated to dryness to afford methyl 2-((1r,4r)-4-(5-nitropyridin-2-yloxy)cyclohexyl)acetate as a yellow solid.

Reaction carried out 7 times, isolated 2.99 g (10.17 mmol, 40.69%) in total.

1H NMR (400 MHz, CDCl₃) δ 1.15-1.27 (2H, m), 1.46-1.56 (2H, m), 1.80-1.91 (3H, m), 2.14-2.19 (2H, m), 2.25 (2H, d), 3.69 (3H, s), 5.05-5.13 (1H, m), 6.74 (1H, d), 8.32 (1H, d), 9.04 (1H, d); m/z 295 (M+H)⁺.

### Intermediate 95

### methyl 4-(5-(5-(3,4-difluorophenylamino)-1,3,4-oxadiazole-2-carboxamido)pyridin-2-yloxy)benzoate

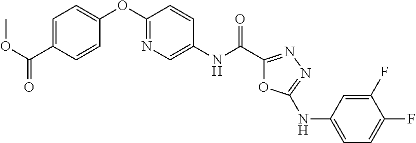

3,4-Difluorophenyl isothiocyanate (249 mg, 1.45 mmol) was added to methyl 4-(5-(2-hydrazinyl-2-oxoacetamido)pyridin-2-yloxy)benzoate (intermediate 97) (400 mg, 1.21 mmol) in DMA (10 mL) at 20° C. The resulting solution was stirred at 45° C. for 30 minutes. 1-(3-Dimethylaminopropyl)-3-ethylcarbodiimide hydrochloride (279 mg, 1.45 mmol) was added to the reaction and the temperature was increased to 85° C. The reaction was stirred at this temperature for one hour then cooled to ambient temperature. The reaction mixture was treated with water (40 mL) and the resulting precipitate was filtered off, washed with water (40 mL) then dried to yield the crude title compound (566 mg, 100%). m/z (ESI+) (M+H)+=468.

This material was carried through to the next reaction without further purification.

### Intermediate 96

### methyl 4-(5-(5-(3-chloro-4-fluorophenylamino)-1,3,4-oxadiazole-2-carboxamido)pyridin-2-yloxy)benzoate

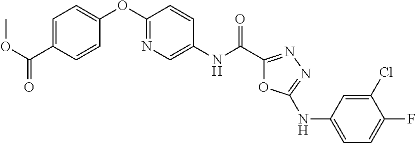

3-Chloro-4-fluorophenyl isothiocyanate (227 mg, 1.21 mmol) was added to methyl 4-(5-(2-hydrazinyl-2-oxoacetamido)pyridin-2-yloxy)benzoate (intermediate 97) (400 mg, 1.21 mmol) in DMA (10 mL) at 20° C. The resulting solution was stirred at 45° C. for 30 minutes. 1-(3-Dimethylaminopropyl)-3-ethylcarbodiimide hydrochloride (279 mg, 1.45 mmol) was added to the reaction and the temperature was increased to 85° C. The reaction was stirred at this temperature for one hour then cooled to ambient temperature. The reaction mixture was treated with water (40 mL) and the resulting precipitate was filtered off, washed with water (40 mL) then dried to yield the crude title compound (586 mg, 100%). m/z (ESI+) (M+H)+=484.

This material was carried through to the next reaction without further purification.

### Intermediate 97

### methyl 4-(5-(2-hydrazinyl-2-oxoacetamido)pyridin-2-yloxy)benzoate

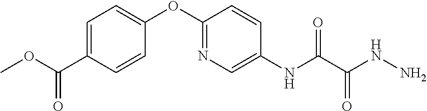

Hydrazine hydrate (0.676 mL, 13.89 mmol) was added to methyl 4-(5-(2-methoxy-2-oxoacetamido)pyridin-2-yloxy)benzoate, intermediate 98, (4.17 g, 12.63 mmol) in ethanol (500 mL) at 75° C. under nitrogen. The resulting solution was stirred at 75° C. for 1 hour then cooled to ambient temperature and stirred overnight. The resulting suspension was filtered, the solid was washed with more EtOH then dried under high vac to yield the title compound (3.95 g, 95%) as an off white solid. m/z (ESI+) (M+H)+=331.

### Intermediate 98

### methyl 4-(5-(2-methoxy-2-oxoacetamido)pyridin-2-yloxy)benzoate

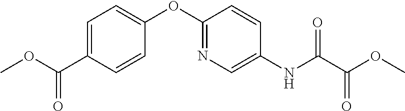

Methyl chlorooxoacetate (2.67 mL, 29.03 mmol) was added portionwise to methyl 4-(5-aminopyridin-2-yloxy)benzoate, intermediate 99, (7.09 g, 29.03 mmol) and pyridine (2.348 mL, 29.03 mmol) in DCM (100 mL) at ambient temperature under nitrogen. The resulting solution was stirred at ambient temperature for 1 hour. The reaction mixture was evaporated to dryness and redissolved in EtOAc (400 mL), and washed sequentially with saturated brine (200 mL). The organic layer was dried over MgSO₄, filtered and evaporated to afford crude product. The crude product was recrystallised from MeOH to yield 3.47 g of pure product. The MeOH filtrate was evaporated in vacuo and the resulting material was purified by column chromatography (silica, eluting with 30 to 90% EtOAc in isohexane) to yield a further 700 mg of pure product. The pure product samples were combined to give the title compound (4.17 g, 43.5%) as an off white solid.

m/z (ESI+) (M+Na)+=331.

### Intermediate 99

### methyl 4-(5-aminopyridin-2-yloxy)benzoate

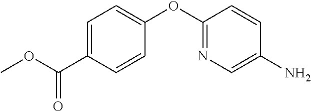

A suspension of methyl 4-(5-nitropyridin-2-yloxy)benzoate (intermediate 100) (9 g, 32.82 mmol) and palladium (5% on carbon) (500 mg, 4.70 mmol) in MeOH (100 mL) was stirred under an atmosphere of hydrogen at 1 atm and 45° C. for 18 hours. The reaction mixture was filtered through celite and the solvent was evaporated in vacuo to yield crude product. A portion of this crude product was purified by flash silica chromatography, elution gradient 30 to 70% EtOAc in isohexane. Pure fractions were evaporated to dryness to afford methyl 4-(5-aminopyridin-2-yloxy)benzoate (1.05 g, 13%) as a tan solid.

¹H NMR (400 MHz, DMSO-d6) δ 3.84 (3H, s), 5.20-5.22 (2H, m), 6.86 (1H, d), 7.02 (2H, d), 7.11-7.14 (1H, m), 7.62 (1H, d), 7.93 (2H, d). m/z (ESI+) (M+H)+=245.

The remaining material was carried through to the next reaction crude.

### Intermediate 100

### methyl 4-(5-nitropyridin-2-yloxy)benzoate

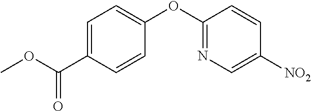

Sodium hydride (2.056 g, 85.66 mmol) was added portionwise to 2-chloro-5-nitropyridine (7.76 g, 48.95 mmol) and methyl 4-hydroxybenzoate (7.45 g, 48.95 mmol) in THF (50 mL) at 0° C. over a period of 10 minutes under nitrogen. The resulting suspension was stirred at ambient temperature for 70 hours. The reaction mixture was treated with 0.5 N citric acid (100 mL) then extracted with EtOAc (2×100 mL). The organic layers were combined, washed with water (50 mL) and brine (50 mL) then dried (MgSO₄), filtered and evaporated to a brown solid. This solid was triturated with hot MeOH (˜100 mL) then cooled to ambient temperature. The resulting suspension was filtered, washed with MeOH (˜100 mL) then dried under vacuum to yield the title compound (9.00 g, 67.1%) as a pale brown solid. A second crop precipitated from the filtrate overnight to yield a further 876 mg of material.

¹H NMR (400 MHz, DMSO-d6) δ 3.88 (3H, s), 7.34-7.40 (3H, m), 8.05-8.08 (2H, m), 8.65-8.68 (1H, m), 9.04-9.05 (1H, m). m/z (ESI+) (M+H)+=275.

### Intermediate 101

### methyl 4-(5-(2-hydrazinyl-2-oxoacetamido)pyridin-2-yloxy)benzoate

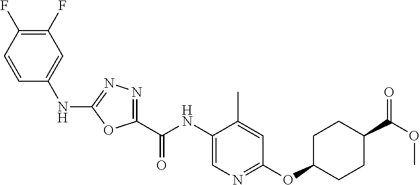

3,4-Difluorophenyl isothiocyanate (0.189 mL, 1.43 mmol) was added to (1s,4s)-methyl 4-(5-(2-hydrazinyl-2-oxoacetamido)-4-methylpyridin-2-yloxy)cyclohexanecarboxylate, intermediate 102, (500 mg, 1.43 mmol) in DMA (8 mL) at 45° C. The resulting solution was stirred at 45° C. for 30 minutes. 1-(3-Dimethylaminopropyl)-3-ethylcarbodiimide hydrochloride (301 mg, 1.57 mmol) was added to the reaction and the temperature was increased to 85° C. The reaction was stirred at this temperature for one hour then cooled to ambient temperature. The reaction mixture was treated with water (60 mL) and the resulting precipitate was filtered off, washed with water (30 mL) then dried to yield the crude title compound (696 mg, 100%). m/z (ESI+) (M+H)+=488.

This material was carried through to the next reaction without further purification.

### Intermediate 102

### (1s,4s)-methyl 4-(5-(2-hydrazinyl-2-oxoacetamido)-4-methylpyridin-2-yloxy)cyclohexanecarboxylate

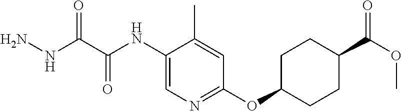

Hydrazine hydrate (0.878 mL, 18.05 mmol) was added to (1s,4s)-methyl 4-(5-(2-methoxy-2-oxoacetamido)-4-methylpyridin-2-yloxy)cyclohexanecarboxylate (intermediate 103) (5.75 g, 16.41 mmol) in EtOH (200 mL) at ambient temperature under nitrogen. The resulting solution was stirred at ambient temperature for 16 hours during which time a precipitate crashed out. This solid was filtered off and dried under high vac to yield the title compound (5.11 g, 89%) as a white solid.

¹H NMR (400 MHz, DMSO-d6) δ 1.66-1.71 (4H, m), 1.75-1.85 (4H, m), 2.13 (3H, s), 3.61 (3H, s), 4.59 (2H, s), 5.08-5.15 (1H, m), 6.72 (1H, s), 7.96 (1H, s), 10.13 (1H, s), 10.19 (1H, s). m/z (ESI+) (M+H)+=351.

### Intermediate 103

### (1s,4s)-methyl 4-(5-(2-methoxy-2-oxoacetamido)-4-methylpyridin-2-yloxy)cyclohexanecarboxylate

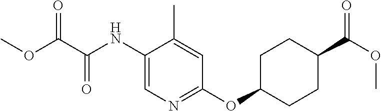

Methyl oxalyl chloride (1.510 mL, 16.42 mmol) was added portionwise to (1s,4s)-methyl 4-(5-amino-4-methylpyridin-2-yloxy)cyclohexanecarboxylate (intermediate 104) (4.34 g, 16.42 mmol) and pyridine (1.328 mL, 16.42 mmol) in DCM (100 mL) at ambient temperature under nitrogen. The resulting solution was stirred at ambient temperature for 1 hour. The reaction mixture was evaporated to dryness and redissolved in EtOAc (150 mL), and washed sequentially with water (50 mL) and saturated brine (50 mL). The organic layer was dried over MgSO₄, filtered and evaporated to afford the title compound (5.75 g, 100%) as an orange gum.

¹H NMR (400 MHz, CDCl₃) δ 1.56-1.64 (2H, m), 1.65-1.74 (2H, m), 1.83-1.95 (4H, m), 2.20 (3H, s), 2.33-2.40 (1H, m), 3.62 (3H, s), 3.91 (3H, s), 5.12-5.16 (1H, m), 6.56 (1H, s), 8.36 (1H, s), 8.45 (1H, s). m/z (ESI+) (M+H)+=351.

### Intermediate 104

### (1s,4s)-methyl 4-(5-amino-4-methylpyridin-2-yloxy)cyclohexanecarboxylate

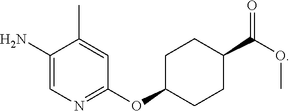

(1s,4s)-methyl-4-(4-methyl-5-nitropyridin-2-yloxy)cyclohexanecarboxylate (5.43 g, 18.45 mmol) (intermediate 105) and palladium (10% on carbon) (550 mg, 0.52 mmol) in MeOH (300 mL) were stirred under an atmosphere of hydrogen at 1 atm and ambient temperature for 16 hours. The reaction mixture was filtered through celite and the solvent was evaporated in vacuo to yield the title compound (4.71 g, 97%) as an orange oil.

¹H NMR (400 MHz, CDCl₃) δ 1.52-1.60 (2H, m), 1.64-1.70 (2H, m), 1.83-1.93 (4H, m), 2.08 (3H, s), 2.31-2.37 (1H, m), 3.20 (2H, s), 3.62 (3H, s), 4.97-5.01 (1H, m), 6.44 (1H, s), 7.50 (1H, s). m/z (ESI+) (M+H)+=265.

### Intermediate 105

### (1s,4s)-methyl 4-(4-methyl-5-nitropyridin-2-yloxy)cyclohexanecarboxylate

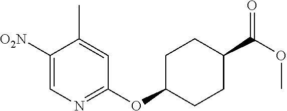

Diisopropyl azodicarboxylate (7.98 mL, 40.55 mmol) was added portionwise to 4-methyl-5-nitropyridin-2-ol (5 g, 32.44 mmol), (1r,4r)-methyl 4-hydroxycyclohexanecarboxylate (5.13 g, 32.44 mmol) and triphenylphosphine (10.64 mL, 48.66 mmol) in THF (140 mL) at ambient temperature over a period of 3 minutes under nitrogen. The resulting solution was stirred at ambient temperature for 60 hours. The reaction mixture was evaporated to dryness and redissolved in EtOAc (150 mL), and washed sequentially with water (50 mL) and saturated brine (50 mL). The organic layer was dried over MgSO₄, filtered and evaporated to afford crude product. The crude product was purified by flash silica chromatography, elution gradient 10 to 30% EtOAc in isohexane. Pure fractions were evaporated to dryness to afford the title compound (5.62 g, 58.9%) as an off white solid.

¹H NMR (400 MHz, CDCl₃) δ 1.61-1.76 (4H, m), 1.82-1.97 (4H, m), 2.36-2.42 (1H, m), 2.54 (3H, s), 3.63 (3H, s), 5.25-5.28 (1H, m), 6.56 (1H, s), 8.83 (1H, s). m/z (ESI+) (M+H)+=294.

Intermediates 106-107 were prepared in the same way as intermediate 101, by reaction of intermediate 102 with the appropriately substituted phenylisothiocyanate.

| Intermediate | Structure | Mass Spec |
|---|---|---|
| 106- (1s,4s)-methyl 4-(5-(5- (3-chloro-4- fluorophenylamino)-1,3,4- oxadiazole-2- carboxamido)-4- methylpyridin-2- yloxy) cyclohexanecarboxylate | 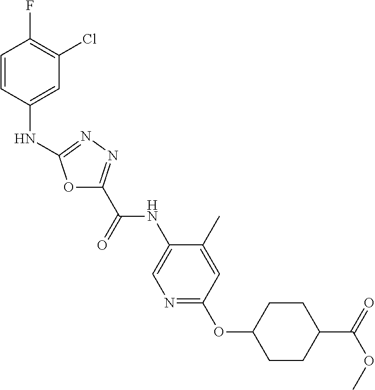 | m/z (ESI+) (M + H)+ = 504 |
| 107- (1s,4s)-methyl 4-(5-(5- (4-chloro-3- fluorophenylamino)-1,3,4- oxadiazole-2- carboxamido)-4- methylpyridin-2- yloxy) cyclohexanecarboxylate | 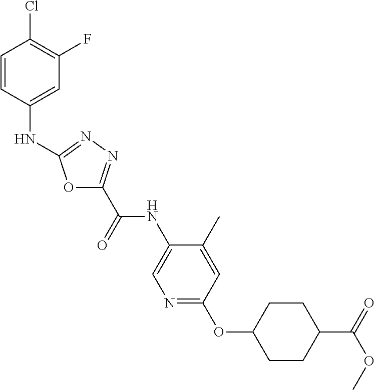 | m/z (ESI+) (M + H)+ = 504 |

### Intermediate 108

### methyl (1s,4s)-methyl 4-(5-(5-(3,4-difluorophenylamino)-1,3,4-oxadiazole-2-carboxamido)-4-methoxypyridin-2-yloxy)cyclohexanecarboxylate

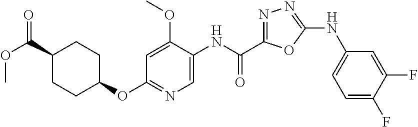

3,4-Difluorophenyl isothiocyanate (0.098 mL, 0.74 mmol) was added to (1s,4s)-methyl 4-(5-(2-hydrazinyl-2-oxoacetamido)-4-methoxypyridin-2-yloxy)cyclohexanecarboxylate (intermediate 109) (300 mg, 0.82 mmol) in DMA (8 mL). The resulting solution was stirred at 45° C. for 30 minutes. 1-(3-Dimethylaminopropyl)-3-ethylcarbodiimide hydrochloride (188 mg, 0.98 mmol) was added to the reaction and the temperature was increased to 85° C. The reaction was stirred at this temperature for one hour then cooled to ambient temperature. The reaction mixture was treated with water (60 mL) and the resulting precipitate was filtered off, washed with water (50 mL) then dried to yield the crude title compound (335 mg, 81%). This material was carried through to the next reaction without further purification.

¹H NMR (400 MHz, DMSO-d6) δ 1.69-1.74 (4H, m), 1.75-1.90 (4H, m), 3.63 (3H, s), 3.87 (3H, s), 5.18 (1H, s), 6.52 (1H, s), 7.30-7.40 (1H, m), 7.46-7.55 (1H, m), 7.69-7.73 (1H, m), 8.10 (1H, m), 10.12 (1H, s), 11.27 (1H, s). m/z (ESI+) (M+H)+=504.

### Intermediate 109

### (1s,4s)-methyl 4-(5-(2-hydrazinyl-2-oxoacetamido)-4-methoxypyridin-2-yloxy)cyclohexanecarboxylate

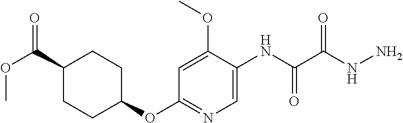

Hydrazine hydrate (0.185 mL, 3.81 mmol) was added to (1s,4s)-methyl 4-(4-methoxy-5-(2-methoxy-2-oxoacetamido)pyridin-2-yloxy)cyclohexanecarboxylate (intermediate 110) (1.27 g, 3.47 mmol) in ethanol (50 mL) at ambient temperature under nitrogen. The resulting solution was stirred at ambient temperature for 2 hours. Further hydrazine hydrate (0.092 mL, 1.90 mmol) was added to the reaction and it was left to stir over night. The reaction was filtered, the solid was washed with EtOH (50 mL) then dried under high vac to yield the title compound (1.090 g, 86%) as a white solid

¹H NMR (400 MHz, DMSO-d6) δ 1.68-1.76 (4H, m), 1.77-1.87 (4H, m), 3.62 (3H, s), 3.90 (3H, s), 4.65 (2H, s), 5.16 (1H, s), 6.51 (1H, s), 8.40 (1H, s), 9.61 (1H, s), 10.38 (1H, s). m/z (ESI+) (M+H)+=367.

### Intermediate 110

### (1s,4s)-methyl 4-(4-methoxy-5-(2-methoxy-2-oxoacetamido)pyridin-2-yloxy)cyclohexanecarboxylate

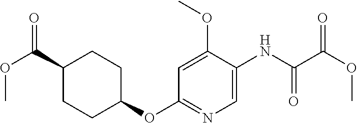

Methyl oxalyl chloride (0.320 mL, 3.48 mmol) was added portionwise to (1s,4s)-methyl 4-(5-amino-4-methoxypyridin-2-yloxy)cyclohexanecarboxylate (intermediate 111) (975 mg, 3.48 mmol) and pyridine (0.281 mL, 3.48 mmol) in DCM (30 mL) at ambient temperature under nitrogen. The resulting solution was stirred at ambient temperature for 1 hour. The reaction mixture was evaporated to dryness and redissolved in EtOAc (100 mL), and washed sequentially with water (50 mL) and saturated brine (50 mL). The organic layer was dried over MgSO₄, filtered and evaporated to afford the title compound (1.27 g, 100%) as a yellow solid.

¹H NMR (400 MHz, CDCl₃) δ 1.55-1.67 (2H, m), 1.68-1.78 (2H, m), 1.83-1.97 (4H, m), 2.34-2.41 (1H, m), 3.63 (3H, s), 3.86 (3H, s), 3.90 (3H, s), 5.16-5.19 (1H, m), 6.20 (1H, s), 8.94 (1H, s), 8.97 (1H, s). m/z (ESI+) (M+H)+=367.

### Intermediate 111

### (1s,4s)-methyl 4-(5-amino-4-methoxypyridin-2-yloxy)cyclohexanecarboxylate

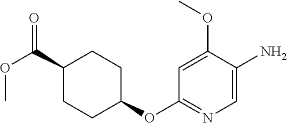

(1s,4s)-methyl 4-(4-methoxy-5-nitropyridin-2-yloxy)cyclohexanecarboxylate (1.27 g, 4.09 mmol) (intermediate 112) and palladium (10% on charcoal) (127 mg, 1.19 mmol) in methanol (30 mL) were stirred under an atmosphere of hydrogen at 1 atm and ambient temperature for 16 hours. The reaction mixture was filtered through celite and the solvent was evaporated in vacuo to yield the title compound (0.985 g, 86%) as a pale yellow oil.

¹H NMR (400 MHz, CDCl₃) δ 1.53-1.61 (2H, m), 1.65-1.71 (2H, m), 1.83-1.92 (4H, m), 2.32-2.38 (1H, m), 3.30 (2H, s), 3.62 (3H, s), 3.80 (3H, s), 5.02-5.05 (1H, m), 6.12 (1H, s), 7.44 (1H, s). m/z (ESI+) (M+H)+=281.

### Intermediate 112

### (1s,4s)-methyl 4-(4-methoxy-5-nitropyridin-2-yloxy)cyclohexanecarboxylate

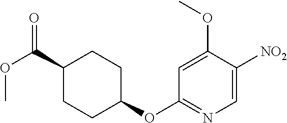

Diisopropyl azodicarboxylate (1.447 mL, 7.35 mmol) was added portionwise to 4-methoxy-5-nitropyridin-2-ol (1 g, 5.88 mmol), (1r,4r)-methyl 4-hydroxycyclohexanecarboxylate (0.930 g, 5.88 mmol) and triphenylphosphine (1.927 mL, 8.82 mmol) in dioxane (30 mL) at ambient temperature over a period of 3 minutes under nitrogen. The resulting solution was stirred at 70° C. for 3 days. The reaction was cooled to ambient temperature then diluted with Et₂O (100 mL), and washed sequentially with water (50 mL) and saturated brine (50 mL). The organic layer was dried over MgSO₄, filtered and evaporated to afford crude product. The crude product was purified by flash silica chromatography, elution gradient 5 to 40% EtOAc in isohexane. Pure fractions were evaporated to dryness to afford the title compound (1.290 g, 70.7%) as a pale yellow oil.

¹H NMR (400 MHz, DMSO-d6) δ 1.70-1.89 (8H, m), 3.62 (3H, s), 3.98 (3H, s), 5.28-5.30 (1H, m), 6.68 (1H, s), 8.77 (1H, s). m/z (ESI+) (M+H)+=311.

### Intermediate 113

### (1s,4s)-methyl 4-(5-(5-(4-bromo-2-chlorophenylamino)-1,3,4-oxadiazole-2-carboxamido)-4-methoxypyridin-2-yloxy)cyclohexanecarboxylate

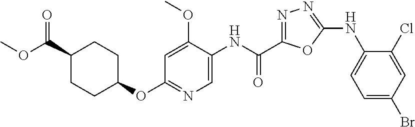

4-Bromo-2-chlorophenyl isothiocyanate (204 mg, 0.82 mmol) was added to (1s,4s)-methyl 4-(5-(2-hydrazinyl-2-oxoacetamido)-4-methoxypyridin-2-yloxy)cyclohexanecarboxylate (intermediate 109) (300 mg, 0.82 mmol) in DMA (8 mL). The resulting solution was stirred at 45° C. for 30 minutes. 1-(3-Dimethylaminopropyl)-3-ethylcarbodiimide hydrochloride (188 mg, 0.98 mmol) was added to the reaction and the temperature was increased to 85° C. The reaction was stirred at this temperature for one hour then cooled to ambient temperature. The reaction mixture was treated with water (60 mL) and the resulting precipitate was filtered off, washed with water (50 mL) then dried to yield the crude title compound (476 mg, 100%). m/z (ESI+) (M+H)+=582.

This material was carried through to the next reaction without further purification.

### Intermediate 114

### (1s,4s)-methyl 4-(3-chloro-5-(5-(2,4-dichlorophenylamino)-1,3,4-oxadiazole-2-carboxamido)pyridin-2-yloxy)cyclohexanecarboxylate

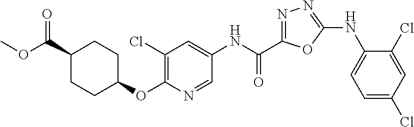

2,4-Dichloroisothiocyanatobenzene (168 mg, 0.82 mmol) was added to (1s,4s)-methyl 4-(3-chloro-5-(2-hydrazinyl-2-oxoacetamido)pyridin-2-yloxy)cyclohexanecarboxylate (intermediate 115) (305 mg, 0.82 mmol) in DMF (4 mL). The resulting suspension was stirred at 55° C. for 1 hour. 1-(3-Dimethylaminopropyl)-3-ethylcarbodiimide hydrochloride (189 mg, 0.99 mmol) was added to the mixture. The resulting solution was stirred at 75° C. for 16 hours. The reaction mixture was concentrated and diluted with water (5 mL). The precipitate was collected by filtration, washed with water (5 mL), ether (10 mL) and dried under vacuum to afford (1s,4s)-methyl 4-(3-chloro-5-(5-(2,4-dichlorophenylamino)-1,3,4-oxadiazole-2-carboxamido)pyridin-2-yloxy)cyclohexanecarboxylate (413 mg). m/z (ESI+) (M+H)⁺=542.

### Intermediate 115

### (1s,4s)-methyl 4-(3-chloro-5-(2-hydrazinyl-2-oxoacetamido)pyridin-2-yloxy)cyclohexanecarboxylate

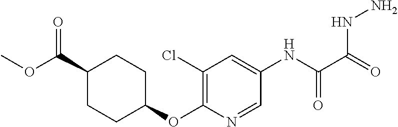

Hydrazine monohydrate (0.198 mL, 4.09 mmol) was added to (1s,4s)-methyl 4-(3-chloro-5-(2-methoxy-2-oxoacetamido)pyridin-2-yloxy)cyclohexanecarboxylate (intermediate 116) (1.379 g, 3.72 mmol), in ethanol (50 mL). The resulting suspension was stirred for 16 hours. The reaction mixture was filtered. The solid was dried under vacuum at 60° C. to give (1s,4s)-methyl 4-(3-chloro-5-(2-hydrazinyl-2-oxoacetamido)pyridin-2-yloxy)cyclohexanecarboxylate (1.286 g).

¹H NMR (400 MHz, DMSO) δ 1.66-1.91 (9H, m), 3.62 (3H, s), 4.65 (2H, s), 5.22 (1H, s), 8.30 (1H, d), 8.54 (1H, d), 10.35 (1H, s), 10.88 (1H, s). m/z (ESI−) (M−H)⁻=369.

### Intermediate 116

### (1s,4s)-methyl 4-(3-chloro-5-(2-methoxy-2-oxoacetamido)pyridin-2-yloxy)cyclohexanecarboxylate

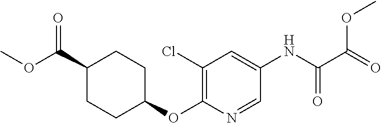

Methyl chlorooxoacetate (0.414 mL, 4.50 mmol) was added dropwise to (1s,4s)-methyl 4-(5-amino-3-chloropyridin-2-yloxy)cyclohexanecarboxylate (intermediate 117) (1.386 g, 4.09 mmol), and pyridine (0.397 mL, 4.91 mmol) in DCM (50 mL). The resulting solution was stirred for 2 hours. The reaction mixture was diluted with DCM (50 mL). Washed sequentially with water (20 mL), and saturated brine (20 mL). The organic layer was dried over MgSO₄, filtered and evaporated to afford crude product. The crude product was purified by flash silica chromatography, elution gradient 0 to 70% EtOAc in isohexane. Pure fractions were evaporated to dryness to afford (1s,4s)-methyl 4-(3-chloro-5-(2-methoxy-2-oxoacetamido)pyridin-2-yloxy)cyclohexanecarboxylate (1.379 g). ¹H NMR (400 MHz, DMSO) δ 1.64-1.90 (9H, m), 3.62 (3H, s), 3.86 (3H, s), 5.22 (1H, s), 8.24 (1H, d), 8.46 (1H, d), 11.03 (1H, s), m/z (ESI−) (M−H)⁻=369.

### Intermediate 117

### (1s,4s)methyl 4-(5-amino-3-chloropyridin-2-yloxy)cyclohexanecarboxylate

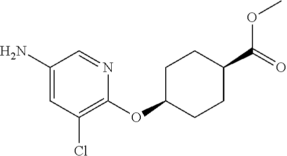

Zinc (3.21 g, 49.09 mmol) was added to (1s,4s)-methyl 4-(3-chloro-5-nitropyridin-2-yloxy)cyclohexanecarboxylate (intermediate 118) (1.545 g, 4.91 mmol) and iron(III) chloride hexahydrate (3.981 g, 14.73 mmol) in DMF (30 mL) & water (15 mL). The resulting mixture was warmed to 100° C. and stirred for 45 minutes. The reaction mixture was diluted with water (25 mL), then filtered. Reduced under vacuum. The residue was dissolved in EtOAc (200 mL), and washed with water (50 mL), then brine (50 mL). The organic layer was dried over MgSO₄, filtered and evaporated to afford crude product. The crude product was purified by flash silica chromatography, elution gradient 0 to 50% EtOAc in isohexane. Pure fractions were evaporated to dryness to afford (1s,4s)-methyl 4-(5-amino-3-chloropyridin-2-yloxy)cyclohexanecarboxylate (1.386 g). ¹H NMR (400 MHz, DMSO) δ 1.58-1.71 (4H, m), 1.73-1.84 (4H, m), 2.42-2.54 (1H+DMSO, m), 3.61 (3H, s), 5.01 (3H, s), 7.13 (1H, d), 7.45 (1H, d), m/z (ESI+) (M+H)⁺=285.

### Intermediate 118

### (1s,4s)-methyl 4-(3-chloro-5-nitropyridin-2-yloxy)cyclohexanecarboxylate

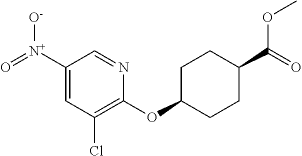

Diisopropyl azodicarboxylate (1.072 mL, 5.44 mmol) was added to a stirred solution of 3-chloro-5-nitropyridin-2-ol (500 mg, 2.86 mmol) and triphenylphosphine (1.728 g, 6.59 mmol) in THF (5 mL), followed by (1r,4r)-methyl 4-hydroxycyclohexanecarboxylate (453 mg, 2.86 mmol). The reaction was heated to 180° C. for 60 minutes in the microwave reactor then allowed to cool to ambient temperature. This was repeated 5 more times on exactly the same scale. The reaction mixtures were combined, evaporated and the crude product was purified by flash silica chromatography (120 g Crawford cartridge, loading in DCM), elution gradient 0 to 20% EtOAc in isohexane. Fractions were evaporated to dryness to afford (1s,4s)-methyl 4-(3-chloro-5-nitropyridin-2-yloxy)cyclohexanecarboxylate (2.525 g), ¹H NMR (400 MHz, DMSO) δ 1.71-1.83 (6H, m), 1.86-1.97 (2H, m), 2.47-2.57 (1H+DMSO, m), 3.62 (3H, s), 5.42 (1H, s), 8.72 (1H, d), 9.03 (1H, d).

### Intermediate 119

### (1s,4s)-methyl 4-(3-chloro-5-(5-(3,4-difluorophenylamino)-1,3,4-oxadiazole-2-carboxamido)pyridin-2-yloxy)cyclohexanecarboxylate

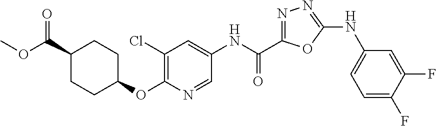

1,2-Difluoro-4-isothiocyanatobenzene (141 mg, 0.82 mmol) was added to (1s,4s)-methyl 4-(3-chloro-5-(2-hydrazinyl-2-oxoacetamido)pyridin-2-yloxy)cyclohexanecarboxylate (intermediate 115) (305 mg, 0.82 mmol), in DMF (8 mL). The resulting suspension was stirred at 55° C. for 1 hour. 1-(3-Dimethylaminopropyl)-3-ethylcarbodiimide hydrochloride (189 mg, 0.99 mmol) was added to the mixture. The resulting solution was stirred at 75° C. for 16 hours. The reaction mixture was concentrated then diluted with water (5 mL). A precipitate was collected by filtration. It was washed with water (5 mL), ether (5 mL) and dried under vacuum to afford (1s,4s)-methyl 4-(3-chloro-5-(5-(3,4-difluorophenylamino)-1,3,4-oxadiazole-2-carboxamido)pyridin-2-yloxy)cyclohexanecarboxylate (403 mg), which was used without further purification. m/z (ESI+) (M+H)⁺=508.

### Intermediate 120

### (1s,4s)-methyl 4-(3-chloro-5-(5-(3-chloro-4-fluorophenylamino)-1,3,4-oxadiazole-2-carboxamido)pyridin-2-yloxy)cyclohexanecarboxylate

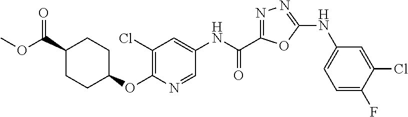

2-chloro-1-fluoro-4-isothiocyanatobenzene (154 mg, 0.82 mmol) was added to (1s,4s)-methyl 4-(3-chloro-5-(2-hydrazinyl-2-oxoacetamido)pyridin-2-yloxy)cyclohexanecarboxylate (intermediate 115) (305 mg, 0.82 mmol), in DMF (8 mL). The resulting suspension was stirred at 55° C. for 1 hour. 1-(3-Dimethylaminopropyl)-3-ethylcarbodiimide hydrochloride (189 mg, 0.99 mmol) was added to the mixture. The resulting solution was stirred at 75° C. for 16 hours. The reaction mixture was concentrated and diluted with water (5 mL). A precipitate was collected by filtration, washed with water (5 mL), ether (5 mL) and dried under vacuum to afford (1s,4s)-methyl 4-(3-chloro-5-(5-(3-chloro-4-fluorophenylamino)-1,3,4-oxadiazole-2-carboxamido)pyridin-2-yloxy)cyclohexanecarboxylate (413 mg) as a solid, which was used without further purification. m/z (ESI+) (M+H)⁺=524.

### Intermediate 121

### (1R,3R)-methyl 3-(5-(5-(3,4-difluorophenylamino)-1,3,4-oxadiazole-2-carboxamido)pyridin-2-yloxy)cyclopentanecarboxylate

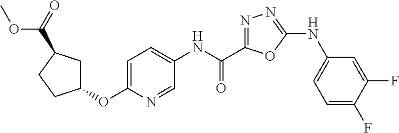

1,2-difluoro-4-isothiocyanatobenzene (159 mg, 0.93 mmol) was added to (1R,3R)-methyl 3-(5-(2-hydrazinyl-2-oxoacetamido)pyridin-2-yloxy)cyclopentanecarboxylate (intermediate 122) (300 mg, 0.93 mmol), in DMF (4 mL). The resulting solution was heated to 55° C. and stirred for 1 hour. 1-(3-Dimethylaminopropyl)-3-ethylcarbodiimide hydrochloride (214 mg, 1.12 mmol) in DMF (1 mL) was added to the mixture. The resulting solution was heated to 85° C. and stirred for 2 hours. The reaction mixture was concentrated, then diluted with water (5 mL). A precipitate was collected by filtration, washed with water (5 mL), ether (5 mL) and dried under vacuum to afford (1R,3R)-methyl 3-(5-(5-(3,4-difluorophenylamino)-1,3,4-oxadiazole-2-carboxamido)pyridin-2-yloxy)cyclopentanecarboxylate (425 mg). m/z (ESI+) (M+H)⁺=460.

### Intermediate 122

### (1R,3R)-methyl 3-(5-(2-hydrazinyl-2-oxoacetamido)pyridin-2-yloxy)cyclopentanecarboxylate

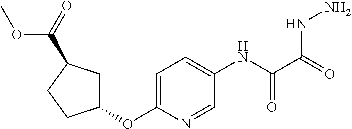

Hydrazine hydrate (0.221 mL, 4.54 mmol) was added to (1R,3R)-methyl 3-(5-(2-methoxy-2-oxoacetamido)pyridin-2-yloxy)cyclopentanecarboxylate (intermediate 123) (1.33 g, 4.13 mmol) in ethanol (50 mL). The resulting suspension was stirred for 16 hours. A solid was filtered off. The solid was washed with EtOH, then dried under vacuum to give (1R,3R)-methyl 3-(5-(2-hydrazinyl-2-oxoacetamido)pyridin-2-yloxy)cyclopentanecarboxylate (1.211 g). ¹H NMR (400 MHz, DMSO) δ 1.68-1.83 (2H, m), 1.94-2.16 (4H, m), 2.95-3.04 (1H, m), 3.60 (3H, s), 4.60 (2H, s), 5.33-5.40 (1H, m), 6.76 (1H, d), 8.04 (1H, dd), 8.54 (1H, d), 10.24 (1H, s), 10.66 (1H, s). m/z (ESI+) (M+H)⁺=323.

### Intermediate 123

### (1R,3R)-methyl 3-(5-(2-methoxy-2-oxoacetamido)pyridin-2-yloxy)cyclopentanecarboxylate

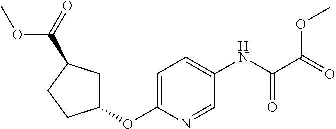

Methyl chlorooxoacetate (0.424 mL, 4.61 mmol) was added dropwise to a stirred solution of (1R,3R)-methyl 3-(5-aminopyridin-2-yloxy)cyclopentanecarboxylate (intermediate 124) (990 mg, 4.19 mmol), and pyridine (0.407 mL, 5.03 mmol) in DCM (50 mL), at ambient temperature. The resulting solution was stirred at ambient temperature for 1.5 hours. The reaction mixture was diluted with DCM (50 mL). Washed sequentially with water (40 mL), then saturated brine (40 mL). The organic layer was dried over MgSO₄, filtered and evaporated to afford (1R,3R)-methyl 3-(5-(2-methoxy-2-oxoacetamido)pyridin-2-yloxy)cyclopentanecarboxylate (1.333 g). ¹H NMR (400 MHz, DMSO) δ 1.52-1.66 (2H, m), 1.78-1.99 (4H, m), 2.78-2.88 (1H, m), 3.44 (3H, s), 3.68 (3H, s), 5.17-5.24 (1H, m), 6.61 (1H, d), 7.83 (1H, dd), 8.30 (1H, d). m/z (ESI+) (M+H)⁺=323.

### Intermediate 124

### (1R,3R)-methyl 3-(5-aminopyridin-2-yloxy)cyclopentanecarboxylate

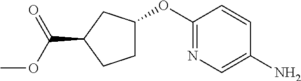

(1R,3R)-Methyl 3-(5-nitropyridin-2-yloxy)cyclopentanecarboxylate (intermediate 125) (1.19 g, 4.47 mmol), and 10% palladium on carbon (150 mg, 0.14 mmol) in MeOH (100 mL) were stirred under an atmosphere of hydrogen supplied by a balloon for 16 hours. The reaction mixture was filtered through celite. The solution was reduced to give (1R,3R)-methyl 3-(5-aminopyridin-2-yloxy)cyclopentanecarboxylate (0.993 g). ¹H NMR (400 MHz, DMSO) δ 1.63-1.79 (2H, m), 1.89-2.07 (4H, m), 2.91-3.01 (1H, m), 3.60 (3H, s), 4.68 (2H, s), 5.18-5.24 (1H, m), 6.47 (1H, d), 6.97 (1H, dd), 7.48 (1H, d). m/z (ESI+) (M+H)⁺=237

### Intermediate 125

### (1R,3R)-methyl 3-(5-nitropyridin-2-yloxy)cyclopentanecarboxylate

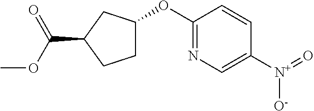

Diisopropyl azodicarboxylate (2.59 mL, 13.18 mmol) was added to 2-hydroxy-5-nitropyridine (0.972 g, 6.94 mmol), and triphenylphosphine (4.18 g, 15.95 mmol) in THF (30 mL) under nitrogen. The resulting suspension was stirred at for 1 hour. (1R,3S)-Methyl 3-hydroxycyclopentanecarboxylate (1 g, 6.94 mmol), in DCM (30 mL) was added and the solution was allowed to stir at ambient temperature for 16 hours. The reaction mixture was heated to 150° C. and stirred in the microwave for 15 minutes. The mixture was reduced under vacuum. The crude product was purified by flash silica chromatography, elution gradient 0 to 20% EtOAc in isohexane. Pure fractions were evaporated to dryness to afford (1R,3R)-methyl 3-(5-nitropyridin-2-yloxy)cyclopentanecarboxylate (1.190 g). ¹H NMR (400 MHz, DMSO) δ 1.75-1.86 (2H, m), 2.01-2.22 (4H, m), 2.99-3.08 (1H, m), 3.61 (3H, s), 5.51-5.57 (1H, m), 6.97 (1H, d), 8.45 (1H, dd), 9.07 (1H, d). m/z (ESI+) (M+H)⁺=267.

### Intermediate 126

### (1s,4s)-Methyl 4-(5-(5-(3,4-difluorophenylamino)-1,3,4-oxadiazole-2-carboxamido)-6-fluoropyridin-2-yloxy)cyclohexanecarboxylate

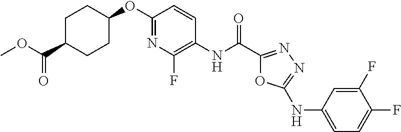

To a solution of (1s,4s)-methyl 4-(6-fluoro-5-(2-hydrazinyl-2-oxoacetamido)pyridin-2-yloxy)cyclohexanecarboxylate (intermediate 127) (700 mg, 1.98 mmol) in DMF (20 mL) was added 3,4-difluorophenyl isothiocyanate (406 mg, 2.37 mmol). The resulting mixture was stirred at 45° C. for 45 minutes, 1-(3-dimethylaminopropyl)-3-ethylcarbodiimide hydrochloride (549 mg, 2.86 mmol) was added and the mixture was stirred at 85° C. for 45 minutes. The reaction mixture was allowed to cool to ambient temperature and the mixture was concentrated by half. Water (20 mL) was added and the precipitate was collected by filtration, washed with water (10 mL) and air dried to afford the desired product as a yellow solid, which was used without further purification.

¹H NMR (400 MHz, DMSO-d₆) δ 1.68-1.87 (8H, m), 3.61 (3H, s), 5.01 (1H, s), 6.80 (1H, d), 7.32-7.35 (1H, m), 7.43-7.48 (1H, m), 7.65-7.71 (1H, m), 7.87-7.92 (1H, m), 10.77 (1H, s), 11.23 (1H, s) 1H obscurred by dmso (cyclohexyl proton). m/z 490 (M−H)⁻

### Intermediate 127

### (1s,4s)-methyl 4-(6-fluoro-5-(2-hydrazinyl-2-oxoacetamido)pyridin-2-yloxy)cyclohexanecarboxylate

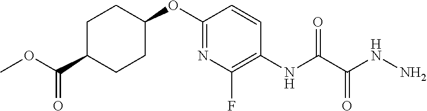

To a solution of (1s,4s)-methyl 4-(6-fluoro-5-(2-methoxy-2-oxoacetamido)pyridin-2-yloxy)cyclohexanecarboxylate (intermediate 128) (1.495 g, 4.22 mmol) in ethanol (100 mL) was added hydrazine monohydrate (0.225 mL, 4.64 mmol). The resulting suspension was stirred at ambient temperature for 90 minutes. The reaction mixture was evaporated to dryness to afford (1s,4s)-methyl 4-(6-fluoro-5-(2-hydrazinyl-2-oxoacetamido)pyridin-2-yloxy)cyclohexanecarboxylate (1.400 g, 94%) as a white solid.

¹H NMR (400 MHz, DMSO-d₆) δ 1.67-1.85 (8H, m), 3.61 (3H, s), 4.61 (1H, s), 4.99 (1H, m), 6.76-6.78 (1H, m), 7.87-7.91 (1H, m), 10.3 (2H, s), two protons not observed. m/z 355 (M+H)⁺

### Intermediate 128

### (1s,4s)-methyl 4-(6-fluoro-5-(2-methoxy-2-oxoacetamido)pyridin-2-yloxy)cyclohexanecarboxylate

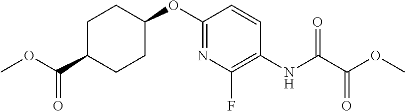

To a solution of (1s,4s)-methyl 4-(5-amino-6-fluoropyridin-2-yloxy)cyclohexanecarboxylate (intermediate 129) (2.24 g, 8.35 mmol) in DCM (60 mL) was added pyridine (0.810 mL, 10.02 mmol) and methyl oxalyl chloride (0.998 mL, 10.85 mmol). The resulting solution was stirred at ambient temperature for overnight. The reaction mixture was diluted with DCM (100 mL), and washed with saturated brine (100 mL), the aqueous layer was washed with DCM (50 mL). The organic extracts were combined, dried over MgSO₄, filtered and evaporated to afford an orange oil. The crude product was purified by flash silica chromatography, elution gradient 10 to 50% EtOAc in isohexane to yield (1s,4s)-methyl 4-(6-fluoro-5-(2-methoxy-2-oxoacetamido)pyridin-2-yloxy)cyclohexanecarboxylate (1.495 g, 4.22 mmol, 50.5%) as a yellow solid.

¹H NMR (400 MHz, CDCl₃) δ 1.56-1.65 (2H, m), 1.68-1.75 (2H, m), 1.82-1.95 (4H, m), 2.33-2.40 (1H, m), 3.63 (3H, s), 3.92 (3H, s), 5.02-5.05 (1H, m), 6.58 (1H, d), 8.48 (1H, t), 8.74 (1H, s). m/z 355 (M+H)⁺

### Intermediate 129

### (1s,4s)-methyl 4-(5-amino-6-fluoropyridin-2-yloxy)cyclohexanecarboxylate

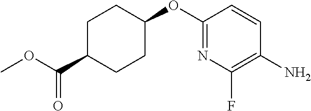

10% Palladium on charcoal (200 mg, 0.19 mmol) was weighed into a round bottom flask which was evacuated and purged with nitrogen 3 times. EtOAc (250 mL) was added followed by (1s,4s)-methyl 4-(6-fluoro-5-nitropyridin-2-yloxy)cyclohexanecarboxylate (intermediate 130) (1.9 g, 6.37 mmol) under nitrogen. The reaction flask was evacuated and purged with hydrogen a further 3 times, then the suspension was stirred under an atmosphere of hydrogen at room temperature for 4 hours. The reaction mixture was filtered through celite and evaporated to afford crude product which was purified by flash silica chromatography, elution gradient 0 to 30% EtOAc in isohexane. Pure fractions were evaporated to dryness to afford (1s,4s)-methyl 4-(5-amino-6-fluoropyridin-2-yloxy)cyclohexanecarboxylate (304 mg, 1.13 mmol, 17.79%) as an orange oil.

¹H NMR (400 MHz, CDCl₃) δ 1.60-1.68 (2H, m), 1.72-1.77 (2H, m), 1.89-2.00 (4H, m), 2.39-2.44 (1H, m), 3.69 (3H, s), 4.98-5.01 (1H, m), 6.45-6.48 (1H, m), 7.09-7.14 (1H, m), 7.26 (2H, s). m/z 269 (M+H)⁺

### Intermediate 130

### (1s,4s)-methyl 4-(6-fluoro-5-nitropyridin-2-yloxy)cyclohexanecarboxylate

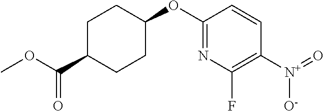

To a solution of tetrabutylammonium cyanide (22.03 g, 82.06 mmol) in DMSO (80 mL) on an ice bath was added dropwise hexafluorobenzene (9.47 mL, 82.06 mmol) and the resulting red brown solution was stirred at ambient temperature under nitrogen for 2.5 hours. The reaction mixture was cooled to 0° C. (ice water bath) and (1s,4s)-methyl 4-(6-chloro-5-nitropyridin-2-yloxy)cyclohexanecarboxylate (intermediate 131) (9.46 g, 30.06 mmol) was added. The reaction mixture was allowed to stir at ambient temperature for 2 minutes and was then diluted with water (500 mL), extracted with EtOAc (2×500 mL). The organic extracts were combined, washed with brine (200 mL) and evaporated to afford crude product which was purified by flash silica chromatography, elution gradient 0 to 30% EtOAc in isohexane. Yielded (1s,4s)-methyl 4-(6-fluoro-5-nitropyridin-2-yloxy)cyclohexanecarboxylate (6.8 g, 22.80 mmol, 76%) as a yellow solid.

¹H NMR (400 MHz, CDCl₃) δ 1.70-1.85 (4H, m), 1.89-1.96 (2H, m), 2.00-2.05 (2H, m), 2.44-2.50 (1H, m), 3.70 (3H, s), 5.26-5.30 (1H, m), 6.70 (1H, s), 8.42 (1H, t). No mass ion observed.

### Intermediate 131

### (1s,4s)-methyl 4-(6-chloro-5-nitropyridin-2-yloxy)cyclohexanecarboxylate

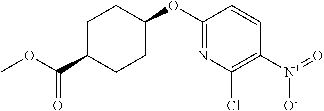

Diisopropyl azodicarboxylate (21.01 mL, 106.71 mmol) was added to a stirred solution of 6-chloro-5-nitropyridin-2-ol (intermediate 132) (14.9 g, 85.37 mmol) and triphenylphosphine (33.6 g, 128.05 mmol) in THF (200 mL) under nitrogen. The reaction mixture was stirred at ambient temperature for 10 minutes and then (1r,4r)-methyl 4-hydroxycyclohexanecarboxylate (13.50 g, 85.37 mmol) in THF (50 mL) was added and the resulting solution was stirred at ambient temperature for 2 days. The reaction mixture was filtered and the filtrate evaporated to leave a viscous oil which was stirred with ether (200 mL) to produce a suspension. The solid was filtered and washed with ether (100 mL). The filtrate was concentrated and the residue was purified by flash silica chromatography, elution gradient 0 to 20% EtOAc in isohexane. Yielded (1s,4s)-methyl 4-(6-chloro-5-nitropyridin-2-yloxy)cyclohexanecarboxylate (9.46 g, 35.2%) as a yellow solid.

¹H NMR (400 MHz, CDCl₃) δ 1.69-1.77 (2H, m), 1.79-1.85 (2H, m), 1.88-1.95 (2H, m), 2.00-2.06 (2H, m), 2.43-2.50 (1H, m), 3.70 (3H, s), 5.33-5.37 (1H, m), 6.74 (1H, d), 8.24 (1H, d). No mass ion observed.

### Intermediate 132

### 6-chloro-5-nitropyridin-2-ol

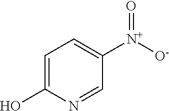

A suspension of 2-chloro-6-methoxy-3-nitropyridine (20.85 g, 110.57 mmol) in concentrated hydrochloric acid (37%) (171 mL, 5528.49 mmol) was stirred at 90° C. for 3 hours and allowed to cool to ambient temperature. The suspension was adjusted to pH 5 with 2M NaOH and the aqueous mixture was then continually extracted with EtOAc (250 mL). The organics were concentrated to afford a yellow solid which was filtered off and washed with Et₂O (200 mL). The solid was stirred in EtOAc (200 mL) and the green insoluble solid filtered off. Both filtrates were combined and dried (MgSO₄) before concentrating to yield as 6-chloro-5-nitropyridin-2-ol a yellow solid (14.7 g, 84.22 mmol, 76%).

¹H NMR (400 MHz, DMSO-d₆) δ 6.52 (1H, d), 8.26 (1H, d), no OH peak observed. m/z 173 (M−H)−

### Intermediate 133

### (1s,4s)-methyl 4-(5-(5-(3,4-difluorophenylamino)-1,3,4-oxadiazole-2-carboxamido)-6-methoxypyridin-2-yloxy)cyclohexanecarboxylate

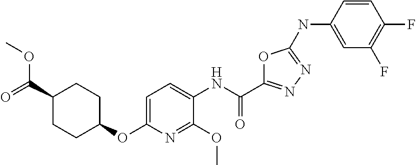

3,4-Difluorophenyl isothiocyanate (0.112 g, 0.66 mmol) was added to (1s,4s)-methyl 445-(2-hydrazinyl-2-oxoacetamido)-6-methoxypyridin-2-yloxy)cyclohexanecarboxylate (Intermediate 134) (0.2 g, 0.55 mmol) in DMF (5 mL) at 20° C. The resulting solution was stirred at 50° C. for 1 hour. 1-(3-Dimethylaminopropyl)-3-ethylcarbodiimide hydrochloride (0.126 g, 0.66 mmol) was added and the solution stirred at 85° C. for 3 hours. The reaction mixture was allowed to cool to ambient temperature and half the DMF was evaporated off before adding water (8 mL). The precipitate was collected by filtration, washed with water (25 mL) and dried under vacuum to afford (1s,4s)-methyl 4-(5-(5-(3,4-difluorophenylamino)-1,3,4-oxadiazole-2-carboxamido)-6-methoxypyridin-2-yloxy)cyclohexanecarboxylate (0.260 g, 95%) as a yellow solid.

1H NMR (400.132 MHz, DMSO) δ 1.69-1.92 (8H, m), 2.54-2.57 (1H, m), 3.63 (3H, s), 3.91 (3H, s), 5.07-5.14 (1H, m), 6.42 (1H, d), 7.33-7.38 (1H, m), 7.48 (1H, q), 7.66-7.74 (1H, m), 7.90 (1H, d), 9.90 (1H, s), 11.23 (1H, s). m/z 504 (M+H)⁺

### Intermediate 134

### (1s,4s)-methyl 4-(5-(2-hydrazinyl-2-oxoacetamido)-6-methoxypyridin-2-yloxy)cyclohexanecarboxylate

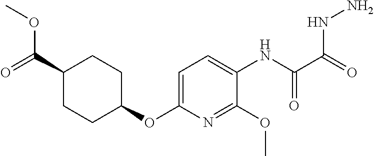

Hydrazine monohydrate (0.094 mL, 1.93 mmol) was added to (1s,4s)-methyl 4-(6-methoxy-5-(2-methoxy-2-oxoacetamido)pyridin-2-yloxy)cyclohexanecarboxylate (Intermediate 135) (0.59 g, 1.61 mmol) in ethanol (30 mL) at ambient temperature. The resulting suspension was stirred at ambient temperature for 1 hour and at 60° C. for 2 hours. The precipitate was collected by filtration, washed with EtOH (100 mL) and ether (200 mL) and dried under vacuum to afford (1s,4s)-methyl 4-(5-(2-hydrazinyl-2-oxoacetamido)-6-methoxypyridin-2-yloxy)cyclohexanecarboxylate (0.400 g, 67.8%) as a white solid, which was used without further purification.

1H NMR (400.132 MHz, DMSO) δ 1.66-1.91 (8H, m), 2.47-2.49 (1H, m), 3.62 (3H, s), 3.91 (3H, s), 4.64 (2H, d), 5.04-5.10 (1H, m), 6.42 (1H, d), 8.15 (1H, d), 9.51 (1H, s), 10.35 (1H, t). m/z 367 (M+H)⁺

### Intermediate 135

### (1s,4s)-methyl 4-(6-methoxy-5-(2-methoxy-2-oxoacetamido)pyridin-2-yloxy)cyclohexanecarboxylate

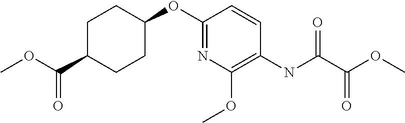

Methyl oxalyl chloride (0.287 mL, 3.13 mmol) was added to (1s,4s)-methyl 4-(5-amino-6-methoxypyridin-2-yloxy)cyclohexanecarboxylate (Intermediate 136) (0.73 g, 2.60 mmol) and Pyridine (0.421 mL, 5.21 mmol) in DCM (20 mL) cooled to 0° C. under nitrogen. The resulting solution was stirred at 20° C. for 1 hour.

The reaction mixture was quenched with water (50 mL), extracted with DCM (2×75 mL), the organic layer was washed with citric acid (50 mL), brine (50 mL), dried over MgSO₄, filtered and evaporated to afford crude product.

The crude product was purified by flash silica chromatography, elution gradient 20 to 50% EtOAc in isohexane. Pure fractions were evaporated to dryness to afford (1s,4s)-methyl 4-(6-methoxy-5-(2-methoxy-2-oxoacetamido)pyridin-2-yloxy)cyclohexanecarboxylate (0.596 g, 62.5%) as a colourless oil.

1H NMR (400.132 MHz, CDCl3) δ 1.61-1.70 (2H, m), 1.74-1.79 (2H, m), 1.92-2.06 (4H, m), 2.39-2.49 (1H, m), 3.70 (3H, s), 3.96 (3H, s), 3.98 (3H, s), 5.10-5.15 (1H, m), 6.34 (1H, d), 8.50 (1H, d), 9.05 (1H, s). m/z 367 (M+H)⁺

### Intermediate 136

### (1s,4s)-methyl 4-(5-amino-6-methoxypyridin-2-yloxy)cyclohexanecarboxylate

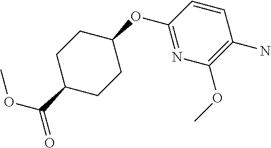

(1s,4s)-methyl 4-(6-methoxy-5-nitropyridin-2-yloxy)cyclohexanecarboxylate (Intermediate 137) (1.11 g, 3.58 mmol) and 10% palladium on carbon (111 mg, 0.10 mmol) in ethanol (30 mL) and THF (30.0 mL) were stirred under an atmosphere of hydrogen for 20 hours. The reaction mixture was filtered through celite and the solvent evaporated to give crude product. The crude product was purified by flash silica chromatography, elution gradient 20 to 70% EtOAc in isohexane. Pure fractions were evaporated to dryness to afford (1s,4s)-methyl 4-(5-amino-6-methoxypyridin-2-yloxy)cyclohexanecarboxylate (0.730 g, 72.8%) as a beige oil.

¹H NMR (400 MHz, CDCl₃) δ 1.58-1.68 (2H, m), 1.71-1.77 (2H, m), 1.92-2.02 (4H, m), 2.38-2.47 (1H, m), 3.35 (2H, s), 3.69 (3H, s), 3.91 (3H, s), 4.96-5.03 (1H, m), 6.17 (1H, d), 6.92 (1H, d). m/z 281 (M+H)⁺

### Intermediate 137

### (1s,4s)-methyl 4-(6-methoxy-5-nitropyridin-2-yloxy)cyclohexanecarboxylate

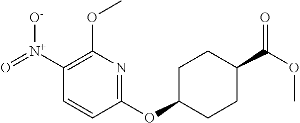

Palladium(II) acetate (0.171 g, 0.76 mmol), 2-(di-tert-butylphosphino)-1,1′-binaphthyl (0.380 g, 0.95 mmol) and caesium carbonate (9.33 g, 28.64 mmol) were added to an oven dried flask that had been cooled under nitrogen. The flask was evacuated twice and filled with nitrogen. 6-chloro-2-methoxy-3-nitropyridine (J. Het. chem, 1996, 1995-2005) (1.8 g, 9.55 mmol) was added followed by the addition of toluene (25 mL). (1s,4s)-methyl 4-hydroxycyclohexanecarboxylate (Intermediate 138) (1.510 g, 9.55 mmol) was added portionwise followed by the addition of toluene (25 mL) under nitrogen. The resulting suspension was stirred at 90° C. for 16 hours. The reaction mixture was diluted with ethyl acetate, filtered through celite and washed with ethyl acetate. The crude product was purified by flash silica chromatography, elution gradient 10 to 35% EtOAc in isohexane. Pure fractions were evaporated to dryness to afford (1s,4s)-methyl 4-(6-methoxy-5-nitropyridin-2-yloxy)cyclohexanecarboxylate (1.110 g, 37.5%) as a white solid.

¹H NMR (400 MHz, CDCl₃) δ 1.68-1.76 (2H, m), 1.78-1.85 (2H, m), 1.89-2.00 (2H, m), 2.04-2.10 (2H, m), 2.42-2.49 (1H, m), 3.70 (3H, s), 4.07 (3H, s), 5.26-5.31 (1H, m), 6.37 (1H, d), 8.35 (1H, d). m/z 311 (M+H)⁺

### Intermediate 138

### (1s,4s)-methyl 4-(5-(5-(3-chloro-4-fluorophenylamino)-1,3,4-oxadiazole-2-carboxamido)-6-methoxypyridin-2-yloxy)cyclohexanecarboxylic acid

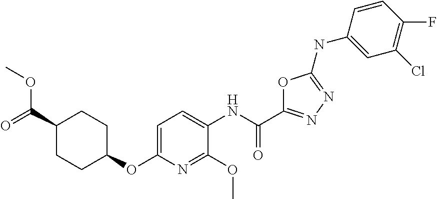

Prepared by the method as for Intermediate 133 by reaction of Intermediate 134 with the 3-chloro-4-fluorophenyl isothiocyanate.

¹H NMR (400 MHz, DMSO) δ 1.69-1.95 (8H, m), 2.55-2.59 (1H, m), 3.63 (3H, s), 3.91 (3H, s), 5.07-5.16 (1H, m), 6.42 (1H, d), 7.43-7.54 (2H, m), 7.83-7.85 (1H, m), 7.90 (1H, d), 9.89 (1H, s), 11.21 (1H, s). m/z 520 (M+H)⁺

### Intermediate 139

### Methyl 2-(1s,4s)-4-(5-(5-(3-chloro-4-fluorophenylamino)-1,3,4-oxadiazole-2-carboxamido)pyridin-2-yloxy)cyclohexyl)acetate

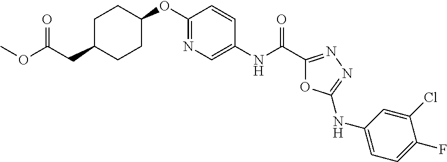

To a solution of methyl 2-((1s,4s)-4-(5-(2-hydrazinyl-2-oxoacetamido)pyridin-2-yloxy)cyclohexyl)acetate (intermediate 84) (700 mg, 2.00 mmol) in DMF (5 mL) was added 3-chloro-4-fluorophenyl isothiocyanate (375 mg, 2.00 mmol). The resulting mixture was stirred at 45° C. for 90 minutes, 1-(3-dimethylaminopropyl)-3-ethylcarbodiimide hydrochloride (460 mg, 2.40 mmol) was added and the mixture was stirred at 85° C. for 45 minutes. The reaction mixture was allowed to cool to ambient temperature, water (15 mL) was added and the suspension stirred for 2 hours and then filtered, washed with water (10 mL) and dried to leave methyl 2-(1s,4s)-4-(5-(5-(3-chloro-4-fluorophenylamino)-1,3,4-oxadiazole-2-carboxamido)pyridin-2-yloxy)cyclohexyl)acetate (1.007 g, 2.00 mmol) as a pale brown solid.

¹H NMR (400 MHz, DMSO) δ 1.31-1.42 (2H, m), 1.51-1.66 (4H, m), 1.79-1.92 (3H, m), 2.27 (2H, d), 3.59 (3H, s), 5.14-5.17 (1H, m), 6.82 (1H, d), 7.48 (1H, d), 7.51-7.56 (1H, m), 7.82-7.84 (1H, m), 8.05 (1H, d), 8.50 (1H, s), 11.08 (1H, s), 11.22 (1H, s). m/z 504 (M+H)⁺.

### Intermediate 140

### methyl 2-(1s,4s)-4-(5-(5-(2,4-dichlorophenylamino)-1,3,4-oxadiazole-2-carboxamido)pyridin-2-yloxy)cyclohexyl)acetate

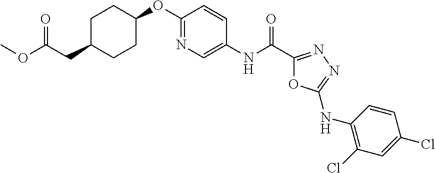

To a solution of methyl 2-((1s,4s)-4-(5-(2-hydrazinyl-2-oxoacetamido)pyridin-2-yloxy)cyclohexyl)acetate (intermediate 84) (700 mg, 2.00 mmol) in DMF (5 mL) was added 2,4-dichlorophenyl isothiocyanate (408 mg, 2.00 mmol). The resulting mixture was stirred at 45° C. for 90 minutes, 1-(3-dimethylaminopropyl)-3-ethylcarbodiimide hydrochloride (460 mg, 2.40 mmol) was added and the mixture was stirred at 85° C. for 45 minutes. The reaction mixture was allowed to cool to ambient temperature, water (15 mL) was added and the suspension stirred for 2 hours and then filtered, washed with water (10 mL) and dried to leave methyl 2-((1s,4s)-4-(5-(5-(2,4-dichlorophenylamino)-1,3,4-oxadiazole-2-carboxamido)pyridin-2-yloxy)cyclohexyl)acetate (1.04 g, 2.00 mmol, 100%) as a yellow solid. ¹H NMR (400 MHz, DMSO) δ 1.31-1.41 (2H, m), 1.51-1.66 (4H, m), 1.80-1.92 (3H, m), 2.27 (2H, d), 3.60 (3H, s), 5.14-5.17 (1H, m), 6.82 (1H, d), 7.52 (1H, d), 7.71 (1H, s), 8.01-8.06 (2H, m), 8.49 (1H, s), 10.54 (1H, s), 11.07 (1H, s). m/z 520 (M+H)⁺.

### Intermediate 141

### (1s,4s)-methyl 4-(5-(5-(3-chloro-4-fluorophenylamino)-1,3,4-oxadiazole-2-carboxamido)-6-fluoropyridin-2-yloxy)cyclohexanecarboxylate

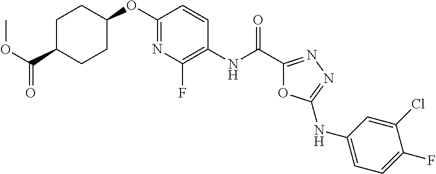

To a solution of (1s,4s)-methyl 4-(6-fluoro-5-(2-hydrazinyl-2-oxoacetamido)pyridin-2-yloxy)cyclohexanecarboxylate (intermediate 127) (315 mg, 0.89 mmol) in DMF (10 mL) was added 3-chloro-4-fluorophenylisothiocyanate (200 mg, 1.07 mmol). The resulting mixture was stirred at 45° C. for 45 minutes, 1-(3-dimethylaminopropyl)-3-ethylcarbodiimide hydrochloride (247 mg, 1.29 mmol) was added and the mixture was stirred at 85° C. for 45 minutes. The reaction mixture was allowed to cool to ambient temperature, water (10 mL) was added and the precipitate was collected by filtration, washed with water (10 mL) and air dried to afford the desired product as a beige solid (0.452 g, 100%), which was used without further purification. ¹H NMR (400 MHz, DMSO) δ 1.68-1.86 (8H, m), 3.61 (3H, s), 4.98-5.04 (1H, m), 6.80 (1H, d), 7.43-7.54 (2H, m), 7.81-7.84 (1H, m), 7.89 (1H, t), 10.77 (1H, s), 11.22 (1H, s); 1H obscurred by DMSO (cyclohexyl proton). m/z 508 (M+H)⁺.

### Intermediate 142

### (1s,4s)-methyl 4-(5-(5-(3-chloro-4-fluorophenylamino)-1,3,4-oxadiazole-2-carboxamido)-3-methoxypyridin-2-yloxy)cyclohexanecarboxylate

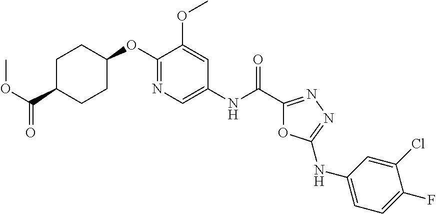

To a solution of (1s,4s)-methyl 4-(5-(2-hydrazinyl-2-oxoacetamido)-3-methoxypyridin-2-yloxy)cyclohexanecarboxylate (intermediate 144) (280 mg, 0.76 mmol) in DMF (10 mL) was added 3-chloro-4-fluorophenylisothiocyanate (172 mg, 0.92 mmol). The resulting mixture was stirred at 45° C. for 45 minutes, 1-(3-dimethylaminopropyl)-3-ethylcarbodiimide hydrochloride (212 mg, 1.11 mmol) was added and the mixture was stirred at 85° C. for 45 minutes. The reaction mixture was allowed to cool to ambient temperature, water (10 mL) was added and the precipitate was collected by filtration, washed with water (10 mL) and air dried to afford the desired product as a yellow solid (0.395 g, 100%), which was used without further purification. ¹H NMR (400 MHz, DMSO) δ 1.65-1.86 (8H, m), 3.61 (3H, s), 3.78 (3H, s), 5.14-5.18 (1H, m), 7.46 (1H, t), 7.50-7.55 (1H, m), 7.72-7.73 (1H, m), 7.81-7.84 (1H, m), 8.11 (1H, s), 11.03 (1H, s), 11.22 (1H, s); 1H obscurred by DMSO (cyclohexyl proton). m/z 520 (M+H)⁺.

### Intermediate 143

### (1s,4s)-Methyl 4-(5-(5-(3,4-difluorophenylamino)-1,3,4-oxadiazole-2-carboxamido)-3-methoxypyridin-2-yloxy)cyclohexanecarboxylate

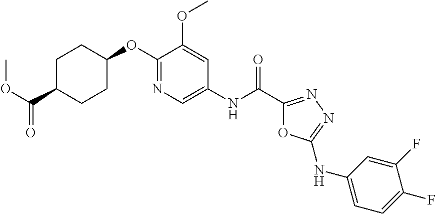

To a solution of (1s,4s)-methyl 4-(5-(2-hydrazinyl-2-oxoacetamido)-3-methoxypyridin-2-yloxy)cyclohexanecarboxylate (intermediate 144) (283 mg, 0.77 mmol) in DMF (10 mL) was added 3,4-difluorophenyl isothiocyanate (159 mg, 0.93 mmol). The resulting mixture was stirred at 45° C. for 45 minutes, 1-(3-dimethylaminopropyl)-3-ethylcarbodiimide hydrochloride (215 mg, 1.12 mmol) was added and the mixture was stirred at 85° C. for 45 minutes. The reaction mixture was allowed to cool to ambient temperature, water (10 mL) was added and the precipitate was collected by filtration, washed with water (10 mL) and air dried to afford the desired product as a yellow solid (0.388 g, 100%), which was used without further purification. ¹H NMR (400 MHz, DMSO) δ 1.64-1.85 (8H, m), 3.61 (3H, s), 3.78 (3H, s), 5.13-5.18 (1H, m), 7.32-7.37 (1H, m), 7.47 (1H, q), 7.66-7.71 (1H, m), 7.73 (1H, s), 8.11 (1H, s), 11.03 (1H, s), 11.23 (1H, s); 1H obscurred by DMSO (cyclohexyl proton). m/z 504 (M+H)⁺.

### Intermediate 144

### (1s,4s)-methyl 4-(5-(2-hydrazinyl-2-oxoacetamido)-3-methoxy-pyridin-2-yloxy)cyclohexanecarboxylate

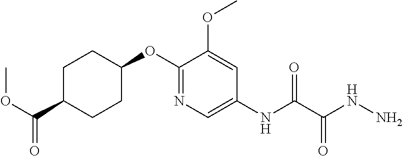

To a solution of (1s,4s)-methyl 4-(3-methoxy-5-(2-methoxy-2-oxoacetamido)pyridin-2-yloxy)cyclohexanecarboxylate (intermediate 145) (842 mg, 2.30 mmol) in ethanol (100 mL) was added hydrazine monohydrate (0.123 mL, 2.53 mmol). The resulting suspension was stirred at ambient temperature for 90 minutes. The reaction mixture was evaporated to dryness to afford (1s,4s)-methyl 4-(5-(2-hydrazinyl-2-oxoacetamido)-3-methoxypyridin-2-yloxy)cyclohexanecarboxylate (849 mg, 100%) as an orange solid. ¹H NMR (400 MHz, DMSO) δ 1.63-1.86 (8H, m), 3.61 (3H, s), 3.76 (3H, s), 4.61 (2H, s), 5.11-5.15 (1H, m), 7.76 (1H, s), 8.15 (1H, s), 10.58 (1H, s); one proton obscurred by DMSO and one NH not seen. m/z 367 (M+H)⁺.

### Intermediate 145

### (1s,4s)-methyl 4-(3-methoxy-5-(2-methoxy-2-oxoacetamido)pyridin-2-yloxy)cyclohexanecarboxylate

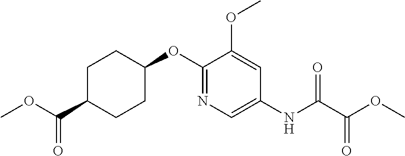

To a solution of (1s,4s)-methyl 4-(5-amino-3-methoxypyridin-2-yloxy)cyclohexanecarboxylate (intermediate 146) (2.03 g, 7.24 mmol) in DCM (50 mL) was added pyridine (0.702 mL, 8.69 mmol) and methyl oxalyl chloride (0.866 mL, 9.41 mmol). The resulting solution was stirred at ambient temperature for 1 hour. The reaction mixture was diluted with DCM (100 mL), and washed with saturated brine (100 mL), the aqueous layer was washed with DCM (50 mL). The organic extracts were combined, dried over MgSO₄, filtered and evaporated to afford crude product. The crude product was purified by flash silica chromatography, elution gradient 40 to 80% EtOAc in isohexane. Pure fractions were evaporated to dryness to afford (1s,4s)-methyl 4-(3-methoxy-5-(2-methoxy-2-oxoacetamido)pyridin-2-yloxy)cyclohexanecarboxylate (0.842 g, 31.7%) as an orange solid. ¹H NMR (400 MHz, DMSO) δ 1.62-1.85 (8H, m), 3.61 (3H, s), 3.76 (3H, s), 3.85 (3H, s), 5.11-5.16 (1H, m), 7.66 (1H, s), 8.07 (1H; s), 10.77 (1H, s); 1H obscurred by DMSO—cyclohexyl. m/z 367 (M+H)⁺.

### Intermediate 146

### (1s,4s)-methyl 4-(5-amino-3-methoxypyridin-2-yloxy)cyclohexanecarboxylate

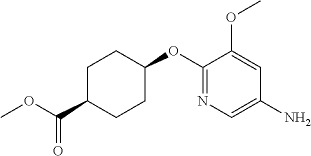

A mixture of (1s,4s)-methyl 4-(3-methoxy-5-nitropyridin-2-yloxy)cyclohexanecarboxylate (intermediate 147) (2.91 g, 9.38 mmol) and 10% palladium on carbon (0.279 g, 2.63 mmol) in EtOAc (200 mL) and EtOH (100 mL) was evacuated with hydrogen (3 cycles) and then stirred under an atmosphere of hydrogen at ambient temperature, overnight. The reaction mixture was filtered and evaporated to afford (1s,4s)-methyl 4-(5-amino-3-methoxypyridin-2-yloxy)cyclohexanecarboxylate (2.040 g, 78%) as a black gum, which was used without further purification. ¹H NMR (400 MHz, DMSO) δ 1.58-1.84 (8H, m), 2.42-2.48 (1H, m), 3.61 (3H, s), 3.69 (3H, s), 4.66 (2H, s), 4.90-4.94 (1H, m), 6.63 (1H, s), 7.01 (1H, s). m/z 281 (M+H)⁺.

### Intermediate 147

### (1s,4s)-Methyl 4-(3-methoxy-5-nitropyridin-2-yloxy)cyclohexanecarboxylate

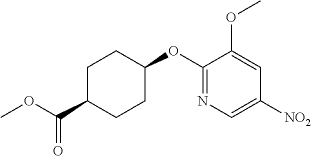

A solution of trimethylsilyldiazomethane (2M solution in ether) (9.82 mL, 19.64 mmol) was added dropwise to a stirred solution of (1s,4s)-4-(3-methoxy-5-nitropyridin-2-yloxy)cyclohexanecarboxylic acid (intermediate 148) (2.91 g, 9.82 mmol) in toluene (100 mL) and methanol (50 mL) over a period of 1 minute. The resulting solution was stirred at ambient temperature for 1 hour. The reaction mixture was evaporated to afford (1s,4s)-methyl 4-(3-methoxy-5-nitropyridin-2-yloxy)cyclohexanecarboxylate (2.91 g, 95%) of a black oil which was used without further purification. ¹H NMR (400 MHz, DMSO) δ 1.70-1.87 (8H, m), 3.62 (3H, s), 3.92 (3H, s), 5.31-5.37 (1H, m), 7.91 (1H, s), 8.65 (1H, s); 1H obscured by DMSO. m/z 311 (M+H)⁺.

### Intermediate 148

### (1r,4r)-4-(3-methoxy-5-nitropyridin-2-yloxy)cyclohexanecarboxylic acid

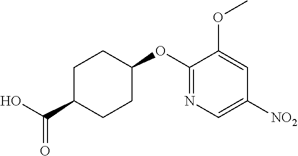

To an ice water cooled suspension of sodium hydride (60% in oil) (0.919 g, 22.98 mmol) in DMA (20 mL) was added a solution of (1s,4s)-4-hydroxycyclohexanecarboxylic acid (1.506 g, 10.45 mmol) in DMA (20 mL) over a period of 4 minutes under nitrogen. The resulting suspension was stirred at 0° C. for 1 hour. A solution of 2-chloro-3-methoxy-5-nitropyridine (1.97 g, 10.45 mmol) in DMA (10 mL) was added and the resulting mixture was stirred at ambient temperature over the weekend. The reaction mixture was concentrated, water (50 mL) was added and the mixture was acidified with 2M HCl. The precipitate was collected by filtration and dried under vacuum to afford (1r,4r)-4-(3-methoxy-5-nitropyridin-2-yloxy)cyclohexanecarboxylic acid (2.91 g, 94%) as a brown solid, which was used without further purification. ¹H NMR (400 MHz, DMSO) δ 1.66-1.88 (8H, m), 2.36-2.43 (1H, m), 3.92 (3H, s), 5.30-5.35 (1H, m), 7.91 (1H, s), 8.65 (1H, s). m/z 295 (M−H)⁻.

### Intermediate 149

### (1s,4s)-Methyl-4-(4-fluoro-5-(5-(2,4,5-trifluorophenylamino)-1,3,4-oxadiazole-2-carboxamido)pyridin-2-yloxy)cyclohexanecarboxylate

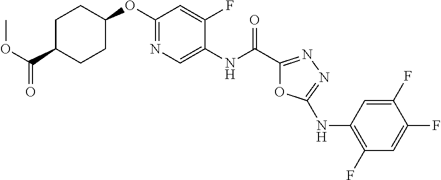

A solution of 2,4,5-trifluorophenyl isothiocyanate (303 mg, 1.60 mmol) in DMF (2 mL) was added to a stirred suspension of (1s,4s)-methyl 4-(4-fluoro-5-(2-hydrazinyl-2-oxoacetamido)pyridin-2-yloxy)cyclohexanecarboxylate (intermediate 151) (473 mg, 1.33 mmol) in DMF (10 mL). The resulting solution was stirred at 45° C. for 45 minutes, 1-(3-dimethylaminopropyl)-3-ethylcarbodiimide hydrochloride (371 mg, 1.94 mmol) was added and the mixture was stirred at 85° C. for 70 minutes. The reaction mixture was allowed to cool to ambient temperature, water (10 mL) was added and the gum was dissolved in EtOAc (75 mL), and washed sequentially with saturated brine (2×75 mL). The aqueous phase was re extracted with EtOAc (50 mL) and the combined organic layers were dried over MgSO₄, filtered and evaporated to afford crude product. The crude product was purified by flash silica chromatography, elution gradient 0 to 30 to 50% EtOAc in isohexane. Pure fractions were evaporated to dryness to afford (1s,4s)-methyl 4-(4-fluoro-5-(5-(2,4,5-trifluorophenylamino)-1,3,4-oxadiazole-2-carboxamido)pyridin-2-yloxy)cyclohexanecarboxylate (415 mg, 61.0%) as a cream solid. ¹H NMR (400 MHz, DMSO) δ 1.68-1.87 (8H, m), 2.50-2.52 (1H, m), 3.61 (3H, s), 5.16-5.18 (1H, m), 6.87 (1H, d), 7.66-7.73 (1H, m), 8.11-8.17 (1H, m), 8.20 (1H, d), 10.84 (1H, s), 11.07 (1H, s). m/z 510 (M+H)⁺.

### Intermediate 150

### (1s,4s)-methyl 4-(5-(5-(3,4-difluorophenylamino)-1,3,4-oxadiazole-2-carboxamido)-4-fluoropyridin-2-yloxy)cyclohexanecarboxylate

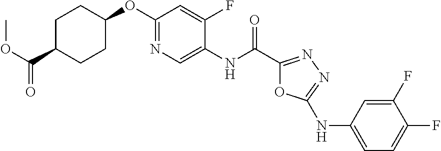

A solution of 3,4-difluorophenyl isothiocyanate (217 mg, 1.27 mmol) in DMF (2 mL) was added to a stirred suspension of (1s,4s)-methyl 4-(4-fluoro-5-(2-hydrazinyl-2-oxoacetamido)pyridin-2-yloxy)cyclohexanecarboxylate (intermediate 151) (450 mg, 1.27 mmol) in DMF (10 mL). The resulting solution was stirred at 45° C. for 45 minutes, 1-(3-dimethylaminopropyl)-3-ethylcarbodiimide hydrochloride (353 mg, 1.84 mmol) was added and the mixture was stirred at 85° C. for 60 minutes. The reaction mixture was allowed to cool to ambient temperature, water (10 mL) was added and the precipitate was collected by filtration, washed with water (10 mL) and dried under vacuum at 60° C. to afford (1s,4s)-methyl 4-(5-(5-(3,4-difluorophenylamino)-1,3,4-oxadiazole-2-carboxamido)-4-fluoropyridin-2-yloxy)cyclohexanecarboxylate (411 mg, 65.9%) as a beige solid, which was used without further purification. ¹H NMR (400 MHz, DMSO-d6) δ 1.68-1.86 (8H, m), 2.50-2.52 (1H, m), 3.61 (3H, s), 5.13-5.19 (1H, m), 6.86-6.88 (1H, m), 7.33-7.35 (1H, m), 7.43-7.48 (1H, m), 7.67-7.71 (1H, m), 8.20 (1H, d), 10.83 (1H, s), 11.24 (1H, s). m/z 492 (M+H)⁺.

### Intermediate 151

### (1s,4s)-Methyl 4-(4-fluoro-5-(2-hydrazinyl-2-oxoacetamido)pyridin-2-yloxy)cyclohexanecarboxylate

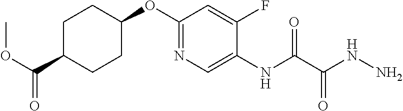

Hydrazine monohydrate (0.077 mL, 1.60 mmol) was added to a stirred solution of (1s,4s)-methyl 4-(4-fluoro-5-(2-methoxy-2-oxoacetamido)pyridin-2-yloxy)cyclohexanecarboxylate (intermediate 152) (514 mg, 1.45 mmol) in ethanol (30 mL). The resulting suspension was stirred at ambient temperature for 90 minutes. The reaction mixture was evaporated to dryness to afford (1s,4s)-methyl 4-(4-fluoro-5-(2-hydrazinyl-2-oxoacetamido)pyridin-2-yloxy)cyclohexanecarboxylate (473 mg, 92%) as a white solid. ¹H NMR (400 MHz, DMSO) δ 1.66-1.85 (8H, m), 2.56-2.58 (1H, m), 3.61 (3H, s), 4.67 (1H, s), 4.68 (1H, s), 5.20-521 (1H, m), 6.83 (1H, d), 8.18 (1H, d), 10.27 (1H, s), 10.32 (1H, s). m/z 355 (M+H)⁺.

### Intermediate 152

### (1s,4s)-methyl-4-(4-fluoro-5-(2-methoxy-2-oxoacetamido)pyridin-2-yloxy)cyclohexanecarboxylate

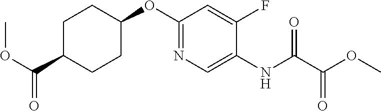

Methyl oxalyl chloride (0.210 mL, 2.29 mmol) was added to a stirred solution of (1s,4s)-methyl 4-(5-amino-4-fluoropyridin-2-yloxy)cyclohexanecarboxylate (intermediate 153) (472 mg, 1.76 mmol), and pyridine (0.171 mL, 2.11 mmol) in DCM (20 mL). The resulting solution was stirred at ambient temperature under nitrogen for 1 hour. The reaction mixture was diluted with DCM (100 mL), and washed with saturated brine (100 mL), the aqueous layer was washed with DCM (50 mL). The organic extracts were combined, dried over MgSO₄, filtered and evaporated to afford crude product. The crude product was purified by flash silica chromatography, elution gradient 20 to 70% EtOAc in isohexane. Pure fractions were evaporated to dryness to afford (1s,4s)-methyl 4-(4-fluoro-5-(2-methoxy-2-oxoacetamido)pyridin-2-yloxy)cyclohexanecarboxylate (514 mg, 82%) as a pale yellow oil which solidified on standing. ¹H NMR (400 MHz, CDCl₃) δ 1.65-1.81 (4H, m), 1.89-2.02 (4H, m), 2.42-2.46 (1H, m), 3.69 (3H, s), 3.98 (3H, s), 5.24-5.26 (1H, m), 6.52-6.55 (1H, m), 8.74 (1H, s), 8.95 (1H, d). m/z 355 (M+H)⁺.

### Intermediate 153

### (1s,4s)-Methyl 4-(5-amino-4-fluoropyridin-2-yloxy)cyclohexanecarboxylate

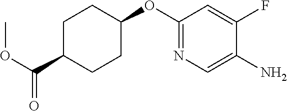

A mixture of (1s,4s)-methyl 4-(4-fluoro-5-nitropyridin-2-yloxy)cyclohexanecarboxylate (intermediate 154) (525 mg, 1.76 mmol) and 10% palladium on carbon (52.4 mg, 0.49 mmol) in EtOAc (20 mL) was evacuated with hydrogen (3 cycles) and then stirred under an atmosphere of hydrogen at ambient temperature overnight. The reaction mixture was filtered and evaporated to afford (1s,4s)-methyl 4-(5-amino-4-fluoropyridin-2-yloxy)cyclohexanecarboxylate (472 mg, 100%) as a brown gum. ¹H NMR (400 MHz, CDCl₃) δ 1.61-1.68 (2H, m), 1.71-1.79 (2H, m), 1.89-1.99 (4H, m), 2.38-2.44 (1H, m), 3.35 (2H, s), 3.69 (3H, s), 5.06-5.09 (1H, m), 6.42 (1H, d), 7.68 (1H, d). m/z 269 (M+H)⁺.

### Intermediate 154

### (1s,4s)-methyl 4-(4-fluoro-5-nitropyridin-2-yloxy)cyclohexanecarboxylate

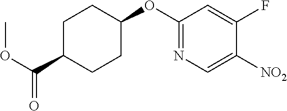

A solution of 1M tetrabutylammonium fluoride in THF (6.35 mL, 6.35 mmol) was added to a stirred solution of (1s,4s)-methyl 4-(4-chloro-5-nitropyridin-2-yloxy)cyclohexanecarboxylate (intermediate 155) (1.0 g, 3.18 mmol) in DMF (6.3 mL) under nitrogen. The resulting solution was stirred at ambient temperature for 10 minutes. The reaction mixture was diluted with EtOAc (100 mL), and washed with saturated brine (3×75 mL). The organic layer was dried over MgSO₄, filtered and evaporated to afford crude product. The crude product was purified by flash silica chromatography, elution gradient 0 to 20% EtOAc in isohexane. Pure fractions were evaporated to dryness to afford (1s,4s)-methyl 4-(4-fluoro-5-nitropyridin-2-yloxy)cyclohexanecarboxylate (0.282 g, 29.8%) as a pale yellow oil which solidified on standing. ¹H NMR (400 MHz, CDCl₃) δ 1.69-1.84 (4H, m), 1.89-2.04 (4H, m), 2.43-2.50 (1H, m), 3.70 (3H, s), 5.36-5.39 (1H, m), 6.54-6.57 (1H, m), 8.95 (1H, d). m/z 299 (M+H)⁺.

### Intermediate 155

### (1s,4s)-methyl 4-(4-chloro-5-nitropyridin-2-yloxy)cyclohexanecarboxylate

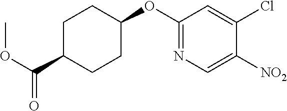

Diisopropyl azodicarboxylate (9.87 mL, 50.13 mmol) was added to a stirred solution of 4-chloro-5-nitropyridin-2-ol (intermediate 156) (7 g, 40.10 mmol) and triphenylphosphine (15.78 g, 60.16 mmol) in THF (180 mL) under nitrogen. The reaction mixture was stirred at ambient temperature for 10 minutes and then (1r,4r)-methyl 4-hydroxycyclohexanecarboxylate (6.34 g, 40.10 mmol) in THF (45 mL) was added and the resulting solution was stirred at ambient temperature over the weekend. The reaction mixture was evaporated and the residue was purified by flash silica chromatography, elution gradient 0 to 20% EtOAc in isohexane. Yielded (1s,4s)-methyl 4-(4-chloro-5-nitropyridin-2-yloxy)cyclohexanecarboxylate (2.86 g, 22.66%). ¹H NMR (400 MHz, DMSO) δ 1.70-1.87 (8H, m), 3.61 (3H, s), 5.28 (1H, d), 7.30 (1H, s), 8.96 (1H, s). 1H obscured by DMSO peak. No mass ion.

### Intermediate 156

### 4-Chloro-5-nitropyridin-2-ol

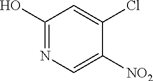

Ammonia (74.0 mL, 3418.64 mmol) was condensed into THF (170 mL) cooled to −78° C. under nitrogen. Potassium tert-butoxide (23.98 g, 213.67 mmol) was added as a solid and the reaction mixture warmed to −35° C. tert-Butyl hydroperoxide (5.5 M soln. in decane) (16.32 mL, 89.74 mmol) was added dropwise to 4-chloro-3-nitropyridine (13.55 g, 85.47 mmol) in THF (200 mL) cooled to 0° C. over a period of 5 minutes under nitrogen. The resulting solution was added slowly to the other flask and the mixture stirred at −35° C. for 1.5 hours. The reaction mixture was quenched with saturated NH₄Cl (50 mL) and allowed to warm to room temperature overnight. The reaction mixture was concentrated in vacuo and the brown precipitate filtered and washed with cold water to yield crude product. The solid was dried overnight under vacuum to yield 4-chloro-5-nitropyridin-2-ol (14.66 g, 83.99 mmol, 98%) as a yellow solid. ¹H NMR (400 MHz, DMSO) δ 6.36 (1H, s), 8.73 (1H, s). m/z 173 (M−H)⁻.

### Intermediate 157

### (1s,4s)-methyl 4-(5-(5-(3,4-difluorophenylamino)-1,3,4-oxadiazole-2-carboxamido)-3-fluoropyridin-2-yloxy)cyclohexanecarboxylate

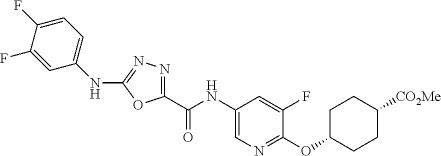

3,4-Difluorophenyl isothiocyanate (175 mg, 1.02 mmol) was added to a stirred solution of (1s,4s)-methyl 4-(3-fluoro-5-(2-hydrazinyl-2-oxoacetamido)pyridin-2-yloxy)cyclohexanecarboxylate (intermediate 158) (301 mg, 0.85 mmol) in DMF (10 mL) at 65° C. under nitrogen. The resulting solution was stirred at 65° C. for 30 minutes. 1-(3-Dimethylaminopropyl)-3-ethylcarbodiimide hydrochloride (196 mg, 1.02 mmol) was added to the reaction mixture and the resulting solution was stirred at 85° C. for 30 minutes. The reaction mixture was allowed to cool to room temperature before adding water (50 mL). The precipitate was collected by filtration, washed with water (25 mL) and dried under vacuum to afford (1s,4s)-methyl 4-(5-(5-(3,4-difluorophenylamino)-1,3,4-oxadiazole-2-carboxamido)-3-fluoropyridin-2-yloxy)cyclohexanecarboxylate (418 mg, 100%) as a yellow solid, which was used without further purification.

¹H NMR (400 MHz, DMSO) δ1.67-1.92 (m, 9H), 3.63 (s, 3H), 5.20-5.26 (m, 1H), 7.33-7.39 (m, 1H), 7.45-7.55 (m, 1H), 7.67-7.74 (m, 1H), 8.07-8.12 (m, 1H), 8.39 (d, 1H), 11.32 (s, 1H), 11.34 (s, 1H). m/z (ESI+) (M+H)+=492.27.

### Intermediate 158

### (1s,4s)-methyl 4-(3-fluoro-5-(2-hydrazinyl-2-oxoacetamido)pyridin-2-yloxy)cyclohexanecarboxylate

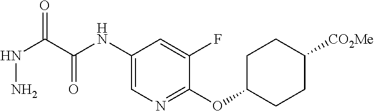

Hydrazine hydrate (0.271 mL, 5.58 mmol) was added to a stirred solution of (1s,4s)-methyl 4-(3-fluoro-5-(2-methoxy-2-oxoacetamido)pyridin-2-yloxy)cyclohexanecarboxylate (intermediate 159) (1.796 g, 5.07 mmol) in ethanol (100 mL) warmed to 70° C. The resulting solution was stirred at 70° C. for 10 minutes.

The precipitate was collected by filtration, washed with Et₂O (50 mL) and dried under vacuum to afford (1s,4s)-methyl 4-(3-fluoro-5-(2-hydrazinyl-2-oxoacetamido)pyridin-2-yloxy)cyclohexanecarboxylate (1.411 g, 79%) as a white solid, which was used without further purification.

¹H NMR (400 MHz, DMSO) δ1.66-1.89 (m, 9H), 3.62 (s, 3H), 4.65 (s, 2H), 5.18-5.24 (m, 1H), 8.07-8.13 (m, 1H), 8.43 (d, 1H), 10.35 (s, 1H), 10.89 (s, 1H). m/z (ESI+) (M+H)+=355.32.

### Intermediate 159

### (1s,4s)-methyl 4-(3-fluoro-5-(2-methoxy-2-oxoacetamido)pyridin-2-yloxy)cyclohexanecarboxylate

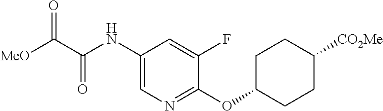

Methyl oxalyl chloride (0.560 mL, 6.08 mmol) was added to (1s,4s)-methyl 4-(5-amino-3-fluoropyridin-2-yloxy)cyclohexanecarboxylate (intermediate 160) (1.360 g, 5.07 mmol), and N-ethyldiisopropylamine (1.754 mL, 10.14 mmol) in DCM (20 mL) at 0° C. under nitrogen. The resulting solution was stirred at room temperature for 1 hour.

The reaction mixture was washed with water (20 mL), and saturated brine (10 mL). The organic layer was dried over MgSO₄, filtered and evaporated to afford crude product that was used without further purification.

¹H NMR (400 MHz, DMSO) δ1.67-1.90 (m, 9H), 3.62 (s, 3H), 3.86 (s, 3H), 5.19-5.24 (m, 1H), 8.01-8.07 (m, 1H), 8.35 (d, 1H), 11.06 (s, 1H). m/z (ESI+) (M+H)+=355.32.

### Intermediate 160

### (1s,4s)-methyl 4-(5-amino-3-fluoropyridin-2-yloxy)cyclohexanecarboxylate

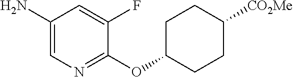

(1s,4s)-Methyl 4-(3-fluoro-5-nitropyridin-2-yloxy)cyclohexanecarboxylate (intermediate 161) (5873 mg, 19.69 mmol) and palladium 10% on carbon (200 mg) in methanol was stirred under an atmosphere of hydrogen at room temperature for 16 hours.

The reaction mixture was filtered through celite and evaporated.

The crude product was purified by flash silica chromatography, elution gradient 0 to 60% EtOAc in isohexane. Pure fractions were evaporated to dryness to afford (1s,4s)-methyl 4-(5-amino-3-fluoropyridin-2-yloxy)cyclohexanecarboxylate (1.36 g, 25.7%) as a brown oil.

¹H NMR (400 MHz, DMSO) δ1.60-1.84 (m, 8H), 2.44-2.49 (m, 1H), 3.61 (s, 3H), 4.95-5.01 (m, 1H), 5.03 (s, 2H), 6.87-6.93 (m, 1H), 7.31 (d, 1H). m/z (ESI+) (M+H)+=269.32.

### Intermediate 161

### (1s,4s)-methyl 4-(3-fluoro-5-nitropyridin-2-yloxy)cyclohexanecarboxylate

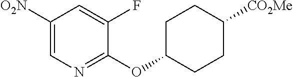

(1s,4s)-Methyl 4-(3-fluoropyridin-2-yloxy)cyclohexanecarboxylate (intermediate 162) (4.99 g, 19.69 mmol) was added dropwise to trifluoroacetic anhydride (6.88 mL, 49.23 mmol), cooled to 0° C. under nitrogen. The resulting solution was stirred at 0° C. for 2 hours. Nitric acid (1.861 mL, 41.35 mmol) was added dropwise and the resulting solution was stirred at room temperature for 16 h. The reaction mixture was added slowly to a stirred, cooled (0° C.) solution of sodium metabisulfite (3.74 g, 19.69 mmol) in water (50 mL). After stirring at room temperature for 24 hours the reaction mixture was neutralised with 2M NaOH and extracted with DCM (3×100 mL). The organic layers were combined and dried over MgSO₄, filtered and evaporated to afford crude product containing starting material, product, hydrolysed starting material and hydrolysed product. This was taken up in MeOH (50 mL) and a few drops conc. H₂SO₄ was added before stirring for 16 hours at room temperature. The reaction mixture was neutralised with saturated NaHCO₃ solution and the methanol was removed by evaporation. The aqueous residue was extracted with EtOAc (3×50 mL) and dried over MgSO₄, filtered and evaporated to afford crude product that was used without further purification.

¹H NMR (400 MHz, DMSO) δ1.67-1.97 (m, 8H), 2.49-2.53 (m, 1H), 3.63 (s, 3H), 5.39-5.44 (m, 1H), 8.53-8.58 (m, 1H), 8.93 (d, 1H).

### Intermediate 162

### (1s,4s)-methyl 4-(3-fluoropyridin-2-yloxy)cyclohexanecarboxylate

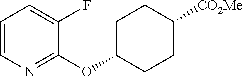

2M aq. Hydrogen chloride (1 mL, 2.00 mmol) was added to a stirred solution of (1s,4s)-4-(3-fluoropyridin-2-yloxy)cyclohexanecarboxylic acid (intermediate 163) (13.61 g, 56.9 mmol) in methanol at room temperature. This was stirred at room temperature for 2 hours. The reaction mixture was adjusted to pH 10 with 2M NaOH. The reaction mixture was evaporated, and the resulting aqueous solution was extracted with EtOAc (50 mL). The organic layer was dried over MgSO₄, filtered and evaporated to afford crude product.

The crude product was purified by flash silica chromatography, elution gradient 0 to 30% EtOAc in isohexane. Pure fractions were evaporated to dryness to afford (1s,4s)-methyl 4-(3-fluoropyridin-2-yloxy)cyclohexanecarboxylate (6.79 g, 47.1%) as a colourless oil.

¹H NMR (400 MHz, DMSO) δ1.66-1.90 (m, 8H), 2.48-2.54 (m, 1H), 3.62 (s, 3H), 5.22-5.29 (m, 1H), 6.96-7.03 (m, 1H), 7.63-7.70 (m, 1H), 7.93-7.98 (m, 1H).

m/z (ESI+) (M+H)+=254.17.

### Intermediate 163

### (1s,4s)-4-(3-fluoropyridin-2-yloxy)cyclohexanecarboxylic acid

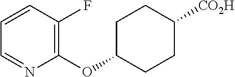

A solution of (1s,4s)-4-hydroxycyclohexanecarboxylic acid (0.500 g, 3.47 mmol) in DMA (2 mL) was added to a stirred mixture of sodium hydride (0.278 g, 6.94 mmol) in DMA (5 mL) cooled to 0° C. under nitrogen. The resulting solution was stirred at room temperature for 30 minutes. 2,3-Difluoropyridine (0.399 g, 3.47 mmol) was added and the resulting suspension was stirred at 100° C. for 2 h. The reaction mixture was evaporated to dryness and redissolved in EtOAc (20 mL), and washed sequentially with saturated NH₄Cl (10 mL) and saturated brine (10 mL). The organic layer was dried over MgSO₄, filtered and evaporated to afford crude product that was used without further purification.

¹H NMR (400 MHz, DMSO) δ1.17-1.30 (m, 4H), 1.38-1.70 (m, 4H), 1.96-2.20 (m, 1H), 4.96-5.04 (m, 1H), 6.70-6.78 (m, 1H), 7.36-7.44 (m, 1H), 7.68-7.75 (m, 1H), 11.73 (s, 1H). m/z (ESI+) (M+H)+=240.34.

### Intermediate 164

### (1s,4s)-methyl 4-(5-(5-(3-chloro-4-fluorophenylamino)-1,3,4-oxadiazole-2-carboxamido)-3-fluoropyridin-2-yloxy)cyclohexanecarboxylate

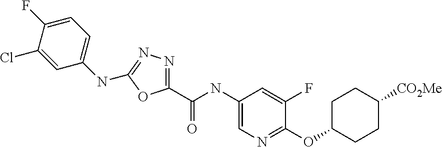

2-Chloro-1-fluoro-4-isothiocyanatobenzene (159 mg, 0.85 mmol) was added to a stirred solution of (1s,4s)-methyl 4-(3-fluoro-5-(2-hydrazinyl-2-oxoacetamido)pyridin-2-yloxy)cyclohexanecarboxylate (intermediate 158) (301 mg, 0.85 mmol) in DMF (10 mL) at 65° C. under nitrogen. The resulting solution was stirred at 65° C. for 30 minutes. 1-(3-Dimethylaminopropyl)-3-ethylcarbodiimide hydrochloride (196 mg, 1.02 mmol) was added to the reaction mixture and the resulting solution was stirred at 85° C. for 30 minutes. The reaction mixture was allowed to cool to room temperature before adding water (50 mL). The precipitate was collected by filtration, washed with water (25 mL) and dried under vacuum to afford (1s,4s)-methyl-4-(5-(5-(3-chloro-4-fluorophenylamino)-1,3,4-oxadiazole-2-carboxamido)-3-fluoropyridin-2-yloxy)cyclohexanecarboxylate (432 mg, 100%) as a yellow solid, which was used without further purification.

¹H NMR (400 MHz, DMSO) δ1.67-1.92 (m, 9H), 3.63 (s, 3H), 5.20-5.26 (m, 1H), 7.45-7.57 (m, 2H), 7.81-7.85 (m, 1H), 8.07-8.13 (m, 1H), 8.39 (d, 1H), 11.31 (s, 1H), 11.34 (s, 1H). m/z (ESI+) (M+H)+=508.19.

### Intermediate 165

### (1s,4s)-methyl 4-(5-(5-(3,4-difluorophenylamino)-1,3,4-oxadiazole-2-carboxamido)-6-methylpyridin-2-yloxy)cyclohexanecarboxylate

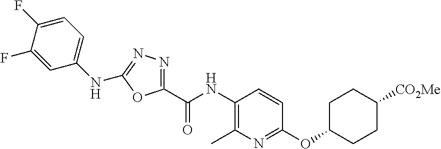

3,4-Difluorophenyl isothiocyanate (83 mg, 0.49 mmol) in was added to a stirred solution of (1s,4s)-methyl 4-(5-(2-hydrazinyl-2-oxoacetamido)-6-methylpyridin-2-yloxy)cyclohexanecarboxylate (intermediate 166) (170 mg, 0.49 mmol) in DMF (10 mL) at 65° C. under nitrogen. The resulting solution was stirred at 65° C. for 30 minutes. 1-(3-Dimethylaminopropyl)-3-ethylcarbodiimide hydrochloride (112 mg, 0.58 mmol) was added to the reaction mixture and the resulting solution was stirred at 85° C. for 30 minutes. The reaction mixture was allowed to cool to room temperature before adding water (50 mL). The precipitate was collected by filtration, washed with water (25 mL) and dried under vacuum to afford (1s,4s)-methyl 4-(5-(5-(3,4-difluorophenylamino)-1,3,4-oxadiazole-2-carboxamido)-6-methylpyridin-2-yloxy)cyclohexanecarboxylate (233 mg, 99%) as a white solid, which was used without further purification.

¹H NMR (500 MHz, DMSO at 373K) δ1.68-1.93 (m, 8H), 2.34 (s, 3H), 2.47-2.53 (m, 1H), 3.64 (s, 3H), 5.12-5.18 (m, 1H), 6.31-6.37 (m, 1H), 6.63 (d, 1H), 7.21-7.69 (m, 3H), 9.29 (d, 1H), 10.07 (s, 1H). m/z (ESI+) (M+H)+=488.46.

### Intermediate 166

### (1s,4s)-methyl 4-(5-(2-hydrazinyl-2-oxoacetamido)-6-methylpyridin-2-yloxy)cyclohexanecarboxylate

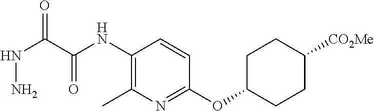

Hydrazine hydrate (0.038 mL, 0.79 mmol) was added to (1s,4s)-methyl 4-(5-(2-methoxy-2-oxoacetamido)-6-methylpyridin-2-yloxy)cyclohexanecarboxylate (intermediate 167) (252 mg, 0.72 mmol) in ethanol (5 mL) at 70° C. The resulting solution was stirred at 70° C. for 30 minutes. The precipitate was collected by filtration, washed with Et₂O (20 mL) and dried under vacuum to afford (1s,4s)-methyl 4-(5-(2-hydrazinyl-2-oxoacetamido)-6-methylpyridin-2-yloxy)cyclohexanecarboxylate (148 mg, 58.7%) as a white solid, which was used without further purification.

¹H NMR (400.132 MHz, DMSO) δ1.64-1.90 (m, 8H), 2.26 (s, 3H), 2.45-2.52 (m, 1H), 3.62 (s, 3H), 4.59 (s, 2H), 5.10-5.17 (m, 1H), 6.18 (d, 1H), 6.64 (d, 1H), 10.04 (s, 1H), 10.19 (s, 1H). m/z (ESI+) (M+H)+=351.40.

### Intermediate 167

### (1s,4s)-methyl 4-(5-(2-methoxy-2-oxoacetamido)-6-methylpyridin-2-yloxy)cyclohexanecarboxylate

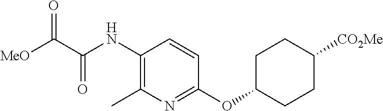

Methyl oxalyl chloride (0.079 mL, 0.86 mmol) was added to (1s,4s)-methyl 4-(5-amino-6-methylpyridin-2-yloxy)cyclohexanecarboxylate (intermediate 168) (190 mg, 0.72 mmol) and pyridine (0.116 mL, 1.44 mmol) in DCM (20 mL) at 0° C. under nitrogen. The resulting solution was stirred at room temperature for 1 hour. The reaction mixture was quenched with water (10 mL), extracted with DCM (2×20 mL), the organic layer was dried over MgSO₄, filtered and evaporated to afford the crude (1s,4s)-methyl 4-(5-(2-methoxy-2-oxoacetamido)-6-methylpyridin-2-yloxy)cyclohexanecarboxylate (252 mg, 100%) that was used without further purification. m/z (ESI+) (M+H)+=351.41.

### Intermediate 168

### (1s,4s)-methyl 4-(5-amino-6-methylpyridin-2-yloxy)cyclohexanecarboxylate

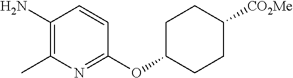

Ammonium formate (1.154 g, 18.30 mmol) was added to (1s,4s)-methyl 4-(5-(diphenylmethyleneamino)-6-methylpyridin-2-yloxy)cyclohexanecarboxylate (intermediate 169) (0.523 g, 1.22 mmol) and palladium (10% on carbon) (0.13 g, 0.12 mmol) in methanol (10 mL) at ambient temperature. The resulting suspension was stirred at 60° C. for 2 hours. The reaction mixture was filtered and evaporated to dryness. The residue was dissolved in EtOAc (20 mL) and washed sequentially with water (10 mL) and saturated brine (10 mL). The organic layer was dried over MgSO₄, filtered and evaporated to afford crude product. This was purified by flash silica chromatography, elution gradient 0 to 80% EtOAc in isohexane. Pure fractions were evaporated to dryness to afford (1s,4s)-methyl 4-(5-amino-6-methylpyridin-2-yloxy)cyclohexanecarboxylate (0.190 g, 58.9%) as a white solid.

¹H NMR (400 MHz, DMSO) δ1.55-1.83 (m, 8H), 2.17 (s, 3H), 2.40-2.48 (m, 1H), 3.61 (s, 3H), 4.48 (s, 2H), 4.90-4.98 (m, 1H), 6.38 (d, 1H), 6.95 (d, 1H). m/z (ESI+) (M+H)+=265.43.

### Intermediate 169

### (1s,4s)-methyl 4-(5-(diphenylmethyleneamino)-6-methylpyridin-2-yloxy)cyclohexanecarboxylate

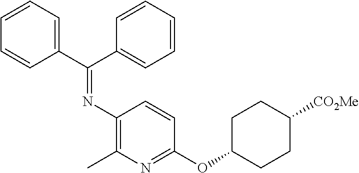

Benzophenone imine (0.307 mL, 1.83 mmol) was added to (1s,4s)-methyl 4-(5-bromo-6-methylpyridin-2-yloxy)cyclohexanecarboxylate (intermediate 170) (400 mg, 1.22 mmol), palladium(II) acetate (16.43 mg, 0.07 mmol), caesium carbonate (0.137 mL, 1.71 mmol) and (S)-(−)-2,2′-bis(diphenylphosphino)-1,1′-binaphthyl (45.6 mg, 0.07 mmol) in THF (10 mL) at room temperature under nitrogen. The resulting mixture was stirred at reflux for 8 hours. The reaction mixture was filtered and evaporated to give crude (1s,4s)-methyl 4-(5-(diphenylmethyleneamino)-6-methylpyridin-2-yloxy)cyclohexanecarboxylate (523 mg, 100%) that was used without further purification. m/z (ESI+) (M+H)+=429.45.

### Intermediate 170

### (1s,4s)-methyl 4-(5-bromo-6-methylpyridin-2-yloxy)cyclohexanecarboxylate

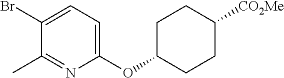

2M aq. Hydrogen chloride (5 mL, 0.00 μmol) was added to a stirred solution of (1s,4s)-4-(5-bromo-6-methylpyridin-2-yloxy)cyclohexanecarboxylic acid (intermediate 171) (6.535 g, 20.8 mmol) in methanol at 20° C. This was stirred at room temperature for 2 hours. The reaction mixture was adjusted to pH10 with 2M NaOH. The reaction mixture was evaporated, and the resulting aqueous solution was extracted with EtOAc (50 mL). The organic layer was dried over MgSO₄, filtered and evaporated to afford crude product.

The crude product was purified by flash silica chromatography, elution gradient 0 to 30% EtOAc in isohexane. Pure fractions were evaporated to dryness to afford (1s,4s)-methyl 4-(5-bromo-6-methylpyridin-2-yloxy)cyclohexanecarboxylate (786 mg, 11.51%) as a yellow oil.

¹H NMR (400 MHz, DMSO) δ1.64-1.87 (m, 8H), 2.46 (s, 3H), 2.49-2.53 (m, 1H), 3.61 (s, 3H), 5.08-5.15 (m, 1H), 6.61 (d, 1H), 7.82 (d, 1H). m/z (ESI+) (M+H)+=330.27.

### Intermediate 171

### (1s,4s)-4-(5-bromo-6-methylpyridin-2-yloxy)cyclohexanecarboxylic acid

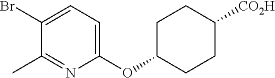

A solution of (1s,4s)-4-hydroxycyclohexanecarboxylic acid (3.00 g, 20.8 mmol) in DMA (5 mL) was added to a stirred mixture of sodium hydride (1.668 g, 41.7 mmol) in DMA (5 mL) cooled to 0° C. The resulting suspension was stirred at room temperature for 30 minutes. 5-Bromo-2-chloro-6-methylpyridine (4.29 g, 20.80 mmol) was added and the resulting suspension was stirred at 100° C. for 2 h. The reaction mixture was allowed to cool to room temperature and diluted with water (10 mL) and acidified with 2M HCl. The precipitate was collected by filtration, washed with water (50 mL) and dried under vacuum to afford (1s,4s)-4-(5-bromo-6-methylpyridin-2-yloxy)cyclohexanecarboxylic acid (6.53 g, 100%) which was used without further purification.

¹H NMR (400 MHz, DMSO) δ1.62-1.89 (m, 8H), 2.33-2.41 (m, 1H), 2.46 (s, 3H), 5.07-5.15 (m, 1H), 6.59 (d, 1H), 7.81 (d, 1H), 12.06 (s, 1H). m/z (ESI+) (M+H)+=316.27.

### Intermediate 172

### phenethyl 3-(5-(5-(4-isopropylphenylamino)-1,3,4-oxadiazole-2-carboxamido)pyridin-2-yloxy)cyclobutanecarboxylate (cis/trans mixture)

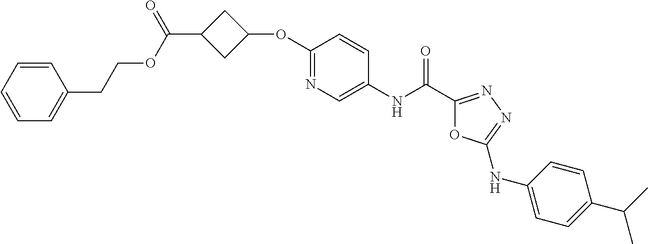

4-Isopropylphenyl isothiocyanate (0.475 mL, 2.76 mmol) was added to phenethyl 3-(5-(2-hydrazinyl-2-oxoacetamido)pyridin-2-yloxy)cyclobutanecarboxylate (cis/trans mixture; intermediate 173) (1 g, 2.51 mmol), in DMF (15 mL) under nitrogen. The resulting solution was stirred at room temp for 1 hour. 1-(3-Dimethylaminopropyl)-3-ethylcarbodiimide hydrochloride (0.577 g, 3.01 mmol) was added and the reaction stirred at room temperature for 18 hours. The reaction mixture was evaporated to dryness and triturated with water. The reaction mixture was filtered and dried in vacuo to afford crude product which was used in the next step without further purification.

m/z (ESI+) (M+H)+=542.

### Intermediate 173

### phenethyl 3-(5-(2-hydrazinyl-2-oxoacetamido)pyridin-2-yloxy)cyclobutanecarboxylate (cis/trans mixture)

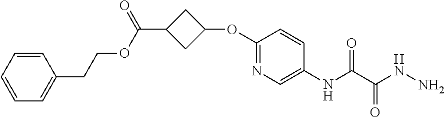

Hydrazine hydrate (0.440 mL, 9.04 mmol) was added to phenethyl 3-(5-(2-methoxy-2-oxoacetamido)pyridin-2-yloxy)cyclobutanecarboxylate (cis/trans mixture; intermediate 174) (3.00 g, 7.53 mmol), in ethanol (100 mL) at room temperature under nitrogen. The resulting solution was stirred at room temperature for 18 hours. The reaction mixture was evaporated to afford crude product, which was used in the next step without further purification. m/z (ESI+) (M+H)+=399.

### Intermediate 174

### phenethyl 3-(5-(2-methoxy-2-oxoacetamido)pyridin-2-yloxy)cyclobutanecarboxylate (cis/trans mixture)

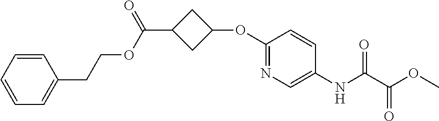

Methyl oxalyl chloride (0.763 mL, 8.29 mmol) was added to phenethyl 3-(5-aminopyridin-2-yloxy)cyclobutanecarboxylate (cis/trans mixture; intermediate 175) (2.355 g, 7.54 mmol) and pyridine (0.732 mL, 9.05 mmol) in DCM (50 mL) at room temperature under nitrogen. The resulting solution was stirred at room temperature for 1 hour. The reaction mixture was evaporated to dryness and redissolved in EtOAc (100 mL), and washed with saturated brine (2×50 mL). The organic layer was dried over MgSO₄, filtered and evaporated to afford crude product. m/z (ESI+) (M+H)+=399.

### Intermediate 175

### phenethyl 3-(5-aminopyridin-2-yloxy)cyclobutanecarboxylate (cis/trans mixture)

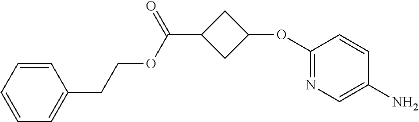

Phenethyl 3-(5-nitropyridin-2-yloxy)cyclobutanecarboxylate (cis/trans mixture; intermediate 176) (3.77 g, 11.01 mmol), and palladium on carbon (0.352 g, 3.30 mmol) in ethanol (80 mL) were stirred under an atmosphere of hydrogen at room temperature for 20 hours. The reaction mixture was filtered through celite, washed with ethanol (50 mL) and evaporated to afford crude product. The crude product was purified by flash silica chromatography, elution gradient 0 to 10% MeOH in DCM. Pure fractions were evaporated to dryness to afford phenethyl 3-(5-aminopyridin-2-yloxy)cyclobutanecarboxylate (2.380 g, 69.2%) as a beige solid. ¹H NMR (400 MHz, CDCl₃) 2.31-2.42 (2H, m), 2.66-2.80 (2.6H, m), 2.92-2.98 (2H, m), 3.09-3.16 (0.4H, m), 3.37 (2H, s), 4.28-4.35 (2H, m), 4.97-5.05 (0.6H, m),5.19-5.26 (0.4H, m) 6.52-6.57 (1H, m), 7.00-7.03 (1H, m), 7.20-7.33 (5H, m), 7.60-7.64 (1H, m). m/z (ESI+) (M+H)+=313.

### Intermediate 176

### phenethyl 3-(5-nitropyridin-2-yloxy)cyclobutanecarboxylate (cis/trans mixture)

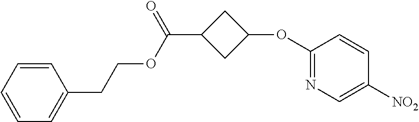

Diisopropyl azodicarboxylate (2.64 mL, 13.38 mmol) was added to a stirred solution of 5-nitropyridin-2-ol (1.5 g, 10.71 mmol), and triphenylphosphine (4.21 g, 16.06 mmol) in THF (80 mL) under nitrogen. The resulting solution was stirred at rt for 30 minutes and then phenethyl 3-hydroxycyclobutanecarboxylate (cis/trans mixture; intermediate 177) (2.95 g, 13.38 mmol) in THF (20 mL) was added. The resulting solution was stirred at rt overnight under nitrogen. The solvent was evaporated and the residue diluted with EtOAc and washed with brine (50 mL). The aqueous layer was extracted with EtOAc (50 mL) and the combined organics were concentrated in vacuo to afford crude product. The crude product was purified by flash silica chromatography, elution gradient 20 to 50% EtOAc in isohexane. Pure fractions were evaporated to dryness to afford phenethyl 3-(5-nitropyridin-2-yloxy)cyclobutanecarboxylate (2.98 g, 81%) as a beige solid.

¹H NMR (300 MHz, CDCl₃) 2.36-2.49 (2H, m), 2.70-2.88 (2.6H, m), 2.93-3.00 (2H, m), 3.10-3.20 (0.4H, m), 4.30-4.39 (2H, m), 5.21 (0.6H d, J=14.4 Hz), 5.42-5.51 (0.4H, m) 6.77-6.81 (1H, m), 7.20-7.34 (5H, m), 8.32-8.36 (1H, m), 9.04 (1H t, J=2.8 Hz). m/z (ESI+) (M+H)+=343.

### Intermediate 177

### Phenethyl 3-hydroxycyclobutanecarboxylate (cis/trans mixture)

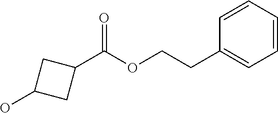

Phenethyl 3-(benzyloxy)cyclobutanecarboxylate (cis/trans mixture; intermediate 178) (7.92 g, 23.26 mmol) and 10% palladium on activated carbon (1.00 g) in EtOAc (100 mL) was stirred under an atmosphere of hydrogen at atmospheric pressure and 50° C. for 48 hours. The catalyst was removed by filtration through a pad of celite and washing through well with EtOAc (100 mL). Evaporation gave phenethyl 3-hydroxycyclobutanecarboxylate (5.40 g) as a colourless clear gum.

¹H NMR (400.13 MHz, CDCl₃) δ 2.00-2.15 (2H, m), 2.41-2.54 (4H, m), 2.85-2.96 (3H, m), 4.01-4.47 (3H, m), 7.13-7.25 (5H, m).

### Intermediate 178

### phenethyl 3-(benzyloxy)cyclobutanecarboxylate (cis/trans mixture)

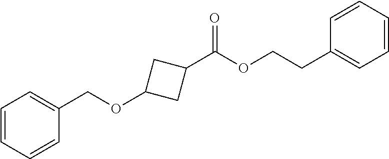

3-(Benzyloxy)cyclobutanecarbonyl chloride (cis/trans mixture) (10.89 g, 48.48 mmol) in DCM (30 mL) was added dropwise over 5 minutes to a solution of 2-phenylethanol (5.79 mL, 48.48 mmol) and pyridine (7.85 mL, 96.96 mmol) in DCM (20 mL) at 0° C. under nitrogen and allowed to warm to ambient temperature then stirred for 18 hours. The reaction mixture was diluted with Et₂O (100 mL) then washed sequentially with 1M HCl aq. (100 mL), water (50 mL) and brine (50 mL). The organic layer was dried over MgSO₄, filtered and evaporated to afford crude product. The crude product was purified by flash silica chromatography, elution gradient 0 to 100% DCM in isohexane. Pure fractions were evaporated to dryness to afford phenethyl 3-(benzyloxy)cyclobutanecarboxylate (11.90 g, 83%) as a colourless clear oil.

¹H NMR (400 MHz, CDCl₃) δ2.10-2.23 (2H, m), 2.34-2.42 (2H, m), 2.47-2.56 (0.5H, m)(cis), 2.84-2.88 (2H, m), 2.91-2.96 (0.5H, m)(trans), 3.82-3.90 (0.5H, m)(cis), 4.12-4.25 (2.5H, m)(trans), 4.32-4.34 (2H, m), 7.12-7.29 (10H, m).

### Intermediate 179

### phenethyl 3-(5-(5-(3,4-difluorophenylamino)-1,3,4-oxadiazole-2-carboxamido)pyridin-2-yloxy)cyclobutanecarboxylate(cis/trans mixture)

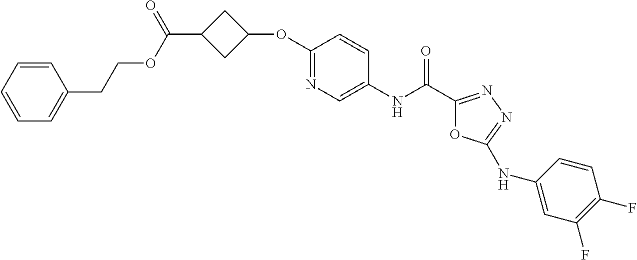

Prepared in an analogous way to intermediate 172 and used without further purification. m/z (ESI+) (M+H)+=536.

### Intermediate 180

### ethyl 4-(5-((5-((2,4-dichlorophenyl)amino)1,3,4-oxadiazole-2-carbonyl)amino)pyridin-2-yl)oxy-1-methylcyclohexane-1-carboxylate

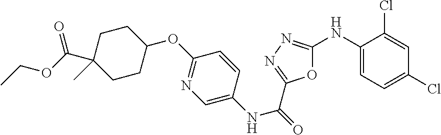

2,4-Dichlorophenyl isothiocyanate (0.158 mL, 1.09 mmol) was added to (1s,4s)-ethyl 4-(5-(2-hydrazinyl-2-oxoacetamido)pyridin-2-yloxy)-1-methylcyclohexanecarboxylate (intermediate 9) (0.38 g, 1.04 mmol) in DMF (10.43 mL). The resulting solution was stirred at 40° C. for 25 minutes. N-(3-Dimethylaminopropyl)-N′-ethylcarbodiimide hydrochloride (0.220 g, 1.15 mmol) was added to the above solution. The resulting solution was stirred at 80° C. for 25 minutes. The reaction was incomplete and further N-(3-dimethylaminopropyl)-N′-ethylcarbodiimide hydrochloride (0.05 g) was added. The mixture was stirred at 80° C. for a further 1 hour. The reaction was cooled to room temperature and water (6 mL) was added to the reaction mixture, precipitate formed. The precipitate was collected by filtration, washed with water and dried under vacuum to yield ethyl 4-(5-((5-((2,4-dichlorophenyl)amino)-1,3,4-oxadiazole-2-carbonyl)amino)pyridin-2-yl)oxy-1-methylcyclohexane-1-carboxylate (0.682 g, 122%) which was used without further purification. m/z 534 (M−H)−.

### Intermediate 181

### (1s,4s)-ethyl 4-(5-(5-(3,4-difluorophenylamino)-1,3,4-oxadiazole-2-carboxamido)pyridin-2-yloxy)-1-methylcyclohexanecarboxylate

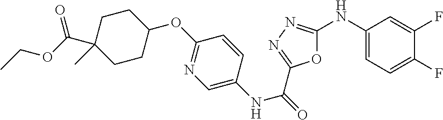

3,4-Difluorophenyl isothiocyanate (0.099 g, 0.58 mmol) was added to (1s,4s)-ethyl 4-(5-(2-hydrazinyl-2-oxoacetamido)pyridin-2-yloxy)-1-methylcyclohexanecarboxylate (intermediate 9) (0.2 g, 0.55 mmol) in DMF (5.49 mL). The resulting solution was stirred at 40° C. for 25 minutes. N-(3-Dimethylaminopropyl)-N′-ethylcarbodiimide hydrochloride (0.116 g, 0.60 mmol) was added to the above solution. The resulting solution was stirred at 80° C. for 90 minutes. The reaction was then cooled to room temperature and water (6 mL) was added to the reaction mixture which caused a precipitate to form. The precipitate was collected by filtration, washed with water (2 mL) and dried under vacuum to afford (1s,4s)-ethyl 4-(5-(5-(3,4-difluorophenylamino)-1,3,4-oxadiazole-2-carboxamido)pyridin-2-yloxy)-1-methylcyclohexanecarboxylate (0.250 g, 91%) as a yellow solid, which was used without further purification. m/z 502 (M+H)+

### Intermediate 182

### ethyl 4-(5-(5-(3-chloro-4-fluorophenylamino)-1,3,4-oxadiazole-2-carboxamido)pyridin-2-yloxy)-1-methylcyclohexanecarboxylate (cis/trans mixture)

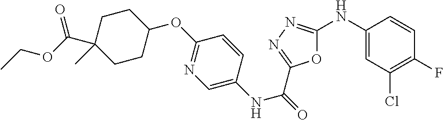

3-Chloro-4-fluorophenylisothiocyanate (0.232 g, 1.23 mmol) was added to ethyl 4-(5-(2-hydrazinyl-2-oxoacetamido)pyridin-2-yloxy)-1-methylcyclohexanecarboxylate (intermediate 9) (0.45 g, 1.23 mmol) in DMF (12.35 mL). The resulting solution was stirred at 40° C. for 25 minutes. N-(3-Dimethylaminopropyl)-N′-ethylcarbodiimide hydrochloride (0.260 g, 1.36 mmol) was added to the above solution. The resulting solution was stirred at 80° C. for 25 minutes. The reaction was incomplete and further N-(3-Dimethylaminopropyl)-N′-ethylcarbodiimide hydrochloride (0.05 g) was added. The mixture was stirred at 80° C. for a further 1 hour. The reaction mixture was cooled to room temperature. Water (6 mL) was added and a precipitate formed. The precipitate was collected by filtration, dried under vacuum and used without further purification.

m/z 518 (M+H)+

### Intermediate 183

### (1s,4s)-methyl 4-(6-fluoro-5-(2-hydrazinyl-2-oxoacetamido)pyridin-2-yloxy)cyclohexanecarboxylate

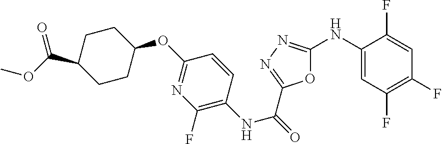

2,4,5-Trifluorophenyl isothiocyanate (0.197 g, 1.04 mmol) was added to (1s,4s)-methyl 4-(6-fluoro-5-(2-hydrazinyl-2-oxoacetamido)pyridin-2-yloxy)cyclohexanecarboxylate (intermediate 127) (0.335 g, 0.95 mmol) in DMF (9.45 mL). The resulting solution was stirred at 40° C. for 30 minutes. N-(3-Dimethylaminopropyl)-N′-ethylcarbodiimide hydrochloride (0.217 g, 1.13 mmol) was added to the above solution. The resulting solution was stirred at 80° C. for 30 minutes. The reaction was then cooled to room temperature and water (5 mL) was added to the solution. The resulting precipitate was collected by filtration and dried under vacuum to yield (1s,4s)-methyl 4-(6-fluoro-5-(5-(2,4,5-trifluorophenylamino)-1,3,4-oxadiazole-2-carboxamido)pyridin-2-yloxy)cyclohexanecarboxylate (0.470 g, 98%) as a brown solid which was used without further purification.

¹H NMR (400 MHz, DMSO-d₆) δ 1.68-1.86 (8H, m), 3.61 (3H, s), 5.01 (1H, s), 6.80 (1H, d), 7.65-7.72 (1H, m), 7.87-7.94 (1H, m), 8.11-8.18 (1H, m), 10.78 (1H, s), 11.05 (1H, s). m/z 508 (M−H)−

### Intermediate 184

### (1s,4s)-methyl 4-(5-(5-(4-chloro-2-methoxyphenylamino)-1,3,4-oxadiazole-2-carboxamido)pyridin-2-yloxy)cyclohexanecarboxylate

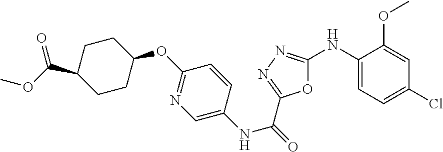

Sodium hydride (0.086 g, 2.14 mmol) was added portionwise to 3-chloro-2-methoxyaniline (0.169 g, 1.07 mmol) in DMF (9.0 mL) at room temperature under air. The resulting suspension was stirred at room temperature for 35 minutes. Di-2-pyridyl thionocarbonate (0.249 g, 1.07 mmol) was added to the above suspension and the resulting solution was stirred at was stirred at room temperature for 15 minutes. (1s,4s)-methyl 4-(5-(2-hydrazinyl-2-oxoacetamido)pyridin-2-yloxy)cyclohexanecarboxylate (intermediate 2) (0.3 g, 0.89 mmol) was then added to the above solution. The resulting solution was stirred at 65° C. for 35 minutes. N-(3-Dimethylaminopropyl)-N′-ethylcarbodiimide hydrochloride (0.205 g, 1.07 mmol) was then added portion wise to the above solution. The resulting solution was stirred at 85° C. for 35 minutes. The reaction was then cooled to room temperature and water (9.0 mL) was added to the reaction mixture, this caused the product to precipitate out of solution. The product was then collected by filtration, washed with water and hexanes and dried under vacuum to afford (1s,4s)-methyl 4-(5-(5-(3-chloro-2-methoxyphenylamino)-1,3,4-oxadiazole-2-carboxamido)pyridin-2-yloxy)cyclohexanecarboxylate (0.223 g, 49.8%) which was used without further purification. m/z 502 (M+H)+.

1. A compound of formula (I), or a pharmaceutically-acceptable salt, or an in-vivo cleavable ester thereof, 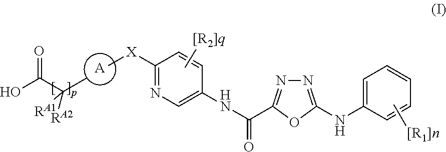
wherein
n is 0, 1, 2 or 3;
R₁ is independently chosen from fluoro, chloro, bromo, cyano, (1-4C)alkyl, (3-4C)cycloalkyl, (2-4C)alkynyl, (1-4C)alkoxy, —CONRaRb, —SO₂Rc and —OSO₂Rc; wherein Ra and Rb are each independently hydrogen or (1-4C)alkyl and Rc is (1-4C)alkyl;
q is 0, 1 or 2;
R₂ is independently chosen from fluoro, chloro, bromo, cyano, (1-4C)alkyl, (3-4C)cycloalkyl, (2-4C)alkynyl and (1-4C)alkoxy;
X is —O—, —S— or —NRa— wherein Ra is hydrogen or (1-4C)alkyl;
p is 0 or 1 and when p is 1, R^{A1} and R^{A2} are each independently hydrogen or (1-4C)alkyl or R^{A1} and R^{A2} are linked together to form a (3-6C)spiroalkyl ring;
Ring A is a di-linked (excluding links via the same or adjacent atoms) ring or ring system chosen from (4-6C)cycloalkane, (7-10C)bicycloalkane and (8-12C)tricycloalkane, each optionally substituted on an available carbon atom, including the ring carbon atom bearing the carboxy-containing group, by one substituent selected from (1-4C)alkyl, (1-4C)alkoxy and (1-4C)alkoxy(1-4C)alkyl;
or Ring A is di-linked (excluding links via adjacent atoms) phenylene optionally substituted on an available carbon atom by up to four substituents independently selected from fluoro, chloro, bromo, cyano, (1-4C)alkyl, (1-4C)alkoxy and (1-4C)alkoxy(1-4C)alkyl;
and wherein any carbon atom in a (1-4C)alkyl or (1-4C)alkoxy group defined above may be optionally substituted by up to 3 fluoro atoms;
and wherein the defined carboxylic acid group linked to Ring A may be replaced by —SO₃H, —S(O)₂NHR¹³, —S(O)₂NHC(O)R¹³, —CH₂S(O)₂R¹³, —C(O)NHS(O)₂R¹³, —C(O)NHOH, —C(O)NHCN, —CH(CF₃)OH, —C(CF₃OH, —P(O)(OH)₂ or a 5-membered heterocyclic ring selected from the group consisting of 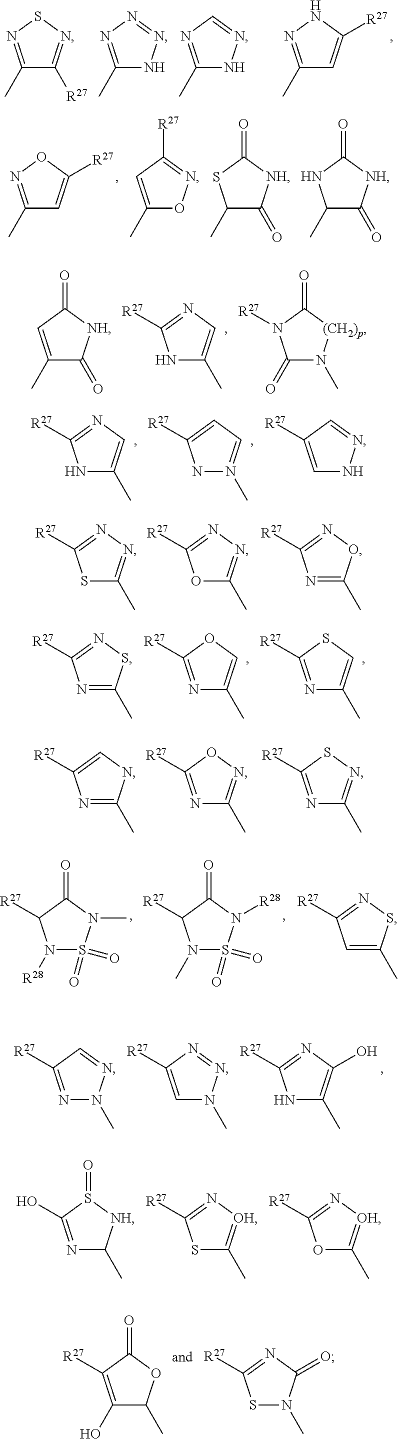
R¹³ is (1-6C)alkyl, aryl or heteroaryl;
R²⁷ and R²⁸ are independently selected from hydrogen, hydroxy, (1-6C)alkoxy, thiol, (1-6C)alkylthio, —C(O)R²⁹, —S(O)R³⁰, —SO₂R³¹, —NR³²R³³, —NHCN, halogen and trihalomethyl;
R²⁹, R³⁰ and R³¹ are —OR³⁴, (1-6C)alkyl, —NR³²R³³ or trihalomethyl,
R³² and R³³ are independently selected from hydrogen, (1-6C)alkyl, —SO₂R³⁴ and —COR³⁵;
R³⁴ is hydrogen, (1-6C)alkyl or trihalomethyl;
R³⁵ is (1-6C)alkyl or trihalomethyl; and
p is 1 or 2.

2. The compound of formula (I), or a pharmaceutically-acceptable salt, or an in-vivo cleavable ester thereof, as claimed in claim 1, wherein
n is 0, 1, 2 or 3;
R₁ is independently chosen from fluoro, chloro, bromo, cyano, (1-4C)alkyl, (3-4C)cycloalkyl, (2-4C)alkynyl, (1-4C)alkoxy, —CONRaRb, —SO₂Rc and —OSO₂Rc; wherein Ra and Rb are each independently hydrogen or (1-4C)alkyl and Rc is (1-4C)alkyl;
q is 0, 1 or 2
R₂ is independently chosen from fluoro, chloro, bromo, cyano, (1-4C)alkyl, (3-4C)cycloalkyl, (2-4C)alkynyl and (1-4C)alkoxy;
X is —O—, —S— or —NRa— wherein Ra is hydrogen or (1-4C)alkyl;
p is 0 or 1 and when p is 1 R^{A1} and R^{A2} are each independently hydrogen or (1-4C)alkyl or R^{A1} and R^{A2} are linked together to form a (3-6C)spiroalkyl ring;
Ring A is a di-linked ring chosen from 1,4-cyclohexane, 1,3-cyclopentane and 1,3-cyclobutane each optionally substituted on an available carbon atom, including the ring carbon atom bearing the carboxy-containing group, by one substituent selected from (1-4C)alkyl, (1-4C)alkoxy and (1-4C)alkoxy(1-4C)alkoxy;
or Ring A is 1,4-phenylene optionally substituted on an available carbon atom by up to four substituents independently selected from fluoro, chloro, bromo, cyano, (1-4C)alkyl, (1-4C)alkoxy and (1-4C)alkoxy(1-4C)alkyl;
and wherein any carbon atom in a (1-4C)alkyl or (1-4C)alkoxy group defined above may be optionally substituted by up to 3 fluoro atoms.

3. A compound of formula (IA), or a pharmaceutically-acceptable salt, or an in-vivo cleavable ester thereof, as claimed in claim 1, 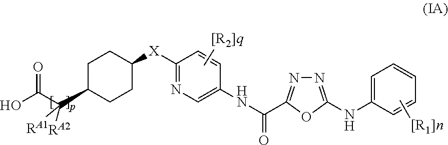
wherein R₁, R₂, R^{A1}, R^{A2}, X, n, p and q are as defined in claim 1.

4. A compound of formula (IB), or a pharmaceutically-acceptable salt, or an in-vivo cleavable ester thereof, as claimed in claim 1, 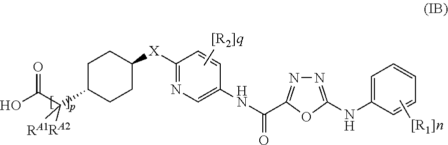
wherein R₁, R₂, R^{A1}, R^{A2}, X, n, p and q are as defined in claim 1.

5. A compound of formula (I), (IA) or (IB), or a pharmaceutically-acceptable salt thereof, as claimed in any one of claims 1 to 4.

6. A compound of formula (I), (IA) or (IB), or a pharmaceutically-acceptable salt, or an in-vivo cleavable ester thereof, as claimed in claim 1, wherein X is —O—.

7. A compound of formula (I), (IA) or (IB), or a pharmaceutically-acceptable salt, or an in-vivo cleavable ester thereof, as claimed in claim 1, wherein p is 1 and R^{A1}, R^{A2} are both hydrogen.

8. A compound of formula (I), (IA) or (IB), or a pharmaceutically-acceptable salt, or a pro drug an in-vivo cleavable ester thereof, as claimed in claim 1, wherein R₁ is fluoro and R₂ is hydrogen.

9. A compound as claimed in claim 1 which is selected from
cis-4-[5-[[5-[(3,4-difluorophenyl)amino]1,3,4-oxadiazole-2-carbonyl]amino]pyridin-2-yl]oxycyclohexane-1-carboxylic acid;
(1s,4s)-4-(5-(5-(2,4-dichlorophenylamino)-1,3,4-oxadiazole-2-carboxamido)pyridin-2-yloxy)cyclohexanecarboxylic acid;
(1s,4s)-4-(5-(5-(3-chloro-4-fluorophenylamino)-1,3,4-oxadiazole-2-carboxamido)pyridin-2-yloxy)cyclohexanecarboxylic acid;
2-((1s,4s)-4-(5-(5-(3,4-difluorophenylamino)-1,3,4-oxadiazole-2-carboxamido)pyridin-2-yloxy)cyclohexyl)acetic acid;
(1s,4s)-4-(5-(5-(3,4-difluorophenylamino)-1,3,4-oxadiazole-2-carboxamido)-6-fluoropyridin-2-yloxy)cyclohexanecarboxylic acid;
(1s,4s)-4-(6-fluoro-5-(5-(2,4,5-trifluorophenylamino)-1,3,4-oxadiazole-2-carboxamido)pyridin-2-yloxy)cyclohexanecarboxylic acid;
or a pharmaceutically-acceptable salt thereof.

10. A pharmaceutical composition which comprises a compound as claimed in claim 1 or a pharmaceutically-acceptable salt, or an in-vivo cleavable ester thereof, in association with a pharmaceutically-acceptable excipient or carrier.

11-12. (canceled)

13. A method of treating diabetes mellitus and/or obesity in a warm-blooded animal in need of such treatment which comprises administering to said animal an effective amount of a compound as claimed in claim 1 or a pharmaceutically-acceptable salt, or an in-vivo cleavable ester thereof.

14. A process for preparing a compound according to claim 1, or a salt or an in-vivo cleavable ester thereof which comprises one of the following steps, wherein all variables are as hereinbefore defined in claim 1 for a compound of formula (I) unless otherwise stated:
a) reacting a compound of formula (I) to form another compound of formula (I);
b) reacting an amine of formula (2) with an activated carboxylic acid derivative of the acid of formula (3) or with a carboxylate salt of the acid of formula (3) using a suitable coupling agent, wherein R is (1-6C)alkyl followed by hydrolysis of the R group; 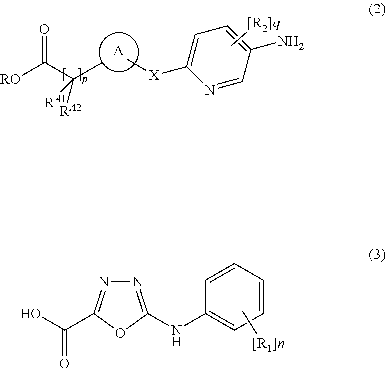
c) cyclisation of a compound of formula (4) where X₁ is S or O wherein R is (1-6C)alkyl, followed by hydrolysis of the R group; 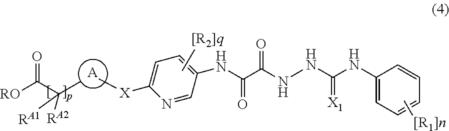
and optionally thereafter:
1) removing any protecting groups; and/or
2) forming a salt and/or an in-vivo cleavable ester thereof.

15. A process for preparing a compound of formula (2) 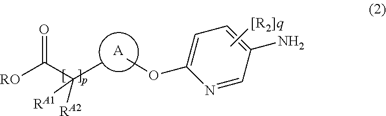
which comprises reacting a compound of formula (2-1) with a compound of formula (2-2) under Mitsunobu conditions to give a compound of formula (2A1); followed by reduction of the nitro group to an amine group
wherein R is a (1-6C)alkyl group and all variables are as defined in claim 1 for a compound of formula (I) unless otherwise stated.
